(19) United States
(12) Patent Application Publication
(10) Pub. No.: US 2010/0029657 A1
(43) Pub. Date: Feb. 4, 2010

(54) Bridged, Bicyclic Heterocyclic or Spiro Bicyclic Heterocyclic Derivatives of Pyrazolo[1, 5-A]Pyrimidines, Methods for Preparation and Uses Thereof

(75) Inventors: Jeremy Ian Levin, New City, NY (US); Darrin William Hopper, New York, NY (US); Nancy Torres, Nyack, NY (US); Minu Dhanjisha Dutia, West Nyack, NY (US); Dan Maarten Berger, New City, NY (US); Xiaolun Wang, River Vale, NJ (US); Martin Joseph Di Grandi, Goshen, NY (US); Chunchun Zhang, River Vale, NJ (US); Alejandro Lee Dunnick, Long Beach, CA (US)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/393,565

(22) Filed: Feb. 26, 2009

Related U.S. Application Data
(60) Provisional application No. 61/067,843, filed on Feb. 29, 2008.
(60) Provisional application No. 61/116,809, filed on Nov. 21, 2008.

(51) Int. Cl. A61K 31/4985; C07D 487/04; A61K 31/519; A61P 35/00; A61P 29/00

(52) U.S. Cl. 514/249; 544/281; 514/259.3

(57) ABSTRACT

Compounds of formula A:

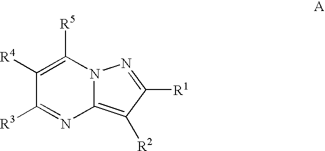

and pharmaceutically acceptable salts thereof are described, which selectively inhibit Raf kinase activity and are useful for treating disorders mediated by Raf kinases.

### CROSS-REFERENCE TO RELATED APPLICATIONS

This application claims the benefit under 35 U.S.C. §119(e) to co-pending U.S. Provisional Application Ser. No. 61/067,843, filed Feb. 29, 2008 and U.S. Provisional Application Ser. No. 61/116,809, filed Nov. 21, 2008, which are hereby incorporated by reference in their entirety.

### FIELD OF THE INVENTION

The present invention relates to new pyrazolo[1,5-a]pyrimidine compositions that are useful for inhibiting abnormal growth of certain cell types. The invention is directed to certain bridged, bicyclic heterocyclic or spiro bicyclic heterocyclic derivatives of pyrazolo[1,5-a]pyrimidines, their corresponding pharmaceutically acceptable salts and methods for their preparation and use. The bridged, bicyclic heterocyclic or spiro bicyclic heterocyclic derivatives of pyrazolo[1,5-a]pyrimidines inhibit growth of tumor cells, which contain oncogenic forms of Receptor Tyrosine Kinases, K-Ras and Raf kinases.

### BACKGROUND OF THE INVENTION

Raf is a multigene family expressing oncoprotein kinases: A-Raf, B-Raf and C-Raf (also known as Raf-1), as described in publications by McCubrey et al., in Leukemia, 12(I2), 1903-1929 (1998); by Ikawa et al., in Mol. and Cell. Biol. 8(6), 2651-2654 (1988); by Sithanandarn et al., in Oncogene 5, 1775-1780 (1990); by Konishi et al., in Biochem. and Biophys. Res. Comm. 216(2), 526-534 (1995). All three Raf kinases are functionally present in certain human hematopoietic cells, and their aberrant expression can result in abrogation of cytokine dependency. Their regulatory mechanisms differ in that C-Raf and A-Raf appear to require additional serine and tyrosine phosphorylation within the N region of the kinase domain for full activity, as described by Mason et al., in EMBO J. 18, 2137-2148 (1999). In addition, B-Raf kinase appears to have a much higher basal kinase activity than either A-Raf kinase or C-Raf kinase. The three Raf kinases play critical roles in the transmission of mitogenic and anti-apoptotic signals. B-Raf kinase is frequently mutated in various human cancers, as described by Wan et al., in Cell 116, 855-867 (2004), indicating that specific Raf kinases are associated with cancer. The cytoplasmic serine/threonine kinase B-Raf kinases and receptor tyrosine kinases of the platelet-derived growth factor receptor (PDGFR) family are frequently activated in cancer by mutations of an equivalent amino acid. Structural studies have provided important insights into why these very different kinases share similar oncogenic hot spots and why the PDGFR juxtamembrane region is also a frequent oncogenic target, as described by Dibb in Nature Reviews, Cancer 4(9), 718-27 (2004).

B-Raf encodes a Ras-regulated kinase that mediates cell growth and malignant transformation pathway activation that controls cell growth and survival. Activation of a Ras/Raf/MEK pathway results in a cascade of events from the cell surface to the cell nucleus, ultimately affecting cell proliferation, apoptosis, differentiation and transformation. Activating B-Raf mutations have been found in 66% of malignant melanomas and in a smaller fraction of other cancers including those of the colorectum, as reported by Davies H., et al. (2002) Nature 417:906 and by Rajagopalan H., et al. (2002) Nature 418, 934. Recently, B-Raf has been shown to be frequently mutated in various human cancers, as described by Wan et al. (2004) Cell 116, 855-867. B-Raf mutations also account for the MAP kinase pathway activation common in non-small cell lung carcinomas (NSCLC). Certain B-Raf mutations reported to date in NSCLC are non-V600 (89%; P<10⁻⁷), strongly suggesting that B-Raf mutations in NSCLC are qualitatively different from those in melanomas. Thus, there may be therapeutic differences between lung cancers and melanomas in response to Raf kinase inhibitors, as described by Karasarides et al., in Oncogene 23(37), 6292-6298 (2004) and by Bollag et al., in Current Opinion in Invest. Drugs, 4(12), 1436-1441 (2003). Although uncommon, B-Raf mutations in human lung cancers may identify a subset of tumors sensitive to targeted therapy, as described by Brose et al., in Cancer Research 62(23):6997-7000 (2002) and in U.S. Patent Application Publication No. 2005/267060.

Raf kinases are also key components of signal transduction pathways by which specific extracellular stimuli elicit precise cellular responses in mammalian cells. Activated cell surface receptors activate Ras/Rap proteins at the inner aspect of the plasma membrane, which in turn recruit and activate Raf proteins. Activated Raf proteins phosphorylate and activate the intracellular protein kinases MEK1 and MEK2. In turn, activated MEKs catalyze phosphorylation and activation of p42/p44 mitogen-activated protein kinase (MAPK). A variety of cytoplasmic and nuclear substrates of activated MAPK are directly or indirectly associated with the cellular response to cellular environmental change. In fact, B-Raf mutations have been shown to predict sensitivity to pharmacological MEK inhibition by small molecule inhibitors by limiting tumor growth in B-Raf mutant xenografts, as described by Solit et a., in Nature, Letters to Editor, Nov. 6, 2005. Three distinct genes have been identified in mammals that encode Raf proteins; A-Raf, B-Raf and C-Raf (also known as Raf-1) and isoformic variants that result from differential splicing of mRNA are known. Therefore, it is desirable to identify and characterize compounds that inhibit growth of tumor cells, which include oncogenic forms of Receptor Tyrosine Kinases, K-Ras, A-Raf kinase, B-Raf mutant kinase, B-Raf kinase and C-Raf kinase.

International Patent Publication No. WO 2004/052315 describes certain tyrosine kinase inhibitors, including certain bicyclic substituted, pyrazolo[1,5-a]pyrimidines. However, no bridged, bicyclic pyrazolo[1,5-a]pyrimidines have been described and little is known regarding how other ring systems, including bridged, bicyclic moieties, fused to the pyrazolo[5,1-a]pyrimidine ring framework influence the structure-activity relationship (SAR) of bridged, bicyclic pyrazolo[1,5-a]pyrimidines. There is a need for new compounds that selectively inhibit Raf kinase activity and that are useful for treating disorders mediated by any Raf kinase. Bridged, bicyclic pyrazolo[1,5-a]pyrimidine compositions of the present invention fulfill this unmet need and are useful in the treatment of diseases associated with Raf kinases, including cancer and inflammation, in mammals.

### SUMMARY OF THE INVENTION

Accordingly, the invention provides a compound of formula A:

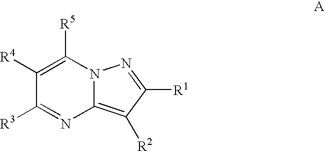

and pharmaceutically acceptable salts thereof;
wherein
R¹ is a 5-7 membered heterocyclic ring or heteroaryl ring, said ring comprising 1-3 heteroatoms selected from N, O or S, and said ring optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, —YR⁸R¹⁰, —YR⁸NR⁷R⁷ and —YR¹⁰;
R² is selected from an aryl ring, a 9-14 membered bicyclic aryl ring, a 5-7 membered heteroaryl ring and a 9-14 membered bicyclic heteroaryl ring, said heteroaryl ring comprising 1-3 heteroatoms selected from N, O and S, said ring optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, —OPO(OR⁷)₂, —YR⁸R¹⁰, —YR⁸NR⁷R⁷ and —YR¹⁰;
R³, R⁴ and R⁵ are each independently selected from carbon-linked R⁶, —X—W—R⁶, H, J, —C(O)OR⁷, —C(O)NR⁷R⁷, —NR⁷C(O)R⁷, —CN, alkyl of 1-6 carbon atoms, branched alkyl of 1-8 carbon atoms, cycloalkyl ring of 3-10 carbons, aryl ring, 5-7 membered heterocyclic ring, and 5-10 membered heteroaryl ring, said heterocyclic ring or heteroaryl ring comprising 1-3 heteroatoms selected from N, O and S, said alkyl of 1-6 carbon atoms, branched alkyl of 1-8 carbon atoms, aryl ring, 5-7 membered heterocyclic ring, and 5-10 membered heteroaryl ring is optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)OR⁷, —OC(O)NR⁷R⁷, NR⁷C(O)R⁷, —NR⁷C(O)OR⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷ —R⁸NR⁷C(O)NR⁷R⁷ and YR¹⁰, wherein at least one of R³, R⁴ and R⁵ comprises R⁶;
R⁶ is a 6-14 membered bridged, bicyclic heterocyclic ring or a 6-14 membered bicyclic spiro heterocyclic ring, said ring optionally substituted with one or more substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸C(O)NR⁷R⁷, —R⁸NR⁷C(O)R⁷, —R⁸NR⁷C(O)OR⁷or —R⁸NR⁷C(O)NR⁷R⁷and YR¹⁰;
R⁷is H or is independently selected from alkyl of 1-6 carbon atoms, branched alkyl of 1-8 carbon atoms, alkenyl of 2-6 carbon atoms, alkynyl of 2-6 carbon atoms, aryl ring and a 5-10 membered heteroaryl ring, optionally substituted with one to four substituents selected from -J, —NO₂, —CN, —N₃, —CHO, —CF₃, —OCF₃, —R, —OR, —S(O)ₘR, —NRR, —NRS(O)ₘR, —OR⁹OR, —OR⁹NRR, —N(R)R⁹OR, —N(R)R⁹NRR, —NRC(O)R, —C(O)R, —C(O)OR, —C(O)NRR, —OC(O)R, —OC(O)OR, —OC(O)NRR, NRC(O)R, —NRC(O)OR, —NRC(O)NRR, —R⁸OR, —R⁸NRR, —R⁸S(O)ₘR, —R⁸C(O)R, —R⁸C(O)OR, —R⁸C(O)NRR, —R⁸OC(O)R, —R⁸OC(O)OR, —R⁸OC(O)NRR, —R⁸NRC(O)R, —R⁸NRC(O)OR, —R⁸NRC(O)NRR and ZR¹⁰, wherein R is selected from alkyl of 1-6 carbon atoms, branched alkyl of 1-8 carbon atoms, alkenyl of 2-6 carbon atoms, alkynyl of 2-6 carbon atoms, cycloalkyl of 3-10 carbon atoms, aryl of 6-10 carbon atoms and heteroaryl of 6-10 atoms, the heteroaryl comprising 1-3 heteroatoms selected from N, O and S;
R⁸ is a divalent group independently selected from alkyl of 1-6 carbon atoms, alkenyl of 2-6 carbon atoms, alkynyl of 2-6 carbon atoms, aryl, heteroaryl, cycloalkyl, and cycloheteroalkyl;
R⁹ is independently a divalent alkyl group of 2-6 carbon atoms;
R¹⁰ is independently selected from cycloalkyl ring of 3-10 carbons, bicycloalkyl ring of 3-10 carbons, aryl ring, heterocyclic ring, heteroaryl ring and a heteroaryl ring fused to one to three aryl or heteroaryl rings, each heterocyclic ring or heteroaryl ring comprising 1-3 heteroatoms selected from N, O and S, each optionally substituted with one to four substituents selected from —H, -aryl, —CH₂-aryl, —NH-aryl, —O-aryl, —S(O)ₘ-aryl, -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹N R⁷R⁷, —NR⁷C(O)R⁷, —C(O)R⁷, —C(O)OR⁷, —C(O)NR⁷R⁷, —OC(O)R⁷—, —OC(O)OR⁷, —OC(O)N R⁷R⁷, —NR⁷C(O)R⁷, —NR⁷C(O)OR⁷, —NR⁷C(O)NR⁷R⁷, —R⁸OR⁷, R⁸NR⁷R⁷, —R⁸S(O)ₘR⁷, —R⁸C(O)R⁷, —R⁸C(O)OR⁷, —R⁸C(O)NR⁷R⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, and —R⁸NR⁷C(O)NR⁷R⁷;
J is fluoro, chloro, bromo, or iodo;
m is an integer of 0-2;
Y is a divalent group independently selected from a bond, alkyl of 1-6 carbon atoms, alkenyl of 2-6 carbon atoms, alkynyl of 2-6 carbon atoms, O, and —NR⁷;
X is selected from a divalent alkyl group of 1-6 carbon atoms, alkenyl of 2-6 carbon atoms, alkynyl of 2-6 carbon atoms, cycloalkyl ring of 3-10 carbons, bicycloalkyl ring of 3-10 carbons, aryl ring, heterocyclic ring and a heteroaryl ring, each heterocyclic ring or heteroaryl ring comprising 1-3 heteroatoms selected from N, O or S; optionally substituted with one to four substituents selected from —H, -aryl, —CH₂-aryl, —NH-aryl, —O-aryl, —S(O)ₘ-aryl, -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷—, —OC(O)OR⁷, —OC(O)NR⁷R⁷, —NR⁷C(O)R⁷, —NR⁷C(O)OR⁷, —NR⁷C(O)NR⁷R⁷, —R⁸OR⁷, R⁸NR⁷R⁷, —R⁸S(O)ₘR⁷, —R⁸C(O)R⁷, —R⁸C(O)OR⁷, —R⁸C(O)NR⁷R⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, and —R⁸NR⁷C(O)NR⁷R⁷;
W is selected from a bond, —OZ—, —ZO— —S(O)ₘZ—, —S(O)₂NR⁷Z—, —NR⁷S(O)ₘZ—, —NR⁷Z—, —ZNR⁷—, —C(O)Z—; —C(O)OZ—, —C(O)NR⁷Z—, —NR⁷C(O)Z—, —NR⁷C(O)NR⁷Z—, —OC(O)Z—, —NR⁷C(O)OZ—, and —OC(O) NR⁷Z—; and
Z is a bond or a divalent alkyl of 1-6 carbon atoms.

The present invention also provides a compound of formula A and pharmaceutically acceptable salts thereof; wherein the bridged, bicyclic heterocyclic ring is selected from:

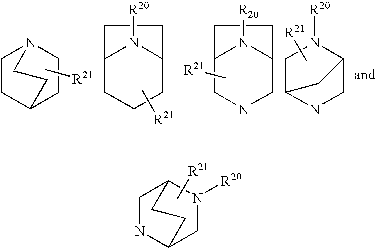

optionally substituted on nitrogen with R²⁰ and optionally substituted on one or more carbons with R²¹, wherein

R²⁰ is selected from H, —C(O)OR⁷, —C(O)NR⁷R⁷, —C(O)R⁷, —S(O)ₘR⁷, alkyl of 1-6 carbon atoms, branched alkyl of 1-8 carbon atoms, cycloalkyl ring of 3-10 carbons, aryl ring, 5-7 membered heterocyclic ring and 5-10 membered heteroaryl ring, each heterocyclic ring or heteroaryl ring comprising 1-3 heteroatoms selected from N, O or S, each of the alkyl of 1-6 carbon atoms, branched alkyl of 1-8 carbon atoms, aryl ring, heterocyclic ring and heteroaryl ring optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷or —R⁸NR⁷C(O)NR⁷R⁷ and YR¹⁰; and
R²¹ is selected from H, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷ or —R⁸NR⁷C(O)NR⁷R⁷ and YR¹⁰; wherein J, Y, m, and R⁷⁻¹⁰ are defined above.

The present invention also provides a pharmaceutical composition comprising a compound of formula A or a pharmaceutically acceptable salt thereof and a pharmaceutically acceptable carrier. The present invention also provides pharmaceutical compositions comprising compounds of formula A or a pharmaceutically acceptable salt thereof in combination with other kinase-inhibiting pharmaceutical compounds or chemotherapeutic agents, and a pharmaceutically acceptable carrier.

The present invention provides a method for making a compound of formula A:

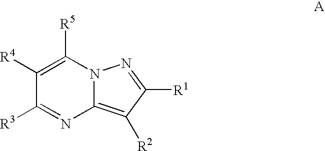

and pharmaceutically acceptable salts thereof; comprising the steps of: (a) reacting a substituted ketone of formula 1

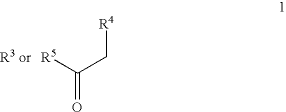

with an acetal of N,Ndialkylformamide or an acetal of N,N-dialkylacetamide, to provide an enaminone compound of formula 2

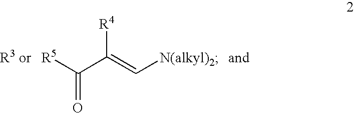

(b) reacting the compound of formula 2 with a substituted 3-aminopyrazole of formula 8,

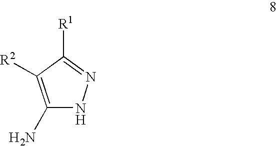

to provide a compound of formula A, wherein R¹⁻¹⁰, J, m, W, X, Y and Z are as defined above.

The present invention also provides a method for making a compound of formula A:

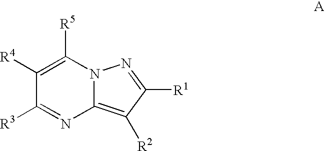

and pharmaceutically acceptable salts thereof; comprising the steps of: (a) reacting an enaminone compound of formula 2

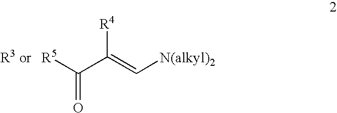

with an aminopyrazole of formula 8a

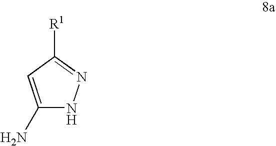

to provide compounds of formula 3c and 3d

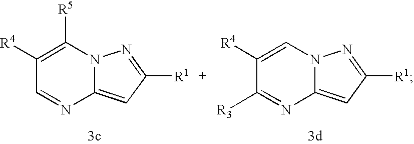

(b) halogenating one or both of the compounds of formula 3c and 3d to provide one or both of compounds of formula 3e and 3f

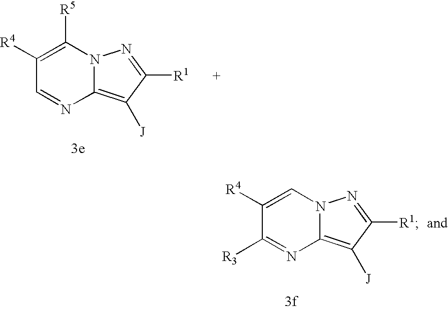

(c) subjecting one or both of the compounds of formula 3e and 3f to a palladium catalyzed, Suzuki coupling using aryl or heteroaryl boronic acids or corresponding boronate esters to provide one or both of the compounds of the invention.

The present invention provides additional independent steps of separating compounds of formula 3c and 3d prior to the halogenation step, separating compounds of formula 3e and 3f prior to the palladium catalyzed, Suzuki coupling step and separating compounds of formula A after the palladium catalyzed, Suzuki coupling step, respectively.

The invention also provides methods for inhibiting Raf kinase activity in a cell comprising contacting a cell with a compound of formula A, whereby the compound inhibits activity of a Raf kinase selected from A-Raf kinase, B-Raf kinase, mutant B-Raf kinase and C-Raf kinase.

The present invention also provides a method of treating an A-Raf kinase, B-Raf kinase, mutant B-Raf kinase or C-Raf kinase dependent condition, said condition comprising cancer or inflammation, by administering to a patient a pharmaceutically effective amount of a compound of formula A.

The present invention provides methods of treating mammalian diseases associated with a Raf kinase selected from A-Raf kinase, B-Raf kinase, mutant B-Raf kinase and C-Raf kinase, by administering to a patient a compound of formula A.

The present invention provides methods of treating a cancer associated with Raf kinase wherein the cancer is selected from breast, kidney, bladder, thyroid, mouth, larynx, esophagus, stomach, colon, ovary, lung, pancreas, skin, liver, prostate and brain cancer.

### DETAILED DESCRIPTION OF THE INVENTION

### Definitions

The following definitions are used in connection with pyrazolo[1,5-a]pyrimidines of the invention. The term “alkyl” refers to saturated aliphatic groups of 1 to 8 carbon atoms, including straight-chain alkyl groups and branched-chain alkyl groups. In one embodiment, a straight chain or branched chain alkyl has 6 or fewer carbon atoms in its backbone. The term “alkyl” can be used alone or as part of a chemical name, such as “alkylamine”. The terms “alkenyl” and “alkynyl” refer to unsaturated aliphatic groups analogous in length and possible substitution to the alkyls described above, but which contain at least one double or triple carbon-carbon bond, respectively. The term “cycloalkyl” refers to saturated cycloaliphatic rings of 3 to 10 carbon atoms, including unbranched cycloalkyl rings and branched cycloalkyl rings. Unless otherwise defined, the term “aryl”, as used herein, refers to an aromatic carbocyclic moiety, e.g. having from 6-20 carbon atoms, including from 6-10 carbon atoms, which may be a single ring (monocyclic) or multiple rings fused together or linked covalently, wherein at least one of the rings is aromatic. Any suitable ring position of the aryl moiety may be covalently linked to the defined chemical structure. Examples of aryl include phenyl and napthyl. The aryl group may be optionally substituted. In addition to other optional substituents, the aryl group may be substituted by an oxo substituent meaning one of the ring carbon atoms is part of a carbonyl group.

Unless otherwise defined, the term “heteroaryl” as used herein means an aromatic heterocyclic ring system, e.g. having from 5-20 ring atoms, which may be a single ring or multiple rings fused together or linked covalently, wherein at least one of the rings is aromatic. The rings may contain one or more heteroatoms, e.g. 1 to 3 heteroatoms, selected from nitrogen, oxygen, or sulfur, wherein the nitrogen or sulfur atom(s) are optionally oxidized, or the nitrogen atom(s) are optionally quaternized. Any suitable ring position of the heteroaryl moiety may be covalently linked to the defined chemical structure. Suitable examples of heteroaryl include 3-pyridinyl, 4-pyridinyl, 1-H-indazol-4-yl or indol-1-yl. The heteroaryl group may be optionally substituted. In addition to other optional substituents, the heteroaryl group may be substituted by an oxo substituent meaning one of the ring carbon atoms is part of a carbonyl group.

The term “heterocyclic”, “heterocycle” or “heterocyclyl” as used herein can be used interchangeably to refer to a stable, saturated or partially unsaturated monocyclic or multicyclic heterocyclic ring system, including a spirocyclic and bridged heterocyclic ring system, e.g. having from 5 to 7 ring members. The heterocyclic ring members are carbon atoms and one or more heteroatoms, e.g. 1 to 3 heteroatoms, selected from nitrogen, oxygen, and sulfur atoms, wherein the nitrogen or sulfur atom(s) are optionally oxidized, or the nitrogen atom(s) are optionally quaternized. The heterocyclic, heterocycle or heterocyclyl group may be optionally substituted. In addition to other optional substituents, the heterocyclic, heterocycle or heterocyclyl group may be substituted by an oxo substituent meaning one of the ring carbon atoms is part of a carbonyl group. The heterocyclic, heterocycle or heterocyclyl group may contain one of more fused rings.

The term “spiro heterocyclic” refers to at least one heterocyclic ring system bonded to another ring system at the same atom.

The term “bridged, bicyclic” refers to a heterocyclic ring system fused to another ring system on non-adjacent atoms, where at least one the ring systems is a heterocyclic ring. Suitable examples of “bridged, bicyclic” ring systems are provided in the Examples section of the specification and include, but are not limited to:

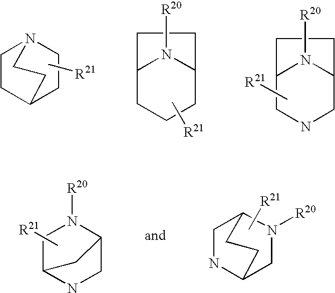

optionally substituted on nitrogen with R²⁰ and optionally substituted on one or more carbons with R²¹, wherein
R²⁰ is selected from H, —C(O)OR⁷, —C(O)NR⁷R⁷, —C(O)R⁷, —S(O)ₘR⁷, alkyl of 1-6 carbon atoms, branched alkyl of 1-8 carbon atoms, cycloalkyl ring of 3-10 carbons, aryl ring, 5-7 membered heterocyclic ring and 5-10 membered heteroaryl ring, each heterocyclic ring or heteroaryl ring comprising 1-3 heteroatoms selected from N, O or S, each of the alkyl of 1-6 carbon atoms, branched alkyl of 1-8 carbon atoms, aryl ring, heterocyclic ring and heteroaryl ring optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷ or —R⁸NR⁷C(O)NR⁷R⁷ and YR¹⁰; and
R²¹ is selected from H, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷ or —R⁸NR⁷C(O)NR⁷R⁷ and YR¹⁰; wherein R⁷⁻¹⁰ are defined above.

The term “bicyclic aryl ring or heteroaryl ring” refers to a ring framework of formula

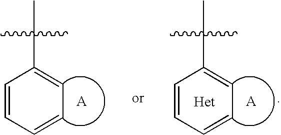

The symbol

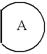

refers to a 5-7 membered heteroaryl ring comprising 1-3 heteroatoms selected from N, O or S. The term “Het” refers to a 6 membered heteroaryl ring containing 1-2 nitrogen atoms. Each of the bicyclic aryl ring or bicyclic heteroaryl ring are optionally substituted with substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, —YR⁸R¹⁰, —YR⁸NR⁷R⁷ and —YR¹⁰.

As used herein, the term “pharmaceutically acceptable carrier” includes pharmaceutically acceptable diluents and excipients.

As used herein, the term “individual”, “subject” or “patient,” used interchangeably, refers to any animal, including mammals, preferably mice, rats, other rodents, rabbits, dogs, cats, swine, cattle, sheep, horses, or primates, and most preferably humans.

According to an exemplary embodiment, the invention provides a compound of formula A:

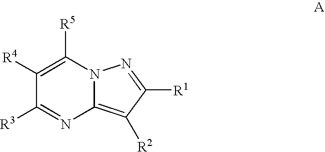

and pharmaceutically acceptable salts thereof;
wherein R¹-R¹⁰, J, m, W, X, Y and Z are as defined above.

Suitable examples of R¹ include, but are not limited to, thienyl, furyl, indolyl, pyrrolyl, thiophenyl, benzofuryl, benzothiophenyl, quinolyl, isoquinolyl, imidazolyl, thiazolyl, oxazolyl, pyridinyl, pyrrolidyl, oxolanyl, thiolanyl, piperidinyl, piperazinyl, thiazolyl, triazolyl, pyrazolyl, pyrazinyl, pyridazinyl, pyrimidinyl, and morpholinyl. The heterocyclic ring or heteroaryl ring may be substituted to the pyrazolo[1,5-a]pyrimidine ring framework in any acceptable position. According to one embodiment, R¹ is 4-pyridinyl or 4-morpholinyl, optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, —OPO(OR⁷)₂, —YR⁸R¹⁰, —YR⁸NR⁷R⁷ and —YR¹⁰.

Suitable examples of R² include, but are not limited to, halogen substituted phenyl, C₁-C₆ alkylsulfonamido substituted phenyl, carbamate substituted phenyl, C₁-C₆ alkoxy substituted phenylcarbamate, benzonitrile, hydroxyl substituted benzonitrile, C₁-C₆ alkoxy substituted benzonitrile, hydroxyphenyl (phenol), C₁-C₆ alkyl substituted hydroxyphenyl (phenol), halogen substituted hydroxyphenyl (phenol), C₁-C₆ alkoxyphenyl, halogen substituted C₁-C₆ alkoxyphenyl, hydroxypyridinyl, C₁-C₆ alkoxypyridinyl, amino phenyl (aniline), halogen substituted amino phenyl (aniline), hydroxyl substituted amino phenyl (aniline), formamide substituted phenyl, hydroxyl substituted phenylformamide, C₁-C₆ alkoxy substituted phenylformamide, C₁-C₆ alkoxy substituted amino phenyl (aniline), urea substituted phenyl, benzamido, C₁-C₆ alkyl substituted benzamido, halogen substituted benzamido, indazolyl, C₁-C₆ alkyl substituted indazolyl, halogen substituted indazolyl, halo C₁-C₆ alkyl substituted indazolyl, perfluoro C₁-C₆ alkyl substituted indazolyl, benzamidazolyl, halogen substituted benzamidazolyl, oxo-dihydro-benzamidazolyl, dihydro-pyrrolodinyl, substituted dihydro-pyrrolodinyl, dihydro-indolyl, substituted dihydro-indolyl, and oxadiazolyl substituted phenyl. Other suitable examples of R² include, but are not limited to, indolyl, benzotriazolyl, oxindolyl, benzothiazolonyl and benzooxazolonyl. The monocyclic aryl ring and the bicyclic heteroaryl ring may be substituted to the pyrazolo[1,5-a]pyrimidine ring framework in any acceptable position.

According to one embodiment, R² is an aryl ring or a bicyclic aryl ring of formula

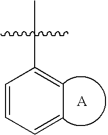

wherein

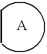

refers to a 5-7 membered heteroaryl ring comprising 1-3 heteroatoms selected from N, O and S, said ring optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, —YR⁸R¹⁰, —YR⁸NR⁷R⁷ and —YR¹⁰.

According to a separate embodiment, R² is a phenyl ring or an indazolyl ring, optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, —YR⁸R¹⁰, —YR⁸NR⁷R⁷ and —YR¹⁰.

According to a separate embodiment, R² is selected from halogen substituted phenyl, C₁-C₆ alkylsulfonamido substituted phenyl, carbamate substituted phenyl, C₁-C₆ alkoxy substituted phenylcarbamate, benzonitrile, hydroxyl substituted benzonitrile, C₁-C₆ alkoxy substituted benzonitrile, hydroxyphenyl, C₁-C₆ alkyl substituted hydroxyphenyl, halogen substituted hydroxyphenyl, C₁-C₆ alkoxyphenyl, halogen substituted C₁-C₆ alkoxyphenyl, hydroxypyridinyl, C₁-C₆ alkoxypyridinyl, amino phenyl, halogen substituted amino phenyl, hydroxyl substituted amino phenyl, formamide substituted phenyl, hydroxyl substituted phenylformamide, C₁-C₆ alkoxy substituted phenylformamide, C₁-C₆ alkoxy substituted amino phenyl, urea substituted phenyl, benzamido, C₁-C₆ alkyl substituted benzamido, halogen substituted benzamido, indazolyl, C₁-C₆ alkyl substituted indazolyl, halogen substituted indazolyl, halo C₁-C₆ alkyl substituted indazolyl, perfluoro C₁-C₆ alkyl substituted indazolyl, benzamidazolyl, halogen substituted benzamidazolyl, dihydro-pyrrolodinyl, substituted dihydro-pyrrolodinyl, dihydro-indolyl, substituted dihydro-indolyl and oxadiazolyl substituted phenyl.

Suitable examples of R⁶ include, but are not limited to bridged, bicyclic heterocyclic rings selected from:

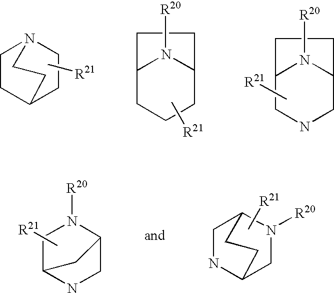

optionally substituted on nitrogen with R²⁰ and optionally substituted on one or more carbons with R²¹, wherein
R²⁰ is selected from H, —C(O)OR⁷, —C(O)NR⁷R⁷, —C(O)R⁷, —C(O)R¹⁰, —S(O)ₘR⁷, alkyl of 1-6 carbon atoms, branched alkyl of 1-8 carbon atoms, cycloalkyl ring of 3-10 carbons, aryl ring, 5-7 membered heterocyclic ring and 5-10 membered heteroaryl ring, each heterocyclic ring or heteroaryl ring comprising 1-3 heteroatoms selected from N, O or S, each of the alkyl of 1-6 carbon atoms, branched alkyl of 1-8 carbon atoms, aryl ring, heterocyclic ring and heteroaryl ring optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷,—N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷ or —R⁸NR⁷C(O)NR⁷R⁷ and YR¹⁰; and
R²¹ is selected from H, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷ or —R⁸NR⁷C(O)NR⁷R⁷ and YR¹⁰; wherein R⁷⁻¹⁰ are defined above.

R⁶ may be directly bonded, via a carbon (referred to as carbon-linked), to the pyrazolo[1,5-a]pyrimidine ring framework in a number of acceptable positions. R⁶ also may be indirectly bonded to the pyrazolo[1,5-a]pyrimidine ring framework in a number of acceptable positions, as joined together using spacer groups defined by X—W—R⁶. According to one embodiment, at least one of R³, R⁴ and R⁵ are each independently selected from carbon-linked R⁶. According to a separate embodiment, at least one of R³, R⁴ and R⁵ are each independently selected from X—W—R⁶.

According to one embodiment, R⁵ is carbon-linked R⁶ and comprises a bridged, bicyclic heterocyclic ring selected from:

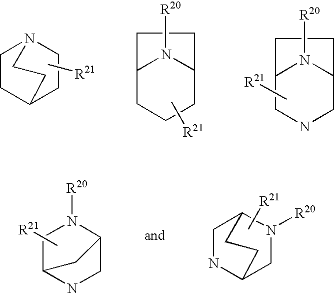

optionally substituted on nitrogen with R²⁰ and optionally substituted on one or more carbons with R²¹.

According to a separate embodiment, R⁵ is X—W—R⁶, wherein R⁶ comprises an aryl ring or a heteroaryl ring substituted with a bridged, bicyclic heterocyclic ring selected from:

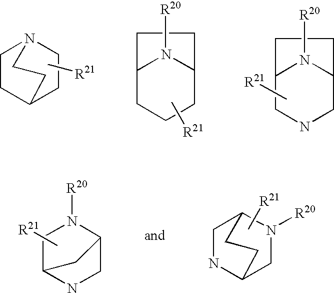

optionally substituted on nitrogen with R²⁰ and optionally substituted on one or more carbons with R²¹, X is aryl or heteroaryl, each further optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —RNR⁷C(O)R⁷, —R⁸NR⁷C(O)OR⁷ or —R⁸NR⁷C(O)NR⁷R⁷ and YR¹⁰ and W is a bond.

According to a separate embodiment, R⁶ is R⁵ is X—W—R⁶, wherein R⁶ comprises a bridged, bicyclic heterocyclic ring selected from:

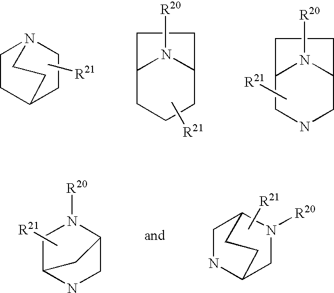

optionally substituted on nitrogen with R²⁰ and optionally substituted on one or more carbons with R²¹, X is aryl or heteroaryl, each further optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷or —R⁸NR⁷C(O)NR⁷R⁷ and YR¹⁰ and W is ZN R⁷ or NR⁷Z.

According to a separate embodiment, R⁶ is a bicyclic spiro heterocyclic ring comprising 1-3 heteroatoms selected from N, O and S, optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)N R⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)N R⁷R⁷, —R⁸NR⁷C(O)R⁷, —R⁸NR⁷C(O)OR⁷or —R⁸NR⁷C(O)NR⁷R⁷ and YR¹⁰.

The compounds of this invention may be prepared from: (a) commercially available starting materials (b) known starting materials which may be prepared as described in literature procedures or (c) new intermediates described in the schemes and experimental procedures herein. Reactions are performed in a solvent appropriate to the reagents and materials employed and suitable for the transformation being effected. It is understood by those skilled in the art of organic synthesis that the various functionalities present on the molecule must be consistent with the chemical transformation proposed. This may necessitate judgement as to the order of synthetic steps.

Compounds of the present invention may be prepared as illustrated in the examples and in following reaction Schemes 1 to 5:

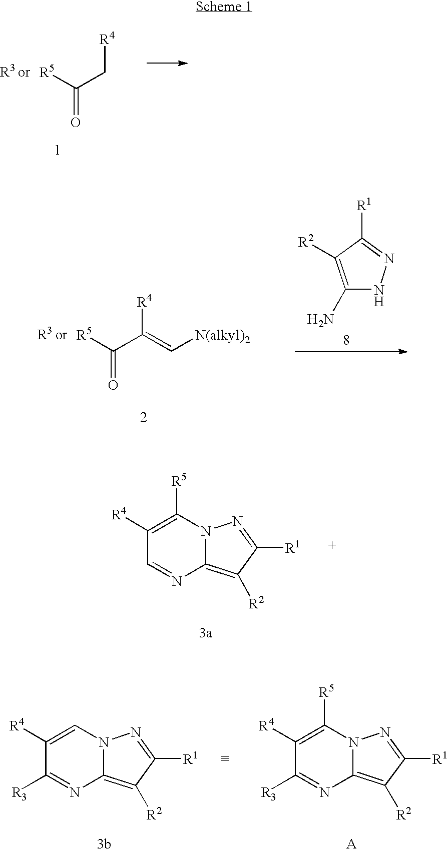

Referring to Scheme 1, the reaction of a ketone of formula 1, optionally substituted with R³, R⁴ and R⁵ with an acetal of N,Ndialkylformamide or an acetal of N,N-dialkylacetamide, carried out in an inert solvent or without a solvent provides an enaminone, namely a 3-dialkylamino-2-propen-1-one, of formula 2. The reaction of the substituted 3-aminopyrazole of formula 8, where R¹ and R² are defined above or are hydrogen, with an appropriately substituted 3-dialkylamino-2-propen-1-one in weak acid such as glacial acetic acid or in an inert solvent such as toluene, acetonitrile or dimethoxyethane, at reflux temperature for several hours, or without solvent at 50-150° C., provides the desired compounds of formula 3a and 3b. In some cases, chemical modification of compounds of formula 3a and 3b, according to methods known by those skilled in the art of organic synthesis, may be performed by those of skill in the art to provide additional compounds of the invention. For example, where any of R³, R⁴, or R⁵ in compounds of formula 3a and 3b is a halogen, or halo aryl group, or the like, palladium catalyzed, Suzuki or Buchwald coupling reactions provide additional compounds of the invention. Furthermore, when any of R³, R⁴, or R⁵ in compounds of formula 3a and 3b is a 2-bromopyridyl moiety, reaction of such a compound at elevated temperature, from 50-150° C. with an amine, alcohol, or thiol, in DMSO or other polar, aprotic solvent, in the presence of a tertiary amine base such as Hunig's base, or sodium hydride or the like, provides compounds of the invention.

Compounds of the invention may also be synthesized according to the route shown in Scheme 2. Thus, the enaminone of formula 2, can react with aminopyrazole compound of formula 8a in weak acid such as glacial acetic acid or in an inert solvent such as toluene, acetonitrile or dimethoxyethane, at reflux temperature for several hours, or without solvent at 50-150° C., to provide one or both of compounds of formula 3c and 3d. Compounds of formula 3c and 3d can be separated, chromatographically or via recrystallization, and halogenated to afford the corresponding halo-pyrazole derivatives, using N-halosuccininmides at room temperature to 50° C. in chlorinated hydrocarbon solvents, to give one or both of compounds of formula 3e or 3f. Alternatively, the mixture of compounds of formula 3c and 3d can be halogenated under these conditions with subsequent separation of compounds of formula 3e or 3f. The halopyrazole compounds of formula 3e or 3f can then undergo palladium catalyzed, Suzuki coupling reactions with aryl or heteroaryl boronic acids or corresponding boronate esters to provide the compounds of the invention.

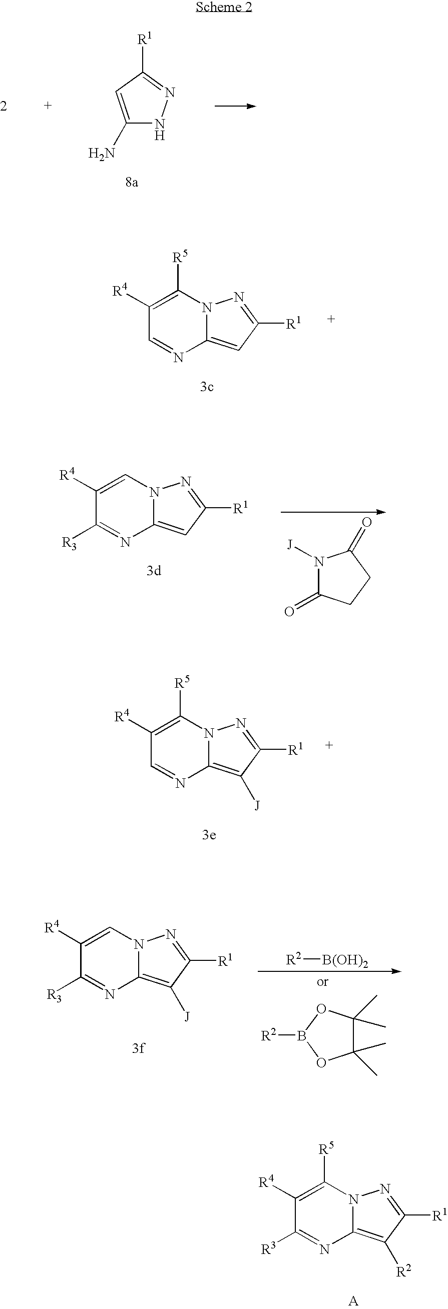

Substituted 3-dimethylamino-1-(3-heteroaryl)-2-propen-1-ones are described in U.S. Pat. Nos. 4,281,000 and 4,521,422 and 3-dialkylamino-1-phenyl-2-propen-1-ones are disclosed in U.S. Pat. Nos. 4,178,449 and 4,236,005. Various 3-amino-4-pyrazoles are disclosed in U.S. Pat. Nos. 4,236,005; 4,281,000; 4,521,422; 4,626,538; 4,654,347; and 4,900,836. Pyrazolo[1,5-a]pyrimidines are prepared by condensation of 3-aminopyrazoles and substituted 3-aminopyrazoles with 1,3-dicarbonyl compounds as described in J. Med. Chem., 18, 645 (1974); J. Med. Chem. 18, 460 (1975); J. Med. Chem., 20, 386 (1977); Synthesis, 673 (1982) and references contained therein.

Additional aminopyrazole intermediate compounds of formula 8, are available according to the route shown in Scheme 3. Referring to Scheme 3, the condensation reaction of substituted acetonitrile compounds of formula 5, wherein R² is as defined above or is hydrogen, with substituted ester compounds of formula 4 can be carried out in the presence of a base such as, but not limited to sodium ethoxide, in a suitable solvent such as ethanol to provide intermediate compounds of formula 6. Intermediate compounds of formula 6 can subsequently be reacted with hydrazine hydrate in a suitable solvent such as ethanol to provide aminopyrazole compounds of formula 8 where R¹ and R² are defined above. For certain substituted intermediate compounds of formula 6, it is necessary to first react with phosphorus oxychloride at elevated temperatures, typically at reflux, to provide intermediate compounds of formula 7. Intermediate compounds of formula 7 can be converted to substituted aminopyrazole compounds of formula 8 by subsequent reaction with hydrazine hydrate in a suitable solvent such as ethanol. Substituted ester compounds of formula 4 and substituted acetonitrile compounds of formula 5 can be obtained from commercial sources or readily prepared by numerous literature procedures by those skilled in the art. Aminopyrazole compounds of formula 8 can also be prepared from an alternative route starting from aldehyde compounds of formula 15, as shown in Scheme 3. In the first step of this alternative route, aldehyde compounds 15, which are commercially available or can be prepared by known methods, are reacted typically at room temperature with phosphonate compounds of formula 16 (which can be prepared according to the procedure of Tet. Lett., 1988, 39, 1717-1720) in a suitable solvent such as tetrahydrofuran, using an appropriate base such as, but not limited to, cesium carbonate to provide intermediate compounds of formula 17. Intermediate compounds of formula 17 are subsequently heated, typically at 80° C., in a mixture of chloroform, phosphorus oxychloride or the like, and dimethylformamide to give the corresponding substituted 3-chloropropenals. The crude 3-chloropropenals are treated with hydroxylamine in a suitable solvent such as dimethylformamide, typically at room temperature, to provide the corresponding 3-chloropropenal oximes, which are then treated with a suitable dehydrating agent such as, but not limited to, phosphorus oxychloride, typically at room temperature, to give the corresponding 3-chloroacrylonitriles. The intermediate 3-chloroacrylonitriles can then be converted into the desired substituted aminopyrazole compounds of formula 8 by subsequent reaction with hydrazine hydrate in a suitable solvent such as ethanol.

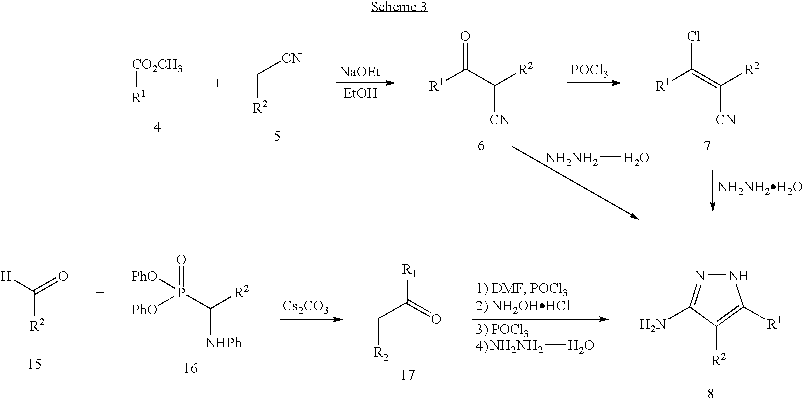

Referring to Scheme 4, compounds of the invention are also available via condensation of the desired aminopyrazole compounds of formula 8, with alkoxymethylene malonates in weak acid such as acetic acid at elevated temperature, typically at reflux, to provide the dihydropyrazolo[1,5-a]pyrimidine derivative compounds of formula 9. Hydrolysis of the ester functionality of compounds of formula 9 mediated by aqueous base such as sodium hydroxide provides pyrimidone compounds of formula 10, that is then decarboxylated at elevated temperature to form compounds of formula 11. Transformation of the pyrimidone compounds of formula 11 into the corresponding halo-pyrimidine compounds of formula 12, is carried out with phosphorus oxychloride, or similar halogenating agent, at elevated temperature in the presence of an amine base such as N,N-diethylaniline. Reaction of halo-pyrimidine compounds of formula 12 with M-X—W—R⁶, where M is a hydrogen, boronic acid, boronate ester, stannane, or silane, in the presence of a transition metal catalyst then gives compounds of formula 13 of the invention which may be further functionalized according to methods known to those skilled in the art. Halo-pyrimidine compounds of formula 12 can similarly be converted into compounds of formula 13 of the invention by reaction with M-X—W—R⁶ where M is a metal including but not limited to zinc, lithium, and magenesium. In the case of compounds wherein R² is a methoxyphenyl moiety, the corresponding phenol (compounds of formula 14, R₂=PhOH) is provided by reaction with pyridine hydrochloride at elevated temperature, or boron tribromide.

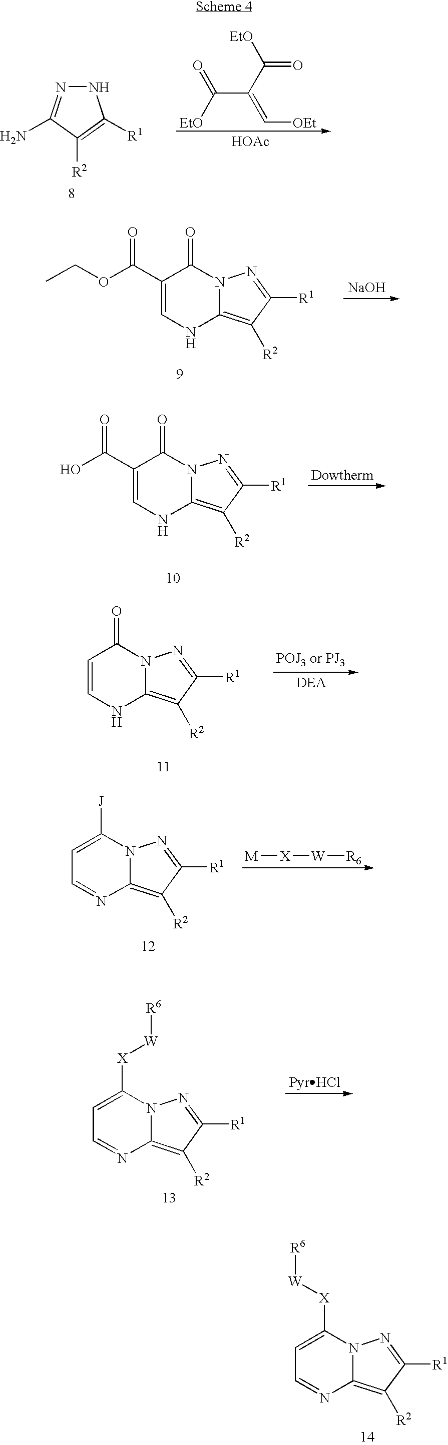

Referring to Scheme 5, certain compounds of the invention wherein R⁶ is a spiro-bicyclic moiety are available starting from ketone-ketal compounds of formula 16, prepared in one step from commercially available methyl ester compounds of formula 15 [D. Tanner, P. Somfai, Syn. Commun., 16(12), 1517-1522 (1986)]. Conversion of methyl ketone compounds of formula 16 into enaminone compounds of formula 17 is carried out by reaction with acetals of N,N-dialkylformamide or acetals of N,N-dialkylacetamide, in an inert solvent or without a solvent at a temperature of 50-100° C. The reaction of the substituted 3-aminopyrazole compounds of formula 8, where R¹ and R² are defined above or are hydrogen, with enaminone compounds of formula 17 in weak acid such as glacial acetic acid or in an inert solvent such as toluene, acetonitrile or dimethoxyethane, at reflux temperature for several hours, or without solvent at 50-150° C., produces the desired ketal compounds of formula 18. Hydrolysis of the ketal compounds of formula 18 under acidic conditions, such as aqueous acetic acid, trifluoroacetic acid, tosic acid, or camphorsulfonic acid at room temperature to 100° C., followed by reaction of the resulting diol with carbocyclic and heterocyclic ketones under acidic conditions provides invented compounds of formula 19.

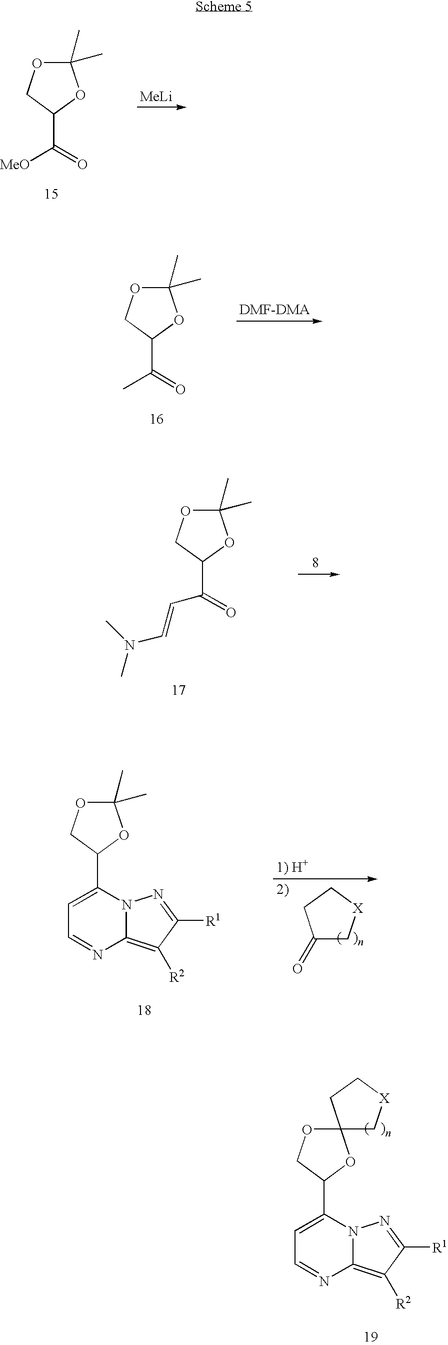

Exemplary compounds of Formula A prepared by methods of the present invention include the following compounds: 3-(7-{6-[(1-azabicyclo[2.2.2]oct-4-ylmethyl)amino]pyridin-3-yl}-2-pyridin-4-ylpyrazolo[1,5-a]pyrimidin-3-yl)phenol, 3-(7-{6-[(3S)-1-azabicyclo[2.2.2]oct-3-ylamino]pyridin-3-yl}-2-pyridin-4-ylpyrazolo[1,5-a]pyrimidin-3-yl)phenol, 3-(7-{6-[(3R)-1-azabicyclo[2.2.2]oct-3-ylamino]pyridin-3-yl}-2-pyridin-4-ylpyrazolo[1,5-a]pyrimidin-3-yl)phenol, (3R)—N-{4-[3-(3-methoxyphenyl)-2-pyridin-4-ylpyrazolo[1,5-a]pyrimidin-7-yl]pyridin-2-yl}quinuclidin-3-amine, (3R)—N-{5-[3-(4-chloro-3-methoxyphenyl)-2-pyridin-4-ylpyrazolo[1,5-a]pyrimidin-7-yl]pyridin-2-yl}quinuclidin-3-amine, 3-{7-[(1-azabicyclo[2.2.2]oct-4-ylmethyl)amino]-2-pyridin-4-ylpyrazolo[1,5-a]pyrimidin-3-yl}phenol, ethyl 3-[3-(3-methoxyphenyl)-2-pyridin-4-ylpyrazolo[1,5-a]pyrimidin-7-yl]-8-azabicyclo[3.2.1]octane-8-carboxylate, ethyl 3-[3-(3-hydroxyphenyl)-2-pyridin-4-ylpyrazolo[1,5-a]pyrimidin-7-yl]-8-azabicyclo[3.2.1]-octane-8-carboxylate, 3-[7-(8-azabicyclo[3.2.1]oct-3-yl)-2-pyridin-4-ylpyrazolo[1,5-a]pyrimidin-3-yl]phenol, ethyl 3-[3-(4-chloro-3-methoxyphenyl)-2-pyridin-4-ylpyrazolo[1,5-a]pyrimidin-7-yl]-8-azabicyclo[3.2.1]octane-8-carboxylate, ethyl 3-[3-(4-chloro-3-hydroxyphenyl)-2-pyridin-4-ylpyrazolo[1,5-a]pyrimidin-7-yl]-8-azabicyclo[3.2.1]octane-8-carboxylate, 5-[7-(8-azabicyclo[3.2.1]oct-3-yl)-2-pyridin-4-ylpyrazolo[1,5-a]pyrimidin-3-yl]-2-chlorophenol, 5-[7-(8-acetyl-8-azabicyclo[3.2.1]oct-3-yl)-2-pyridin-4-ylpyrazolo[1,5-a]pyrimidin-3-yl]-2-chlorophenol, 2-chloro-5-{7-[8-(methylsulfonyl)-8-azabicyclo[3.2.1]oct-3-yl]-2-pyridin-4-ylpyrazolo[1,5-a]pyrimidin-3-yl}phenol, 5-[7-(8-acetyl-8-azabicyclo[3.2.1]oct-3-yl)-2-pyridin-4-ylpyrazolo[1,5-a]pyrimidin-3-yl]-2-chlorophenyl acetate, 7-(8-azabicyclo[3.2.1]oct-3-yl)-3-(4-chloro-3-methoxyphenyl)-2-pyridin-4-ylpyrazolo[1,5-a]pyrimidine, 3-(4-chloro-3-methoxyphenyl)-7-(8-ethyl-8-azabicyclo[3.2.1]oct-3-yl)-2-pyridin-4-ylpyrazolo[1,5-a]pyrimidine, 2-chloro-5-[7-(8-ethyl-8-azabicyclo[3.2.1]oct-3-yl)-2-pyridin-4-ylpyrazolo[1,5-a]pyrimidin-3-yl]phenol, 7-(8-azabicyclo[3.2.1]oct-3-yl)-3-(3-methoxyphenyl)-2-pyridin-4-ylpyrazolo[1,5-a]pyrimidine, 7-(8-ethyl-8-azabicyclo[3.2.1]oct-3-yl)-3-(2-methoxypyridin-4-yl)-2-pyridin-4-ylpyrazolo[1,5-a]pyrimidine, 4-[7-(8-ethyl-8-azabicyclo[3.2.1]oct-3-yl)-2-pyridin-4-ylpyrazolo[1,5-a]pyrimidin-3-yl]pyridin-2-ol, 4-[7-(8-ethyl-8-azabicyclo[3.2.1]oct-3-yl)-2-pyridin-4-ylpyrazolo[1,5-a]pyrimidin-3-yl]aniline, 1-{4-[7-(8-ethyl-8-azabicyclo[3.2.1]oct-3-yl)-2-pyridin-4-ylpyrazolo[1,5-a]pyrimidin-3-yl]phenyl}urea, 3-(3-methoxyphenyl)-7-[4-(8-methyl-3,8-diazabicyclo[3.2.1]oct-3-yl)phenyl]-2-pyridin-4-ylpyrazolo[1,5-a]pyrimidine, 3-{7-[4-(8-methyl-3,8-diazabicyclo[3.2.1]oct-3-yl)phenyl]-2-pyridin-4-ylpyrazolo[1,5-a]pyrimidin-3-yl}phenol, 3-(4-chloro-3-methoxyphenyl)-7-[4-(8-methyl-3,8-diazabicyclo[3.2.1]oct-3-yl)phenyl]-2-pyridin-4-ylpyrazolo[1,5-a]pyrimidine, 2-chloro-5-{7-[4-(8-methyl-3,8-diazabicyclo[3.2.1]oct-3-yl)phenyl]-2-pyridin-4-ylpyrazolo[1,5-a]pyrimidin-3-yl}phenol, 7-{4-[(1S,4S)-2,5-diazabicyclo[2.2.1]hept-2-yl]phenyl}-3-(3-methoxyphenyl)-2-pyridin-4-ylpyrazolo[1,5-a]pyrimidine, 3-(7-{4-[(1S,4S)-2,5-diazabicyclo[2.2.1]hept-2-yl]phenyl}-2-pyridin-4-ylpyrazolo[1,5-a]pyrimidin-3-yl)phenol, 3-(7-{4-[(1S,4S)-5-methyl-2,5-diazabicyclo[2.2.1]hept-2-yl]phenyl}-2-pyridin-4-ylpyrazolo[1,5-a]pyrimidin-3-yl)phenol, 3-(4-chloro-3-methoxyphenyl)-7-{4-[(1S,4S)-2,5-diazabicyclo[2.2.1]hept-2-yl]phenyl}-2-pyridin-4-ylpyrazolo[1,5-a]pyrimidine, 7-{4-[(1S,4S)-2,5-diazabicyclo[2.2.1]hept-2-yl]phenyl}-3-(4-fluoro-3-methoxyphenyl)-2-pyridin-4-ylpyrazolo[1,5-a]pyrimidine, 5-(7-{4-[(1S,4S)-2,5-diazabicyclo[2.2.1]hept-2-yl]phenyl}-2-pyridin-4-ylpyrazolo[1,5-a]pyrimidin-3-yl)-2-fluorophenol, 3-(1H-indazol-4-yl)-7-{6-[(1S,4S)-5-methyl-2,5-diazabicyclo[2.2.1]hept-2-yl]pyridin-3-yl}-2-pyridin-4-ylpyrazolo[1,5-a]pyrimidine, 3-(1H-indazol-4-yl)-7-{4-[(1S,4S)-5-methyl-2,5-diazabicyclo[2.2.1]hept-2-yl]phenyl}-2-pyridin-4-ylpyrazolo[1,5-a]pyrimidine, 3-(3-methoxyphenyl)-7-{4-[(1S,4S)-5-methyl-2,5-diazabicyclo[2.2.1]-hept-2-yl]phenyl}-2-pyridin-4-ylpyrazolo[1,5-a]pyrimidine, 3-(4-fluoro-3-methoxyphenyl)-7-{4-[(1S,4S)-5-methyl-2,5-diazabicyclo[2.2.1]hept-2-yl]phenyl}-2-pyridin-4-ylpyrazolo[1,5-a]pyrimidine, 5-(7-{4-[(1S,4S)-5-ethyl-2,5-diazabicyclo[2.2.1]-hept-2-yl]phenyl}-2-pyridin-4-ylpyrazolo[1,5-a]pyrimidin-3-yl)-2-fluorophenol, 5-(7-{4-[(1S,4S)-5-acetyl-2,5-diazabicyclo[2.2.1]hept-2-yl]phenyl}-2-pyridin-4-ylpyrazolo[1,5-a]pyrimidin-3-yl)-2-fluorophenol, 7-{6-[(1S,4S)-5-methyl-2,5-diazabicyclo[2.2.1]hept-2-yl]pyridin-3-yl}-3-(1H-indazol-4-yl)-2-pyridin-4-ylpyrazolo[1,5-a]pyrimidine, 3-(7-chloro-1H-indazol-4-yl)-7-{6-[(1S,4S)-5-methyl-2,5-diazabicyclo[2.2.1]hept-2-yl]pyridin-3-yl}-2-pyridin-4-ylpyrazolo[1,5-a]pyrimidine, 3-(7-fluoro-1H-indazol-4-yl)-7-{6-[(1S,4S)-5-methyl-2,5-diazabicyclo[2.2.1]hept-2-yl]pyridin-3-yl}-2-pyridin-4-ylpyrazolo[1,5-a]pyrimidine, 3-(7-{6-[(1S,4S)-5-methyl-2,5-diazabicyclo[2.2.1]hept-2-yl]pyridin-3-yl}-2-pyridin-4-ylpyrazolo[1,5-a]pyrimidin-3-yl)benzamide, 7-{4-[(1S,4S)-2,5-diazabicyclo[2.2.1]hept-2-yl]-2-methylphenyl}-3-(1H-indazol-4-yl)-2-pyridin-4-yl pyrazolo[1,5-a]pyrimidine, 3-(1H-indazol-4-yl)-7-{2-methyl-4-[(1S,4S)-5-methyl-2,5-diazabicyclo[2.2.1]hept-2-yl]phenyl}-4-ylpyrazolo[1,5-a]pyrimidine, 7-{2-[(1S,4S)-2,5-diazabicyclo[2.2.1]hept-2-yl]-4-fluorophenyl}-(1H-indazol-4-yl)-2-pyridin-4-ylpyrazolo[1,5-a]pyrimidine, 7-{4-fluoro-2-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, tert-butyl(1S,4S)-5-{2-[3-(4-chloro-3-methoxyphenyl)-2-pyridin-4-ylpyrazolo[1,5-a]pyrimidin-7-yl]-5-fluorophenyl}-2,5-diazabicyclo[2.2.1]heptane-2-carboxylate, 3-(4-chloro-3-methoxyphenyl)-7-{2-[(1S,4S)-2,5-diazabicyclo[2.2.1]hept-2-yl]-4-fluorophenyl}-2-pyridin-4-ylpyrazolo[1,5-a]pyrimidine, 3-(4-chloro-3-methoxyphenyl)-7-{4-fluoro-2-[(1S,4S)-5-methyl-2,5-diazabicyclo[2.2.1]hept-2-yl]phenyl}-2-pyridin-4-ylpyrazolo[1,5-a]pyrimidine, 7-{4-[(1S,4S)-2,5-diazabicyclo-[2.2.1]hept-2-yl]phenyl}-3-(1H-indazol-4-yl)-2-pyridin-4-ylpyrazolo[1,5-a]pyrimidine, 7-{4-[(1S,4S)-2,5-diazabicyclo[2.2.1]hept-2-yl]-2-fluorophenyl}-3-(1H-indazol-4-yl)-2-pyridin-4-yl pyrazolo[1,5-a]pyrimidine, 7-{2-(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, 2-fluoro-5-(7-{4-[(1S,4S)-5-methyl-2,5-diazabicyclo[2.2.1]hept-2-yl]phenyl}-2-pyridin-4-ylpyrazolo[1,5-a]pyrimidin-3-yl)phenol, 3-(4-fluoro-3-methoxyphenyl)-7-{2-methyl-4-[(1S,4S)-5-methyl-2,5-diazabicyclo[2.2.1]hept-2-yl]phenyl}-2-pyridin-4-ylpyrazolo[1,5-a]pyrimidine, 2-fluoro-5-(7-{2-methyl-4-[(1S,4S)-5-methyl-2,5-diazabicyclo[2.2.1]hept-2-yl]phenyl}-2-pyridin-4-ylpyrazolo[1,5-a]pyrimidin-3-yl)phenol, 3-(7-{4-[(1S,4S)-2,5-diazabicyclo[2.2.1]hept-2-yl]-2-methylphenyl}-2-pyridin-4-ylpyrazolo[1,5-a]pyrimidin-3-yl)phenol, 3-(7-{2-methyl-4-[(1S,4S)-5-methyl-2,5-diazabicyclo[2.2.1]hept-2-yl]phenyl}-2-pyridin-4-yl pyrazolo[1,5-a]pyrimidin-3-yl)phenol, 3-(7-{2-chloro-4-[(1S,4S)-2,5-diazabicyclo[2.2.1]hept-2-yl]phenyl}-2-pyridin-4-ylpyrazolo[1,5-a]pyrimidin-3-yl)phenol, 3-(7-{2-chloro-4-[(1S,4S)-5-methyl-2,5-diazabicyclo[2.2.1]hept-2-yl]phenyl}-2-pyridin-4-ylpyrazolo[1,5-a]pyrimidin-3-yl)phenol, 7-{2-chloro-4-[(1S,4S)-2,5-diazabicyclo[2.2.1]hept-2-yl]phenyl}-3-(1H-indazol-4-yl)-2-pyridin-4-ylpyrazolo[1,5-a]pyrimidine, 7-{2-chloro-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, ethyl 3-[3-(3-hydroxy-4-methylphenyl)-2-pyridin-4-ylpyrazolo[1,5-a]pyrimidin-7-yl]-8-azabicyclo[3.2.1]octane-8-carboxylate, 5-[7-(8-ethyl-8-azabicyclo[3.2.1]oct-3-yl)-2-pyridin-4-ylpyrazolo[1,5-a]pyrimidin-3-yl]-2-fluorophenol, ethyl 3-[3-(2,3-difluorophenyl)-2-pyridin-4-ylpyrazolo[1,5-a]pyrimidin-7-yl]-8-azabicyclo[3.2.1]octane-8-carboxylate, ethyl 3-(3-{3-[(methylsulfonyl)amino]-phenyl}-2-pyridin-4-ylpyrazolo[1,5-a]pyrimidin-7-yl)-8-azabicyclo[3.2.1]octane-8-carboxylate, methyl {4-[7-(8-ethyl-8-azabicyclo[3.2.1]oct-3-yl)-2-pyridin-4-ylpyrazolo[1,5-a]pyrimidin-3-yl]phenyl}carbamate, 4-[7-(8-ethyl-8-azabicyclo[3.2.1]-oct-3-yl)-2-pyridin-4-ylpyrazolo[1,5-a]pyrimidin-3-yl]-2-hydroxybenzonitrile, tert-butyl{4-[7-(8-ethyl-8-azabicyclo[3.2.1]oct-3-yl)-2-pyridin-4-ylpyrazolo[1,5-a]pyrimidin-3-yl]-2-methoxyphenyl}carbamate, 4-[7-(8-ethyl-8-azabicyclo[3.2.1]oct-3-yl)-2-pyridin-4-ylpyrazolo[1,5-a]pyrimidin-3-yl]-2-methoxyaniline, 2-amino-5-[7-(8-ethyl-8-azabicyclo[3.2.1]oct-3-yl)-2-pyridin-4-ylpyrazolo[1,5-a]pyrimidin-3-yl]phenol, N-{4-[7-(8-ethyl-8-azabicyclo[3.2.1]oct-3-yl)-2-pyridin-4-ylpyrazolo[1,5-a]pyrimidin-3-yl]-2-hydroxyphenyl}formamide, N-{4-[7-(8-ethyl-8-azabicyclo[3.2.1]oct-3-yl)-2-pyridin-4-ylpyrazolo[1,5-a]pyrimidin-3-yl]-2-methoxyphenyl}formamide, ethyl 3-[3-(1H-indazol-4-yl)-2-pyridin-4-ylpyrazolo[1,5-a]pyrimidin-7-yl]-8-azabicyclo[3.2.1]octane-8-carboxylate, 7-(8-azabicyclo[3.2.1]oct-3-yl)-3-(1H-indazol-4-yl)-2-pyridin-4-ylpyrazolo-[1,5-a]pyrimidine, ethyl 3-[3-(7-chloro-1H-indazol-4-yl)-2-pyridin-4-yl pyrazolo[1,5-a]pyrimidin-7-yl]-8-azabicyclo[3.2.1]octane-8-carboxylate, ethyl 3-{2-pyridin-4-yl-3-[7-(trifluoromethyl)-1H-indazol-4-yl]pyrazolo[1,5-a]pyrimidin-7-yl}-8-azabicyclo[3.2.1]-octane-8-carboxylate, 7-(8-ethyl-8-azabicyclo[3.2.1]oct-3-7-methyl-1H-indazol-4-yl)-2-pyridin-4-ylpyrazolo[1,5-a]pyrimidine, ethyl 3-[3-(7-methyl-1H-indazol-4-yl)-2-pyridin-4-ylpyrazolo[1,5-a]pyrimidin-7-yl]-8-azabicyclo[3.2.1]octane-8-carboxylate, 3-(7-chloro-1H-indazol-4-yl)-7-(8-ethyl-8-azabicyclo[3.2.1]oct-3-yl)-2-pyridin-4-ylpyrazolo[1,5-a]pyrimidine, 7-(8-ethyl-8-azabicyclo[3.2.1]oct-3-yl)-2-pyridin-4-yl-3-[7-(trifluoromethyl)-1H-indazol-4-yl]pyrazolo[1,5-a]pyrimidine, ethyl 3-[3-(7-fluoro-1H-indazol-4-yl)-2-pyridin-4-ylpyrazolo[1,5-a]pyrimidin-7-yl]-8-azabicyclo[3.2.1]octane-8-carboxylate, 3-(7-chloro-6-fluoro-1H-indazol-4-yl)-7-(8-ethyl-8-azabicyclo[3.2.1]oct-3-yl)-2-pyridin-4-ylpyrazolo[1,5-a]pyrimidine, ethyl 3-[3-(2-oxo-2,3-dihydro-1H-benzimidazol-4-yl)-2-pyridin-4-ylpyrazolo[1,5-a]pyrimidin-7-yl]-8-azabicyclo[3.2.1]-octane-8-carboxylate, ethyl 3-[3-(1H-indol-4-yl)-2-pyridin-4-yl pyrazolo[1,5-a]pyrimidin-7-yl]-8-azabicyclo[3.2.1]octane-8-carboxylate, ethyl 3-[3-(1H-indol-6-yl)-2-pyridin-4-ylpyrazolo[1,5-a]pyrimidin-7-yl]-8-azabicyclo[3.2.1]octane-8-carboxylate, ethyl 3-[3-(2-oxo-2,3-dihydro-1H-pyrrolo[2,3-b]pyridin-4-yl)-2-pyridin-4-ylpyrazolo[1,5-a]pyrimidin-7-yl]-8-azabicyclo[3.2.1]octane-8-carboxylate, 7-(8-ethyl-8-azabicyclo-[3.2.1]oct-3-yl)-3-(1H-indol-6-yl)-2-pyridin-4-ylpyrazolo[1,5-a]pyrimidine, ethyl 3-[3-(2-oxo-2,3-dihydro-1H-indol-6-yl)-2-pyridin-4-yl pyrazolo[1,5-a]pyrimidin-7-yl]-8-azabicyclo[3.2.1]octane-8-carboxylate, 2-chloro-5-[7-(2,2-dimethyl-1,3-dioxolan-4-yl)-2-pyridin-4-ylpyrazolo[1,5-a]pyrimidin-3-yl]phenol, 7-(8-Ethyl-8-azabicyclo[3.2.1]-octan-3-yl)-3-(1H-indazol-4-yl)-2-(pyridin-4-yl)pyrazolo[1,5-a]pyrimidine, Ethyl 3-(3-(3-(1,3,4-oxadiazol-2-yl)phenyl)-2-(pyridin-4-yl)pyrazolo[1,5-a]pyrimidin-7-yl)-8-azabicyclo[3.2.1]octane-8-carboxylate, tert-Butyl (1S,4S)-5-{3-fluoro-4-[3-(1H-indazol-4-yl)-2-pyridin-4-ylpyrazolo[1,5-a]pyrimidin-7-yl]phenyl}-2,5-diazabicyclo-[2.2.1]heptane-2-carboxylate, 7-{4-[(1S,4S)-2,5-diazabicyclo[2.2.1]hept-2-yl]-2-fluorophenyl}-3-(1H-indazol-4-yl)-2-pyridin-4-ylpyrazolo[1,5-a]pyrimidine, 7-{2-Fluoro-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, 7-{2-fluoro-4-[(1S,4S)-5-methyl-5-oxido-2,5-diazabicyclo[2.2.1]hept-2-yl]phenyl}-3-(1H-indazol-4-yl)-2-pyridin-4-ylpyrazolo-[1,5-a]pyrimidine, (1S,4S)-5-{3-Chloro-4-[3-( 1H-indazol-4-yl)-2-pyridin-4-yl-pyrazolo[1,5-a]pyrimidin-7-yl]-phenyl}-2,5-diaza-bicyclo[2.2.1]heptane-2-carboxylic acid tert-butyl ester, 7-[2-Chloro-4-((1S,4S)-2,5-diaza-bicyclo[2.2.1]hept-2-yl)-phenyl]-3-(1H-indazol-4-yl)-2-pyridin-4-yl-pyrazolo[1,5-a]pyrimidine bis-hydrochloride salt, 7-[2-Chloro-4-((1S,4S)-5-methyl-2,5-diaza-bicyclo[2.2.1]hept-2-yl)-phenyl]-3-(1H-indazol-4-yl)-2-pyridin-4-yl-pyrazolo[1,5-a]pyrimidine, tert-butyl(1S,4S)-5-{3,5-difluoro-4-[3-(1H-indazol-4-yl)-2-pyridin-4-ylpyrazolo[1,5-a]pyrimidin-7-yl]phenyl}-2,5-diazabicyclo[2.2.1]heptane-2-carboxylate, 7-(4-((1S,4S)-2,5-diazabicyclo[2.2.1]-heptan-2-yl)-2,6-difluorophenyl)-3-(1H-indazol-4-yl)-2-(pyridin-4-yl)pyrazolo[1,5-a]pyrimidine, 7-{2,6-difluoro-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, tert-Butyl (1S,4S)-5-{4-[3-(7-fluoro-1H-indazol-4-yl)-2-pyridin-4-ylpyrazolo[1,5-a]pyrimidin-7-yl]phenyl}-2,5-diazabicyclo[2.2.1]heptane-2-carboxylate, 7-[4-(2,5-Diaza-bicyclo[2.2.1]hept-2-yl)-phenyl]-3-(7-fluoro-1H-indazol-4-yl)-2-pyridin-4-yl-pyrazolo[1,5-a]pyrimidine, 3-(7-fluoro-1H-indazol-4-yl)-7-{4-[(1S,4S)-5-methyl-2,5-diazabicyclo[2.2.1]hept-2-yl]phenyl}-2-pyridin-4-ylpyrazolo[1,5-a]pyrimidine hydrochloride, tert-Butyl (1S,4S)-5-{3-fluoro-4-[3-(7-fluoro-1H-indazol-4-yl)-2-pyridin-4-ylpyrazolo[1,5-a]pyrimidin-7-yl]phenyl}-2,5-diazabicyclo[2.2.1]heptane-2-carboxylate, 7-{4-[(1S,4S)-2,5-diazabicyclo[2.2.1]hept-2-yl]-2-fluorophenyl}-3-(7-fluoro-1H-indazol-4-yl)-2-pyridin-4-ylpyrazolo[1,5-a]pyrimidine, 3-(7-fluoro-1H-indazol-4-yl)-7-{2-fluoro-4-[(1S,4S)-5-methyl-2,5-diazabicyclo[2.2.1]hept-2-yl]phenyl}-2-pyridin-4-ylpyrazolo[1,5-a]pyrimidine, tert-Butyl (1S,4S)-5-{3,5-difluoro-4-[3-(7-fluoro-1H-indazol-4-yl)-2-pyridin-4-ylpyrazolo[1,5-a]pyrimidin-7-yl]phenyl}-2,5-diazabicyclo[2.2.1]heptane-2-carboxylate, 7-{4-[(1S,4S)-2,5-Diazabicyclo[2.2.1]hept-2-yl]-2,6-difluorophenyl}-3-(7-fluoro-1H-indazol-4-yl)-2-pyridin-4-ylpyrazolo[1,5-a]pyrimidine, 7-{2,6-Difluoro-4-[(1S,4S)-5-methyl-2,5-diazabicyclo[2.2.1]hept-2-yl]phenyl}-3-(7-fluoro-1H-indazol-4-yl)-2-pyridin-4-ylpyrazolo[1,5-a]pyrimidine, ethyl 3-[3-(1H-indazol-4-yl)-2-pyridin-4-ylpyrazolo[1,5-a]pyrimidin-5-yl]-8-azabicyclo[3.2.1]-octane-8-carboxylate, 2-{3-[3-(1H-indazol-4-yl)-2-pyridin-4-ylpyrazolo[1,5-a]pyrimidin-7-yl]-8-azabicyclo[3.2.1]oct-8-yl}ethanol, 3-(1H-indazol-4-yl)-7-(8-isopropyl-8-azabicyclo[3.2.1]oct-3-yl)-2-pyridin-4-ylpyrazolo[1,5-a]pyrimidine, 3-(1H-indazol-4-yl)-7-[8-(methylsulfonyl)-8-azabicyclo[3.2.1]oct-3-yl]-2-pyridin-4-ylpyrazolo[1,5-a]pyrimidine, 3-[3-(1H-indazol-4-yl)-2-pyridin-4-ylpyrazolo[1,5-a]pyrimidin-7-yl]-8-azabicyclo[3.2.1]octane-8-carboxamide, 2-{3-[3-(1H-indazol-4-yl)-2-pyridin-4-ylpyrazolo[1,5-a]pyrimidin-7-yl]-3.2.1]oct-8-yl}-N,N-dimethyl-2-oxoethanamine, {3-[3-(1H-indazol-4-yl)-2-pyridin-4-ylpyrazolo[1,5-a]pyrimidin-7-yl]-8-azabicyclo[3.2.1]oct-8-yl}acetonitrile, N-ethyl-3-[3-(1H-indazol-4-yl)-2-pyridin-4-ylpyrazolo[1,5-a]pyrimidin-7-yl]-8-azabicyclo[3.2.1]octane-8-carboxamide, 7-(8-acetyl-8-azabicyclo[3.2.1]oct-3-yl)-3-(1H-indazol-4-yl)-2-pyridin-4-ylpyrazolo[1,5-a]pyrimidine, 3-[3-(1H-indazol-4-yl)-2-pyridin-4-ylpyrazolo[1,5-a]pyrimidin-7-yl]-N,N-dimethyl-8-azabicyclo[3.2.1]octane-8-carboxamide, tert-butyl (1S,4S)-5-{[3-(4-chloro-3-methoxyphenyl)-2-pyridin-4-ylpyrazolo[1,5-a]pyrimidin-7-yl]methyl}-2,5-diazabicyclo[2.2.1]heptane-2-carboxylate, tert-butyl(1S,4S)-5-{4-[3-(7-fluoro-1H-indazol-4-yl)-2-pyridin-4-ylpyrazolo[1,5-a]pyrimidin-5-yl]phenyl}-2,5-diazabicyclo-[2.2.1]heptane-2-carboxylate, tert-butyl (1S,4S)-5-{3-[3-(1H-indazol-4-yl)-2-pyridin-4-ylpyrazolo[1,5-a]pyrimidin-7-yl]phenyl}-2,5-diazabicyclo[2.2.1]heptane-2-carboxylate, 7-{3-[(1S,4S)-2,5-diazabicyclo[2.2.1]hept-2-yl]phenyl}-3-(1H-indazol-4-yl)-2-pyridin-4-ylpyrazolo[1,5-a]pyrimidine, tert-butyl(1S,4S)-5-{4-[3-(7-chloro-1H-indazol-4-yl)-2-pyridin-4-ylpyrazolo[1,5-a]pyrimidin-7-yl]phenyl}-2,5-diazabicyclo[2.2.1]heptane-2-carboxylate, 3-(1H-indazol-4-yl)-7-{3-[(1S,4S)-5-methyl-2,5-diazabicyclo[2.2.1]hept-2-yl]phenyl}-2-pyridin-4-ylpyrazolo[1,5-a]pyrimidine, 3-(7-chloro-1H-indazol-4-yl)-7-{4-[(1S,4S)-2,5-diazabicyclo[2.2.1]hept-2-yl]phenyl}-2-pyridin-4-yl pyrazolo[1,5-a]pyrimidine, tert-butyl(2S)-2-({3-[3-(1H-indazol-4-yl)-2-pyridin-4-ylpyrazolo[1,5-a]pyrimidin-7-yl]-8-azabicyclo[3.2.1]oct-8-yl}carbonyl)pyrrolidine-1-carboxylate, 3-(1H-indazol-4-yl)-7-(8-L-prolyl-8-azabicyclo[3.2.1]oct-3-yl)-2-pyridin-4-ylpyrazolo[1,5-a]pyrimidine, 1-{3-[3-(1H-indazol-4-yl)-2-pyridin-4-ylpyrazolo[1,5-a]pyrimidin-7-yl]-8-azabicyclo[3.2.1]oct-8-yl}propan-2-one, ethyl 3-[3-(7-fluoro-1H-indazol-4-yl)-2-pyridin-4-ylpyrazolo[1,5-a]pyrimidin-5-yl]-8-azabicyclo[3.2.1]octane-8-carboxylate, 7-{4-[(1S,4S)-2,5-diazabicyclo[2.2.1]hept-2-yl]-2-fluorophenyl}-3-(3-methoxyphenyl)-2-pyridin-4-ylpyrazolo[1,5-a]pyrimidine, 3-(7-{4-[(1S,4S)-2,5-diazabicyclo[2.2.1]hept-2-yl]-2-fluorophenyl}-2-pyridin-4-ylpyrazolo[1,5-a]pyrimidin-3-yl)phenol, 3-(7-{2-fluoro-4-[( 1S,4S)-5-methyl-2,5-diazabicyclo[2.2.1]hept-2-yl]phenyl}-2-pyridin-4-ylpyrazolo[1,5-a]pyrimidin-3-yl)phenol, 7-{4-[(1S,4S)-2,5-diazabicyclo[2.2.1]hept-2-yl]-2-methylphenyl}-3-(7-fluoro-1H-indazol-4-yl)-2-pyridin-4-ylpyrazolo[1,5-a]pyrimidine, 3-(7-fluoro-1H-indazol-4-yl)-7-{2-methyl-4-[(1S,4S)-5-methyl-2,5-diazabicyclo-[2.2.1]hept-2-yl]phenyl}-2-pyridin-4-ylpyrazolo[1,5-a]pyrimidine, 7-(8-azabicyclo[3.2.1]oct-3-yl)-3-(7-fluoro-1H-indazol-4-yl)-2-pyridin-4-yl pyrazolo[1,5-a]pyrimidine, {3-[3-(7-fluoro-1H-indazol-4-yl)-2-pyridin-4-yl pyrazolo[1,5-a]pyrimidin-7-yl]-8-azabicyclo[3.2.1]oct-8-yl}acetonitrile, 3-(7-chloro-1H-indazol-4-yl)-5-{4-[(1S,4S)-2,5-diazabicyclo[2.2.1]hept-2-yl]phenyl}-2-pyridin-4-ylpyrazolo[1,5-a]pyrimidine, 3-(1H-indazol-4-yl)-7-[6-(8-methyl-3,8-diazabicyclo[3.2.1]oct-3-yl)pyridin-3-yl]-2-pyridin-4-ylpyrazolo[1,5-a]pyrimidine, 3-(7-chloro-1H-indazol-4-yl)-7-{4-[(1S,4S)-5-methyl-2,5-diazabicyclo[2.2.1]hept-2-yl]phenyl}-2-pyridin-4-ylpyrazolo[1,5-a]pyrimidine, 7-(1,4-dioxaspiro[4.5]dec-8-yl)-3-(1H-indazol-4-yl)-2-pyridin-4-ylpyrazolo[1,5-a]pyrimidine, 3-(1H-indazol-4-yl)-7-{4-[(1S,4S)-5-methyl-2,5-diazabicyclo[2.2.2]oct-2-yl]phenyl}-2-pyridin-4-ylpyrazolo[1,5-a]pyrimidine, 7-{4-[(1S,4S)-2,5-diazabicyclo-[2.2.2]oct-2-yl]phenyl}-3-(1H-indazol-4-yl)-2-pyridin-4-ylpyrazolo[1,5-a]pyrimidine, 7-{2-[(1S,4S)-2,5-diazabicyclo[2.2.1]hept-2-yl]-4,6-difluorophenyl}-3-(1H-indazol-4-yl)-2-pyridin-4-ylpyrazolo[1,5-a]pyrimidine, 5-[(1S,4S)-2,5-diazabicyclo[2.2.1]hept-2-yl]-2-[3-( 1H-indazol-4-yl)-2-pyridin-4-ylpyrazolo[1,5-a]pyrimidin-7-yl]-N,N-dimethylaniline, 7-{2,4-difluoro-6-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, 2-[3-(1H-indazol-4-yl)-2-pyridin-4-ylpyrazolo[1,5]pyrimidin-7-yl]-N,N-dimethyl-5-[(1S,4S)-5-methyl-2,5-diazabicyclo[2.2.1]hept-2-yl]aniline, 7-{cis-4-[(1S,4S)-2,5-diazabicyclo[2.2.1]hept-2-yl]cyclohexyl}-3-(1H-indazol-4-yl)-2-pyridin-4-yl pyrazolo[1,5-a]pyrimidine, 3-(1H-indazol-4-yl)-7-[cis-4-(3-oxa-8-azabicyclo[3.2.1]oct-8-yl)cyclohexyl]-2-pyridin-4-ylpyrazolo[1,5-a]pyrimidine, 3-(1H-indazol-4-yl)-7-[trans-4-(3-oxa-8-azabicyclo[3.2.1]-oct-8-yl)cyclohexyl]-2-pyridin-4-ylpyrazolo[1,5-a]pyrimidine, 3-(1H-indazol-4-yl)-7-[cis-4-yl)-7-[cis-4-(8-oxa-3-azabicyclo[3.2.1]oct-3-yl)cyclohexyl]-2-pyridin-4-ylpyrazolo[1,5-a]pyrimidine, 3-(1H-indazol-4-yl)-7-[trans-4-(8-oxa-3-azabicyclo[3.2.1]oct-3-yl)cyclohexyl]-2-pyridin-4-ylpyrazolo[1,5-a]pyrimidine, 3-(1H-indazol-4-yl)-7-{cis-4-[(1S,4S)-2-oxa-5-azabicyclo[2.2.1]hept-5-yl]cyclohexyl}-2-pyridin-4-ylpyrazolo[1,5-a]pyrimidine, 3-(1H-indazol-4-yl)-7-{trans-4-[(1S,4S)-2-oxa-5-azabicyclo[2.2.1]hept-5-yl]cyclohexyl}-2-pyridin-4-ylpyrazolo[1,5-a]pyrimidine, 7-{trans-4-[(1S,4S)-2,5-diazabicyclo[2.2.1]hept-2-yl]cyclohexyl}-3-(1H-indazol-4-yl)-2-pyridin-4-yl pyrazolo[1,5-a]pyrimidine, 7-{4-[(1S,4S)-2,5-diazabicyclo[2.2.1]hept-2-yl]-3-(trifluoromethyl)phenyl}-3-(1H-indazol-4-yl)-2-pyridin-4-ylpyrazolo[1,5-a]pyrimidine, 7-{4-[(1S,4S)-2,5-diazabicyclo[2.2.1]hept-2-yl]-1-naphthyl}-3-(1H-indazol-4-yl)-2-pyridin-4-ylpyrazolo[1,5-a]pyrimidine, 3-(1H-indazol-4-yl)-7-{4-[(1S,4S)-5-methyl-2,5-diazabicyclo[2.2.1]hept-2-yl]-3-(trifluoromethyl)phenyl}-2-pyridin-4-ylpyrazolo[1,5-a]pyrimidine, 3-(1H-indazol-4-yl)-7-{4-[(1S,4S)-5-methyl-2,5-diazabicyclo[2.2.1]hept-2-yl]-1-naphthyl}-2-pyridin-4-ylpyrazolo[1,5-a]pyrimidine, 7-{4-[(1S,4S)-2,5-diazabicyclo[2.2.1]hept-2-yl]-3,5-difluorophenyl}-3-(1H-indazol-4-yl)-2-pyridin-4-ylpyrazolo[1,5-a]pyrimidine, 7-{4-[(1S,4S)-2,5-diazabicyclo[2.2.1]hept-2-yl]-2,3-difluorophenyl}-3-(1H-indazol-4-yl)-2-pyridin-4-ylpyrazolo[1,5-a]pyrimidine, 7-{4-[(1S,4S)-2,5-diazabicyclo[2.2.1]hept-2-yl]-2,5-difluorophenyl}-3-(1H-indazol-4-yl)-2-pyridin-4-ylpyrazolo[1,5-a]pyrimidine, 7-{3,5-difluoro-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, 7-{2,3-difluoro-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, 7-{2,5-difluoro-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, 7-{4-[(1S,4S)-5-ethyl-2,5-diazabicyclo[2.2.1]-hept-2-yl]-2,6-difluorophenyl}-3-(1H-indazol-4-yl)-2-pyridin-4-ylpyrazolo[1,5-a]pyrimidine, 7-{2,6-difluoro-4-[(1S,4S)-5-isobutyl-2,5-diazabicyclo[2.2.1]hept-2-yl]phenyl}-3-(1H-indazol-4-yl)-2-pyridin-4-ylpyrazolo[1,5-a]pyrimidine, 7-{2,6-difluoro-4-[(1S,4S)-5-isopropyl-2,5-diazabicyclo[2.2.1]hept-2-yl]phenyl}-3-(1H-indazol-4-yl)-2-pyridin-4-ylpyrazolo[1,5-a]pyrimidine, 7-{4-[(1S,4S)-5-cyclobutyl-2,5-diazabicyclo[2.2.1]hept-2-yl]-2,6-difluorophenyl}-3-(1H-indazol-4-yl)-2-pyridin-4-ylpyrazolo[1,5-a]pyrimidine, 7-(1,4-dioxaspiro[4.5]dec-8-yl)-3-(7-fluoro-1H-indazol-4-yl)-2-pyridin-4-yl pyrazolo[1,5-a]pyrimidine, 7-{4-[(1S,4S)-2,5-diazabicyclo[2.2.2]oct-2-yl]-2-fluorophenyl}-3-(1H-indazol-4-2-pyridin-4-ylpyrazolo[1,5-a]pyrimidine, 7-{2-fluoro-4-[(1S,4S)-5-methyl-2,5-diazabicyclo[2.2.2]oct-2-yl]phenyl}-3-(1H-indazol-4-yl)-2-pyridin-4-yl pyrazolo[1,5-a]pyrimidine, 3-(1H-indazol-4-yl)-2-pyridin-4-yl-7-{2,3,5,6-tetrafluoro-4-[(1S,4S)-5-methyl-2,5-diazabicyclo[2.2.1]hept-2-yl]phenyl}pyrazolo[1,5-a]pyrimidine, tert-butyl(1S,4S)-5-{3-chloro-4-[3-(7-fluoro-1H-indazol-4-yl)-2-pyridin-4-ylpyrazolo[1,5-a]pyrimidin-7-yl]phenyl}-2,5-diazabicyclo[2.2.1]heptane-2-carboxylate, 7-{cis-4-[(1S,4S)-2,5-diazabicyclo[2.2.1]hept-2-yl]cyclohexyl}-3-(7-fluoro-1H-indazol-4-yl)-2-pyridin-4-ylpyrazolo[1,5-a]pyrimidine, 3-(7-fluoro-1H-indazol-4-yl)-7-{cis-4-[(1S,4S)-2-oxa-5-azabicyclo[2.2.1]hept-5-yl]cyclohexyl}-2-pyridin-4-ylpyrazolo[1,5-a]pyrimidine, 3-(7-fluoro-1H-indazol-4-yl)-7-{trans-4-[(1S,4S)-2-oxa-5-azabicyclo[2.2.1]hept-5-yl]cyclohexyl}-2-pyridin-4-ylpyrazolo[1,5-a]pyrimidine, 3-(7-fluoro-1H-indazol-4-yl)-7-[cis-4-(8-oxa-3-azabicyclo[3.2.1]oct-3-yl)cyclohexyl]-2-pyridin-4-ylpyrazolo[1,5-a]pyrimidine, 3-(7-fluoro-1H-indazol-4-yl)-7-[trans-4-(8-oxa-3-azabicyclo[3.2.1]oct-3-yl)cyclohexyl]-2-pyridin-4-ylpyrazolo[1,5-a]pyrimidine, 3-(7-fluoro-1H-indazol-4-yl)-7-[cis-4-(3-oxa-8-azabicyclo[3.2.1]oct-8-yl)cyclohexyl]-2-pyridin-4-ylpyrazolo[1,5-a]pyrimidine, 3-(7-fluoro-1H-indazol-4-yl)-7-[trans-4-(3-oxa-8-azabicyclo[3.2.1]oct-8-yl)cyclohexyl]-2-pyridin-4-ylpyrazolo[1,5-a]pyrimidine, 7-{trans-4-[(1S,4S)-2,5-diazabicyclo[2.2.1]hept-2-yl]cyclohexyl}-3-(7-fluoro-1H-indazol-4-yl)-2-pyridin-4-ylpyrazolo[1,5-a]pyrimidine, 7-{2-chloro-4-[(1S,4S)-2,5-diazabicyclo[2.2.1]-hept-2-yl]phenyl}-3-(7-fluoro-1H-indazol-4-yl)-2-pyridin-4-ylpyrazolo[1,5-a]pyrimidine, 7-{4-[(1S,4S)-2,5-diazabicyclo[2.2.1]hept-2-yl]-3-fluorophenyl}-3-(1H-indazol-4-yl)-2-pyridin-4-ylpyrazolo[1,5-a]pyrimidine, 7-{4-[(1S,4S)-2,5-diazabicyclo[2.2.1]hept-2-yl]-2-(trifluoromethyl)phenyl}-3-(1H-indazol-4-yl)-2-pyridin-4-ylpyrazolo[1,5-a]pyrimidine, 7-{2-bromo-4-[(1S,4S)-2,5-diazabicyclo[2.2.1]hept-2-yl]phenyl}-3-(1H-indazol-4-yl)-2-pyridin-4-ylpyrazolo[1,5-a]pyrimidine, 7-{3-fluoro-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, 3-(1H-indazol-4-yl)-7-{4-[(1S,4S)-5-methyl-2,5-diazabicyclo[2.2.1]hept-2-yl]-2-(trifluoromethyl)phenyl}-2-pyridin-4-ylpyrazolo[1,5-a]pyrimidine, 7-{2-bromo-4-[( 1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, 7-(1,4-dioxaspiro[4.5]dec-7-en-8-yl)-3-(1H-indazol-4-yl)-2-pyridin-4-ylpyrazolo[1,5-a]pyrimidine, 7-{2,6-difluoro-4-[(1S,4S)-5-methyl-5-oxido-2,5-diazabicyclo[2.2.1]hept-2-yl]phenyl}-3-(1H-indazol-4-yl)-2-pyridin-4-ylpyrazolo[1,5-a]pyrimidine, 3-(1H-indazol-4-yl)-7-{5-[(1S,4S)-2-oxa-5-azabicyclo[2.2.1]hept-5-ylmethyl]furan-3-yl}-2-pyridin-4-ylpyrazolo[1,5-a]pyrimidine, tert-butyl(1S,4S)-5-({4-[3-(1H-indazol-4-yl)-2-pyridin-4-ylpyrazolo[1,5-a]pyrimidin-7-yl]furan-2-yl}methyl)-2,5-diazabicyclo[2.2.1]heptane-2-carboxylate, 7-{5-[(1S,4S)-2,5-diazabicyclo[2.2.1]hept-2-ylmethyl]furan-3-yl}-3-(1H-indazol-4-yl)-2-pyridin-4-ylpyrazolo[1,5-a]pyrimidine, tert-butyl(1S,4S)-5-({5-[3-(1H-indazol-4-yl)-2-pyridin-4-ylpyrazolo[1,5-a]pyrimidin-7-yl]thiophen-2-yl}methyl)-2,5-diazabicyclo[2.2.1]heptane-2-carboxylate, 3-(1H-indazol-4-yl)-7-{5-[(1S,4S)-2-oxa-5-azabicyclo[2.2.1]hept-5-ylmethyl]thiophen-2-yl}-2-pyridin-4-ylpyrazolo[1,5-a]pyrimidine, 7-{5-[(1S,4S)-2,5-diazabicyclo[2.2.1]hept-2-ylmethyl]thiophen-2-yl}-3-(1H-indazol-4-yl)-2-pyridin-4-ylpyrazolo[1,5-a]pyrimidine, ethyl (3-endo)-3-[3-(1H-indazol-4-yl)-6-methyl-2-pyridin-4-ylpyrazolo[1,5-a]pyrimidin-7-yl]-8-azabicyclo[3.2.1]octane-8-carboxylate, 3-(1H-indazol-4-yl)-7-[6-(8-oxa-3-azabicyclo[3.2.1]oct-3-yl)pyridin-3-yl]-2-pyridin-4-ylpyrazolo[1,5-a]pyrimidine, 3-(1H-indazol-4-yl)-7-{6-[(1S,4S)-2-oxa-5-azabicyclo-[2.2.1]hept-5-yl]pyridin-3-yl}-2-pyridin-4-ylpyrazolo[1,5-a]pyrimidine, tert-butyl(1S,4S)-5-{3-[3-(1H-indazol-4-yl)-2-pyridin-4-ylpyrazolo[1,5-a]pyrimidin-7-yl]benzyl}-2,5-diazabicyclo[2.2.1]heptane-2-carboxylate, tert-butyl(1S,4S)-5-{4-[3-(1H-indazol-4-yl)-2-pyridin-4-ylpyrazolo[1,5-a]pyrimidin-7-yl]benzyl}-2,5-diazabicyclo[2.2.1]-heptane-2-carboxylate, 3-(1H-indazol-4-yl)-7-{4-[(1S,4S)-2-oxa-5-azabicyclo[2.2.1]-hept-5-ylmethyl]phenyl}-2-pyridin-4-ylpyrazolo[1,5-a]pyrimidine, 7-{4-[(1S,4S)-2,5-diazabicyclo[2.2.1]hept-2-ylmethyl]-2-fluorophenyl}-3-(1H-indazol-4-yl)-2-pyridin-4-yl pyrazolo[1,5-a]pyrimidine, 7-{2-fluoro-4-[(1S,4S)-2-oxa-5-azabicyclo[2.2.1]hept-5-ylmethyl]phenyl}-3-(1H-indazol-4-yl)-2-pyridin-4-ylpyrazolo[1,5-a]pyrimidine, 7-{2-fluoro-4-[(1R,4R)-2-oxa-5-azabicyclo[2.2.1]hept-5-ylmethyl]phenyl}-3-(1H-indazol-4-yl)-2-pyridin-4-ylpyrazolo[1,5-a]pyrimidine, 7-{3-[(1S,4S)-2,5-diazabicyclo[2.2.1]hept-2-ylmethyl]phenyl}-3-(1H-indazol-4-yl)-2-pyridin-4-ylpyrazolo[1,5-a]pyrimidine, 7-{4-[(1S,4S)-2,5-diazabicyclo[2.2.1]hept-2-ylmethyl]phenyl}-3-1H-indazol-4-yl)-2-pyridin-4-ylpyrazolo[1,5-a]pyrimidine, 9-{3-fluoro-4-[3-(1H-indazol-4-yl)-2-pyridin-4-ylpyrazolo[1,5-a]pyrimidin-7-yl]benzyl}-3,7-dioxa-9-azabicyclo[3.3.1]nonane and pharmaceutically acceptable salts thereof.

Compounds of Formula A may be obtained as inorganic or organic salts using methods known to those skilled in the art, for example Richard C. Larock, Comprehensive Organic Transformations, VCH publishers, 411-415, 1989. It is well known to one skilled in the art that an appropriate salt form is chosen based on physical and chemical stability, flowability, hydroscopicity and solubility.

Pharmaceutically acceptable salts of the compounds of Formula A with an acidic moiety may be formed from organic and inorganic bases. For example with alkali metals or alkaline earth metals such as sodium, potassium, lithium, calcium, or magnesium or organic bases and N-tetraalkylammonium salts such as N-tetrabutylammonium salts. Similarly, when a compound of this invention contains a basic moiety, salts may be formed from organic and inorganic acids. For example salts may be formed from acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids. Suitable examples of pharmaceutically acceptable salts include, but are not limited, to sulfate; citrate, acetate; oxalate; chloride; bromide; iodide; nitrate; bisulfate; phosphate; acid phosphate; isonicotinate; lactate; salicylate; acid citrate; tartrate; oleate; tannate; pantothenate; bitartrate; ascorbate; succinate; maleate; gentisinate; fumarate; gluconate; glucaronate; saccharate; formate; benzoate; glutamate; methanesulfonate; ethanesulfonate; benzenesulfonate; p-toluenesulfonate; pamoate (i.e., 1,1′-methylene-bis-(2-hydroxy-3-naphthoate)); and salts of fatty acids such as caproate, laurate, myristate, palmitate, stearate, oleate, linoleate, and linolenate salts. The compounds can also be used in the form of esters, carbamates and other conventional prodrug forms, which when administered in such form, convert to the active moiety in-vivo.

The present invention accordingly provides a pharmaceutical composition, which comprises an effective amount of a compound of Formula A in combination or association with a pharmaceutically acceptable carrier. Pharmaceutical compositions are prepared in accordance with acceptable pharmaceutical procedures, such as described in Remingtons Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable. As used herein, the term “effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease; (2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and (3) ameliorating the disease; for example, ameliorating a disease, condition or disorder in an individual that is experiencing or displaying the pathology or symptomatology of the disease, condition or disorder (i.e., reversing the pathology and/or symptomatology).

The compounds of this invention may be formulated neat or may be combined with one or more pharmaceutically acceptable carriers for administration. Suitable carriers include but are not limited to, for example, solvents, diluents and the like, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parenterally in the form of sterile injectable solution or suspension containing from about 0.05 to 5% suspending agent in an isotonic medium. Such pharmaceutical preparations may contain, for example, from about 0.05 up to about 90% of the active ingredient in combination with the carrier, more usually between about 5% and 60% by weight.

In some embodiments, the formulations are administered transdermally which includes all methods of administration across the surface of the body and the inner linings of body passages including epithelial and mucosal tissues. Such administration may be in the form of a lotion, cream, colloid, foam, patch, suspension, or solution.

The effective dosage of active ingredient employed may vary depending on the particular compound employed, the mode of administration and the severity of the condition being treated. However, in general, satisfactory results are obtained when the compounds of the invention are administered at a daily dosage of from about 0.5 to about 1000 mg/kg of animal body weight, optionally given in divided doses two to four times a day, or in sustained release form. For most large mammals the total daily dosage is from about 1 to 1000 mg, preferably from about 2 to 500 mg. Dosage forms suitable for internal use comprise from about 0.5 to 1000 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation.

The compounds of this invention may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA.

The preferred pharmaceutical compositions from the standpoint of ease of preparation and administration are solid compositions, particularly tablets and hard-filled or liquid-filled capsules. Oral administration of the compounds is sometimes desirable. In some cases it may be desirable to administer the compounds directly to the airways in the form of an aerosol.

The compounds of this invention may also be administered parenterally or intraperitoneally. Solutions or suspensions of these active compounds as a free base or pharmacologically acceptable salt may be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (e.g., glycerol, propylene glycol and liquid polyethylene glycol), suitable mixtures thereof, and vegetable oils.

For the treatment of cancer, the compounds of this invention may be administered in combination with other antitumor substances or with radiation therapy. These other substances or radiation treatments may be given at the same or at different times as the compounds of this invention. These combined therapies may effect synergy and result in improved efficacy. For example, the compounds of this invention may be used in combination with mitotic inhibitors such as taxol or vinblastine, alkylating agents such as cisplatin or cyclophosamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, and antiestrogens such as tamoxifen. As used in accordance with this invention, the term an “effective amount” of a compound means either directly administering such compound, or administering a prodrug, derivative, or analog which will form an effective amount of the compound within the body.

As used in accordance with this invention, the term an “effective amount” of a compound means either directly administering such compound, or administering a prodrug, derivative, or analog which will form an effective amount of the compound within the body.

Methods of administration of a pharmaceutical composition of the invention are not specifically restricted, and can be administered in various preparations depending on the age, sex, and symptoms of the patient. For example, tablets, pills, solutions, suspensions, emulsions, granules and capsules may be orally administered. Injection preparations may be administered individually or mixed with injection transfusions such as glucose solutions and amino acid solutions intravenously. If necessary, the injection preparations are administered singly intramuscularly, intracutaneously, subcutaneously or intraperitoneally. Suppositories may be administered into the rectum.

The amount of the compound of formula A contained in a pharmaceutical composition according to the present invention is not specifically restricted, however, the dose should be sufficient to treat, ameliorate, or reduce the targeted symptoms. The dosage of a pharmaceutical composition according to the present invention will depend on the method of use, the age, sex, and condition of the patient.

The present invention also provides methods of inhibition and treatment further comprising administering an additional inhibitor of an oncoprotein kinase of the Ras/Raf/MEK pathway.

The pharmaceutical compositions of the present invention may comprise the compound of the present invention alone or in combination with other oncoprotein kinase-inhibiting compounds or chemotherapeutic agents. Chemotherapeutic agents include, but are not limited to exemestane, formestane, anastrozole, letrozole, fadrozole, taxane and derivatives such as paclitaxel or docetaxel, encapsulated taxanes, CPT-11, camptothecin derivatives, anthracycline glycosides, e.g., doxorubicin, idarubicin, epirubicin, etoposide, navelbine, vinblastine, carboplatin, cisplatin, estramustine, celecoxib, tamoxifen, raloxifen, Sugen SU-5416, Sugen SU-6668, and Herceptin.

Having described the invention, the invention is further illustrated by the following non-limiting examples.

### Examples

### Example 1

### 3-(7-{6-[(1-Azabicyclo[2.2.2]oct-4-ylmethyl)amino]pyridin-3-yl}-2-pyridin-4-ylpyrazolo[1,5-a]pyrimidin-3-yl)phenol

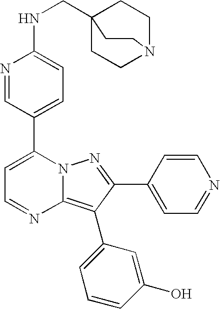

Step 1: A slurry of 5-acetyl-2-bromopyridine (5 g, 0.025 mol) in 45 mL of dimethylformamide dimethyl acetal was heated to 110° C. for 2.5 hrs. The reaction mixture was cooled to room temperature to precipitate a yellow solid, which was filtered, rinsed with ether, and dried at 40° C. under vacuum overnight to provide 5.20 g (82% yield) of (2E)-1-(6-bromopyridin-3-yl)-3-(dimethylamino)prop-2-en-1-one, which was used without further purification.

Step 2: To 5 mL of dry ethanol was added 0.73 g (31.84 mmol) of sodium metal (after removal of mineral oil with hexane) and the mixture was stirred at 45° C. for 1 hour until the solution turned clear. A mixture of 3 g (20.38 mmol) of 3-(methoxyphenyl)acetonitrile and 3.9 g (28.66 mmol) of methyl isonicotinate in 26 mL of dry ethanol was then added and the resulting brown solution was heated at reflux for 3 hours. After cooling, the residue was evaporated and purified by silica gel chromatography eluting with 9:1 to 4:1 methylene chloride/methanol to provide 1.75 g (34% yield) of 2-(3-methoxyphenyl)-3-oxo-3-pyridin-4-yl-propionitrile.

A mixture of 1.7 g (6.74 mmol) of 2-(3-methoxyphenyl)-3-oxo-3-pyridin-4-yl-propionitrile and 17 mL POCl₃ was heated at 80° C. for 18 hours. After cooling, the POCl₃ was evaporated off. To the resulting residue was added toluene, and the mixture was then evaporated to dryness. This step was repeated to fully remove POCl₃. Ice and saturated sodium bicarbonate were added to the residue, and a solid precipitated out, providing 1 g (57% yield) of 3-chloro-2-(3-methoxyphenyl)-3-pyridin-4-yl-acrylonitrile as a white solid. MS: 271.1 [M+H].

A mixture of 1 g (3.69 mmol) of 3-chloro-2-(3-methoxyphenyl)-3-pyridin-4-yl-acrylonitrile and 0.9 mL (18.6 mmol) hydrazine hydrate in 30 mL of ethanol was heated at reflux for 6.5 hours. The mixture was allowed to cool to room temperature and the solvent was removed by evaporation. Aqueous sodium bicarbonate was stirred into the residue, and the resulting solid was collected by filtration. The solid was washed with water, and then dried under vacuum to provide 0.92 g (94% yield) of 4-[3-methoxy-phenyl]-5-pyridin-4-yl-1H-pyrazol-3-amine. MS: 267.2 [M+H].

A mixture of 4-(3-methoxyphenyl)-5-pyridin-4-yl-1H-pyrazol-3-amine (3.0g, 11.27 mmol) and pyridine hydrochloride (6.0g, 51.92 mmol) was heated at 202° C. for one hour. The reaction was then cooled to room temperature, diluted with 10 mL of ammonium hydroxide, stirred for 30 min, and then the solvent was removed under vacuum. The resulting residue was washed with 15% methanol/dichloromethane and the collected washings were dried over sodium sulfate, filtered, and evaporated to a residue that was purified via silica flash chromatography eluting with 5%-12% methanol/dichloromethane to provide 2.21g (78% yield) of 3-(3-amino-5-pyridin-4-yl-1H-pyrazol-4-yl)phenol as a beige solid.

Step 3: 1-(6-Bromo-pyridin-3-yl)-3-dimethylamino-propenone (258 mg, 1.0 mmol), 3-(3-amino-5-pyridin-4-yl-1H-pyrazol-4-yl)phenol (254 mg, 1.0 mmol), and 3 mL glacial acetic acid were combined and heated in the microwave at 120° C. for 3000 sec. Upon cooling, obtained yellow precipitate which was filtered, rinsed with 10% ethyl acetate/ether then with ether, and dried at 40° C. under reduced pressure to give 575 mg (94% yield) of 3-[7-(6-bromopyridin-3-yl)-2-pyridin-4-ylpyrazolo[1,5-a]pyrimidin-3-yl]phenol as an acetic acid salt, a yellow orange solid. MS: 444.1 [M+H].

Step 4: To a stirred suspension of 3-[7-(6-bromopyridin-3-yl)-2-pyridin-4-ylpyrazolo[1,5-a]pyrimidin-3-yl]phenol (117 mg, 0.25 mmol) in 1.2 mL of DMSO was added diisopropylethylamine (0.13 mL, 0.75 mmol) followed by 1-(1-azabicyclo[2.2.2]oct-4-yl)methanamine (74 mg, 0.5 mmol). The mixture was heated at 125° C. for 16 hours, cooled to room temperature, and purified by RP-HPLC on a Gemini™ C18 column eluted with 5-95% acetonitrile/water (0.02% TFA) to give 18 mg of 3-(7-{6-[(1-azabicyclo[2.2.2]oct-4-ylmethyl)amino]pyridin-3-yl}-2-pyridin-4-ylpyrazolo[1,5-a]pyrimidin-3-yl)phenol as a yellow-orange solid, 11% yield. MS: 504.4 [M+H].

### Example 2

### 3-(7-{6-[(3S)-1-Azabicyclo[2.2.2]oct-3-ylamino]pyridin-3-yl}-2-pyridin-4-ylpyrazolo[1,5-a]pyrimidin-3-yl)phenol

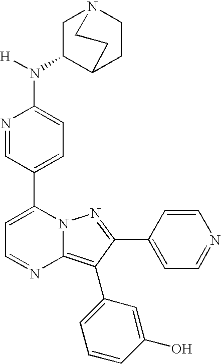

To a stirred suspension of 3-[7-(6-bromopyridin-3-yl)-2-pyridin-4-ylpyrazolo[1,5-a]pyrimidin-3-yl]phenol (117 mg, 0.25 mmol) in 1.0 mL DMSO was added diisopropylethylamine (0.23 mL, 1.35 mmol) and 3-(S)-aminoquinuclidine dihydrochloride (60 mg, 0.3 mmol) and the resulting mixture was microwaved at 150° C. for 1 hour and then purified by RP-HPLC on a Gemini™ C18 column, eluting with 5-95% acetonitrile/water (0.02% TFA) to give 21 mg (14% yield) of 3-(7-{6-[(3S)-1-azabicyclo[2.2.2]oct-3-ylamino]pyridin-3-yl}-2-pyridin-4-ylpyrazolo[1,5-a]pyrimidin-3-yl)phenol as a yellow-orange solid. MS: 490.4 [M+H].

### Example 3

### 3-(7-{6-[(3R)-1-Azabicyclo[2.2.2]oct-3-ylamino]pyridin-3-yl}-2-pyridin-4-ylpyrazolo[1,5-a]pyrimidin-3-yl)phenol

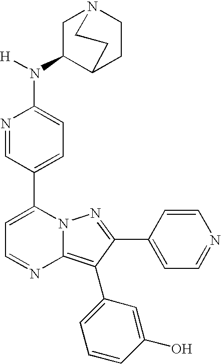

Following the procedure for Example 2, 3-[7-(6-bromopyridin-3-yl)-2-pyridin-4-ylpyrazolo[1,5-a]pyrimidin-3-yl]phenol and 3-(R)-aminoquinuclidine dihydrochloride reacted to give 29 mg (14% yield) of 3-(7-{6-[(3R)-1-azabicyclo[2.2.2]oct-3-ylamino]pyridin-3-yl}-2-pyridin-4-ylpyrazolo[1,5-a]pyrimidin-3-yl)phenol as a yellow solid. MS: 490.4 [M+H].

### Example 4

### (3R)—N-{4-[3-(3-Methoxyphenyl)-2-pyridin-4-ylpyrazolo[1,5-a]pyrimidin-7-yl]pyridin-2-yl}quinuclidin-3-amine

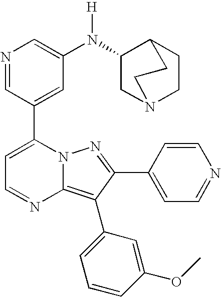

Step 1: To a solution of acetylpyridine (2.2 mL, 20 mmol) in 22 mL dichloromethane was added 3-chloroperoxybenzoic acid (3.45 g, 20 mmol) and the resulting mixture was heated to reflux for 16 hours. The solvent was then evaporated and the crude residue was chromatographed on silica gel, eluting with 0-40% methanol/ethyl acetate to provide 1 g (38% yield) of 1-(1-oxy-pyridin-4-yl)-ethanone as a white solid which was used directly in the next step.

Step 2: A solution of 1-(1-oxy-pyridin-4-yl)-ethanone (231 mg, 1.7 mmol) in 2.2 mL dimethylformamide dimethyl acetal in a 5 mL Smith process vial was microwaved at 110° C. for one hour. The resulting mixture was cooled to RT, and the precipitate was collected by filtration and then rinsed with 2% ethyl acetate/ether followed by ether. The solid was dried at 40° C. under reduced pressure to give 222 mg (68% yield) of 3-dimethylamino-1-(1-oxy-pyridin-4-yl)-propenone as a beige solid that was used directly in the next step.

Step 3: A mixture of 3-dimethylamino-1-(1-oxy-pyridin-4-yl)-propenone (222 mg, 1.15 mmol) and 4-(3-methoxyphenyl)-5-pyridin-4-yl-1H-pyrazol-3-amine (308 mg, 1.15 mmol) in 2 mL of glacial acetic acid was heated in the microwave at 120° C. for one hour. The reaction mixture was then cooled to room temperature, and the resulting yellow precipitate was collected by filtration, and rinsed with 10% ethyl acetate/ether and then with ether. The solid was dried at 40° C. under reduced pressure to give 330 mg (65% yield) of 3-(3-methoxyphenyl)-7-(1-oxidopyridin-4-yl)-2-pyridin-4-ylpyrazolo[1,5-a]pyrimidine as yellow solid that was used directly in the next step. MS: 396.1 [M+H].

Step 4: A solution of 3-(3-methoxyphenyl)-7-(1-oxidopyridin-4-yl)-2-pyridin-4-ylpyrazolo[1,5-a]pyrimidine (264 mg, 0.67 mmol) was refluxed in POCl₃ for one hour and then cooled to room temperature. The mixture was then evaporated with toluene, quenched with cold saturated NaHCO₃, and extracted into ether. The organic phase was dried and evaporated under vacuum to give 272 mg (99% yield) of 7-(2-chloropyridin-4-yl)-3-(3-methoxyphenyl)-2-pyridin-4-ylpyrazolo[1,5-a]pyrimidine as a yellow solid that was used directly in the next step. MS: 414.1 [M+H].

Step 5: A mixture of 7-(2-chloropyridin-4-yl)-3-(3-methoxyphenyl)-2-pyridin-4-ylpyrazolo[1,5-a]pyrimidine (272 mg, 0.66 mmol), 3-(S)-aminoquinuclidine dihydrochloride (654 mg, 3.29 mmol), and DIPEA (1.28 g, 9.9 mmol), in 2.0 mL anhydrous DMSO was microwaved at 170° C. for one hour. The resulting crude reaction mixture was purified by HPLC to give 43 mg (13% yield) of (3R)—N-{4-[3-(3-methoxyphenyl)-2-pyridin-4-ylpyrazolo[1,5-a]pyrimidin-7-yl]pyridin-2-yl}quinuclidin-3-amine as a yellow solid. MS: 504.5 [M+H].

### Example 5

### 5-(7-{6-[(3R)-1-Azabicyclo[2.2.2]oct-3-ylamino]pyridin-3-yl}-3-(4-chloro-3-methoxyphenyl)-2-pyridin-4-ylpyrazolo[1,5-a]pyrimidine

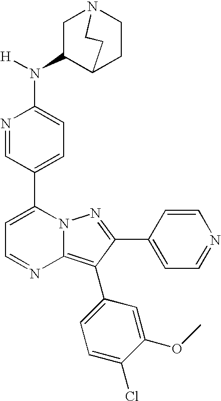

Step 1: A mixture of (2E)-1-(4-bromophenyl)-3-(dimethylamino)prop-2-en-1-one (125 mg, 0.5 mmol), (4-chloro-3-methoxyphenyl)-5-pyridin-4-yl-1H-pyrazol-3-amine (150 mg, 0.5 mmol; prepared following the procedure of Example 1, Step 2, starting with (4-chloro-3-methoxyphenyl)acetonitrile), and 1.5 mL glacial acetic acid was heated in the microwave at 120° C. for 1 hour. The acetic acid was then removed under reduced pressure, saturated NaHCO₃ was added and the resulting mixture was extracted with dicloromethane with 3% MeOH. The organic phase was dried and evaporated under vacuum and the recovered crude product was chromatographed on silica gel to give 98 mg (40% yield) of 7-(6-bromopyridin-3-yl)-3-(4-chloro-3-methoxyphenyl)-2-pyridin-4-ylpyrazolo[1,5-a]pyrimidine as a yellow solid. MS: 494.1 [M+H].

Step 2: A mixture of 7-(6-bromopyridin-3-yl)-3-(4-chloro-3-methoxyphenyl)-2-pyridin-4-ylpyrazolo[1,5-a]pyrimidine (90 mg, 0.18 mmol), 3-(R)-aminoquinuclidine dihydrochloride (35 mg, 0.22 mmol), DIPEA (47 mg, 0.36 mmol), and 1.0 mL anhydrous DMSO was heated in the microwave at 150° C. for 3900 sec. The crude reaction mixture was then purified by HPLC to give 48 mg (44% yield) of 5-(7-{6-[(3R)-1-azabicyclo[2.2.2]oct-3-ylamino]pyridin-3-yl}-3-(4-chloro-3-methoxyphenyl)-2-pyridin-4-ylpyrazolo[1,5-a]pyrimidine as a yellow solid. MS: 538.3 [M+H].

### Example 6

### 3-{7-[(1-Azabicyclo[2.2.21oct-4-ylmethyl)amino]-2-pyridin-4-ylpyrazolo[1,5-a]pyrimidin-3-yl}phenol

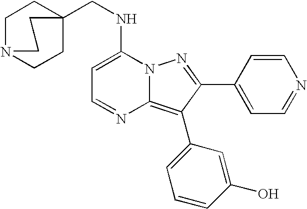

Step 1: A mixture of 4-[3-(methoxy)phenyl]-5-pyridin-4-yl-1H-pyrazol-3-amine (1.5 g, 5.63 mmol) and diethyl ethoxymethylene malonate (1.4 mL, 6.9 mmol) in glacial acetic acid (15 mL) was heated under reflux for 2.5 hours. The mixture was cooled and triturated with ether. The solid was collected by filtration, washed with ether and dried. The crude product was purified by silica gel flash chromatography (methanol/methylene chloride) to give 1.28 g (58% yield) of ethyl 3-(3-methoxyphenyl)-7-oxo-2-pyridin-4-yl-4,7-dihydropyrazolo[1,5-a]pyrimidine-6-carboxylate as a brown solid. MS: 391.2 [M+H].

Step 2: A mixture of ethyl 3-(3-methoxyphenyl)-7-oxo-2-pyridin-4-yl-4,7-dihydropyrazolo[1,5-a]pyrimidine-6-carboxylate (1.2 g, 3.07 mmol) and 2.5 N solution of sodium hydroxide (5.5 mL) was heated at reflux for 4 hours. The mixture was cooled, acidified with 2N HCl and the solid was collected by filtration, then washed with water and dried to yield 1.03 g (92%) of 3-(3-methoxyphenyl)-7-oxo-2-pyridin-4-yl-4,7-dihydropyrazolo[1,5-a]pyrimidine-6-carboxylic acid as a beige solid, 220°-225° C. MS: 363.2 [M+H].

Step 3: To a refluxing Dowtherm™ (30 mL) was added 3-(3-methoxyphenyl)-7-oxo-2-pyridin-4-yl-4,7-dihydropyrazolo[1,5-a]pyrimidine-6-carboxylic acid (1.0 g, 2.76 mmol) in one portion and the resulting mixture was heated at 250° C. for 45 minutes. After cooling to room temperature, the solid was collected by filtration, washed with ether and dried to provide 0.86 g (98% yield) of 3-(3-methoxyphenyl)-2-pyridin-4-ylpyrazolo[1,5-a]pyrimidin-7(4H)-one as yellow solid, 110°-115° C. MS: 319.2 [M+H].

Step 4: A mixture of 3-(3-methoxyphenyl)-2-pyridin-4-ylpyrazolo[1,5-a]pyrimidin-7(4H)-one (0.85 g, 2.6 mmol), N,N-diethylaniline (0.9 mL) and phosphorous oxychloride (9.0 mL) was heated at 110° C. for 2 hours. The mixture was allowed to cool and the excess phosphorous oxychloride was evaporated to dryness, followed by re-evaporation twice from toluene. The residue was cooled in an ice bath, neutralized with saturated solution of sodium bicarbonate and extracted with 10% methanol in methylene chloride. The organic layer was dried over anhydrous sodium sulfate, filtered and the filtrate was evaporated to yield an oil. The crude product was purified by silica gel flash chromatography, eluting with 1% methanol in methylene chloride, to give 1.28 g (58% yield) of 7-chloro-3-(3-methoxyphenyl)-2-pyridin-4-ylpyrazolo[1,5-a]pyrimidine as a yellow solid, 135°-140° C. MS: 337.2 [M+H].

Step 5: To a cold (0°-5° C.) solution of 1-(1-azabicyclo[2.2.2]oct-4-yl)methanamine (0.13 g, 0.9 mmol) and N,N-diisopropylethylamine (0.3 mL, 1.76 mmol) in acetonitrile (5 mL) was added 7-chloro-3-(3-methoxyphenyl)-2-pyridin-4-ylpyrazolo[1,5-a]pyrimidine (0.15 g, 0.44 mmol) in portions over a period of 5 minutes, and the resulting mixture was stirred at 5° C. for 2 hours. The solvent was evaporated and the residue was stirred with a saturated solution of sodium bicarbonate. The solid was collected by filtration, washed with water and dried. The resulting crude solid was purified by preparative reverse phase HPLC (acetonitrile/water/trifluroacetic acid) to give 0.12g (49% yield) of (1-aza-bicyclo [2,2,2]oct-4ylmethyl)-3-(3-methoxy-phenyl)-2-pyridin-4-yl-pyrazolo[1,5,a]pyrimidin-7-yl]-amine as a yellow solid. MS: 441.3 [M+H].

Step 6: A mixture of (1-aza-bicyclo[2,2,2]oct-4ylmethyl)-3-(3-methoxy-phenyl)-2-pyridin-4-yl-pyrazolo[1,5,a]pyrimidin-7-yl]-amine (0.092 g, 0.21 mmol) and pyridine hydrochloride (1.2 g, 10.4 mmol) was heated a 205° C. for 1 hour. After cooling, the mixture was basified with a solution of ammonium hydroxide and the solvent was evaporated to dryness to yield a crude residue. The residue was washed with 10% methanol in methylene chloride and the filtrate was dried over anhydrous sodium sulfate, filtered and the filtrate was evaporated to yield an oil. The crude oil was purified by preparative reverse phase HPLC (acetonitrile/water/trifluroacetic acid) to provide 0.025 g (28% yield) of 3-{7-[(1-azabicyclo[2.2.2]oct-4-ylmethyl)amino]-2-pyridin-4-ylpyrazolo[1,5-a]pyrimidin-3-yl}phenol as a yellow solid. MS: 427.3 [M+H].

### Example 7

### Ethyl 3-(3-(3-methoxyphenyl)-2-(pyridin-4-yl)pyrazolo[1,5-a]pyrimidin-7-yl)-8-azabicyclo[3.2.1]octane-8-carboxylate

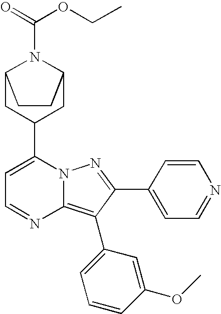

Step 1: A mixture of tosylmethylisocyanide (5 g, 25.6 mmol) and ethyl 3-oxo-8-azabicyclo[3.2.1]octane-8-carboxylate (3.8 g, 19.7 mmol) in DME (60 mL) and Ethanol (1.85 mL) was stirred at −10° C. while adding potassium tert-butoxide portionwise over the course of 1 hour that the temperature was maintained at <5° C. Once the addition was complete the reaction was stirred at −10° C. for 1 hour and then stirred for additional 2 hours at room temperature. The solvents were then removed under reduced pressure to give an orange brown solid. To this solid was added water (200 mL) and extracted with ether (4×, 150 mL). The organics extract was dried over anhydrous magnesium sulfate, filtered and the filtrate was evaporated to yield a brown oil. The crude mixture was purified on a silica column eluting with 30% ethylacetate in hexanes to give 2.43 g of ethyl 3-cyano-8-azabicyclo[3.2.1]octane-8-carboxylate (60% yield). MS: 209.2 [M+H].

Step 2: A 1.4M solution of methyl magnesium bromide (35.4 mL) in THF/toluene was added to a solution of ethyl 3-cyano-8-azabicyclo[3.2.1]octane-8-carboxylate (2.4 g, 11.5 mmol) in THF (50 mL) at rt. The reaction was stirred for 3 hours and quenched with ammonium chloride (100 mL). The mixture was then extracted with ether (4×, 100 mL). The combined organic extracts were dried over anhydrous magnesium sulfate, then filtered and the filtrate was evaporated to yield ethyl 3-acetyl-8-azabicyclo[3.2.1]octane-8-carboxylate as an oil. MS: 226.2 [M+H].

Step 3: A mixture of ethyl 3-acetyl-8-azabicyclo[3.2.1]octane-8-carboxylate (1.7 g, 7.68 mmol) in 25 mL of dimethylformamide dimethyl acetal was heated to 110° C. for 48 hours. The reaction mixture was then cooled to room temperature and the solvent was evaporated to provide an orange oil. The crude product was purified by silica gel flash chromatography, eluting with 50% acetone in dichloromethane to give 0.86 g (50% yield) of (E)-ethyl 3-(3-(dimethylamino)acryloyl)-8-azabicyclo[3.2.1]octane-8-carboxylate, which was used without further purification. MS: 281.2 [M+H].

Step 4: A mixture of (E)-ethyl 3-(3-(dimethylamino)acryloyl)-8-azabicyclo[3.2.1]octane-8-carboxylate (0.37 g, 1.3 mmol) and 4-(3-methoxyphenyl)-3-(pyridin-4-yl)-1H-pyrazol-5-amine (0.35 g, 1.3 mmol) in acetic acid (5 mL) was stirred at 80° C. for 2 h. The reaction was cooled to room temperature and the solvent was evaporated. The crude mixture was purified on silica eluting with 50% acetone in dicholormethane to give ethyl 3-(3-(3-methoxyphenyl)-2-(pyridin-4-yl)pyrazolo[1,5-a]pyrimidin-7-yl)-8-azabicyclo[3.2.1]octane-8-carboxylate as a yellow solid. MS: 484.4 [M+H].

### Example 8

### Ethyl 3-(3-(3-hydroxyphenyl)-2-(pyridin-4-yl)pyrazolo[1,5-a]pyrimidin-7-yl)-8-azabicyclo[3.2.1]octane-8-carboxylate

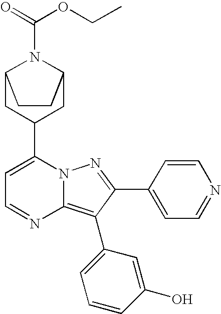

A solution of ethyl 3-(3-(3-methoxyphenyl)-2-(pyridin-4-yl)pyrazolo[1,5-a]pyrimidin-7-yl)-8-azabicyclo[3.2.1]octane-8-carboxylate (0.3 g, 0.6 mmol) in dichloromethane was cooled to 0° C. A 1 M solution of boron tribromide in dichloromethane (3.7 mL) was then added while keeping the temperature at 0° C. The reaction was stirred at 0° C. for 3 hours then allowed to warm to room temperature. The reaction was next quenched with ice water and the pH was adjusted to about 7, followed by extraction using dichloromethane (3×, 100 mL). The combined organic extracts were dried over anhydrous sodium sulfate, filtered and the filtrate was evaporated. The crude mixture was purified on silica eluting with 20% acetone in dichloromethane to give ethyl 3-(3-(3-hydroxyphenyl)-2-(pyridin-4-yl)pyrazolo[1,5-a]pyrimidin-7-yl)-8-azabicyclo[3.2.1]octane-8-carboxylate as yellow solid. ¹H NMR (400 MHz, CDCl₃) [symbol] 8.48 (d, J=4.4 Hz, 1H), 8.41 (d, J=4.8 Hz, 2H), 7.64 (d, J=6 Hz, 2H), 7.38 (t, J=8 Hz, 1H), 7.21 (d, J=7.6 Hz, 1H), 6.92 (m, 2H), 6.74 (d, J=4.4 Hz, 1H), 4.19 (m, 3H), 2.18-1.97 (m, 8H), 1.60 (brs, 2H), 1.26 (t, J=7.2 Hz, 3H). MS: 470.3 [M+H].

### Example 9

### 3-(7-(8-Azabicyclo[3.2.1]octan-3-yl)-2-(pyridin-4-yl)pyrazolo[1,5-a]pyrimidin-3-yl)phenol

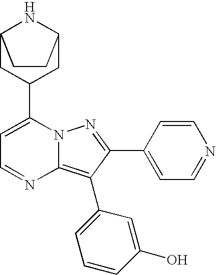

A solution of ethyl 3-(3-(3-hydroxyphenyl)-2-(pyridin-4-yl)pyrazolo[1,5-a]pyrimidin-7-yl)-8-azabicyclo[3.2.1]octane-8-carboxylate (0.1 g, 0.21 mmol) and iodotrimethylsilane (0.64 g, 3.2 mmol) in chloroform was stirred at reflux for 5 hours. The reaction was cooled to room temperature and solvent was evaporated. The crude mixture was purified using prep HPLC to give the trifluoroacetate (TFA) salt of 3-(7-(8-azabicyclo[3.2.1]octan-3-yl)-2-(pyridin-4-yl)pyrazolo[1,5-a]pyrimidin-3-yl)phenol as a yellow solid. ¹H NMR (400 MHz, DMSO) [symbol] 9.49 (s, 1H), 8.75 (s, 1H), 8.67 (d, J=4.8 Hz, 2H), 8.62 (d, J=4 Hz, 1H), 7.68 (d, J=4.8 Hz, 2H), 7.22 (t, J=8 Hz, 1H), 7.08 (d, J=4 Hz, 1H), 6.89 (d, J=4 Hz, 1H), 6.83 (d, J=6 Hz, 1H), 6.77 (d, J=6.8 Hz, 1H), 4.10 (dt, J=4.8 Hz, 7.6 Hz, 1H), 2.48 (m, 2H), 2.30 (m, 2H), 2.15 (m, 6H). MS: 398.3 [M+H].

### Example 10

### Ethyl 3-(3-(4-chloro-3-methoxyphenyl)-2-(pyridin-4-yl)pyrazolo[1,5-a]pyrimidin-7-yl)-8-azabicyclo[3.2.1]octane-8-carboxylate

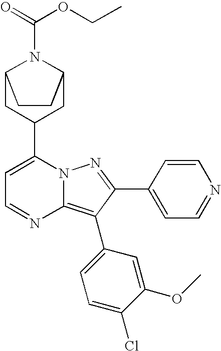

Ethyl 3-(3-(4-chloro-3-methoxyphenyl)-2-(pyridin-4-yl)pyrazolo[1,5-a]pyrimidin-7-yl)-8-azabicyclo[3.2.1]octane-8-carboxylate was synthesized according to the procedure for Example 7, Step 4, starting from (E)-ethyl 3-(3-(dimethylamino)acryloyl)-8-azabicyclo[3.2.1]octane-8-carboxylate and (4-chloro-3-methoxyphenyl)-5-pyridin-4-yl-1H-pyrazol-3-amine. MS: 518.3 [M+H].

### Example 11

### Ethyl 3-(3-(4-chloro-3-hydroxyphenyl)-2-(pyridin-4-yl)pyrazolo[1,5-a]pyrimidin-7-yl)-8-azabicyclo[3.2.1]octane-8-carboxylate

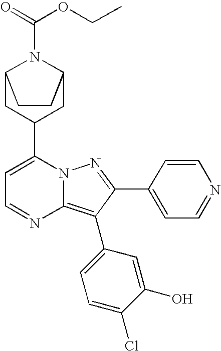

Ethyl 3-(3-(4-chloro-3-hydroxyphenyl)-2-(pyridin-4-yl)pyrazolo[1,5-a]pyrimidin-7-yl)-8-azabicyclo[3.2.1]octane-8-carboxylate was synthesized starting from ethyl 3-(3-(4-chloro-3-methoxyphenyl)-2-(pyridin-4-yl)pyrazolo[1,5-a]pyrimidin-7-yl)-8-azabicyclo[3.2.1]octane-8-carboxylate, the product of Example 10, by reaction with boron tribromide according to the procedure for Example 8. MS: 504.3 [M+H].

### Example 12

### 5-(7-(8-Azabicyclo[3.2.1]octan-3-yl)-2-(pyridin-4-yl)pyrazolo[1,5-a]pyrimidin-3-yl)-2-chlorophenol

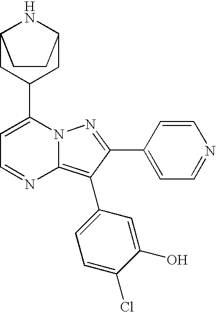

The reaction of ethyl 3-(3-(4-chloro-3-hydroxyphenyl)-2-(pyridin-4-yl)pyrazolo[1,5-a]pyrimidin-7-yl)-8-azabicyclo[3.2.1]octane-8-carboxylate, the product of Example 11, with iodotrimethylsilane, according to the procedure for Example 9, provided 5-(7-(8-azabicyclo[3.2.1]octan-3-yl)-2-(pyridin-4-yl)pyrazolo[1,5-a]pyrimidin-3-yl)-2-chlorophenol. ¹H NMR (400 MHz, DMSO) [symbol] 10.28 (s, 1H) 8.80 (s, 1H), 8.73 (d, J=8 Hz, 2H), 8.65 (d, J=4 Hz, 1 Hz), 7.76 (d, J=4 Hz, 2H), 7.39 (d, J=8 Hz, 1H), 7.12 (m, 2H), 6.87 (d, J=8 Hz, 1H), 4.10 (dt, J=5.2 Hz, 7.2 Hz, 1H), 2.48 (m, 2H), 2.30 (m, 2H), 2.15 (m, 6H). MS: 432.3 [M+H].

### Example 13

### 5-[7-(8-Acetyl-8-azabicyclo[3.2.1]oct-3-yl)-2-pyridin-4-ylpyrazolo[1,5-a]pyrimidin-3-yl]-2-chlorophenol

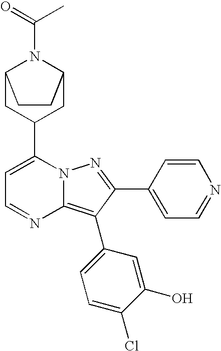

A solution of 5-(7-(8-azabicyclo[3.2.1]octan-3-yl)-2-(pyridin-4-yl)pyrazolo[1,5-a]pyrimidin-3-yl)-2-chlorophenol (0.15 g, 0.35 mmol), the product of Example 12, and triethylamine (140 μL, 1.05 mmol) in 1-methyl-2-pyrrolidinone was cooled to 0° C. Acetyl chloride (23 μL, 0.33 mmol) was then added and the reaction was stirred at 0° C. for 1 hour. The reaction was warmed to room temperature and diluted with water and dichloromethane followed by extraction with saturated sodium bicarbonate (2×50 mL). The combined organic extracts were dried over anhydrous sodium sulfate, filtered and the filtrate was evaporated. The crude mixture was purified by reverse phase HPLC (acetonitrile/water/trifluoroacetic acid) to provide 0.015 g (7% yield) of 5-[7-(8-acetyl-8-azabicyclo[3.2.1]oct-3-yl)-2-pyridin-4-ylpyrazolo[1,5-a]pyrimidin-3-yl]-2-chlorophenol. MS: 474.3 [M+H].

### Example 14

### 2-Chloro-5-{7-[8-(methylsulfonyl)-8-azabicyclo[3.2.1]oct-3-yl]-2-pyridin-4-ylpyrazolo[1,5-a]pyrimidin-3-yl}phenol

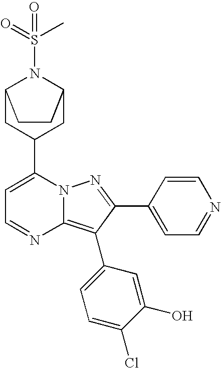

A solution of 5-(7-(8-azabicyclo[3.2.1]octan-3-yl)-2-(pyridin-4-yl)pyrazolo[1,5-a]pyrimidin-3-yl)-2-chlorophenol (0.15 g, 0.35 mmol), the product of Example 12, and triethylamine (140 μL, 1.05 mmol) in 1-methyl-2-pyrrolidinone was cooled to 0° C. Methylsulfonyl chloride (25 μL, 0.33 mmol) was then added and the reaction was stirred at 0° C. for 1 hour. The reaction was warmed to room temperature and diluted with water and dichloromethane followed by extraction with saturated sodium bicarbonate (2×50 mL). The combined organic extracts were dried over anhydrous sodium sulfate, filtered and the filtrate was evaporated. The crude mixture was purified by reverse phase HPLC (acetonitrile/water/trifluoroacetic acid) to provide 0.017 g (9% yield) of 2-chloro-5-{7-[8-(methylsulfonyl)-8-azabicyclo[3.2.1]oct-3-yl]-2-pyridin-4-ylpyrazolo[1,5-a]pyrimidin-3-yl}phenol. MS: 510.3 [M+H].

### Example 15

### 5-[7-(8-Acetyl-8-azabicyclo[3.2.1]oct-3-yl)-2-pyridin-4-ylpyrazolo[1,5-a]pyrimidin-3-yl]-2-chlorophenyl acetate

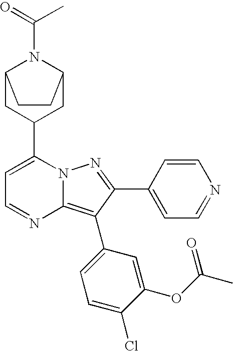

To a solution of 5-(7-(8-azabicyclo[3.2.1]octan-3-yl)-2-(pyridin-4-yl)pyrazolo[1,5-a]pyrimidin-3-yl)-2-chlorophenol (0.10 g, 0.23 mmol), the product of Example 12, in dichloromethane was added triethylamine (200 μL, 1.4 mmol). Acetyl chloride (36 μL, 0.51 mmol) was then added and the reaction was stirred for 1 hour. The reaction was extracted with saturated ammonium chloride (2×50 mL). The combined organic extracts were dried over anhydrous sodium sulfate, filtered and the filtrate was evaporated. The crude mixture was purified by preparatory TLC using 5% methanol/dichloromathane to provide 0.058 g (48% yield) of 5-[7-(8-acetyl-8-azabicyclo[3.2.1]oct-3-yl)-2-pyridin-4-ylpyrazolo[1,5-a]pyrimidin-3-yl]-2-chlorophenyl acetate. MS: 516.4 [M+H].

### Example 16

### 7-(8-Azabicyclo[3.2.1]oct-3-yl)-3-(4-chloro-3-methoxyphenyl)-2-pyridin-4-ylpyrazolo[1,5-a]pyrimidine

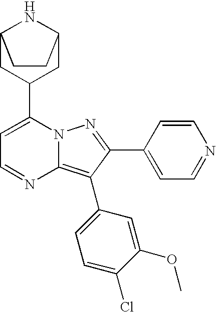

7-(8-Azabicyclo[3.2.1]oct-3-yl)-3-(4-chloro-3-methoxyphenyl)-2-pyridin-4-ylpyrazolo[1,5-a]pyrimidine was prepared from ethyl 3-(3-(4-chloro-3-methoxyphenyl)-2-(pyridin-4-yl)pyrazolo[1,5-a]pyrimidin-7-yl)-8-azabicyclo-[3.2.1]octane-8-carboxylate, the product of Example 10, using iodotrimethylsilane according to the procedure for Example 9. MS 446: [M+H].

### Example 17

### 3-(4-Chloro-3-methoxyphenyl)-7-(8-ethyl-8-azabicyclo[3.2.1]oct-3-yl)-2-pyridin-4-ylpyrazolo[1,5-a]pyrimidine

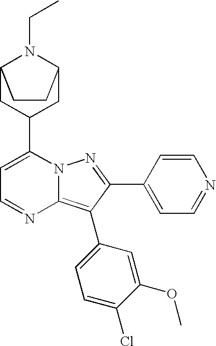

To a solution of 7-(8-azabicyclo[3.2.1]oct-3-yl)-3-(4-chloro-3-methoxyphenyl)-2-pyridin-4-ylpyrazolo[1,5-a]pyrimidine (0.258 g, 0.58 mmol), the product of Example 16, and potassium carbonate (321 mg, 2.3 mmol) in N,N-dimethylformamide was added iodoethane (92 μL, 1.16 mmol) and the reaction was stirred for 3 hours. The reaction was then added to water (25 mL) and the resulting crude solid was filtered and dried. The crude solid was purified by silica gel chromatography eluting with a 5%-10%-15%-20% gradient of methanol in dichloromathane to provide 0.172 g (63% yield) of 3-(4-chloro-3-methoxyphenyl)-7-(8-ethyl-8-azabicyclo[3.2.1]oct-3-yl)-2-pyridin-4-ylpyrazolo[1,5-a]pyrimidine. MS: 474.3 [M+H].

### Example 18

### 2-Chloro-5-[7-(8-ethyl-8-azabicyclo[3.2.1]oct-3-yl)-2-pyridin-4-ylpyrazolo[1,5-a]pyrimidin-3-yl]phenol

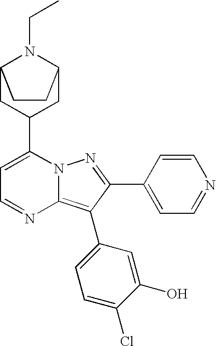

A solution of 3-(4-chloro-3-methoxyphenyl)-7-(8-ethyl-8-azabicyclo[3.2.1]oct-3-yl)-2-pyridin-4-ylpyrazolo[1,5-a]pyrimidine (0.09 g, 0.19 mmol), the product of Example 17, in dichloromethane was cooled to 0° C. A 1 M solution of boron tribromide in dichloromethane (1.12 mL) was then added while keeping the temperature at 0° C. The reaction was stirred at 0° C. for 3 hours, then was allowed to warm to room temperature. The reaction was next quenched with ice water and the pH was adjusted to about 7 followed by extraction with dichloromethane (3×100 mL). The combined organic extracts were extracted with aqueous 10% HCl (2×15 mL). The pH of the combined aqueous extracts was adjusted to about pH 10 with sodium carbonate. The resulting solid was filtered and dried to give the crude product. The remaining aqueous extracts were concentrated and the resulting solid was washed with 10% methanol/dichloromethane. The organics were concentrated in-vacuo to give additional crude product. The crude products were combined and purified on silica gel eluting with 10% methanol/dichloromethane to give 56 mg (64% yield) of 2-chloro-5-[7-(8-ethyl-8-azabicyclo[3.2.1]oct-3-yl)-2-pyridin-4-ylpyrazolo[1,5-yl]phenol. MS: 460.4 [M+H].

### Example 19

### 7-(8-Azabicyclo[3.2.1]oct-3-yl)-3-(3-methoxyphenyl)-2-pyridin-4-ylpyrazolo[1,5-a]pyrimidine

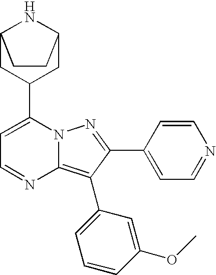

Ethyl 3-(3-(3-methoxyphenyl)-2-(pyridin-4-yl)pyrazolo[1,5-a]pyrimidin-7-yl)-8-azabicyclo[3.2.1]octane-8-carboxylate (0.4 g, 0.8 mmol), the product of Example 7, was heated to reflux in chloroform. Iodotrimethylsilane was added in two aliquots (0.8 g+0.4 g, 6.0 mmol total) as the reaction was monitored to completion within 5 hours. The resultant orange solid was collected by filtration, dissolved in dichloromethane/methanol (9:1), adsorbed onto silica, and chromatographed in 5-15% methanol/dichloromethaneto provide 0.11 g (33% yield) of 7-(8-azabicyclo[3.2.1]oct-3-yl)-3-(3-methoxyphenyl)-2-pyridin-4-ylpyrazolo[1,5-a]pyrimidine as a yellow solid. MS: 412.4 [M+H].

### Example 20

### 7-(8-Ethyl-8-azabicyclo[3.2.1]octan-3-yl)-3-(2-methoxypyridin-4-yl)-2-(pyridin-4-yl)pyrazolo[1,5-a]pyrimidine

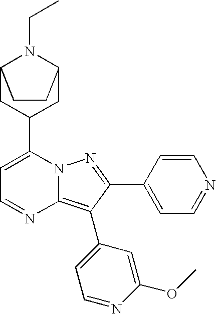

Step 1: Ethyl 3-(2-(pyridin-4-yl)pyrazolo[1,5-a]pyrimidin-7-yl)-8-azabicyclo[3.2.1]octane-8-carboxylate was synthesized according to the procedure for Example 7, Step 4, staring from (E)-ethyl 3-(3-(dimethylamino)acryloyl)-8-azabicyclo[3.2.1]octane-8-carboxylate and 3-(pyridin-4-yl)-1H-pyrazol-5-amine. MS: 378.4 [M+H].

Step 2: 7-(8-azabicyclo[3.2.1]octan-3-yl)-2-(pyridin-4-yl)pyrazolo[1,5-a]pyrimidine was synthesized according to the procedure for Example 9 starting from ethyl 3-(2-(pyridin-4-yl)pyrazolo[1,5-a]pyrimidin-7-yl)-8-azabicyclo[3.2.1]octane-8-carboxylate. MS: 306.3 [M+H].

Step 3: A mixture of 7-(8-azabicyclo[3.2.1]octan-3-yl)-2-(pyridin-4-yl)pyrazolo[1,5-a]pyrimidine (3.1 g, 10.2 mmol), trifluoroacetic anhydride (1.48 mL, 10.7 mmol) and triethylamine (4.26 mL, 30.6 mmol) in dicholoromethane (100 mL) was stirred for 1 hour. The reaction was then extracted once with saturated sodium bicarbonate (200 mL) and saturated ammonium chloride (200 mL). The organic layer was dried over anhydrous sodium sulfate, filtered and the filtrate was evaporated to yield a solid. The crude product was purified by silica gel flash chromatography, eluting with 5-20% methanol in dichloromethane, to give 2.1 g (51% yield) of 2,2,2-trifluoro-1-(3-(2-(pyridin-4-yl)pyrazolo[1,5-a]pyrimidin-7-yl)-8-azabicyclo[3.2.1]octan-8-yl)ethanone. MS: 402.3 [M+H].

Step 4: To solution of 2,2,2-trifluoro-1-(3-(2-(pyridin-4-yl)pyrazolo[1,5-a]pyrimidin-7-yl)-8-azabicyclo[3.2.1]octan-8-yl)ethanone (2.1 g, 5.2 mmol) in dichloromethane (125 mL) was added N-iodosuccinamide (17 g, 52.3 mmol) in three portions over a 3 hour period and the reaction was then stirred for an additional 16 hours. The reaction was extracted with saturated sodium thiosulfate (2×200 mL). The organic layer was dried over anhydrous sodium sulfate, filtered and the filtrate was evaporated to give 3.0 g of 2,2,2-trifluoro-1-(3-(3-iodo-2-(pyridin-4-yl)pyrazolo[1,5-a]pyrimidin-7-yl)-8-azabicyclo[3.2.1]octan-8-yl)ethanone as a yellow solid. This product was used in the next step without further purification. MS: 528.1 [M+H].

Step 5: A mixture of 2,2,2-trifluoro-1-(3-(3-iodo-2-(pyridin-4-yl)pyrazolo[1,5-a]pyrimidin-7-yl)-8-azabicyclo[3.2.1]octan-8-yl)ethanone (3.0 g, 5.7 mmol), potassium carbonate (3.5 g, 25 mmol), methanol (50 mL) and water (10 mL) was stirred for 4 days. The solvent was then removed and the remaining crude solid was stirred in 10% methanol in dichloromethane. The remaining solids were removed by filtration and washed with dichloromethane. The filtrate was concentrated in vacuo to give 7-(8-azabicyclo[3.2.1]octan-3-yl)-3-iodo-2-(pyridin-4-yl)pyrazolo[1,5-a]pyrimidine as a yellow solid that was used directly in the next step.

Step 6: Following the procedure of Example 17, 7-(8-azabicyclo[3.2.1]octan-3-yl)-3-iodo-2-(pyridin-4-yl)pyrazolo[1,5-a]pyrimidine (2.9 g, 5.7 mmol) was reacted with potassium carbonate and iodoethane in dimethylformamide to provide 2.3 g (86% yield) of 7-(8-ethyl-8-azabicyclo[3.2.1]octan-3-yl)-3-iodo-2-(pyridin-4-yl)pyrazolo[1,5-a]pyrimidine as a yellow solid. MS 460.3 [M+H].

Step 7: 7-(8-ethyl-8-azabicyclo[3.2.1]octan-3-yl)-3-iodo-2-(pyridin-4-yl)pyrazolo[1,5-a]pyrimidine (150 mg, 0.32 mmol) and 2-methoxypyridin-4-ylboronic acid (100 mg, 0.65 mmol) were dissolved in ethylene glycol dimethyl ether (3 mL) and to the resulting solution was added (1,1′-bis(diphenylphosphino)ferrocene) dichloropalladium(II) dichloromethane complex (53 mg). A solution of potassium carbonate (90 mg, 2.24 mmol) in water (0.5 mL) was next added to the reaction mixture, and the reaction was heated to 80° C. for 3 hours. The reaction was then cooled and saturated sodium bicarbonate (20 mL) was added. The mixture was then extracted with dichloromethane (2×50 mL). The organic layer was dried over anhydrous sodium sulfate, filtered, and the filtrate was evaporated to yield a solid. The crude solid was purified by silica gel flash chromatography, eluting with 15% methanol in dichloromethane, to give 0.07 g (50% yield) of 7-(8-ethyl-8-azabicyclo[3.2.1]octan-3-yl)-3-(2-methoxypyridin-4-yl)-2-(pyridin-4-yl)pyrazolo[1,5-a]pyrimidine. MS: 441.3 [M+H].

### Example 21

### 4-(7-(8-Ethyl-8-azabicyclo[3.2.1]octan-3-yl)-2-(pyridin-4-yl)pyrazolo[1,5-a]pyrimidin-3-yl)pyridin-2-ol

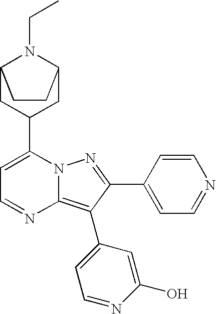

A mixture of 7-(8-ethyl-8-azabicyclo[3.2.1]octan-3-yl)-3-(2-methoxypyridin-4-yl)-2-(pyridin-4-yl)pyrazolo[1,5-a]pyrimidine (0.68 g, 0.15mmol) and pyridine hydrochloride (0.68 g) was stirred at 180° C. for 30 minutes. The reaction was then cooled and diluted with saturated sodium bicarbonate. The solvent was removed and the remaining crude solid was washed with 10% methanol in dichloromethane. The organic layer was dried over anhydrous sodium sulfate, filtered and the filtrate was evaporated to give 0.049 g (75% yield) of 4-(7-(8-ethyl-8-azabicyclo[3.2.1]octan-3-yl)-2-(pyridin-4-yl)pyrazolo[1,5-a]pyrimidin-3-yl)pyridin-2-ol as a yellow solid. MS: 427.3 [M+H].

### Example 22

### 4-(7-(8-Ethyl-8-azabicyclo[3.2.1]octan-3-yl)-2-(pyridin-4-yl)pyrazolo[1,5-a]pyrimidin-3-yl)aniline

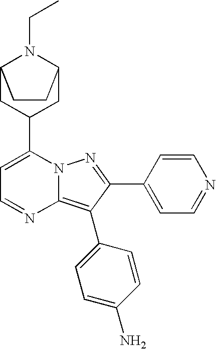

Following the procedure of Example 20, Step 7, 7-(8-ethyl-8-azabicyclo[3.2.1]octan-3-yl)-3-iodo-2-(pyridin-4-yl)pyrazolo[1,5-a]pyrimidine was reacted with 4-(4,4,5,5-tetramethyl-1,3,2-dioxaborolan-2-yl)aniline to provide 81 mg (60% yield) of 4-(7-(8-ethyl-8-azabicyclo[3.2.1]octan-3-yl)-2-(pyridin-4-yl)pyrazolo[1,5-a]pyrimidin-3-yl)aniline as a yellow solid. MS: 425.3 [M+H].

### Example 23

### 1-(4-(7-(8-Ethyl-8-azabicyclo[3.2.1]octan-3-yl)-2-(pyridin-4-yl)pyrazolo[1,5-a]pyrimidin-3-yl)phenyl)urea

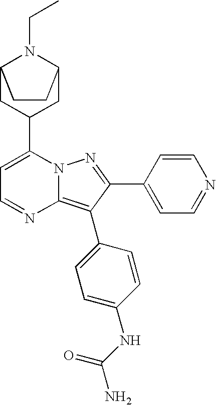

A mixture of 4-(7-(8-ethyl-8-azabicyclo[3.2.1]octan-3-yl)-2-(pyridin-4-yl)pyrazolo[1,5-a]pyrimidin-3-yl)aniline (23 mg, 0.05 mmol), the product of Example 22, and triethylamine (22 μL, 0.16 mmol) in dichloromethane (1.5 mL) was added to a solution of triphosgene (8 mg, 0.025 mmol) in dichloromethane (0.5 mL) and stirred for 10 minutes. To this mixture was added a 2M solution of ammonia in dioxane (2 mL) and the resulting mixture was stirred for an additional 30 minutes. The reaction was diluted with dichloromethane (5 mL) and extracted with saturated sodium bicarbonate (2×, 5 ml). The organic layer was dried over anhydrous sodium sulfate, filtered, and the filtrate was evaporated to give a solid. The crude solid was purified by silica gel flash chromatography, eluting with 15% methanol in dichloromethane, to give 5.1 mg (20% yield) of 1-(4-(7-(8-ethyl-8-azabicyclo[3.2.1]octan-3-yl)-2-(pyridin-4-yl)pyrazolo[1,5-a]pyrimidin-3-yl)phenyl)urea as a yellow solid. MS: 468.3 [M+H].

### Example 24

### 3-(3-Methoxy-phenyl)-7-[4-(8-methyl-3,8-diaza-bicyclo[3.2.1]oct-3-yl)-phenyl]-2-pyridin-4-yl-pyrazolo[1,5-a]pyrimidine

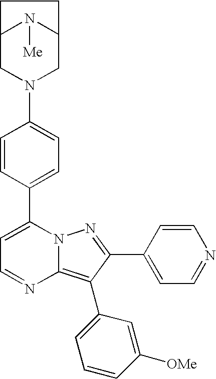

Step 1: Following the procedure of Example 1, Step 1, 1-(4-bromo-phenyl)-ethanone was reacted with dimethylformamide dimethyl acetal to provide 1-(4-bromo-phenyl)-3-dimethylamino-propenone. MS: 254.2 [M+H].

Step 2: Following the procedure of Example 7, Step 4, 1-(4-bromo-phenyl)-3-dimethylamino-propenone was reacted with 4-(3-methoxyphenyl)-3-(pyridin-4-yl)-1H-pyrazol-5-amine to provide 7-(4-bromo-phenyl)-3-(3-methoxy-phenyl)-2-pyridin-4-yl-pyrazolo[1,5-a]pyrimidine. MS: 457.3 [M+H].

Step 3: A sealed tube was charged with 7-(4-bromo-phenyl)-3-(3-methoxy-phenyl)-2-pyridin-4-yl-pyrazolo[1,5-a]pyrimidine (65 mg, 0.14 mmol), 8-methyl-3,8-diaza-bicyclo[3.2.1]octane dihydrochloride (25 mg, 0.13 mmol), sodium tert-butoxide (37 mg, 0.39 mmol), tris(dibenzylideneacetone)dipalladium(0) (25 mg, 0.027 mmol,), BINAP (66 mg, 0.1 mmol), and THF (3 mL) under nitrogen. The tube was heated to 100° C. overnight. The solution was then allowed to cool to room temperature, concentrated, dissolved in DMSO, filtered, and purified by H PLC to give 3-(3-methoxy-phenyl)-7-[4-(8-methyl-3,8-diaza-bicyclo[3.2.1]oct-3-yl)-phenyl]-2-pyridin-4-yl-pyrazolo[1,5-]pyrimidine TFA salt as a yellow solid. MS: 503.5 [M+H].

### Example 25

### 3-{7-[4-(8-Methyl-3,8-diaza-bicyclo[3.2.1]oct-3-yl)-phenyl]-2-pyridin-4-yl-pyrazolo[1,5-a]pyrimidin-3-yl}-phenol

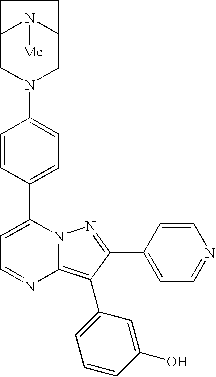

Following the procedure of Example 8, 3-(3-methoxy-phenyl)-7-[4-(8-methyl-3,8-diaza-bicyclo[3.2.1]oct-3-yl)-phenyl]-2-pyridin-4-yl-pyrazolo[1,5-]pyrimidine was reacted with boron tribromide and then purified using HPLC to provide 3-{7-[4-(8-methyl-3,8-diaza-bicyclo[3.2.1]oct-3-yl)-phenyl]-2-pyridin-4-yl-pyrazolo[1,5-a]pyrimidin-3-yl}-phenol. MS: 489.5 [M+H].

### Example 26

### 3-(4-Chloro-3-methoxy-phenyl)-7-[4-(8-methyl-3,8-diaza-bicyclo[3.2.1]oct-3-yl)-phenyl]-2-pyridin-4-yl-pyrazolo[1,5-a]pyrimidine

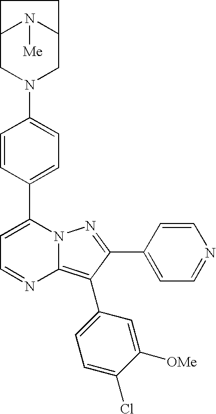

Following the procedure of Example 24, starting from 4-(4-chloro-3-methoxy-phenyl)-5-pyridin-4-yl-2H-pyrazol-3-ylamine, 3-(4-chloro-3-methoxy-phenyl)-7-[4-(8-methyl-3,8-diaza-bicyclo[3.2.1]oct-3-yl)-phenyl]-2-pyridin-4-yl-pyrazolo[1,5-a]pyrimidine in the form of the TFA salt was obtained as a yellow solid. MS: 537.5 [M+H].

### Example 27

### 2-Chloro-5-{7-[4-(8-methyl-3,8-diaza-bicyclo[3.2.1]oct-3-yl)-phenyl]-2-pyridin-4-yl-pyrazolo[1,5-a]pyrimidin-3-yl}-phenol

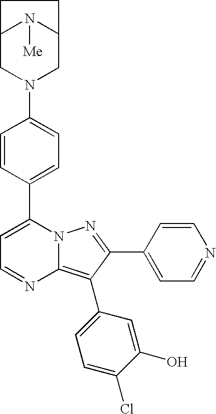

Following the procedure of Example 8, 3-(4-chloro-3-methoxy-phenyl)-7-[4-(8-methyl-3,8-diaza-bicyclo[3.2.1]oct-3-yl)-phenyl]-2-pyridin-4-yl-pyrazolo[1,5-a]pyrimidine was reacted with boron tribromide and then purified using HPLC to provide 2-chloro-5-{7-[4-(8-methyl-3,8-diaza-bicyclo[3.2.1]oct-3-yl)-phenyl]-2-pyridin-4-yl-pyrazolo[1,5-a]pyrimidin-3-yl}-phenol. MS: 523.5 [M+H].

### Example 28

### 7-[4-((1S,4S)-2,5-Diaza-bicyclo[2.2.1]hept-2-yl)-phenyl]-3-(3-methoxy-phenyl)-2-pyridin-4-yl-pyrazolo[1,5-a]pyrimidine

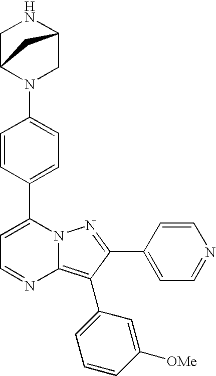

Following the procedure of Example 24, starting from 4-(3-methoxy-phenyl)-5-pyridin-4-yl-2H-pyrazol-3-ylamine and (1S,4S)-2,5-diaza-bicyclo[2.2.1]heptane dihydrobromide, 7-[4-((1S,4S)-2,5-diaza-bicyclo[2.2.1]hept-2-yl)-phenyl]-3-(3-methoxy-phenyl)-2-pyridin-4-yl-pyrazolo[1,5-a]pyrimidine trifluoroacetate salt was obtained as a yellow solid. MS: 475.5 [M+H].

### Example 29

### 3-{7-[4-((1S,4S)-2,5-Diaza-bicyclo[2.2.1]hept-2-yl)-phenyl]-2-pyridin-4-yl-pyrazolo[1,5-a]pyrimidin-3-yl}-phenol

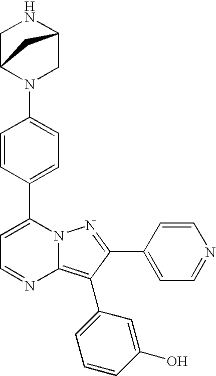

Following the procedure of Example 8, 7-[4-((1S,4S)-2,5-diaza-bicyclo[2.2.1]hept-2-yl)-phenyl]-3-(3-methoxy-phenyl)-2-pyridin-4-yl-pyrazolo[1,5-a]pyrimidine was reacted with boron tribromide and then purified using HPLC to provide 3-{7-[4-((1S,4S)-2,5-diaza-bicyclo[2.2.1]hept-2-yl)-phenyl]-2-pyridin-4-yl-pyrazolo[1,5-a]pyrimidin-3-yl}-phenol trifluoroacetate salt. MS: 461.4 [M+H].

### Example 30

### 3-{7-[4-((1S,4S)-5-Methyl-2,5-diaza-bicyclo[2.2.1]hept-2-yl)-phenyl]-2-pyridin-4-yl-pyrazolo[1,5-a]pyrimidin-3-yl}-phenol

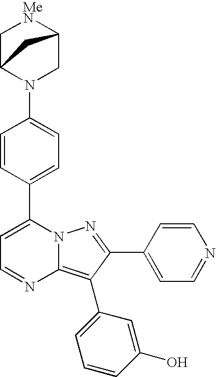

Three drops of formaldehyde (37% in water) was added to a solution of 3-{7-[4-((1S,4S)-2,5-diaza-bicyclo[2.2.1]hept-2-yl)-phenyl]-2-pyridin-4-yl-pyrazolo[1,5-a]pyrimidin-3-yl}-phenol (18 mg), the product of Example 25, and excess sodium triacetoxyborohydride in 2 mL of DMF. After 3 hours, the reaction mixture was filtered and the crude product was purified by HPLC to provide 3-{7-[4-((1S,4S)-5-methyl-2,5-diaza-bicyclo[2.2.1]hept-2-yl)-phenyl]-2-pyridin-4-yl-pyrazolo[1,5-a]pyrimidin-3-yl}-phenol trifluoroacetate salt. MS: 475.5 [M+H].

### Example 31

### 7-(4-((1S,4S)-2,5-Diaza-bicyclo[2.2.1]heptan-2-yl)phenyl)-3-(4-chloro-3-methoxyphenyl)-2-(pyridin-4-yl)pyrazolo[1,5-a]pyrimidine

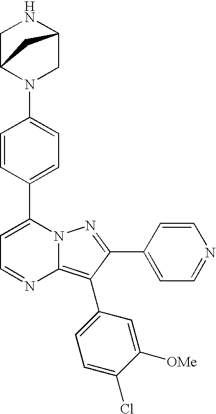

Following the procedure of Example 24, Steps 1-3, 4-(4-chloro-3-methoxyphenyl)-5-pyridin-4-yl-2H-pyrazol-3-ylamine and (1S,4S)-2,5-diaza-bicyclo[2.2.1]heptane dihydrobromide gave 7-(4-((1S,4S)-2,5-diaza-bicyclo[2.2.1]heptan-2-yl)phenyl)-3-(4-chloro-3-methoxyphenyl)-2-(pyridin-4-yl)pyrazolo[1,5-a]pyrimidine as a trifluoroacetate salt. MS: 509.3 [M+H].

### Example 32

### 7-(4-((1S,4S)-2,5-Diaza-bicyclo[2.2.1]heptan-2-yl)phenyl)-3-(4-fluoro-3-methoxyphenyl)-2-(pyridin-4-yl)pyrazolo[1,5-a]pyrimidine

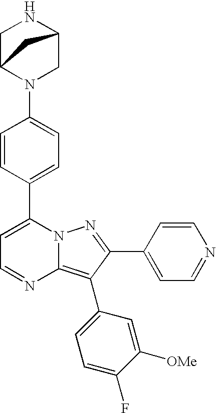

The compound 4-(4-fluoro-3-methoxyphenyl)-5-pyridin-4-yl-2H-pyrazol-3-ylamine was prepared according to the procedure of Example 1, Step 2, starting from 4-fluoro-3-methoxy-benzeneacetonitrile. Following the procedure of Example 24, Steps 1-3, and using (1S,4S)-2,5-diaza-bicyclo[2.2.1]heptane dihydrobromide in step 3, 4-(4-fluoro-3-methoxyphenyl)-5-pyridin-4-yl-2H-pyrazol-3-ylamine, gave 7-(4-((1S,4S)-2,5-diaza-bicyclo[2.2.1]heptan-2-yl)phenyl)-3-(4-fluoro-3-methoxyphenyl)-2-(pyridin-4-yl)pyrazolo[1,5-a]pyrimidine trifluoroacetate salt. MS: 493.5 [M+H].

### Example 33

### 5-(7-(4-((1S,4S)-2,5-Diazabicyclo[2.2.1]heptan-2-yl)phenyl)-2-(pyridin-4-yl)pyrazolo[1,5-a]pyrimidin-3-yl)-2-fluorophenol

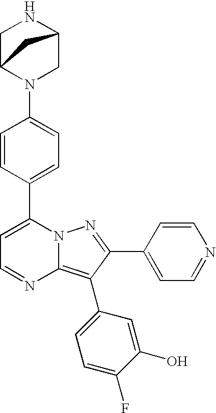

Following the procedure of Example 8, 7-(4-((1S,4S)-2,5-diaza-bicyclo[2.2.1]heptan-2-yl)phenyl)-3-(4-fluoro-3-methoxyphenyl)-2-(pyridin-4-yl)pyrazolo[1,5-a]pyrimidine was reacted with boron tribromide and the resulting product was purified by HPLC to provide 5-(7-(4-((1S,4S)-2,5-diazabicyclo[2.2.1]heptan-2-yl)phenyl)-2-(pyridin-4-yl)pyrazolo[1,5-a]pyrimidin-3-yl)-2-fluorophenol trifluoroacetate salt. MS: 479.5 [M+H].

### Example 34

### 3-(1H-Indazol-4-yl)-7-(6-((1S,4S)-5-methyl-2,5-diazabicyclo[2.2.1]heptan-2-yl)pyridin-3-yl)-2-(pyridin-4-yl)pyrazolo[1,5-a]pyrimidine

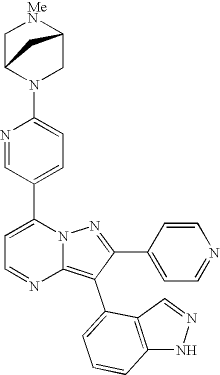

Step 1: Following the procedure of Example 1, Step 3, 1-(6-bromo-pyridin-3-yl)-3-dimethylamino-propenone was reacted with 3-(pyridin-4-yl)-1H-pyrazol-5-amine to give 7-(6-bromopyridin-3-yl)-2-(pyridin-4-yl)pyrazolo[1,5-a]pyrimidine as a yellow solid.

Step 2: Following the procedure of Example 1, Step 4, 7-(6-bromopyridin-3-yl)-2-(pyridin-4-yl)pyrazolo[1,5-a]pyrimidine was reacted with (1S,4S)-2,5-diaza-bicyclo[2.2.1]heptane dihydrobromide and purified by silica gel chromatography to give 7-(6-((1S,4S)-2,5-diazabicyclo[2.2.1]heptan-2-yl)pyridin-3-yl)-2-(pyridin-4-yl)pyrazolo[1,5-a]pyrimidine. MS: 370.4 [M+H].

Step 3: 7-(6-((1S,4S)-2,5-Diazabicyclo[2.2.1]heptan-2-yl)pyridin-3-yl)-2-(pyridin-4-yl)pyrazolo[1,5-a]pyrimidine (100 mg, 0.27 mmol) was dissolved in 5 mL of DMF, and then 37% formaldehyde (0.10 mL, 1.35 mmol) and a drop of acetic acid were added. The solution was stirred for 5 minutes and then sodium triacetoxyborohydride (286 mg, 1.35 mmol) was added. The reaction was quenched with 2mL of a solution of methanolic ammonia after one hour. Then the mixture was concentrated and purified by silica gel chromatography to give 7-(6-((1S,4S)-5-methyl-2,5-diazabicyclo[2.2.1]heptan-2-yl)pyridin-3-yl)-2-(pyridin-4-yl)pyrazolo[1,5-a]pyrimidine. MS: 384.4 [M+H].

Step 4: 7-(6-((1S,4S)-5-Methyl-2,5-diazabicyclo[2.2.1]heptan-2-yl)pyridin-3-yl)-2-(pyridin-4-yl)pyrazolo[1,5-a]pyrimidine (200 mg, 0.52 mmol) was dissolved in 10 mL of dichloromethane and 1 mL of acetic acid and then N-iodosuccinimide (175 mg, 0.78 mmol) was added. The resulting reaction was quenched with a solution of methanolic ammonia after one hour, concentrated and purified by silica gel chromatography to give 3-iodo-7-(6-((1S,4S)-5-methyl-2,5-diazabicyclo[2.2.1]heptan-2-yl)pyridin-3-yl)-2-(pyridin-4-yl)pyrazolo[1,5-a]pyrimidine. MS: 510.4 [M+H].

Step 5: To a suspension of 3-iodo-7-(6-((1S,4S)-5-methyl-2,5-diazabicyclo[2.2.1]heptan-2-yl)pyridin-3-yl)-2-(pyridin-4-yl)pyrazolo[1,5-a]pyrimidine (110 mg, 0.22 mmol) in 3 mL of dimethoxyethane was added 2M sodium carbonate (0.22 mL, 0.44 mmol), 4-(4,4,5,5-tetramethyl-1,3,2-dioxaborolan-2-yl)-1H-indazole (100 mg [79% purity], 0.32 mmol) and catalytic amount of tetrakis(triphenylphosphine)palladium(0). The mixture was heated to 130° C. for 50 minutes in a microwave reactor. The crude product was purified by HPLC and then silica gel chromatography to give the desired product as a free base. The free base was dissolved in methanol and then 1 mL of 1.25 M methanolic HCl was added. The solution was concentrated in vacuo and dried to give 3-(1H-indazol-4-yl)-7-(6-((1S,4S)-5-methyl-2,5-diazabicyclo[2.2.1]heptan-2-yl)pyridin-3-yl)-2-(pyridin-4-yl)pyrazolo[1,5-a]pyrimidine hydrochloride salt. MS: 500.3 [M+H].

### Example 35

### 3-(1H-indazol-4-yl)-7-(4-((1S,4S)-5-methyl-2,5-diazabicyclo[2.2.1]heptan-2-yl)phenyl)-2-(pyridin-4-yl)pyrazolo[1,5-a]pyrimidine

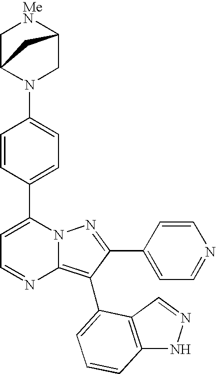

Step 1: Following the procedure of Example 1, Step 3, (E)-1-(4-bromophenyl)-3-(dimethylamino)prop-2-en-1-one was reacted with 3-(pyridin-4-yl)-1H-pyrazol-5-amine to give 7-(4-bromophenyl)-2-(pyridin-4-yl)pyrazolo[1,5-a]pyrimidine as a yellow solid.

Step 2: Following the procedure of Example 24, Step 3, 7-(4-bromophenyl)-2-(pyridin-4-yl)pyrazolo[1,5-a]pyrimidine was coupled with (1S,4S)-2,5-diaza-bicyclo[2.2.1]heptane dihydrobromide to give 7-(4-((1S,4S)-2,5-diazabicyclo[2.2.1]heptan-2-yl)phenyl)-2-(pyridin-4-yl)pyrazolo[1,5-a]pyrimidine. MS: 369.4 [M+H].

Steps 3-5: Following the procedure of Example 34, Steps 3-5, 7-(4-((1S,4S)-2,5-diazabicyclo[2.2.1]heptan-2-yl)phenyl)-2-(pyridin-4-yl)pyrazolo[1,5-a]pyrimidine was converted into 3-(1H-indazol-4-yl)-7-(4-((1S,4S)-5-methyl-2,5-diazabicyclo[2.2.1]heptan-2-yl)phenyl)-2-(pyridin-4-yl)pyrazolo[1,5-a]pyrimidine hydrochloride salt, obtained as a deep red solid. ¹H NMR (400 MHz, CDCl₃) δ 8.73-8.70 (m, 2H), 8.57 (d, J=4.4 Hz, 1H), 8.41-8.36 (m, 2H), 8.26-8.21 (m, 2H), 7.73-7.67 (m, 2H), 7.58 (dd, J=7.0, 8.2 HZ, 1H), 7.38 (d, J=4.8 Hz, 1H), 7.35-7.31 (m, 1H), 6.99-6.93 (m, 2H), 4.51-4.47 (m, 1H), 3.92-3.82 (m, 2H), 3.60 (d, J=11.2 Hz, 1H), 3.04 (s, 3H), 2.55-2.49 (m, 1H), 2.41-2.34 (m, 1H). MS: 499.5 [M+H].

### Example 36

### 3-(3-Methoxyphenyl)-7-(4-((1S,4S)-5-methyl-2,5-diazabicyclo[2.2.1]heptan-2-yl)phenyl)-2-(pyridin-4-yl)pyrazolo[1,5-a]pyrimidine

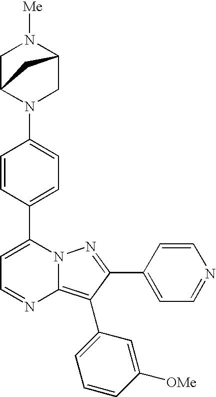

Following the procedure of Example 30, 7-[4-((1S,4S)-2,5-diaza-bicyclo[2.2.1]hept-2-yl)-phenyl]-3-(3-methoxy-phenyl)-2-pyridin-4-yl-pyrazolo[1,5-a]pyrimidine was converted into 3-(3-methoxyphenyl)-7-(4-((1S,4S)-5-methyl-2,5-diazabicyclo[2.2.1]heptan-2-yl)phenyl)-2-(pyridin-4-yl)pyrazolo[1,5-a]pyrimidine, obtained as a trifluoroacetate salt. MS: 489.5 [M+H].

### Example 37

### 3-(4-Fluoro-3-methoxyphenyl)-7-(4-((1S,4S)-5-methyl-2,5-diazabicyclo[2.2.1]heptan-2-yl)phenyl)-2-(pyridin-4-yl)pyrazolo[1,5-a]pyrimidine

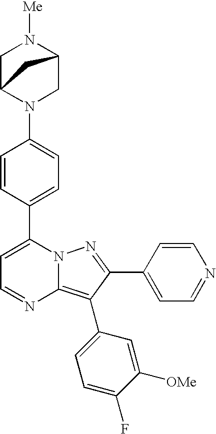

Following the procedure of Example 30, 7-(4-((1S,4S)-2,5-diaza-bicyclo[2.2.1]heptan-2-yl)phenyl)-3-(4-fluoro-3-methoxyphenyl)-2-(pyridin-4-yl)pyrazolo[1,5-a]pyrimidine was converted into 3-(4-fluoro-3-methoxyphenyl)-7-(4-((1S,4S)-5-methyl-2,5-diazabicyclo[2.2.1]heptan-2-yl)phenyl)-2-(pyridin-4-yl)pyrazolo[1,5-a]pyrimidine, obtained as a trifluoroacetate salt. MS: 507.5 [M+H].

### Example 38

### 5-(7-(4-((1S,4S)-5-Ethyl-2,5-diazabicyclo[2.2.1]heptan-2-yl)phenyl)-2-(pyridin-4-yl)pyrazolo[1,5-a]pyrimidin-3-yl)-2-fluorophenol

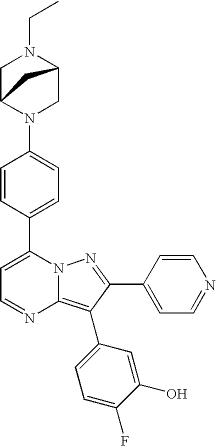

Following the procedure of Example 30, acetaldehyde and 5-(7-(4-((1S,4S)-2,5-diazabicyclo[2.2.1]heptan-2-yl)phenyl)-2-(pyridin-4-yl)pyrazolo[1,5-a]pyrimidin-3-yl)-2-fluorophenol provided 5-(7-(4-((1S,4S)-5-ethyl-2,5-diazabicyclo[2.2.1]heptan-2-yl)phenyl)-2-(pyridin-4-yl)pyrazolo[1,5-a]pyrimidin-3-yl)-2-fluorophenol as a trifluoroacetate salt. MS: 507.3 [M+H].

### Example 39

### 1-((1S,4S)-5-(4-(3-(4-Fluoro-3-hydroxyphenyl)-2-(pyridin-4-yl)pyrazolo[1,5-a]pyrimidin-7-yl)phenyl)-2,5-diazabicyclo[2.2.1]heptan-2-yl)ethanone

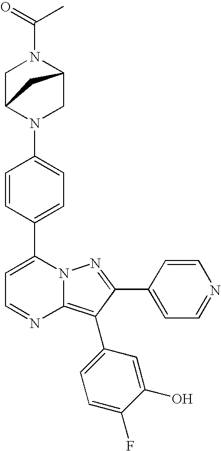

The compound 5-(7-(4-((1S,4S)-2,5-diazabicyclo[2.2.1]heptan-2-yl)phenyl)-2-(pyridin-4-yl)pyrazolo[1,5-a]pyrimidin-3-yl)-2-fluorophenol was acetylated to give 1-((1S,4S)-5-(4-(3-(4-fluoro-3-hydroxyphenyl)-2-(pyridin-4-yl)pyrazolo[1,5-a]pyrimidin-7-yl)phenyl)-2,5-diazabicyclo[2.2.1]heptan-2-yl)ethanone. MS: 521.5 [M+H].

### Example 40

### 3-(7-Methyl-1H-indazol-4-yl)-7-(6-((1S,4S)-5-methyl-2,5-diazabicyclo[2.2.1]heptan-2-yl)pyridin-3-yl)-2-(pyridin-4-yl)pyrazolo[1,5-a]pyrimidine

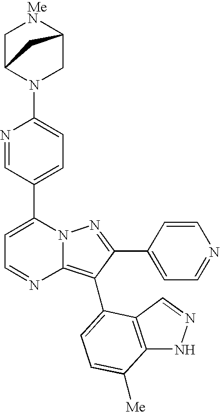

Step 1: A mixture of nitro-m-xylene (3.02 g, 20.0 mmol), iodine (2.04 g, 8.0 mmol), periodic acid (4.1 g, 18.0 mmol), and concentrated sulfuric acid (1.2 mL) in acetic acid (2.4 mL) was heated at 90° C. for 3 days. The reaction was then cooled, poured in to water and extracted with dichloromethane. The combined organics were cooled and washed with a cold solution of 2N sodium hydroxide, and brine, dried over anhydrous magnesium sulfate, filtered, and concentrated in vacuo. The residue was triturated with hexanes and the solid was collected by filtration, washed with hexanes and dried to yield 2.3 g (42%) of 1-iodo-2,4-dimethyl-3-nitrobenzene as a white solid. MS: 278.1 [M+H]⁺

Step 2: To a hot suspension of iron powder (2.3 g, 8.3 mmol), ammonium chloride (2.16 g, 38.7 mmol) and water (18 mL) in ethanol (50 mL) was added 1-iodo-2,4-dimethyl-3-nitrobenzene in portions over a period of 10 minutes. The resulting mixture was heated at reflux for 1 hour, and filtered hot through a pad of Celite™. The Celite was washed with ethanol and ethyl acetate and the filtrate was concentrated in vacuo. The residue was extracted with dichloromethane, the organics were dried over anhydrous sodium sulfate and filtered and the filtrate was evaporated to yield 2.0 g (98%) of 3-iodo-2,6-dimethyl-phenylamine as a white solid. MS: 248.1 [M+H]⁺

Step 3: To a cold (0°-5° C.) solution of 3-iodo-2,6-dimethyl-phenylamine (2.0 g, 8.09 mmol) in chloroform (20 mL) was dropwise added acetic anhydride (1.8 mL, 18.63 mmol) and the resulting mixture was stirred for 5 minutes. The reaction was allowed to warm to room temperature and stirred for 1 hour and then potassium acetate (0.24 g, 2.45 mmol) and isoamyl nitrite (2.3 mL, 17.4 mmol) were added. The reaction was then heated at reflux for 20 hours. After cooling to room temperature the solvent was evaporated to yield a brown solid that was then diluted with water. After evaporating the water, the resulting brown solid residue was treated with concentrated hydrochloric acid and the mixture was heated at 50° C. for 2 hours and then cooled in an ice bath and basified to pH 14 with 50% potassium hydroxide solution. The solid was collected by filtration, washed with water and dried to yield 1.96 g of solid as 1:1 mixture of two isomers. The isomers were separated and purified by RP-HPLC to give 0.35g (17%) of the desired isomer, 4-iodo-7-methyl-1H-indazole, as a white solid, MS: 259.0 [M+H]⁺

Step 4: To a solution of 4-iodo-7-methyl-1H-indazole (0.113 mg, 0.44 mmol) in DMSO (5 mL) was added potassium acetate (0.17 g, 1.73 mmol), 1,1′-bis(diphenyl phosphino)ferrocene palladium chloride (11 mg, 0.013 mmol) and bis(pinacolato)diboron (0.14 g, 0.55 mmol). The mixture was degassed and heated in a microwave reactor at 120° C. for 1.5 hours. The reaction mixture was then filtered through a pad of celite, and the filtrate was diluted with water and then extracted with ethyl acetate (3×50 mL). The combined organic extracts were dried over sodium sulfate, filtered, and concentrated in-vacuo. The residue was purified by silica gel chromatography to provide 7-methyl-4-(4,4,5,5-tetramethyl-[1,3,2]dioxaborolan-2-yl)-1H-indazole as a white solid in 79% yield. MS: 259.2 [M+H]⁺

Step 5: Following the procedure of Example 34, Step 5, 3-iodo-7-(6-((1S,4S)-5-methyl-2,5-diazabicyclo[2.2.1]heptan-2-yl)pyridin-3-yl)-2-(pyridin-4-yl)pyrazolo[1,5-a]pyrimidine and 7-methyl-4-(4,4,5,5-tetramethyl-1,3,2-dioxaborolan-2-yl)-1H-indazole provided 3-(7-methyl-1H-indazol-4-yl)-7-(6-((1S,4S)-5-methyl-2,5-diazabicyclo[2.2.1]heptan-2-yl)pyridin-4-yl)pyrazolo[1,5-a]pyrimidine as a hydrochloride salt. MS: 514.7 [M+H].

### Example 41

### 3-(7-Chloro-1H-indazol-4-yl)-7-(6-((1S,4S)-5-methyl-2,5-diazabicyclo[2.2.1]heptan-2-yl)pyridin-3-yl)-2-(pyridin-4-yl)pyrazolo[1,5-a]pyrimidine

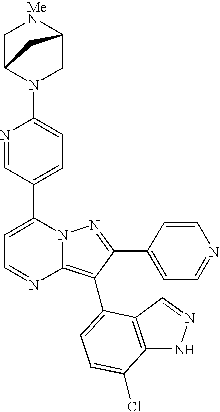

Step 1: To a solution of 2,2,6,6-tetramethylpiperidine (811 mg, 5.8 mmol) in THF (10 mL) at −78° C. was added a solution of 2.5 M butyllithium in hexanes (2.31 mL, 5.8 mmol) and 4-bromo-1-chloro-2-fluoro-benzene (1.0 g, 4.8 mmol). The mixture was warmed to −20° C. for 2 hours, then DMF (0.54 mL, 6.9 mmol) was added and the reaction was then stirred for 2 hours at room temperature. The reaction was quenched with water (100 mL) and the mixture was neutralized with 1M HCl and then extracted with ether (3×30 mL). The combined organic extracts were dried over sodium sulfate, filtered, concentrated in-vacuo. The residue was purified by silica gel chromatography to provide 6-bromo-3-chloro-2-fluorobenzaldehyde in 85% yield. MS: 270.0 [M+H].

Step 2: To a solution of 6-bromo-3-chloro-2-fluorobenzaldehyde (1.0 g, 4.24 mmol) in DME (5 mL) was added hydrazine hydrate (5 mL). The mixture was refluxed for 3 hours and then cooled to room temperature. The solvent was evaporated, water (100 mL) was added, and the organic product was extracted with ethyl acetate (3×30 mL). The combined organic extracts were dried over sodium sulfate, filtered, and concentrated in-vacuo. The resulting residue was purified by silica gel chromatography to provide 4-bromo-7-chloro-1H-indazole in 51% yield. MS: 230.9 [M+H].

Step 3: To a solution 4-bromo-7-chloro-1H-indazole (500 mg, 2.16 mmol) in dimethylsulfoxide, DMSO (2 mL) was added potassium acetate (697 mg, 7.12 mmol), 1,1′-bis(diphenyl phosphino)ferrocene palladium chloride (77 mg, 0.10 mmol) and bis(pinacolato)diboron (1.1 g, 4.32 mmol). The mixture was degassed and heated in a microwave reactor for 2 hours at 120° C. The solvent was then filtered through a pad of celite, water (60 mL) was added, and the product was extracted with ethyl acetate (3×30 mL). The combined organic extracts were dried over sodium sulfate, concentrated in-vacuo, and the resulting 7-chloro-4-(4,4,5,5-tetramethyl-1,3,2-dioxaborolan-2-yl)-1H-indazole was used in the next step without further purification.

Step 4: Following the procedure of Example 34, Step 5, 3-iodo-7-(6-((1S,4S)-5-methyl-2,5-diazabicyclo[2.2.1]heptan-2-yl)pyridin-3-yl)-2-(pyridin-4-yl)pyrazolo[1,5-a]pyrimidine and 7-chloro-4-(4,4,5,5-tetramethyl-1,3,2-dioxaborolan-2-yl)-1H-indazole provided 3-(7-chloro-1H-indazol-4-yl)-7-(6-((1S,4S)-5-methyl-2,5-diazabicyclo-[2.2.1]heptan-2-yl)pyridin-3-yl)-2-(pyridin-4-yl)pyrazolo[1,5-a]pyrimidine as a hydrochloride salt. MS: 534.3 [M+H].

### Example 42

### 3-(7-Fluoro-1H-indazol-4-yl)-7-(6-((1S,4S)-5-methyl-2,5-diazabicyclo[2.2.1]heptan-2-yl)pyridin-3-yl)-2-(pyridin-4-yl)pyrazolo[1,5-a]pyrimidine

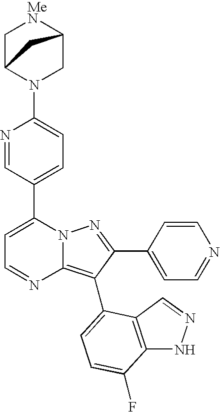

Step 1: To a solution of 2,2,6,6-tetramethylpiperidine (3.12 mL, 18.4 mmol) in THF (35 mL) at −78° C. was added a solution of 1.6M butyl lithium in hexanes (11.5 mL, 18.4 mmol) and 1-bromo-3,4-difluorobenzene (3.38 g, 17.5 mmol). The mixture warmed to −20° C. for 2 hours, and then DMF (1.42 mL, 18.4 mmol) was added and the reaction was warmed to room temperature and stirred for for 2 hours. The reaction was quenched with water (5 mL), neutralized with 1M HCl and extracted with ether (3×30 mL). The combined organic extracts were dried over sodium sulfate, filtered, and concentrated in-vacuo. The crude material 6-bromo-2,3-difluorobenzaldehyde was used as is in the next step.

Step 2: To a solution of 6-bromo-2,3-difluorobenzaldehyde (5.0 g, 22.6 mmol) in DME (20 mL) was added hydrazine hydrate (20 mL). The mixture was refluxed for 3 hours, and cooled to room temperature. The solvent was evaporated, water (100 mL) was added and the mixture was extracted with ethyl acetate (3×40 mL). The combined organic extracts were dried over sodium sulfate, filtered, and concentrated in vacuo. The residue was recrystallized from hot dichloromethane to provide 4-bromo-7-fluoro-1H-indazole as a white solid in 21% yield. MS: 215.0 [M+H].

Step 3: According to the procedure of Example 40, Step 4, 4-bromo-7-fluoro-1H-indazole provided 7-fluoro-4-(4,4,5,5-tetramethyl-1,3,2-dioxaborolan-2-yl)-1H-indazole as a white solid in 74% yield. MS: 263.1 [M+H].

Step 4: Following the procedure of Example 34, Step 5, 3-iodo-7-(6-((1S,4S)-5-methyl-2,5-diazabicyclo[2.2.1]heptan-2-yl)pyridin-3-yl)-2-(pyridin-4-yl)pyrazolo[1,5-a]pyrimidine and 7-fluoro-4-(4,4,5,5-tetramethyl-1,3,2-dioxaborolan-2-yl)-1H-indazole provided 3-(7-fluoro-1H-indazol-4-yl)-7-(6-((1S,4S)-5-methyl-2,5-diazabicyclo[2.2.1]heptan-2-yl)pyridin-3-yl)-2-(pyridin-4-yl)pyrazolo[1,5-a]pyrimidine as a hydrochloride salt. ¹H NMR (400 MHz, CDCl₃) δ 9.40 (d, J=2 Hz, 1H), 8.81-8.75 (m, 3H), 8.69 (d, J=4 Hz, 1H), 8.30-8.25 (m, 2H), 7.75 (d, J=3.2 Hz, 1H), 7.57 (d, J=4.4 Hz, 1H), 7.34-7.24 (m, 3H), 5.34 (s, 1H), 4.64 (s, 1H), 4.10-3.95 (m, 3H), 3.10 (s, 3H), 2.64 (d, J=11.6, 1H), 2.45 (d, J=11.6, 1H). MS: 518.7 [M+H].

### Example 43

### 3-(7-(6-((1S,4S)-5-Methyl-2,5-diazabicyclo[2.2.1]heptan-2-yl)pyridin-3-yl)-2-(pyridin-4-yl)pyrazolo[1,5-a]pyrimidin-3-yl)benzamide

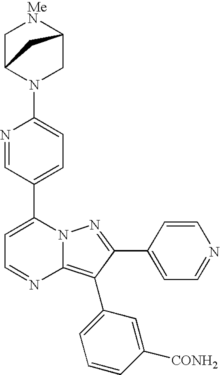

Following the procedure of Example 34, Step 5, 3-iodo-7-(6-((1S,4S)-5-methyl-2,5-diazabicyclo[2.2.1]heptan-2-yl)pyridin-3-yl)-2-(pyridin-4-yl)pyrazolo[1,5-a]pyrimidine and 3-carbamoylphenylboronic acid provided 3-(7-(6-((1S,4S)-5-methyl-2,5-diazabicyclo[2.2.1]heptan-2-yl)pyridin-3-yl)-2-(pyridin-4-yl)pyrazolo[1,5-a]pyrimidin-3-yl)benzamide as a hydrochloride salt. MS: 503.3 [M+H].

### Example 44

### 7-(4-((1S,4S)-2,5-Diazabicyclo[2.2.1]heptan-2-yl)-2-methylphenyl)-3-(1H-indazol-4-yl)-2-(pyridin-4-yl)pyrazolo[1,5-a]pyrimidine

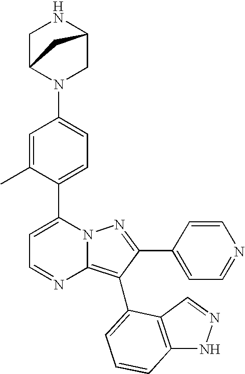

Step 1: To a solution of (1S,4S)-tert-butyl 2,5-diazabicyclo[2.2.1]heptane-2-carboxylate (200 mg, 1.3 mmol) in 2 mL of 1-methylpyrrolidin-2-one, 1-(4-fluoro-2-methylphenyl)ethanone (387 mg, 1.95 mmol) and N-ethyl-N-isopropylpropan-2-amine (0.45 mL, 2.6 mmol) were added. This solution was heated at 240° C. for 1 hour in a microwave reactor. The mixture was then cooled down to room temperature and then 0.54 g di-tert-butyl dicarbonate and 0.2 mL triethylamine were added. After stirring for 30 minutes, this mixture was diluted with 80 mL of dichloromethane and the organics were washed with water twice. The organic layer was dried and concentrated and the residue was purified by silica gel chromatography to give (1S,4S)-tert-butyl 5-(4-acetyl-3-methylphenyl)-2,5-diazabicyclo[2.2.1]heptane-2-carboxylate. MS: 331.2 [M+H].

Step 2: A mixture of (1S,4S)-tert-butyl 5-(4-acetyl-3-methylphenyl)-2,5-diazabicyclo[2.2.1]heptane-2-carboxylate (95 mg, 0.29 mmol) and 2 mL of 1,1-dimethoxy-N,N-dimethylmethanamine were heated at 190° C. for 1 hour in a microwave reactor. The reaction mixture was then diluted with 80 mL dichloromethane and the organics were washed with water twice. The organic layer was dried and concentrated and the residue was purified by silica gel chromatography to give (1S,4S)-tert-butyl 5-(4-((E)-3-(dimethylamino)acryloyl)-3-methyl phenyl)-2,5-diazabicyclo[2.2.1]heptane-2-carboxylate. MS: 386.2 [M+H].

Step 3: To a cold (0°-5° C.) suspension of sodium hydride (0.6 g, 15.0 mmol) in DMF (20 mL) was added methyl 1H-indazole-4-carboxylate (2.4 g, 13.62 mmol) [D. Batt, et al. J. Med. Chem., 2000, 43, 41-58] in portions over a period of 5 minutes and the resulting mixture was stirred at 5° C. for 15 minutes. A solution of benzene sulfonyl chloride (1.9 mL, 15.0 mmol) was then added dropwise and the resulting mixture was stirred at 5° C. for 30 minutes and then at room temperature for 3 hours. The mixture was poured on to ice and the solid was collected by filtration, washed with water and dried to yield 3.91 g (91%) of methyl 1-(phenylsulfonyl)-1H-indazole-4-carboxylate as a beige solid. MS: 317.1 [M+H]⁺

Step 4: To a suspension of methyl 1-(phenylsulfonyl)-1H-indazole-4-carboxylate (3.11 g, 9.83 mmol) in mixture of THF (30 mL) and toluene (15 mL) was added lithium borohydride as a 2.0M solution in THF (2.7 mL, 5.5 mmol) and the resulting mixture was stirred and heated at 70° C. for 30 minutes. Additional 2.0 M lithium borohydride solution (2.0 mL. 4.0 mmol) was added in portions over a period of 2.5 hours until all of the starting ester was consumed. The mixture was then cooled and poured on to ice water and the resulting two layers were separated. The aqueous layer was extracted with ethyl acetate. The combined organic layers were dried over anhydrous sodium sulfate, filtered and concentrated in vacuo. The crude oil was purified by silica gel chromatography to yield 2.0 g (71%) of [1-(phenylsulfonyl)-1H-indazol-4-yl]methanol as a white solid. MS: 289.1 [M+H].

Step 5: A mixture of [1-(phenylsulfonyl)-1H-indazol-4-yl]methanol (13.0 g, 45.08 mmol) and Dess-Martin periodinane (22.9 g, 54.0 mmol) in dichloromethane (420 mL) was stirred at room temperature for 1 hour. The reaction was quenched by stirring for 20 minutes with a saturated sodium thiosulfate solution (100 mL) and a saturated solution of sodium bicarbonate (75 mL). The two layers were separated and the aqueous layer was extracted with methylene chloride. The combined organic layers were dried over anhydrous sodium sulfate, filtered and concentrated in vacuo. The resulting crude solid was purified by silica gel chromatography to yield 12.65 g (98%) of 1-(phenylsulfonyl)-1H-indazole-4-carbaldehyde as a white solid. MS: 287.1 [M+H].

Step 6: A mixture of 1-(phenylsulfonyl)-1H-indazole-4-carbaldehyde (6.4 g, 22.4 mmol) and 8.5 g (20.3 mmol) of diphenyl(phenylamino)(pyridin-4-yl)methylphosphonate (prepared according to the procedure of Tet. Lett., 1988, 39, 1717-1720) in THF (50 mL) and isopropyl alcohol (10 mL) was stirred at room temperature and cesium carbonate (8.6 g, 26.4 mmol) was added in portions. After the reaction was stirred for 15 hours, 3N HCl (20 mL) was added and the mixture was stirred for an additional 4 hours. The reaction was then diluted with ether (150 mL) and extracted with 10% HCl (3×, 150 mL). The aqueous layer was neutralized to pH 7-8 using NaOH. The aqueous layer was next extracted with ethyl acetate (3×, 150 mL) and the combined organic layers were dried over anhydrous sodium sulfate, filtered, and concentrated in-vacuo to give 4.6 g (55% yield) of 2-(1-(phenylsulfonyl)-1H-indazol-4-yl)-1-(pyridin-4-yl)ethanone as a white solid. MS: 378.1 [M+H].

Step 7: Phosphorus oxychloride (1.4 mL, 14.9 mmol) was added to DMF (1.84 mL) at 0° C. and the mixture was stirred for 15 minutes. To this solution was added 2-(1-(phenylsulfonyl)-1H-indazol-4-yl)-1-(pyridin-4-yl)ethanone (1.13 g, 3.0 mmol) in dichloromethane (10 mL), and the reaction was then heated to 80° C. for 15 hours. The reaction was then cooled to room temperature, quenched with saturated sodium bicarbonate (300 mL), and extracted with 2% methanol in dichloromethane (4×250 mL). The organic layer was dried over anhydrous sodium sulfate, filtered, and then concentrated in-vacuo. The resulting residue was dissolved in dimethyl formamide, DMF (3 mL), hydroxylamine hydrochloride (0.15 mL, 3.6 mmol) was added and the reaction was stirred for 12 hours. The reaction was then cooled to 0° C., phosphorus oxychloride (0.64 mL, 6.0 mmol) was added and the mixture was stirred overnight at room temperature. The reaction was quenched with saturated sodium bicarbonate and extracted with 3% methanol in dichloromethane (4×200 mL). The combined organic layers were dried over anhydrous sodium sulfate, filtered, and then concentrated in-vacuo to give crude (E)-3-chloro-2-(1H-indazol-4-yl)-3-(pyridin-4-yl)acrylonitrile. The crude (E)-3-chloro-2-(1H-indazol-4-yl)-3-(pyridin-4-yl)acrylonitrile was dissolved in ethanol (16 mL) and hydrazine monohydrate (0.44 mL, 9.0 mmol) was added and the resulting reaction was stirred at 80° C. for 6 hours. The reaction was cooled to room temperature and the solvent was removed by evaporation. The crude product was purified by silica gel flash chromatography, eluting with 2-12% methanol in dichloromethane, to give 0.58 g (71% yield) of 4-(1H-indazol-4-yl)-3-(pyridin-4-yl)-1H-pyrazol-5-amine. MS: 277.2 [M+H].

Step 8: Following the procedure of Example 7, Step 4, (1S,4S)-tert-butyl 5-(4-((E)-3-(dimethylamino)acryloyl)-3-methylphenyl)-2,5-diazabicyclo[2.2.1]heptane-2-carboxylate (41 mg, 0.11 mmol) and 4-(1H-indazol-4-yl)-3-(pyridin-4-yl)-1H-pyrazol-5-amine (34 mg, 0.12 mmol) provided (1S,4S)-tert-butyl 5-(4-(3-(1H-indazol-4-yl)-2-(pyridin-4-yl)pyrazolo[1,5-a]pyrimidin-7-yl)-3-methylphenyl)-2,5-diazabicyclo-[2.2.1]heptane-2-carboxylate, which was used as a crude product for the next step. MS: 599.8 [M+H].

Step 9: Crude 5-(4-(3-(1H-indazol-4-yl)-2-(pyridin-4-yl)pyrazolo[1,5-a]pyrimidin-7-yl)-3-methylphenyl)-2,5-diazabicyclo[2.2.1]heptane-2-carboxylate was dissolved in 3 mL of 4 N HCl (diluted from concentrated HCl with methanol) and stirred for 1 hour at room temperature. The mixture was then concentrated, basified with methanolic ammonia solution, and purified by HPLC. The free base was dissolved in methanol and then 1 mL of 1.25 M methanolic HCl was added. The solution was concentrated in-vacuo and then dried to give 7-(4-((1S,4S)-2,5-diazabicyclo[2.2.1]heptan-2-yl)-2-methylphenyl)-3-(1H-indazol-4-yl)-2-(pyridin-4-yl)pyrazolo[1,5-a]pyrimidine-hydrochloride salt. MS: 499.4 [M+H].

### Example 45

### 3-(1H-Indazol-4-yl)-7-(2-methyl-4-((1S,4S)-5-methyl-2,5-diazabicyclo[2.2.1]heptan-2-yl)phenyl)-2-(pyridin-4-yl)pyrazolo[1,5-a]pyrimidine

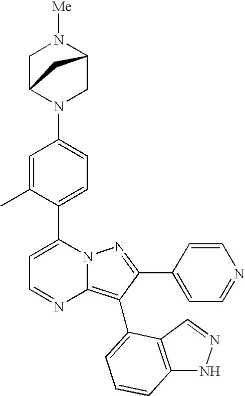

Following the procedure of Example 30, 7-(4-((1S,4S)-2,5-diazabicyclo[2.2.1]heptan-2-yl)-2-methylphenyl)-3-(1H-indazol-4-yl)-2-(pyridin-4-yl)pyrazolo[1,5-a]pyrimidine provided 3-(1H-indazol-4-yl)-7-(2-methyl-4-((1S,4S)-5-methyl-2,5-diazabicyclo[2.2.1]-heptan-2-yl)phenyl)-2-(pyridin-4-yl)pyrazolo[1,5-a]pyrimidine as a hydrochloride salt. MS: 513.4 [M+H].

### Example 46

### 7-(2-((1S,4S)-2,5-Diazabicyclo[2.2.1]heptan-2-yl)-4-fluorophenyl)-3-(1H-indazol-4-yl)-2-(pyridin-4-yl)pyrazolo[1,5-a]pyrimidine

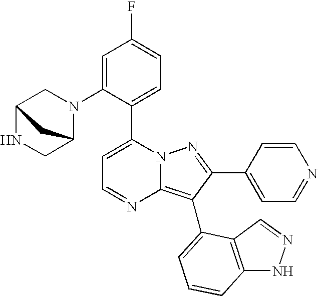

Following the procedure of Example 44, 1-(2,4-difluorophenyl)ethanone provided 7-(2-((1S,4S)-2,5-diazabicyclo[2.2.1]heptan-2-yl)-4-fluorophenyl)-3-(1H-indazol-4-yl)-2-(pyridin-4-yl)pyrazolo[1,5-a]pyrimidine as a hydrochloride salt. ¹H NMR (400 MHz, CD₃OD) δ 8.73-8.68 (m, 3H), 8.19-8.14 (m, 2H), 7.75-7.66 (m, 3H), 7.59 (dd, J=7.4, 8.2 Hz, 1H), 7.38-7.28 (m, 2H), 7.00 (dd, J=2.2, 11.8 Hz, 1H), 6.85 (dt, J=2.0, 8.0 Hz, 1H), 4.65 (s, 1H), 4.22 (s, 1H), 3.57-3.5 (m, 1H), 2.20-2.08 (m, 1H), 2.02-1.94 (m, 1H). MS: 503.7 [M+H].

### Example 47

### 7-(4-Fluoro-2-((1S,4S)-5-methyl-2,5-diazabicyclo[2.2.1]heptan-2-yl)phenyl)-3-(1H-indazol-4-yl)-2-(pyridin-4-yl)pyrazolo[1,5-a]pyrimidine

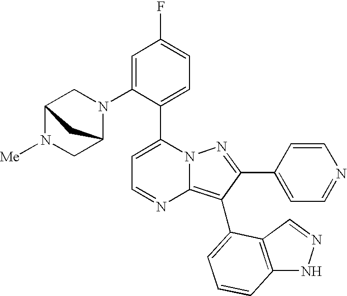

Following the procedure of Example 30, 7-(2-((1S,4S)-2,5-diazabicyclo[2.2.1]heptan-2-yl)-4-fluorophenyl)-3-(1H-indazol-4-yl)-2-(pyridin-4-yl)pyrazolo[1,5-a]pyrimidine provided 7-(4-fluoro-2-((1S,4S)-5-methyl-2,5-diazabicyclo[2.2.1]heptan-2-yl)phenyl)-3-(1H-indazol-4-yl)-2-(pyridin-4-yl)pyrazolo[1,5-a]pyrimidine as a hydrochloride salt. MS: 517.7 [M+H].

### Example 48

### (1S,4S)-tert-Butyl 5-(2-(3-(4-chloro-3-methoxyphenyl)-2-(pyridin-4-yl)pyrazolo[1,5-a]pyrimidin-7-yl)-5-fluorophenyl)-2,5-diazabicyclo[2.2.1]-heptane-2-carboxylate

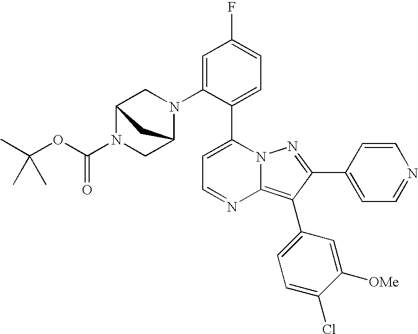

Following the procedure of Example 44, Steps 1-2, starting from 1-(2,4-difluorophenyl)ethanone, followed by the procedure of Example 7, Step 4, using 4-(4-chloro-3-methoxyphenyl)-3-(pyridin-4-yl)-1H-pyrazol-5-amine provided (1S,4S)-tert-butyl 5-(2-(3-(4-chloro-3-methoxyphenyl)-2-(pyridin-4-yl)pyrazolo[1,5-a]pyrimidin-7-yl)-5-fluorophenyl)-2,5-diazabicyclo[2.2.1]heptane-2-carboxylate as a trifluoroacetate salt. MS: 627.3 [M+H].

### Example 49

### 7-(2-((1S,4S)-2,5-Diazabicyclo[2.2.1]heptan-2-yl)-4-fluorophenyl)-3-(4-chloro-3-methoxyphenyl)-2-(pyridin-4-yl)pyrazolo[1,5-a]pyrimidine

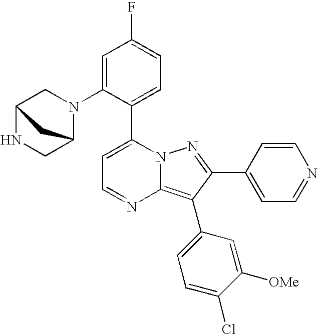

Following the procedure of Example 44, Step 9, (1S,4S)-tert-butyl 5-(2-(3-(4-chloro-3-methoxyphenyl)-2-(pyridin-4-yl)pyrazolo[1,5-a]pyrimidin-7-yl)-5-fluorophenyl)-2,5-diazabicyclo[2.2.1]heptane-2-carboxylate gave 7-(2-((1S,4S)-2,5-diazabicyclo[2.2.1]heptan-2-yl)-4-fluorophenyl)-3-(4-chloro-3-methoxyphenyl)-2-(pyridin-4-yl)pyrazolo[1,5-a]pyrimidine as a trifluoroacetate salt. MS: 527.1 [M+H].

### Example 50

### 3-(4-Chloro-3-methoxyphenyl)-7-(4-fluoro-2-((1S,4S)-5-methyl-2,5-diazabicyclo[2.2.1]heptan-2-yl)phenyl)-2-(pyridin-4-yl)pyrazolo[1,5-a]pyrimidine

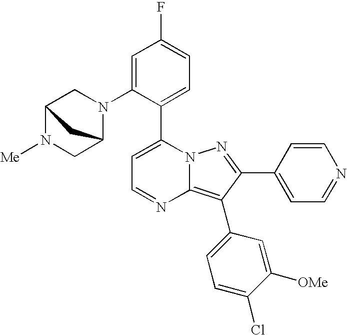

Following the procedure of Example 30, reductive alkylation of 7-(2-((1S,4S)-2,5-diazabicyclo[2.2.1]heptan-2-yl)-4-fluorophenyl)-3-(4-chloro-3-methoxyphenyl)-2-(pyridin-4-yl)pyrazolo[1,5-a]pyrimidine provided 3-(4-chloro-3-methoxyphenyl)-7-(4-fluoro-2-((1S,4S)-5-methyl-2,5-diazabicyclo[2.2.1]heptan-2-yl)phenyl)-2-(pyridin-4-yl)pyrazolo[1,5-a]pyrimidine as a trifluoroacetate salt. MS: 541.1 [M+H].

### Example 51

### 7-(4-((1S,4S)-2,5-Diazabicyclo[2.2.1]heptan-2-yl)phenyl)-3-(1H-indazol-4-yl)-(pyridin-4-yl)pyrazolo[1,5-a]pyrimidine

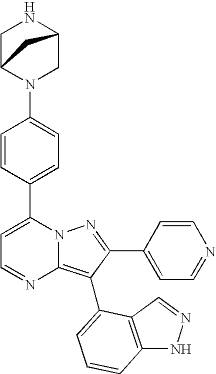

Following the procedure of Example 44, starting from 1-(4-fluorophenyl)ethanone, 7-(4-((1S,4S)-2,5-diazabicyclo[2.2.1]heptan-2-yl)phenyl)-3-(1H-indazol-4-yl)-2-(pyridin-4-yl)pyrazolo[1,5-a]pyrimidine was obtained as a hydrochloride salt. MS: 485.7 [M+H].

### Example 52

### 7-(4-((1S,4S)-2,5-Diazabicyclo[2.2.1]heptan-2-yl)-2-fluorophenyl)-3-(1H-indazol-4-yl)-2-(pyridin-4-yl)pyrazolo[1,5-a]pyrimidine

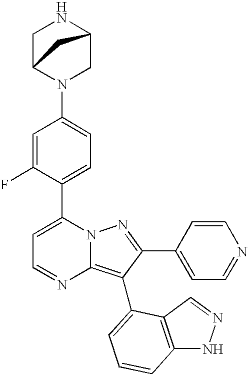

Following the procedure of Example 44, starting from 1-(2,4-difluorophenyl)ethanone, 7-(4-((1S,4S)-2,5-diazabicyclo[2.2.1]heptan-2-yl)-2-fluorophenyl)-3-(1H-indazol-4-yl)-2-(pyridin-4-yl)pyrazolo[1,5-a]pyrimidine was obtained as a hydrochloride salt. MS: 503.1 [M+H].

### Example 53

### 7-(2-Fluoro-4-((1S,4S)-5-methyl-2,5-diazabicyclo[2.2.1]heptan-2-yl)phenyl)-3-(1H-indazol-4-yl)-2-(pyridin-4-yl)pyrazolo[1,5-a]pyrimidine

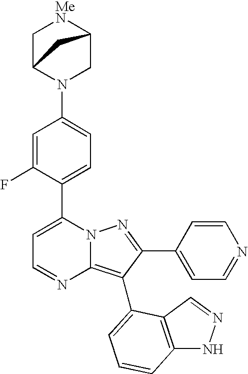

Following the procedure of Example 30, reductive alkylation of 7-(4-((1S,4S)-2,5-diazabicyclo[2.2.1]heptan-2-yl)-2-fluorophenyl)-3-(1H-indazol-4-yl)-2-(pyridin-4-yl)pyrazolo[1,5-a]pyrimidine provided 7-(2-fluoro-4-((1S,4S)-5-methyl-2,5-diazabicyclo[2.2.1]heptan-2-yl)phenyl)-3-(1H-indazol-4-yl)-2-(pyridin-4-yl)pyrazolo[1,5-a]pyrimidine, isolated as a hydrochloride salt. ¹H NMR (400 MHz, CD₃OD) δ 8.72-8.68 (m, 2H), 8.62 (d, J=4.4 Hz, 1H), 8.2-8.16 (m, 2H), 8.05-7.96 (m, 1H), 7.79 (d, J=0.8 Hz, 1H), 7.75-7.70 (m, 1H), 7.61 (dd, J=6.8, 8.4 Hz, 1H), 7.35 (dd, J=0.8, 7.2 Hz, 1H), 7.30 (dd, J=1.2, 4.0 Hz, 1H), 6.82-6.70 (m, 2H), 4.50 (s, 1H), 3.92-3.80 (m, 2H), 3.63 (d, J=11.2 Hz, 1H), 3.04 (s, 3H), 2.55-2.48 (m, 1H), 2.40-2.33 (m, 1H). MS: 517.1 [M+H].

### Example 54

### 5-(7-(4-((1S,4S)-2,5-Diazabicyclo[2.2.1]heptan-2-yl)phenyl)-2-(pyridin-4-yl)pyrazolo[1,5-a]pyrimidin-3-yl)-2-fluorophenol

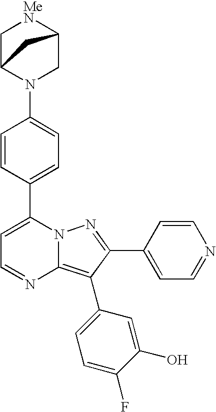

Following the procedure of Example 8, 3-(4-fluoro-3-methoxyphenyl)-7-(4-((1S,4S)-5-methyl-2,5-diazabicyclo[2.2.1]heptan-2-yl)phenyl)-2-(pyridin-4-yl)pyrazolo[1,5-a]pyrimidine and boron tribromide afforded 2-fluoro-5-(7-(4-((1S,4S)-5-methyl-2,5-diazabicyclo[2.2.1]heptan-2-yl)phenyl)-2-(pyridin-4-yl)pyrazolo[1,5-a]pyrimidin-3-yl)phenol, isolated as a hydrochloride salt. ¹H NMR (400 MHz, CD₃OD) δ 8.84-8.79 (m, 2H), 8.58 (d, J=4.8 Hz, 1H), 8.38-8.32 (m, 4H), 7.35 (d, J=4.8 Hz, 1H), 7.20 (dd, J=8.0, 10.8 Hz, 1H), 7.13 (dd, J=2, 8.4 Hz, 1H), 6.98-6.90 (m, 3H), 4.49 (s, 1H), 3.91-3.78 (m, 3H), 3.63 (d, J=11.2 Hz, 1H), 3.03 (s, 3H), 2.54-2.44 (m, 1H), 2.39-2.28 (m, 1H). MS: 493.2 [M+H].

### Example 55

### 3-(4-Fluoro-3-methoxyphenyl)-7-(2-methyl-4-((1S,4S)-5-methyl-2,5-diazabicyclo[2.2.1]heptan-2-yl)phenyl)-2-(pyridin-4-yl)pyrazolo[1,5-a]pyrimidine

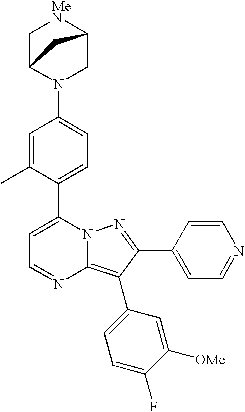

Step 1: Following the procedure of Example 7, Step 4, (1S,4S)-tert-butyl 5-(4-((E)-3-(dimethylamino)acryloyl)-3-methylphenyl)-2,5-diazabicyclo[2.2.1]heptane-2-carboxylate (41 mg, 0.11 mmol) and 4-(4-fluoro-3-methoxyphenyl)-3-(pyridin-4-yl)-1H-pyrazol-5-amine, afforded 7-(4-((1S,4S)-2,5-diazabicyclo[2.2.1]heptan-2-yl)-2-methylphenyl)-3-(4-fluoro-3-methoxyphenyl)-2-(pyridin-4-yl)pyrazolo[1,5-a]pyrimidine. MS: 507.2 [M+H].

Step 5: Following the procedure of Example 30, reductive alkylation of 7-(4-((1S,4S)-2,5-diazabicyclo[2.2.1]heptan-2-yl)-2-methylphenyl)-3-(4-fluoro-3-methoxyphenyl)-2-(pyridin-4-yl)pyrazolo[1,5-a]pyrimidine gave 3-(4-fluoro-3-methoxyphenyl)-7-(2-methyl-4-((1S,4S)-5-methyl-2,5-diazabicyclo[2.2.1]heptan-2-yl)phenyl)-2-(pyridin-4-yl)pyrazolo[1,5-a]pyrimidine, isolated as a trifluoroacetate salt. MS: 521.3 [M+H].

### Example 56

### 2-Fluoro-5-(7-(2-methyl-4-((1S,4S)-5-methyl-2,5-diazabicyclo[2.2.1]heptan-2-yl)phenyl)-2-(pyridin-4-yl)pyrazolo[1,5-a]pyrimidin-3-yl)phenol

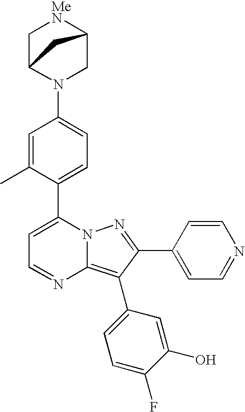

Following the procedure of Example 8, 3-(4-fluoro-3-methoxyphenyl)-7-(2-methyl-4-((1S,4S)-5-methyl-2,5-diazabicyclo[2.2.1]heptan-2-yl)phenyl)-2-(pyridin-4-yl)pyrazolo[1,5-a]pyrimidine and boron tribromide gave 2-fluoro-5-(7-(2-methyl-4-((1S,4S)-5-methyl-2,5-diazabicyclo[2.2.1]heptan-2-yl)phenyl)-2-(pyridin-4-yl)pyrazolo[1,5-a]pyrimidin-3-yl)phenol, isolated as a trifluoroacetate salt. MS: 507.3 [M+H].

### Example 57

### 3-(7-(4-((1S,4S)-2,5-Diazabicyclo[2.2.1]heptan-2-yl)-2-methylphenyl)-2-(pyridin-4-yl)pyrazolo[1,5-a]pyrimidin-3-yl)phenol

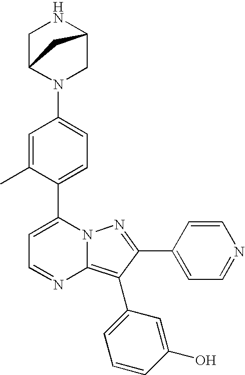

Following the procedure of Example 7, Step 4, 3-(3-amino-5-pyridin-4-yl-1H-pyrazol-4-yl)phenol and (1S,4S)-tert-butyl 5-(4-((E)-3-(dimethylamino)acryloyl)-3-methylphenyl)-2,5-diazabicyclo[2.2.1]heptane-2-carboxylate afforded 3-(7-(4-((1S,4S)-2,5-diazabicyclo[2.2.1]heptan-2-yl)-2-methylphenyl)-2-(pyridin-4-yl)pyrazolo[1,5-a]pyrimidin-3-yl)phenol, isolated as a trifluoroacetate salt. MS: 475.3 [M+H].

### Example 58

### 3-(7-(2-Methyl-4-((1S,4S)-5-methyl-2,5-diazabicyclo[2.2.1]heptan-2-yl)phenyl)-2-(pyridin-4-yl)pyrazolo[1,5-a]pyrimidin-3-yl)phenol

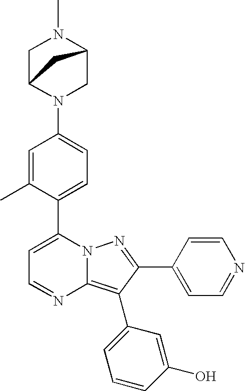

Following the procedure of Example 30, reductive alkylation of 3-(7-(4-((1S,4S)-2,5-diazabicyclo[2.2.1]heptan-2-yl)-2-methylphenyl)-2-(pyridin-4-yl)pyrazolo[1,5-a]pyrimidin-3-yl)phenol afforded 3-(7-(2-methyl-4-((1S,4S)-5-methyl-2,5-diazabicyclo[2.2.1]heptan-2-yl)phenyl)-2-(pyridin-4-yl)pyrazolo[1,5-a]pyrimidin-3-yl)phenol, isolated as a trifluoroacetate salt. MS: 489.3 [M+H].

### Example 59

### 3-(7-(4-((1S,4S)-2,5-Diazabicyclo[2.2.1]heptan-2-yl)-2-chlorophenyl)-2-(pyridin-4-yl)pyrazolo[1,5-a]pyrimidin-3-yl)phenol

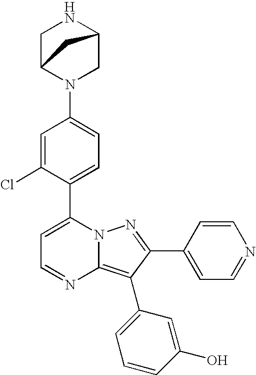

Step 1: Following the procedure of Example 44, Steps 1-2, (1S,4S)-tert-butyl 5-(3-chloro-4-((E)-3-(dimethylamino)acryloyl)phenyl)-2,5-diazabicyclo[2.2.1]heptane-2-carboxylate was prepared starting from 1-(2-chloro-4-fluorophenyl)ethanone.

Step 2: Following the procedure of Example 7, Step 4, (1S,4S)-tert-butyl 5-(3-chloro-4-((E)-3-(dimethylamino)acryloyl)phenyl)-2,5-diazabicyclo[2.2.1]heptane-2-carboxylate and 3-(3-amino-5-pyridin-4-yl-1H-pyrazol-4-yl)phenol afforded 3-(7-(4-((1S,4S)-2,5-diazabicyclo[2.2.1]heptan-2-yl)-2-chlorophenyl)-2-(pyridin-4-yl)pyrazolo[1,5-a]pyrimidin-3-yl)phenol as a trifluoroacetate salt. MS: 495.3 [M+H].

### Example 60

### 3-(7-(2-Chloro-4-((1S,4S)-5-methyl-2,5-diazabicyclo[2.2.1]heptan-2-yl)phenyl)-2-(pyridin-4-yl)pyrazolo[1,5-a]pyrimidin-3-yl)phenol

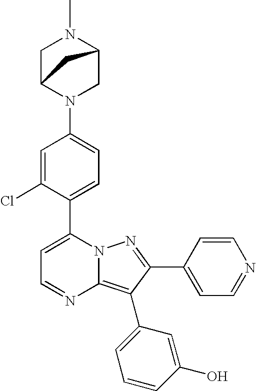

Following the procedure of Example 30, reductive alkylation of 3-(7-(4-((1S,4S)-2,5-diazabicyclo[2.2.1]heptan-2-yl)-2-chlorophenyl)-2-(pyridin-4-yl)pyrazolo[1,5-a]pyrimidin-3-yl)phenol afforded 3-(7-(2-chloro-4-((1S,4S)-5-methyl-2,5-diazabicyclo[2.2.1]heptan-2-yl)phenyl)-2-(pyridin-4-yl)pyrazolo[1,5-a]pyrimidin-3-yl)phenol, isolated as a trifluoroacetate salt. MS: 509.3 [M+H].

### Example 61

### 7-(4-((1S,4S)-2,5-Diazabicyclo[2.2.1]heptan-2-yl)-2-chlorophenyl)-3-(1H-indazol-4-yl)-2-(pyridin-4-yl)pyrazolo[1,5-a]pyrimidine

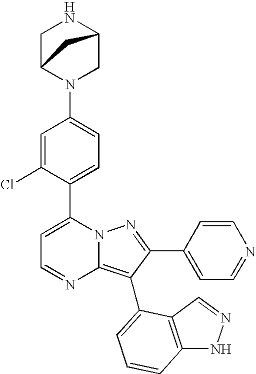

Following the procedure of Example 44, Step 8, (1S,4S)-tert-butyl 5-(3-chloro-4-((E)-3-(dimethylamino)acryloyl)phenyl)-2,5-diazabicyclo[2.2.1]heptane-2-carboxylate and 4-(1H-indazol-4-yl)-3-(pyridin-4-yl)-1H-pyrazol-5-amine (34 mg, 0.12 mmol) provided 7-(4-((1S,4S)-2,5-diazabicyclo[2.2.1]heptan-2-yl)-2-chlorophenyl)-3-(1H-indazol-4-yl)-2-(pyridin-4-yl)pyrazolo[1,5-a]pyrimidine as a trifluoroacetate salt after deprotection with trifluoroacetic acid. MS: 519.2 [M+H].

### Example 62

### 7-(2-Chloro-4-((1S,4S)-5-methyl-2,5-diazabicyclo[2.2.1]heptan-2-yl)phenyl)-3-(1H-indazol-4-yl)-2-(pyridin-4-yl)pyrazolo[1,5-a]pyrimidine

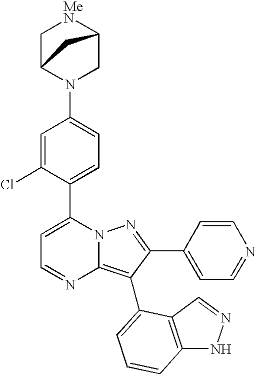

Following the procedure of Example 30, reductive alkylation of 7-(4-((1S,4S)-2,5-diazabicyclo[2.2.1]heptan-2-yl)-2-chlorophenyl)-3-(1H-indazol-4-yl)-2-(pyridin-4-yl)pyrazolo[1,5-a]pyrimidine gave 7-(2-chloro-4-((1S,4S)-5-methyl-2,5-diazabicyclo[2.2.1]heptan-2-yl)phenyl)-3-(1H-indazol-4-yl)-2-(pyridin-4-yl)pyrazolo[1,5-a]pyrimidine, isolated as a trifluoroacetate salt. MS: 533.3 [M+H].

### Example 63

### Ethyl 3-[3-(3-hydroxy-4-methyl phenyl)-2-pyridin-4-ylpyrazolo[1,5-a]pyrimidin-7-yl]-8-azabicyclo[3.2.1]octane-8-carboxylate trifluoroacetate salt

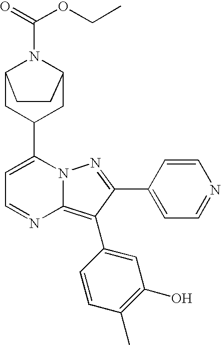

Step 1: According to the procedure of Example 7, Step 4, (E)-ethyl 3-(3-(dimethylamino)acryloyl)-8-azabicyclo[3.2.1]octane-8-carboxylate (0.37 g, 1.3 mmol) and 3-(pyridin-4-yl)-1H-pyrazol-5-amine provided ethyl 3-(2-(pyridin-4-yl)pyrazolo[1,5-a]pyrimidin-7-yl)-8-azabicyclo[3.2.1]octane-8-carboxylate.

Step 2: To a solution of ethyl 3-(2-(pyridin-4-yl)pyrazolo[1,5-a]pyrimidin-7-yl)-8-azabicyclo[3.2.1]octane-8-carboxylate (0.8 g, 2.1 mmol) in dichloromethane (75 mL) was added N-iodosuccinimide (5.7 g, 25 mmol) in four portions over a 3 hour period and the reaction was then stirred for an additional 16 hours. The reaction mixture was washed with saturated sodium thiosulfate (2×200 mL) and the organic layer was dried over anhydrous sodium sulfate, filtered and then concentrated in-vacuo to give 0.8 g of ethyl 3-(3-iodo-2-(pyridin-4-yl)pyrazolo[1,5-a]pyrimidin-7-yl)-8-azabicyclo[3.2.1]octane-8-carboxylate as a yellow solid. This product was used in the next step without further purification. MS: 504.3 [M+H].

Step 3: To a suspension of ethyl 3-(3-iodo-2-pyridin-4-ylpyrazolo[1,5-a]pyrimidin-7-yl)-8-azabicyclo[3.2.1]octane-8-carboxylate (100 mg, 0.20 mmol) in 1.5 mL of dimethoxyethane was added 2M sodium carbonate (0.5 mL), 3-methoxy-4-methylboronic acid (40 mg, 0.24 mmol) and a catalytic amount of (1,1′-bis(diphenylphosphino)ferrocene)dichloropalladium(II)dichloromethane complex (16 mg, 0.02 mmol). The mixture was heated to 100° C. for 60 minutes in a microwave reactor. Additional portions of 3-methoxy-4-methylboronic acid (20 mg, 0.12 mmol) and (1,1′-bis(diphenylphosphino)ferrocene)dichloropalladium(II)dichloromethane complex (10 mg, 0.012 mmol) were added, and the mixture was reacted at 100° C. for an additional 40 minutes in the microwave reactor. The crude product was purified by silica gel chromatography eluting with a gradient of 99:1 to 97:3 dichloromethane/methanol to provide 67 mg (68% yield) of 3-[3-(3-methoxy-4-methyl-phenyl)-2-pyridin-4-yl-pyrazolo[1,5-a]pyrimidin-7-yl]-8-aza-bicyclo[3.2.1]octane-8-carboxylic acid ethyl ester as an orange oil. MS: 498.4 [M+H].

Step 4: Following the procedure of Example 8, 3-[3-(3-methoxy-4-methyl-phenyl)-2-pyridin-4-yl-pyrazolo[1,5-a]pyrimidin-7-yl]-8-aza-bicyclo[3.2.1]octane-8-carboxylic acid ethyl ester was reacted with boron tribromide in dichloromethane to provide ethyl 3-[3-(3-hydroxy-4-methylphenyl)-2-pyridin-4-ylpyrazolo[1,5-a]pyrimidin-7-yl]-8-azabicyclo[3.2.1]octane-8-carboxylate trifluoroacetate salt, as a white solid (15% yield) after purification by preparative HPLC (H₂O/acetonitrile/trifluoroacetic acid). MS: 484.0 [M+H].

### Example 64

### 5-(7-(8-Ethyl-8-azabicyclo[3.2.1]octan-3-yl)-2-(pyridin-4-yl)pyrazolo[1,5-a]pyrimidin-3-yl)-2-fluorophenol

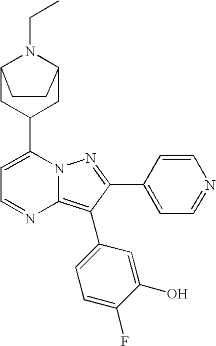

Step 1: Following the procedure of Example 20, Step 7, 7-(8-ethyl-8-azabicyclo[3.2.1]octan-3-yl)-3-iodo-2-(pyridin-4-yl)pyrazolo[1,5-a]pyrimidine (150 mg, 0.326 mmol) reacted with 4-fluoro-3-methoxyphenylboronic acid (167 mg, 0.983 mmol) to yield 7-(8-ethyl-8-azabicyclo[3.2.1]octan-3-yl)-3-(4-fluoro-3-methoxyphenyl)-2-(pyridin-4-yl)pyrazolo[1,5-a]pyrimidine as a yellow solid. MS 458.1 [M+H].

Step 2: Following the procedure of Example 8, 7-(8-ethyl-8-azabicyclo[3.2.1]octan-3-yl)-3-(4-fluoro-3-methoxyphenyl)-2-(pyridin-4-yl)pyrazolo[1,5-a]pyrimidine was reacted with boron tribromide and then purified using preparative TLC using a 5-10% methanol/dichloromethane gradient to provide 4.8 mg (3.3% yield) 5-(7-(8-ethyl-8-azabicyclo[3.2.1]octan-3-yl)-2-(pyridin-4-yl)pyrazolo[1,5-a]pyrimidin-3-yl)-2-fluorophenol as a yellow solid. MS 444.3 [M+H].

### Example 65

### Ethyl 3-(3-(2,3-difluorophenyl)-2-(pyridin-4-yl)pyrazolo[1,5-a]pyrimidin-7-yl)-8-azabicyclo[3.2.1]octane-8-carboxylate

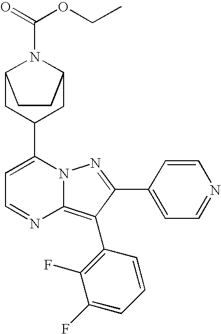

Following the procedure of Example 20, Step 7, ethyl 3-(3-iodo-2-(pyridin-4-yl)pyrazolo[1,5-a]pyrimidin-7-yl)-8-azabicyclo[3.2.1]octane-8-carboxylate (100 mg, 0.199 mmol) was reacted with 2,3-difluorophenylboronic acid (141 mg, 0.89 mmol) to yield 47.1 mg (48.5%) of ethyl 3-(3-(2,3-difluorophenyl)-2-(pyridin-4-yl)pyrazolo[1,5-a]pyrimidin-7-yl )-8-azabicyclo[3.2.1]octane-8-carboxylate as a yellow solid after preparative TLC eluting with 4% methanol in dichloromethane. MS 490.3 [M+H].

### Example 66

### Ethyl 3-(3-(3-(methylsulfonamido)phenyl)-2-(pyridin-4-yl)pyrazolo[1,5-a]pyrimidin-7-yl)-8-azabicyclo[3.2.1]octane-8-carboxylate

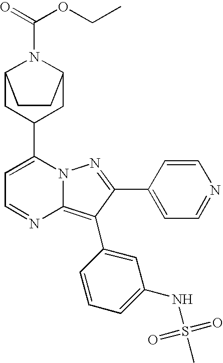

Following the procedure of Example 20, Step 7, ethyl 3-(3-iodo-2-(pyridin-4-yl)pyrazolo[1,5-a]pyrimidin-7-yl)-8-azabicyclo[3.2.1]octane-8-carboxylate (100 mg, 0.199 mmol) was reacted with 3-(methylsulfonamido)phenylboronic acid (192 mg, 0.89 mmol) to yield 76.3 mg (70.3%) of ethyl 3-(3-(3-(methylsulfonamido)phenyl)-2-(pyridin-4-yl)pyrazolo[1,5-a]pyrimidin-7-yl)-8-azabicyclo[3.2.1]octane-8-carboxylate as a yellow solid after preparative TLC eluting with 4% methanol in dichloromethane. MS 547.3 [M+H].

### Example 67

### Methyl 4-(7-(8-ethyl-8-azabicyclo[3.2.1]octan-3-yl)-2-(pyridin-4-yl)pyrazolo[1,5-a]pyrimidin-3-yl)phenylcarbamate

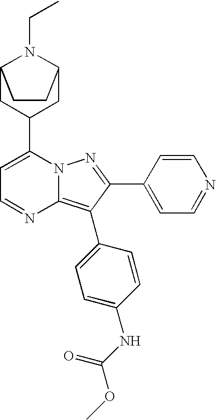

A mixture of 4-(7-(8-ethyl-8-azabicyclo[3.2.1]octan-3-yl)-2-(pyridin-4-yl)pyrazolo[1,5-a]pyrimidin-3-yl)aniline (65 mg, 0.15 mmol) and triethylamine (62 □L, 0.45 mmol) in dichloromethane (3 mL) was added to a solution of triphosgene (23 mg, 0.075 mmol) in dichloromethane (0.5 mL) and the resulting mixture was stirred for 10 minutes. Methanol (2 mL) was then added to the reaction and the resulting mixture was stirred for an additional 30 minutes. The reaction was diluted with dichloromethane (5 mL) and washed with saturated sodium bicarbonate (2×5 mL). The organic layer was dried over anhydrous sodium sulfate, filtered, and concentrated in-vacuo to give a solid. The crude solid was purified by silica gel flash chromatography, eluting with 15% methanol in dichloromethane, to give 48 mg (66% yield) of methyl 4-(7-(8-ethyl-8-azabicyclo[3.2.1]octan-3-yl)-2-(pyridin-4-yl )pyrazolo[1,5-a]pyrimidin-3-yl)phenylcarbamate as a yellow solid. MS 483.3 [M+H].

### Example 68

### 4-(7-(8-Ethyl-8-azabicyclo[3.2.1]octan-3-yl)-2-(pyridin-4-yl)pyrazolo[1,5-a]pyrimidin-3-yl)-2-hydroxybenzonitrile

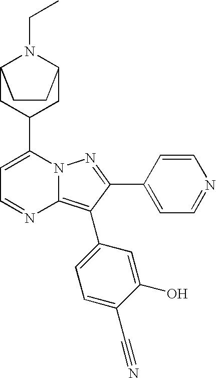

Step 1: To a solution of 4-bromo-2-fluorobenzonitrile (5 g, 25 mmol) in tetrahydrofuran was added sodium methoxide (125 mmol) in methanol (20 mL) and the reaction was stirred at 40° C. for 3 hours. The reaction was then cooled to room temperature and Amberlyst™ 15 was added and the mixture was stirred for 2 hours. The reaction was filtered and the organics were concentrated in-vacuo to give 4-bromo-2-methoxybenzonitrile as a white solid that was used directly in the next reaction. MS 212.1 [M+H].

Step 2: According to the procedure of Example 40, Step 4, 4-bromo-2-methoxybenzonitrile was converted into 2-methoxy-4-(4,4,5,5-tetramethyl-1,3,2-dioxaborolan-2-yl)benzonitrile which was used directly in the next reaction.

Step 3: The reaction of 7-(8-ethyl-8-azabicyclo[3.2.1]octan-3-yl)-3-iodo-2-(pyridin-4-yl)pyrazolo[1,5-a]pyrimidine with 2-methoxy-4-(4,4,5,5-tetramethyl-1,3,2-dioxaborolan-2-yl)benzonitrile, according to the procedure of Example 20, Step 7, provided 4-(7-(8-ethyl-8-azabicyclo[3.2.1]octan-3-yl)-2-(pyridin-4-yl)pyrazolo[1,5-a]pyrimidin-3-yl )-2-methoxybenzonitrile as a brown solid that was used directly in the next reaction.

Step 4: Following the procedure of Example 8, 4-(7-(8-ethyl-8-azabicyclo[3.2.1]octan-3-yl)-2-(pyridin-4-yl)pyrazolo[1,5-a]pyrimidin-3-yl)-2-methoxybenzonitrile (0.066 g, 0.14 mmol) was reacted with a 1 M solution of boron tribromide in dichloromethane (4.6 mL) to provide 0.019 g (30% yield) of 4-(7-(8-ethyl-8-azabicyclo[3.2.1]octan-3-yl )-2-(pyridin-4-yl)pyrazolo[1,5-a]pyrimidin-3-yl)-2-hydroxybenzonitrile as a yellow solid. MS 451.3 [M+H].

### Example 69

### tert-Butyl 4-(7-(8-ethyl-8-azabicyclo[3.2.1]octan-3-yl)-2-(pyridin-4-yl)pyrazolo[1,5-a]pyrimidin-3-yl)-2-methoxyphenylcarbamate

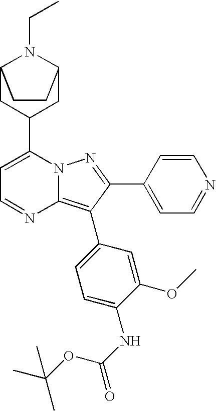

According to the procedure of Example 20, Step 7, the reaction of 7-(8-ethyl-8-azabicyclo[3.2.1]octan-3-yl)-3-iodo-2-(pyridin-4-yl)pyrazolo[1,5-a]pyrimidine with tert-butyl 2-methoxy-4-(4,4,5,5-tetramethyl-1,3,2-dioxaborolan-2-yl)phenylcarbamate, provided 0.28 g (39% yield) of tert-butyl 4-(7-(8-ethyl-8-azabicyclo[3.2.1]octan-3-yl)-2-(pyridin-4-yl)pyrazolo[1,5-a]pyrimidin-3-yl)-2-methoxyphenylcarbamate as a yellow solid. MS 555.5 [M+H].

### Example 70

### 4-(7-(8-Ethyl-8-azabicyclo[3.2.1]octan-3-yl)-2-(pyridin-4-yl )pyrazolo[1,5-a]pyrimidin-3-yl)-2-methoxyaniline

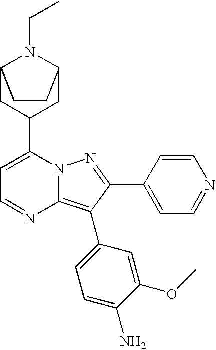

To a solution of tert-butyl 4-(7-(8-ethyl-8-azabicyclo[3.2.1]octan-3-yl)-2-(pyridin-4-yl)pyrazolo[1,5-a]pyrimidin-3-yl)-2-methoxyphenylcarbamate (281 mg, 0.51 mmol) in dichloromethane (5 mL) was added trifluoroacetic acid (0.5 mL). The reaction was stirred for 5 hours and then concentrated in-vacuo. The crude solid was suspended in saturated sodium bicarbonate (100 mL) and extracted with 5% methanol in dichloromethane. The combined organic layers were dried over anhydrous sodium sulfate, filtered, and concentrated in vacuo to give 0.22 g (94% yield) of 4-(7-(8-ethyl-8-azabicyclo[3.2.1]octan-3-yl)-2-(pyridin-4-yl)pyrazolo[1,5-a]pyrimidin-3-yl)-2-methoxyaniline as a yellow solid. MS 455.4 [M+H].

### Example 71

### 2-Amino-5-(7-(8-ethyl-8-azabicyclo[3.2.1]octan-3-yl)-2-(pyridin-4-yl)pyrazolo[1,5-a]pyrimidin-3-yl)phenol

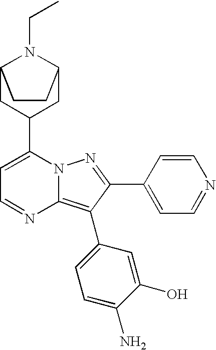

According to the procedure of Example 8, 4-(7-(8-ethyl-8-azabicyclo[3.2.1]octan-3-yl)-2-(pyridin-4-yl)pyrazolo[1,5-a]pyrimidin-3-yl)-2-methoxyaniline (0.21 g, 0.46 mmol) was reacted with a 1 M solution of boron tribromide in dichloromethane (4.6 mL) to provide 0.068 g (34% yield) of 2-amino-5-(7-(8-ethyl-8-azabicyclo[3.2.1]octan-3-yl )-2-(pyridin-4-yl)pyrazolo[1,5-a]pyrimidin-3-yl)phenol as a yellow solid. MS 441.3 [M+H].

### Examples 72a & 72b

### N-(4-(7-(8-Ethyl-8-azabicyclo[3.2.1]octan-3-yl)-2-(pyridin-4-yl)pyrazolo[1,5-a]pyrimidin-3-yl)-2-hydroxyphenyl)formamide (WYE-126925) N-(4-(7-(8-ethyl-8-azabicyclo[3.2.1]octan-3-yl)-2-(pyridin-4-yl)pyrazolo[1,5-a]pyrimidin-3-yl)-2-methoxyphenyl)formamide

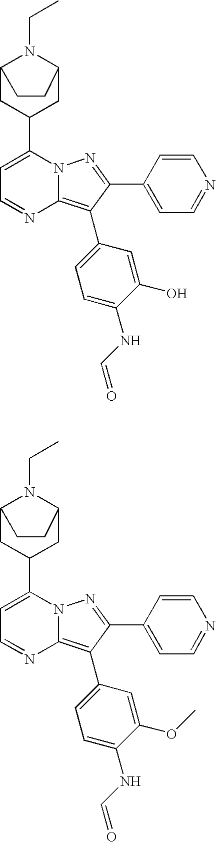

A mixture of formic acid (19 mg, 0.42 mmol) and acetic anhydride (34.5 mg, 0.34 mmol) was stirred at 60° C. for 2 hours and then cooled to room temperature and added to a solution of a mixture of 2-amino-5-(7-(8-ethyl-8-azabicyclo[3.2.1]octan-3-yl)-2-(pyridin-4-yl)pyrazolo[1,5-a]pyrimidin-3-yl)phenol and 4-(7-(8-ethyl-8-azabicyclo[3.2.1]octan-3-yl)-2-(pyridin-4-yl)-pyrazolo[1,5-a]pyrimidin-3-yl)-2-methoxyaniline (1:1, 58 mg, 0.13 mmol) in 1 mL of THF at 0° C. The reaction was stirred for 2 hours and then quenched with saturated potassium carbonate. The mixture was extracted with 5% methanol in dichloromethane and the combined organic layers were dried over anhydrous sodium sulfate, filtered, and then concentrated in-vacuo. The crude product was purified by preparatory TLC eluting with 10% methanol in dichloromethane to give 4.1 mg of Example 72a N-(4-(7-(8-ethyl-8-azabicyclo[3.2.1]octan-3-yl)-2-(pyridin-4-yl)pyrazolo[1,5-a]pyrimidin-3-yl)-2-hydroxyphenyl)formamide as an orange solid (MS 469.4 [M+H]) and 6.4 mg of Example 72b N-(4-(7-(8-ethyl-8-azabicyclo[3.2.1]octan-3-yl)-2-(pyridin-4-yl)pyrazolo[1,5-a]pyrimidin-3-yl)-2-methoxyphenyl)formamide as a orange solid (MS 483.3 [M+H]).

### Example 73

### Ethyl 3-[3-(1H-indazol-4-yl)-2-pyridin-4-ylpyrazolo[1,5-a]pyrimidin-7-yl]-8-azabicyclo[3.2.1]octane-8-carboxylate

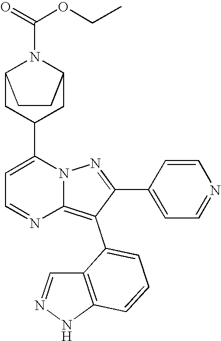

According to the procedure of Example 63, Step 3, 6-(4,4,5,5-tetramethyl-[1,3,2]dioxaborolan-2-yl)-and 3-(3-iodo-2-pyridin-4-yl-pyrazolo[1,5-a]pyrimidin-7-yl)-8-azabicyclo[3.2.1]octane-8-carboxylic acid ethyl ester provided ethyl 3-[3-(1H-indazol-4-yl)-2-pyridin-4-ylpyrazolo[1,5-a]pyrimidin-7-yl]-8-azabicyclo[3.2.1]octane-8-carboxylate in 40% yield after purification by RP-HPLC. MS 494.3 [M+H].

### Example 74

### 7-(8-Azabicyclo[3.2.1]oct-3-yl)-3-(1H-indazol-4-yl)-2-pyridin-4-ylpyrazolo[1,5-a]pyrimidine

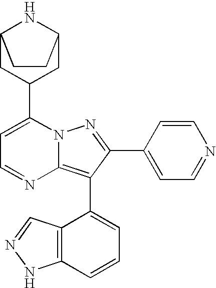

Following the procedure of Example 9, ethyl 3-[3-(1H-indazol-4-yl)-2-pyridin-4-ylpyrazolo[1,5-a]pyrimidin-7-yl]-8-azabicyclo[3.2.1]octane-8-carboxylate was reacted with iodotrimethylsilane in refluxing chloroform to provide 7-(8-azabicyclo[3.2.1]oct-3-yl)-3-(1H-indazol-4-yl)-2-pyridin-4-ylpyrazolo[1,5-a]pyrimidine as a yellow solid in 90% yield after silica gel chromatography, eluting with a gradient of 95:5 to 4:1 dichloromethane/methanol followed by 80:20:1 dichloromethane/methanol/aqueous ammonium hydroxide. MS: 422.2 [M+H].

### Example 75

### Ethyl 3-[3-(7-chloro-1H-indazol-4-yl)-2-pyridin-4-ylpyrazolo[1,5-a]pyrimidin-7-yl]-8-azabicyclo[3.2.1]octane-8-carboxylate

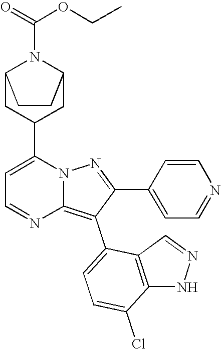

According to the procedure of Example 63, Step 3, 3-(3-iodo-2-pyridin-4-yl-pyrazolo[1,5-a]pyrimidin-7-yl)-8-aza-bicyclo[3.2.1]octane-8-carboxylic acid ethyl ester and 7-chloro-4-(4,4,5,5-tetramethyl-[1,3,2]dioxaborolan-2-yl)-1H-indazole gave ethyl 3-[3-(7-chloro-1H-indazol-4-yl)-2-pyridin-4-ylpyrazolo[1,5-a]pyrimidin-7-yl]-8-azabicyclo[3.2.1]octane-8-carboxylate in 42% yield following purification by RP-HPLC. MS 528.0 [M+H].

### Example 76

### Ethyl 3-{2-pyridin-4-yl-3-[7-(trifluoromethyl)-1H-indazol-4-yl]pyrazolo[1,5-a]pyrimidin-7-yl}-8-azabicyclo[3.2.1]octane-8-carboxylate

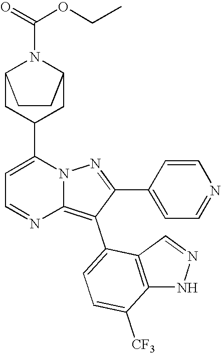

Step 1: To a solution of 2,2,6,6-tetramethylpiperidine (725 mg, 5.18 mmol) in THF (10 mL) at −78° C. was added a solution of 2.5M butyllithium in hexanes (2.07 mL, 5.18 mmol) and 4-bromo-2-fluoro-1-trifluoromethyl-benzene (1.2 g, 4.9 mmol). The mixture warmed to −20° C. for 2 hours, and then the reaction was quenched with water (100 mL), neutralized with 1 M HCl and extracted with ether (3×30 mL). The combined organic extracts were dried over sodium sulfate, filtered, and concentrated in vacuo. The residue was purified by silica gel chromatography to provide 6-bromo-2-fluoro-3-trifluoromethyl-benzaldehyde in 82% yield. MS: 270.0 [M+H].

Step 2: To a solution of 6-bromo-2-fluoro-3-trifluoromethyl-benzaldehyde (1.0 g, 3.7 mmol) in dimethoxyethane, DME (5 mL) was added hydrazine hydrate (5 mL). The mixture was refluxed for 3 hours, and then cooled to room temperature. The solvent was evaporated, water (100 mL) was added and the reaction mixture was extracted with ethyl acetate (3×30 mL). The combined organic layers were dried over sodium sulfate, filtered, and then concentrated in-vacuo. The residue was purified by silica gel chromatography to afford 4-bromo-7-(trifluoromethyl)-1H-indazole in 42% yield. MS 264.9 [M+H].

Step 3: To a solution of 4-bromo-7-(trifluoromethyl)-1H-indazole (500 mg, 1.89 mmol) in DMSO (5 mL) was added potassium acetate (610 g, 6.23 mmol), 1,1′-bis(diphenyl phosphino)ferrocene palladium chloride (77 mg, 0.09 mmol) and bis(pinacolato)diboron (576 g, 2.27 mmol). The mixture was degassed and heated in oil bath overnight at 100° C. The reaction was filtered through a pad of Celite™, water (60 mL) was added to the filtrate, and the mixture was extracted with ethyl acetate (3×30 mL). The combined organic extracts were dried over sodium sulfate, filtered, and then concentrated in-vacuo to provide 4-(4,4,5,5-tetramethyl-[1,3,2]dioxaborolan-2-yl)-7-trifluoromethyl-1H-indazole which was used in the next step without further purification.

Step 4: According to the procedure of Example 63, Step 3, 3-(3-iodo-2-pyridin-4-ylpyrazolo[1,5-a]pyrimidin-7-yl)-8-aza-bicyclo[3.2.1]octane-8-carboxylic acid ethyl ester and 4-(4,4,5,5-tetramethyl-[1,3,2]dioxaborolan-2-yl)-7-trifluoromethyl-1H-indazole gave ethyl 3-{2-pyridin-4-yl-3-[7-(trifluoromethyl )-1H-indazol-4-yl]pyrazolo[1,5-a]pyrimidin-7-yl}-8-azabicyclo[3.2.1]octane-8-carboxylate (32 mg, 37% yield) after purification by RP-HPLC. MS: 562.3 [M+H].

### Example 77

### 7-(8-ethyl-8-azabicyclo[3.2.1]oct-3-yl)-3-(7-methyl-1H-indazol-4-yl)-2-pyridin-4-ylpyrazolo[1,5-a]pyrimidine, trifluoroacetate salt

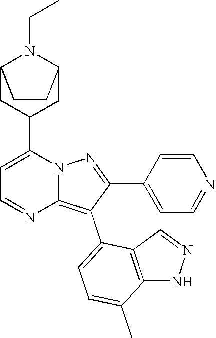

According to the procedure for Example 63, Step 3, 7-methyl-4-(4,4,5,5-tetramethyl-[1,3,2]dioxaborolan-2-yl)-1H-indazole and 7-(8-ethyl-8-azabicyclo[3.2.1]oct-3-yl)-3-iodo-2-pyridin-4-ylpyrazolo[1,5-a]pyrimidine provided 7-(8-ethyl-8-azabicyclo[3.2.1]oct-3-yl)-3-(7-methyl-1H-indazol-4-yl)-2-pyridin-4-ylpyrazolo[1,5-a]pyrimidine, trifluoro acetate salt, as a yellow solid in 18% yield following purification by RP-HPLC. MS: 464.3 [M+H].

### Example 78

### Ethyl 3-[3-(7-Methyl-1H-indazol-4-yl)-2-pyridin-4-ylpyrazolo[1,5-a]pyrimidin-7-yl]-8-azabicyclo[3.2.1]octane-8-carboxylate, trifuoroacetate salt

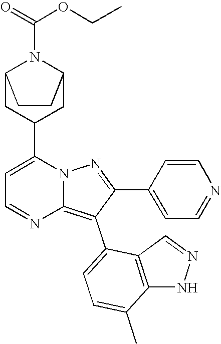

According to the procedure of Example 63, Step 3, 7-methyl-4-(4,4,5,5-tetramethyl-[1,3,2]dioxaborolan-2-yl)-1H-indazole and 3-(3-iodo-2-pyridin-4-yl-pyrazolo[1,5-a]pyrimidin-7-yl)-8-aza-bicyclo[3.2.1]octane-8-carboxylic acid ethyl ester provided ethyl 3-[3-(7-methyl-1H-indazol-4-yl)-2-pyridin-4-ylpyrazolo[1,5-a]pyrimidin-7-yl]-8-azabicyclo[3.2.1]octane-8-carboxylate, trifluoro acetate salt, as a yellow solid in 8% yield following purification by RP-HPLC. MS: 508.3 [M+H].

### Example 79

### 3-(7-Chloro-1H-indazol-4-yl)-7-(8-ethyl-8-azabicyclo[3.2.1]oct-3-yl)-2-pyridin-4-ylpyrazolo[1,5-a]pyrimidin, trifluoroacetate salt

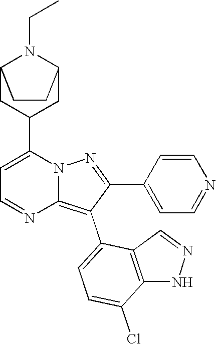

According to the procedure of Example 63, Step 3, 7-chloro-4-(4,4,5,5-tetramethyl-[1,3,2]dioxaborolan-2-yl)-1H-indazole and 7-(8-ethyl-8-azabicyclo[3.2.1]oct-3-yl)-3-iodo-2-pyridin-4-ylpyrazolo[1,5- a]pyrimidine provided 3-(7-chloro-1H-indazol-4-yl)-7-(8-ethyl-8-azabicyclo[3.2.1]oct-3-yl)-2-pyridin-4-ylpyrazolo[1,5-a]pyrimidin, trifluoro acetate salt, as a yellow solid in 35% yield following purification by RP-HPLC. MS: 484.1 [M+H].

### Example 80

### 7-(8-Ethyl-8-azabicyclo[3.2.1]oct-3-yl)-2-pyridin-4-yl-3-[7-(trifluoromethyl)-1H-indazol-4-yl]pyrazolo[1,5-a]pyrimidine, trifluoroacetate salt

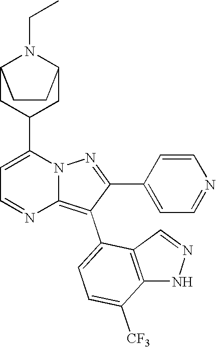

According to the procedure of Example 63, Step 3, 4-(4,4,5,5-tetramethyl-[1,3,2]dioxaborolan-2-yl)-7-trifluoromethyl-1H-indazole and 7-(8-ethyl-8-azabicyclo[3.2.1]oct-3-yl)-3-iodo-2-pyridin-4-ylpyrazolo[1,5-a]pyrimidine provided 7-(8-ethyl-8-azabicyclo[3.2.1]oct-3-yl)-2-pyridin-4-yl-3-[7-(trifluoromethyl)-1H-indazol-4-yl]pyrazolo[1,5-a]pyrimidine, trifluoro acetate salt, as a yellow solid in 8% yield following purification by RP-HPLC. MS: 518.3 [M+H].

### Example 81

### Ethyl 3-[3-(7-fluoro-1H-indazol-4-yl)-2-pyridin-4-ylpyrazolo[1,5-a]pyrimidin-7-yl]-8-azabicyclo[3.2.1]octane-8-carboxylate, trifluoroacetate salt

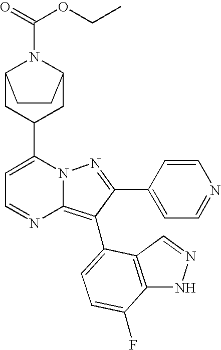

According to the procedure of Example 63, Step 3, 7-fluoro-4-(4,4,5,5-tetramethyl-1,3,2-dioxaborolan-2-yl)-1H-indazole and 3-(3-iodo-2-pyridin-4-yl-pyrazolo[1,5-a]pyrimidin-7-yl)-8-aza-bicyclo[3.2.1]octane-8-carboxylic acid ethyl ester gave ethyl 3-[3-(7-fluoro-1H-indazol-4-yl)-2-pyridin-4-ylpyrazolo[1,5-a]pyrimidin-7-yl]-8-azabicyclo[3.2.1]octane-8-carboxylate, trifluoroacetate salt, as a yellow solid in 19% yield following purification by RP-HPLC. MS: 512.3 [M+H].

### Example 82

### 3-(7-Chloro-6-fluoro-1H-indazol-4-yl)-7-(8-ethyl-8-azabicyclo[3.2.1]oct-3-yl)-2-pyridin-4-ylpyrazolo[1,5-a]pyrimidine, trifluoro acetate salt

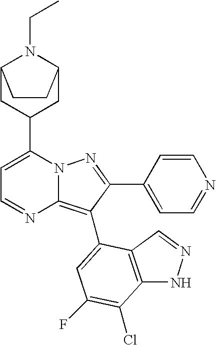

Step 1: According to the procedure of Example 41, Steps 1-3, 5-bromo-2-chloro-1,3-difluorobenzne afforded 7-chloro-6-fluoro-4-(4,4,5,5-tetramethyl-1,3,2-dioxaborolan-2-yl)-1H-indazole as a white solid. MS: 297.1 [M+H].

Step 2: According to the procedure of Example 63, Step 3, 7-chloro-6-fluoro-4-(4,4,5,5-tetramethyl-1,3,2-dioxaborolan-2-yl)-1H-indazole and 7-(8-ethyl-8-azabicyclo[3.2.1]oct-3-yl)-3-iodo-2-pyridin-4-ylpyrazolo[1,5- a]pyrimidine provided 3-(7-chloro-6-fluoro-1H-indazol-4-yl)-7-(8-ethyl-8-azabicyclo[3.2.1]oct-3-yl)-2- pyridin-4-ylpyrazolo[1,5a]pyrimidine, trifluoro acetate salt, as a yellow solid in 7% yield following purification by RP-HPLC. MS: 502.1 [M+H].

### Example 83

### Ethyl 3-[3-(2-oxo-2,3-dihydro-1H-benzimidazol-4-yl)-2-pyridin-4-ylpyrazolo[1,5-a]pyrimidin-7-yl]-8-azabicyclo[3.2.1]octane-8-carboxylate

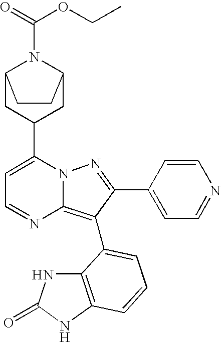

Step 1: To a solution of 4-bromo-benzo[1,2,5]thiadiazole (1.15 g, 5.35 mmol) in methanol (10 mL) was added sodium borohydride (203 mg, 5.35 mmol) and cobalt(II) chloride hexahydrate (120 mg, 0.533 mmol). The mixture was refluxed for 3 hours, then cooled to room temperature and filtered to remove the black solid. The solvent was evaporated, water (100 mL) was added and the mixture was extracted with ether (3×30 mL). The combined organic extracts were dried over sodium sulfate, filtered, and then concentrated in-vacuo, to provide 3-bromo-benzene-1,2-diamine (810 mg, 81% yield). MS: 187.0 [M+H].

Step 2: To a solution of 3-bromo-benzene-1,2-diamine (810 mg, 4.33 mmol) in THF (10 mL) was added triphosgene (2.57 g, 8.66 mmol) and triethylamine (1.15 mL, 13 mmol), and the resulting reaction was heated at 50° C. overnight. The solvent was then evaporated, water (60 mL) was added, and the mixture was extracted with ethyl acetate (3×30 mL). The combined organic extracts were dried over sodium sulfate, filtered, and then concentrated in-vacuo. The residue was purified by silica gel chromatography to give 4-bromo-1,3-dihydro-benzoimidazol-2-one (701 mg) in 76% yield. MS: 211.0 [M−H].

Step 3: To a solution of 4-bromo-1,3-dihydro-benzoimidazol-2-one (701 mg, 3.29 mmol) in DMSO (2 mL) was added potassium acetate (803 g, 10.9 mmol), 1,1′-bis(diphenyl phosphino)ferrocene palladium chloride (134 mg, 0.16 mmol) and bis(pinacolato)diboron (1.67 g, 6.58 mmol), and the reaction was degassed and heated in a microwave reactor for 30 minutes at 150° C. The reaction mixture was then filtered through a pad of Celite™, water (60 mL) was added, and the mixture was extracted with ethyl acetate (3×30 mL). The combined organic extracts were dried over sodium sulfate, filtered, and then concentrated in-vacuo to give 4-(4,4,5,5-tetramethyl-[1,3,2]dioxaborolan-2-yl)-1,3-dihydro-benzoimidazol-2-one, which was used in the next step without further purification.

Step 4: According to the procedure of Example 63, Step 3, 3-(3-iodo-2-pyridin-4-yl-pyrazolo[1,5-a]pyrimidin-7-yl)-8-aza-bicyclo [3.2.1]octane-8-carboxylic acid ethyl ester and 4-(4,4,5,5-tetramethyl-[1,3,2dioxaborolan-2-yl)-1,3-dihydro-benzoimidazol-2-one afforded 3-[3-(2-oxo-2,3-dihydro-1H-benzoimidazol-4-yl)-2-pyridin-4-yl-pyrazolo[1,5-a]pyrimidin-7-yl]-8-aza-bicyclo[3.2.1]octane-8-carboxylic acid ethyl ester (47 mg, 45%) after purification by RP-HPLC. MS: 510.4 [M+H].

### Example 84

### Ethyl 3-[3-(1H-indol-4-yl)-2-pyridin-4-ylpyrazolo[1,5-a]pyrimidin-7-yl]-8-azabicyclo[3.2.1]octane-8-carboxylate

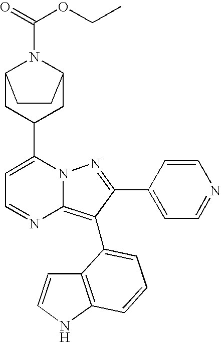

According to the procedure of Example 63, Step 3, 3-(3-iodo-2-pyridin-4-yl-pyrazolo[1,5-a]pyrimidin-7-yl)-8-aza-bicyclo[3.2.1]octane-8-carboxylic acid ethyl ester and 4-(4,4,5,5-tetramethyl-[1,3,2]dioxaborolan-2-yl)-1H-indole provided to give ethyl 3-[3-(1H-indol-4-yl)-2-pyridin-4-ylpyrazolo[1,5-a]pyrimidin-7-yl]-8-azabicyclo[3.2.1]octane-8-carboxylate in 51% yield following purification by RP-HPLC. MS: 493.4 [M+H].

### Example 85

### Ethyl 3-[3-(1H-indol-6-yl)-2-pyridin-4-ylpyrazolo[1,5-a]pyrimidin-7-yl]-8-azabicyclo[3.2.1]octane-8-carboxylate

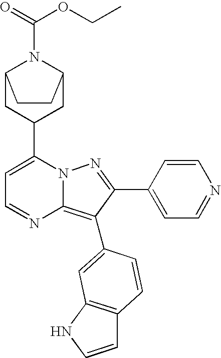

According to the procedure of Example 63, Step 3, 3-(3-iodo-2-pyridin-4-yl-pyrazolo[1,5-a]pyrimidin-7-yl)-8-aza-bicyclo[3.2.1]octane-8-carboxylic acid ethyl ester and 6-(4,4,5,5-tetramethyl-[1,3,2]dioxaborolan-2-yl)-1H-indole provided ethyl 3-[3-(1H-indol-6-yl)-2-pyridin-4-ylpyrazolo[1,5-a]pyrimidin-7-yl]-8-azabicyclo[3.2.1]octane-8-carboxylate in 47% yield following purification by RP-HPLC. MS: 493.3 [M+H].

### Example 86

### Ethyl 3-[3-(2-oxo-2,3-dihydro-1H-pyrrolo[2,3-b]pyridin-4-yl)-2-pyridin-4-ylpyrazolo[1,5-a]pyrimidin-7-yl-8-azabicyclo[3.2.1]octane-8-carboxylate

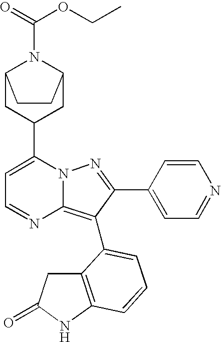

Step 1: According to the procedure of Example 40, Step 4, 4-bromooxindole provided 4-(4,4,5,5-tetramethyl-[1,3,2]dioxaborolan-2-yl)-1,3-dihydro-indol-2-one, which was used in the next step without purification.

Step 2: According to the procedure of Example 63, Step 3, 3-(3-iodo-2-pyridin-4-yl-pyrazolo[1,5-a]pyrimidin-7-yl)-8-aza-bicyclo[3.2.1]octane-8-carboxylic acid ethyl ester and 4-(4,4,5,5-tetramethyl-[1,3,2]dioxaborolan-2-yl )-1,3-di hydro-indol-2-one provided ethyl 3-[3-(2-oxo-2,3-dihydro-1H-pyrrolo[2,3-b]pyridin-4-yl )-2-pyridin-4-ylpyrazolo[1,5-a]pyrimidin-7-yl]-8-azabicyclo[3.2.1]octane-8-carboxylate in 41% yield following purification by RP-HPLC. MS: 511.2 [M+H].

### Example 87

### 7-(8-Ethyl-8-azabicyclo[3.2.1]oct-3-yl)-3-(1H-indol-6-yl)-2-pyridin-4-ylpyrazolo[1,5-a]pyrimidine

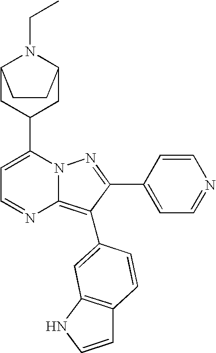

Following the procedure of Example 63, Step 3, 7-(8-ethyl-8-azabicyclo[3.2.1]oct-3-yl)-3-iodo-2-pyridin-4-ylpyrazolo[1,5-a]pyrimidine and 6-(4,4,5,5-tetramethyl-[1,3,2]dioxaborolan-2-yl)-1H-indole gave 7-(8-ethyl-8-azabicyclo[3.2.1]oct-3-yl)-3-(1H-indol-6-yl)-2-pyridin-4-ylpyrazolo[1,5-a]pyrimidine in 31% yield following purification by RP-HPLC. MS: 449.3 [M+H].

### Example 88

### Ethyl 3-[3-(2-oxo-2 3-dihydro-1H-indol-6-yl)-2-pyridin-4-ylpyrazolo[1,5-a]pyrimidin-7-yl]-8-azabicyclo[3.2.1]octane-8-carboxylate

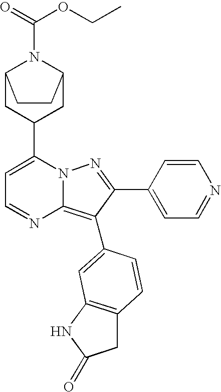

Step 1: According to the procedure of Example 40, Step 4, 6-bromooxindole provided 6-(4,4,5,5-tetramethyl-[1,3,2]dioxaborolan-2-yl)-1,3-dihydro-indol-2-one, which was used in the next step without purification.

Step 2: Following the procedure of Example 63, Step 3, 3-(3-iodo-2-pyridin-4-yl-pyrazolo[1,5-a]pyrimidin-7-yl)-8-aza-bicyclo[3.2.1]octane-8-carboxylic acid ethyl ester and 6-(4,4,5,5-tetramethyl-[1,3,2]dioxaborolan-2-yl)-1,3-dihydro-indol-2-one afforded ethyl 3-[3-(2-oxo-2,3-dihydro-1H-indol-6-yl)-2-pyridin-4-yl pyrazolo[1,5-a]pyrimidin-7-yl]-8-azabicyclo[3.2.1]octane-8-carboxylate in 42% yield following purification by RP-HPLC. MS: 509.3 [M+H].

### Example 89

### 2-Chloro-5-[7-(2,2-dimethyl-1,3-dioxolan-4-yl)-2-pyridin-4-ylpyrazolo[1,5-a]pyrimidin-3-yl]phenol

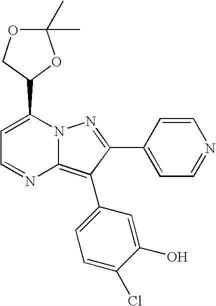

Step 1: A solution of 4 g (27.74 mmol) of 1-[(4R)-2,2-dimethyl-1, 3-dioxolan-4-yl]ethanone, (prepared according to the procedure of Synthetic Communications, 16(12), 1517-22, 1986) in DMF-DMA (40 mL) was heated to 100° C. for 19 hours. The solvent was then removed under reduced pressure to give a brown, viscous oil. The crude oil was purified by Biotage™ chromatography (cartridge 40s), eluting with a gradient of ethyl acetate/hexanes (1:2) and 100% ethyl acetate to afford (2E)-3-(dimethylamino)-1-[(4R)-2,2-dimethyl-1,3-dioxolan-4-yl]prop-2-en-1-one as a light brown oil (1.4 g, 25.3 %). MS: 200.2 [M+H].

Step 2: A solution of (2E)-3-(dimethylamino)-1-[(4R)-2,2-dimethyl-1,3-dioxolan-4-yl]prop-2-en-1-one (0.100 g, 0.5 mmol) and 3-(5-amino-3-pyridin-4-yl-1H-pyrazol-4-yl)-phenol (0.173 g, 0.606 mmol) in acetic acid (5 mL) was heated at 100° C. for 19 hours. The solvent was then removed in-vacuo. The resulting crude oil (0.194 g) was diluted with dichloromethane (20 mL) and the organics were washed with saturated aqueous sodium bicarbonate (2×5 mL) and brine (5 mL). The organics were dried over magnesium sulfate, filtered, and concentrated in-vacuo. The residue was purified by Biotage™ chromatography (cartridge 40s), eluting with ethyl acetate to afford 2-chloro-5-{7-[(4S)-2,2-dimethyl-1,3-dioxolan-4-yl]-2-pyridin-4-ylpyrazolo [1,5-a]pyrimidin-3-yl}phenol as a yellow crystalline solid (0.048 g, 4.7 %). MS: 423 [M+H].

### Example 90

### 7-(8-Ethyl-8-azabicyclo[3.2.1]octan-3-yl)-3-(1H-indazol-4-yl)-2-(pyridin-4-yl)pyrazolo[1,5-a]pyrimidine

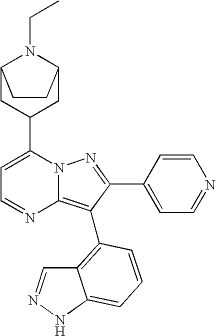

Step 1: Using the procedure of Example 76, Step 3, 4-bromo-1H-indazole was converted into 4-(4,4,5,5-tetramethyl-1,3,2-dioxaborolan-2-yl)-1H-indazole which was used in the next reaction without further purification.

Step 2: The reaction of 7-(8-ethyl-8-azabicyclo[3.2.1]octan-3-yl)-3-iodo-2-(pyridin-4-yl)pyrazolo[1,5-a]pyrimidine with 24-(4,4,5,5-tetramethyl-1,3,2-dioxaborolan-2-yl)-1H-indazole, using the procedure of Example 20, Step 7, provided 6.3 mg (4% yield) of 7-(8-ethyl-8-azabicyclo[3.2.1]octan-3-yl)-3-(1H-indazol-4-yl)-2-(pyridin-4-yl)pyrazolo[1,5-a]pyrimidine as a yellow solid. ¹H NMR (400 MHz, CD₃OD) δ 8.57 (d, J=4.4 Hz, 1H), 8.51 (m, 2H), 7.71 (d, J=5.2 Hz, 2H), 7.65 (d, J=8.4 Hz, 1H), 7.52 (m, 2H), 7.24 (d, J=7.2 Hz, 1H), 7.13 (d, J=4.4 Hz, 1H), 4.42 (m, 1H), 3.22 (q, J=7.2, 2H), 2.68 (m, 2H), 2.30-2.49 (m, 8H), 1.46 (t, J=7.2 Hz, 3H). MS: 450.3 [M+H].

### Example 91

### Ethyl 3-(3-(3-(1,3,4-oxadiazol-2-yl)phenyl)-2-(pyridin-4-yl)pyrazolo[1,5-a]pyrimidin-7-yl)-8-azabicyclo[3.2.1]octane-8-carboxylate

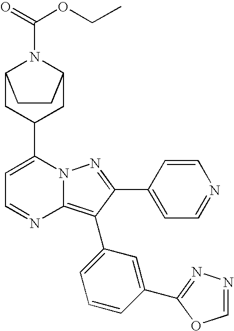

Step 1: A suspension of 3-bromobenzohydrazide (6.01 g, 27.9 mmol) in triethyl orthoformate (40 ml, 240 mmol) was brought to reflux under a nitrogen atmosphere and stirred vigorously overnight. After cooling to room temperature, the solvent was removed in-vacuo to give a pale, yellow syrup that crystallized on standing. Recrystallization from ethyl acetate/hexanes gave 2-(3-bromophenyl)-1,3,4-oxadiazole (4.86 g; 77 %). MS: 223/225 [M+H].

Step 2: To a mixture of 2-(3-bromophenyl)-1,3,4-oxadiazole (1.06 g, 4.71 mmol), 4,4,4′,4′,5,5,5′,5′-octamethyl-2,2′-bi(1,3,2-dioxaborolane) (1.40 g, 5.51 mmol), and potassium acetate (1.32 g, 13.45 mmol) was added DMSO (30 mL) and ([1,1′-bis(diphenylphosphino)ferrocene]dichloropalladium(ii) (0.0993 g, 0.136 mmol). The vessel was capped and placed under a nitrogen atmosphere, heated to 80° C. and stirred vigorously for about 4.5 hours. The reaction was cooled to room temperature overnight and then poured into water and ethyl acetate and the resulting mixture was filtered through a pad of Celite™. The layers were separated and the aqueous phase was washed with a second portion of ethyl acetate. The combined organics were dried over magnesium sulfate, then filtered, and concentrated in-vacuo to yield a brown syrup that solidified on standing. The crude solid was dissolved in a mixture of dichloromethane and ethyl acetate, adsorbed onto silica, and purified on a 40 g silica column to give the desired boronate ester, 2-(3-(4,4,5,5-tetramethyl-1,3,2-dioxaborolan-2-yl)phenyl)-1,3,4-oxadiazole (0.28 g; 21.8 %) as an off-white solid. MS: 273.2 [M+H].

Step 3: A small vial was charged with ethyl 3-(3-iodo-2-(pyridin-4-yl)pyrazolo[1,5-a]pyrimidin-7-yl)-8-azabicyclo[3.2.1]octane-8-carboxylate (0.1006 g, 0.200 mmol), 2-(3-(4,4,5,5-tetramethyl-1,3,2-dioxaborolan-2-yl)phenyl)-1,3,4-oxadiazole (0.0779 g, 0.286 mmol), [1,1′-bis(diphenylphosphino)ferrocene]dichloropalladium(II) (0.0102 g, 0.012 mmol), and DME (2 mL). To this mixture was added 2M aqueous sodium carbonate (0.3 mL, 0.600 mmol) and the resulting heterogeneous orange mixture was rigorously degassed, placed under a nitrogen atmosphere, and heated to 80° C. After 3 hours, the reaction was cooled to room temperature and stirred overnight. The crude reaction was diluted with acetonitrile and filtered through a pad of magnesium sulfate and Celite™. After removing the solvent in-vacuo, the resulting dark brown syrup was purified by semi-preparative RP HPLC to afford ethyl 3-(3-(3-(1,3,4-oxadiazol-2-yl)phenyl)-2-(pyridin-4-yl)pyrazolo[1,5-a]pyrimidin-7-yl)-8-azabicyclo[3.2.1]octane-8-carboxylate as a bright yellow solid (0.0408 g; 39 %). MS: 522.1 [M+H].

### Example 92

### tert-Butyl (1S,4S)-5-{3-fluoro-4-[3-(1H-indazol-4-yl)-2-pyridin-4-pyrazolo[1,5-a]pyrimidin-7-yl]phenyl}-2,5-diazabicyclo[2.2.1]heptane-2-carboxylate

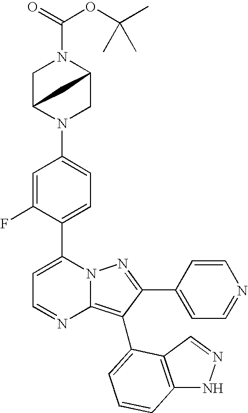

### Step 1: tert-Butyl (1S,4S)-5-(4-acetyl-3-fluorophenyl)-2,5-diazabicyclo[2.2.1]heptane-2-carboxylate

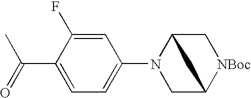

To a solution of (1S,4S)-tert-butyl 2,5-diazabicyclo[2.2.1]heptane-2-carboxylate (0.2 g, 1 mmol) in 4 mL of HMPA, 1-(2,4-difluorophenyl)ethanone (0.151 mL, 1.2 mmol) and potassium carbonate (0.552 g, 4 mmol) were added. This solution was heated at 70° C. for 36 hours in an oil bath. The mixture was then cooled to room temperature, and diluted with 100mL of ether, and washed with water three times. The aqueous layer was then washed with ether, and the organic layers combined. The combined ether layers were dried over sodium sulfate and concentrated to yield a residue which was purified by silica gel chromatography (12:88, ⁱPrOH:Hexanes) to give 0.290 g (87%) of tert-butyl (1S,4S)-5-(4-acetyl-3-fluorophenyl)-2,5-diazabicyclo[2.2.1heptane-2-carboxylate as a white solid. MS: 335.2 [M+H].

### Step 2: tert-butyl (1S,4S)-5-{4-[(2E)-3-(dimethylamino)prop-2-enoyl]-3-fluorophenyl}-2,5-diazabicyclo[2.2.1]heptane-2-carboxylate

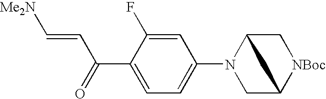

A solution of tert-butyl (1S,4S)-5-(4-acetyl-3-fluorophenyl)-2,5-diazabicyclo[2.2.1]heptane-2-carboxylate (0.42 g, 1.26 mmol) and tert-butoxybis(dimethylamino)methane (0.8 mL, 3.8 mmol) in 3 mL THF was heated at 100° C. overnight in a sealed tube. The reaction mixture was concentrated and then diluted with 1 mL water to precipitate the desired product. The aqueous layer was decanted and the residue was washed with water. The residue was dissolved in EtOAc, washed with water, dried over NaSO₄, and filtered. The filtrate was evaporated to give 0.455 g (93%) of tert-butyl (1S,4S)-5-{4-[(2E)-3-(dimethylamino)prop-2-enoyl]-3-fluorophenyl}-2,5-diazabicyclo[2.2.1]heptane-2-carboxylate as a yellow solid. MS: 390.2 [M+H].

### Step 3: methyl 1H-indazole-4-carboxylate

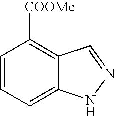

To a cold (0°-5° C.) solution of methyl-3-amino-2-methyl benzoate (16.6 mL, 19.0 g, 0.12 mol) in chloroform (200 mL) was added dropwise acetic anhydride (24.8 mL, 0.26 mol) followed by stirring for 5 minutes. The resulting mixture was allowed to warm to room temperature and stirred for 1 hour and then was added potassium acetate (3.35g, 0.034 mol) and isoamyl nitrite (33.0 mL, 0.25 mol) and heated under reflux for 20 hours. The mixture was cooled to room temperature and solvent was evaporated to yield a brown solid. Water was added to the solid, followed by evaporation to yield a solid residue. The residue was treated with concentrated hydrochloric acid and the resulting mixture was heated at 50° C. for 2 hours. After cooling with an ice bath, the solution was basified to pH 14 with a 50% potassium hydroxide solution. The resulting solid was collected by filtration, washed with water and dried to yield 17.8 g of methyl 1H-indazole-4-carboxylate as a beige solid. MS: 177.0 [M+H].

### Step 4: methyl 1-(phenylsulfonyl)-1H-indazole-4-carboxylate

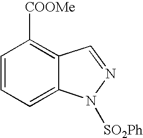

To a cold (0°-5° C.) suspension of sodium hydride (0.6 g, 15.0 mmol) in DMF (20 mL) was added methyl 1H-indazole-4-carboxylate (2.4 g, 13.62 mmol) [D. Batt, et al. J. Med. Chem., 2000, 43, 41-58] in portions over a period of 5 minutes and the resulting mixture was stirred at 5° C. for 15 minutes. A solution of benzenesulfonyl chloride (1.9 mL, 15.0 mmol) was then added dropwise and the resulting mixture was stirred at 5° C. for 30 minutes and then at room temperature for 3 hours. The mixture was poured on to ice and the solid was collected by filtration, washed with water and dried to yield 3.91 g (91%) of methyl 1-(phenylsulfonyl)-1H-indazole-4-carboxylate as a beige solid. MS: 317.0 [M+H].

### Step 5: [1-(phenylsulfonyl)-1H-indazol-4-yl]methanol

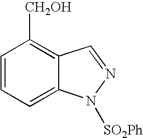

To a suspension of methyl 1-(phenylsulfonyl)-1H-indazole-4-carboxylate (3.11 g, 9.83 mmol) in a mixture of THF (30 mL) and toluene (15 mL) was added lithium borohydride as a 2.0M solution in THF (2.7 mL, 5.5 mmol), and the resulting mixture was stirred and heated at 70° C. for 30 minutes. Additional 2.0 M lithium borohydride solution (2.0 mL. 4.0 mmol) was added in portions over a period of 2.5 hours until all of the starting ester was consumed. The mixture was then cooled and poured on to ice water and the resulting two layers were separated. The aqueous layer was extracted with ethyl acetate. The combined organic layers were dried over anhydrous sodium sulfate, filtered and concentrated in vacuo. The crude oil was purified by silica gel chromatography (3:1 hexane/ethyl acetate, then 3:2 hexane/ethyl acetate) to yield 2.0 g (71%) of [1-(phenylsulfonyl)-1H-indazol-4-yl]methanol as a white solid. MS: 289.1 [M+H].

### Step 6: 1-(phenylsulfonyl)-1H-indazole-4-carbaldehyde

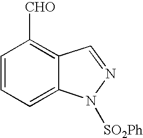

A mixture of [1-(phenylsulfonyl)-1H-indazol-4-yl]methanol (13.0 g, 45.08 mmol) and Dess-Martin periodinane (22.9 g, 54.0 mmol) in dichloromethane (420 mL) was stirred at room temperature for 1 hour. The reaction was quenched by stirring for 20 minutes with a saturated sodium thiosulfate solution (100 mL) and a saturated solution of sodium bicarbonate (75 mL). The two layers were separated and the aqueous layer was extracted with methylene chloride. The combined organic layer was dried over anhydrous sodium sulfate, filtered and concentrated in vacuo. The resulting crude solid was dissolved in methylene chloride and run through a silica gel plug to yield 12.65 g (98%) of 1-(phenylsulfonyl)-1H-indazole-4-carbaldehyde as a white solid. MS: 287.1 [M+H].

### Step 7: 2-[1-(phenylsulfonyl)-1H-indazol-4-yl]-1-pyridin-4-ylethanone

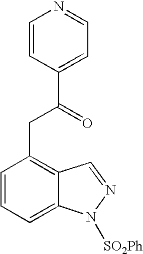

A mixture of 1-(phenylsulfonyl)-1H-indazole-4-carboxaldehyde (12.33 g, 43.07 mmol), diphenyl(phenylamino)(pyridin-4-yl)methylphosphonate (17.6 g, 42.27 mmol, prepared according to the procedure of Tet. Letters 39, 1717-1720, 1988), cesium carbonate (16.44 g, 50.46 mmol), tetrahydrofuran (246 mL) and isopropyl alcohol (82 mL) was heated at 45° C. for 3.5 hours. The yellow mixture was cooled to room temperature and poured in to ice-cold solution of 3N HCl (250 mL) and stirred at room temperature for 18 hours. The yellow solution was extracted with ether (2×200 mL) and ether extract was re-extracted with 10% HCl (2×100 mL). The combined HCl extracts were cooled to 0°-5° C. and neutralized to pH 7-8 using 2.5 N NaOH. The solid was collected by filtration, washed with ice cold water and dried to yield 15.5 g (97%) of 2-(1-(phenylsulfonyl)-1H-indazol-4-yl)-1-(pyridin-4-yl)ethanone as a beige solid. MS: 378.0 [M+H].

### Step 8: 4-(1H-indazol-4-yl)-3-pyridin-4-yl-1H-pyrazol-5-amine

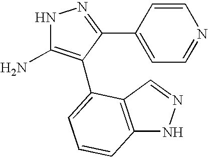

To a cold (0°-5° C.) solution dimethylformamide (13.0 mL, 254.3 mmol)) was added dropwise a solution of phosphorus oxychloride (9.9 mL, 106.2 mmol), and the resulting mixture was stirred for 20 min. To this mixture was added dropwise solution of 2-(1-(phenylsulfonyl)-1H-indazol-4-yl)-1-(pyridin-4-yl)ethanone (8.0 g, 21.2 mmol) in chloroform (80 mL), heated to 80° C. and stirred for 18 hours. The reaction was cooled to room temperature and quenched with ice-cold saturated solution of sodium bicarbonate (500 mL). After extraction with 5% methanol in dichloromethane (4×150 mL), the organic layer was dried over anhydrous sodium sulfate, filtered, and the filtrate was evaporated to yield semi-solid. The crude mixture was dissolved in dimethylformamide 26 mL) followed by the addition of hydroxylamine hydrochloride (1.77 g, 25.45 mmol) and stirred at room temperature for 2.5 hours. After cooling to 0° C., phosphorus oxychloride (3.0 mL, 32.2 mmol) was added and the mixture stirred overnight at room temperature. The reaction was quenched with ice-cold saturated solution of sodium bicarbonate. The solid was collected by filtration, washed with small amount of ice-cold water and dried. The crude solid 3-chloro-2-(1H-indazol-4-yl)-3-(pyridin-4-yl)acrylonitrile was dissolved in ethanol (80 mL) followed by the addition of hydrazine monohydrate (3.0 mL, 95.6 mmol) and heated under reflux for 2.5 h. After cooling to room temperature, the solvent was removed by evaporation. The crude product was purified by silica gel flash chromatography, eluting with methanol in dichloromethane (2-12% gradient), to give 4.7 g (80% yield) of 4-(1H-indazol-4-yl)-3-(pyridin-4-yl)-1H-pyrazol-5-amine as a beige solid. MS: 277.1 [M+H].

### Step 9: tert-butyl(1S,4S)-5-{3-fluoro-4-[3-(1H-indazol-4-yl)-2-pyridin-4-ylpyrazolo[1,5-a]pyrimidin-7-yl]phenyl}-2,5-diazabicyclo[2.2.1]heptane-2-carboxylate

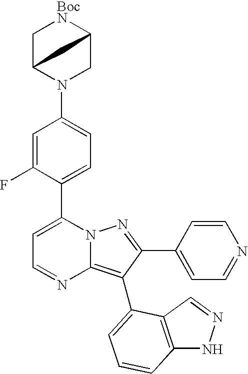

A mixture of tert-butyl(1S,4S)-5-{4-[(2E)-3-(dimethylamino)prop-2-enoyl]-3-fluorophenyl}-2,5-diazabicyclo[2.2.1]heptane-2-carboxylate (2.3g, 5.9 mmol), 4-(1H-indazol-4-yl)-3-(pyridin-4-yl)-1H-pyrazol-5-amine (1.59 g, 5.75 mmol) and trifluoroacetic acid (4.4 mL, 57.5 mmol) in 25 ml of ethanol was stirred at room temperature overnight. The reaction mixture was evaporated, cooled and stirred with saturated solution of sodium bicarbonate. The solid was collected by filtration, washed with water and dried. The crude solid was purified by silica gel chromatography (3-6% ⁱPrOH/CH₂Cl₂). Further purification was achieved by recrystallization from hot EtOH or EtOH/hexane to yield 2.43 g (70%) of tert-butyl (1S,4S)-5-{3-fluoro-4-[3-(1H-indazol-4-yl)-2-pyridin-4-ylpyrazolo[1,5-a]pyrimidin-7-yl]phenyl}-2,5-diazabicyclo[2.2.1]heptane-2-carboxylate as a yellow solid. MS: 603.2 [M+H].

### Example 93

### 7-{4-[(1S,4S)-2,5-diazabicyclo[2.2.1]hept-2-yl]-2-fluorophenyl}-3-(1H-indazol-4-yl)-2-pyridin-4-ylpyrazolo[1,5-a]pyrimidine

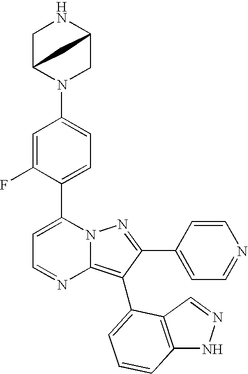

tert-Butyl (1S,4S)-5-{3-fluoro-4-[3-(1H-indazol-4-yl)-2-pyridin-4-ylpyrazolo[1,5-a]pyrimidin-7-yl]phenyl}-2,5-diazabicyclo[2.2.1]heptane-2-carboxylate (2.43 g, 4.0 mmol) was dissolved in 60 mL of methanol and then 30 mL of concentrated hydrochloric acid was added and the resulting mixture was stirred at room temperature for 3 hours. The precipitated solid was collected by filtration, then washed with a small amount of methanol. The filtrate was evaporated to dryness, and the residue was taken up in a minimal amount of methanol. The resulting solid was collected by filtration, washed with a minimal quantity of methanol and the solids combined. An ice-cooled saturated solution of sodium bicarbonate was added to the crude material. After filtration, the crude solid was purified by a short silica gel column (80:18:2, methylene chloride:methanol:ammonium hydroxide) to give 1.9 g (95%) of 7-{4-[(1S,4S)-2,5-diazabicyclo[2.2.1]hept-2-yl]-2-fluorophenyl}-3-(1H-indazol-4-yl)-2-pyridin-4-ylpyrazolo[1,5-a]pyrimidine as a yellow solid. MS: 503.3 [M+H].

### Example 94

### 7-{2-Fluoro-4-[(1S,4S)-5-methyl-2,5-diazabicyclo[2.2.1]hept-2-yl]phenyl}-3-(1H-indazol-4-yl )-2-pyridin-4-yl pyrazolo[1,5-a]pyrimidine

A mixture of 7-{4-[(1S,4S)-2,5-diazabicyclo[2.2.1]hept-2-yl]-2-fluorophenyl}-3-(1H-indazol-4-yl)-2-pyridin-4-ylpyrazolo[1,5-a]pyrimidine (1.9 g, 3.78 mmol), 37% HCHO (1.0 mL, 13.3 mmol), NaBH(OAc)₃ (2.12 g,10.04 mmol) and 6 drops of acetic acid in 35 mL of DMF was stirred at room temperature for 3 hours. The DMF was evaporated to dryness and 80 mL 7N ammonia methanol solution was added to the residue, stirred for 2 hours and the solvent was evaporated to dryness. A saturated solution of sodium bicarbonate was stirred into the resulting residue. The solid was collected by filtration, washed with water and dried. The crude solid was purified by flash chromatography (80:20, CH₂Cl₂/methanol) to give 1.7g (87%) of a yellow solid. This solid was re-crystallized from hot ethanol to yield 1.4 g (72%) of 7-{2-fluoro-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine as a yellow solid. MS: 279.6 [M+ACN+2H].

### Example 95

### 7-{2-fluoro-4-[(1S,4S)-5-methyl-5-oxido-2,5-diazabicyclo[2.2.1]hept-2-yl]phenyl}-3-(1H-indazol-4-yl)-2-pyridin-4-ylpyrazolo[1,5-a]pyrimidine

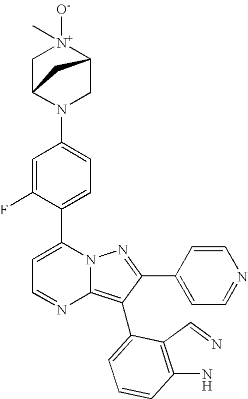

A mixture of 7-{2-fluoro-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine (0.23 g, 0.45 mmol) and 3-chloroperbenzoic acid (0.09g, 0.40 mmol) in methylene chloride was stirred at room temperature for 3 hours. The solvent was evaporated and the residue was stirred with a saturated solution of sodium bicarbonate. The resulting solid was collected by filtration, washed with water and dried. The crude solid was purified by silica gel chromatography, eluting with 20% methanol in methylene chloride, then with mixture of 10% methanol and 1% ammonium hydroxide in methylene chloride to yield 0.121 g (51%) of 7-{2-fluoro-4-[(1S,4S)-5-methyl-5-oxido-2,5-diazabicyclo[2.2.1]hept-2-yl]phenyl}-3-(1H-indazol-4-yl)-2-pyridin-4-ylpyrazolo[1,5-a]pyrimidine as a yellow solid. MS: 287.7 [M+ACN+2H].

### Example 96

### (1S,4S)-5-{3-Chloro-4-[3-(1H-indazol-4-yl)-2-pyridin-4-yl-pyrazolo[1,5-a]pyrimidin-7-yl]-phenyl}-2,5-diaza-bicyclo[2.2.1]heptane-2-carboxylic acid tert-butyl ester

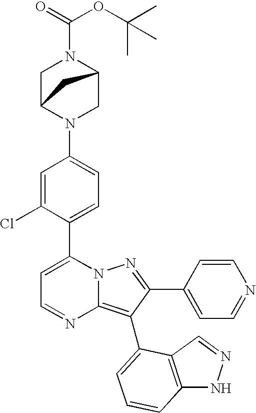

### Step 1: tert-butyl(1S,4S)-5-(4-acetyl-3-chlorophenyl)-2,5-diazabicyclo[2.2.1]heptane-2-carboxylate

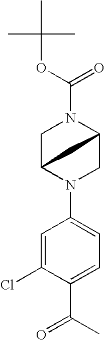

To a solution of (1S,4S)-tert-butyl 2,5-diazabicyclo[2.2.1]heptane-2-carboxylate (4.13 g, 20.86 mmol) in 20 mL of DMF, 2′-chloro-4′-fluoroacetophenone (3.0 g, 17.4 mmol) and potassium carbonate (7.2 g, 52.14 mmol) were added. This mixture was heated at 100° C. for 16 hours, cooled to room temperature and diluted with 200 mL of methylene chloride. The organic layer was dried and concentrated to yield a residue which was purified by silica gel chromatography (eluting with a gradient of 15:85 to 30:70 EtOAc/hexane), to yield 4.02 g (66%) tert-butyl(1S,4S)-5-(4-acetyl-3-chlorophenyl)-2,5-diazabicyclo[2.2.1]heptane-2-carboxylate as a beige solid. MS 351.1 [M+H].

### Step 2: (1S,4S)-tert-butyl 5-(4-((E)-3-(di methylamino)acryloyl)-3-chlorophenyl)-2,5-diazabicyclo[2.2.1]heptane-2-carboxylate

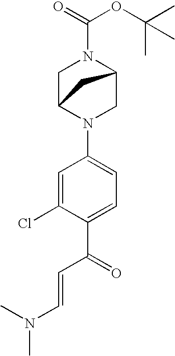

A mixture of (1S,4S)-tert-butyl 5-(4-acetyl-3-chlorophenyl)-2,5-diazabicyclo[2.2.1]heptane-2-carboxylate (2.457g, 7.00 mmol) and 5.00 mL of C-tert-butoxy-N,N,N′,N′-tetramethylmethanediamine was heated to 100° C. for 3 hours. The mixture was then concentrated in vacuo and the gummy residue was digested with 75 mL of diethyl ether. The solution was washed with 75 mL of water and 75 mL of saturated NaCl solution, dried over anhydrous magnesium sulfate, filtered and the solvent was removed in vacuo to give 2.679g (97%) of (1S,4S)-tert-butyl 5-(4-((E)-3-(dimethylamino)acryloyl)-3-chlorophenyl)-2,5-diazabicyclo[2.2.1]heptane-2-carboxylate as a light yellow foam used without purification. MS: 406.3 [M+H].

Step 3: (1S,4S)-5-{3-Chloro-4-[3-(1H-indazol-4-yl)-2-pyridin-4-yl-pyrazolo[1,5-a]pyrimidin-7-yl]-phenyl}-2,5-diaza-bicyclo[2.2.1]heptane-2-carboxylic acid tert-butyl ester To a solution of (1S,4S)-tert-butyl 5-(4-((E)-3-(dimethylamino)acryloyl)-3-chlorophenyl)-2,5-diazabicyclo[2.2.1]heptane-2-carboxylate (2.638 g, 6.50 mmol) and 4-(1H-indazol-4-yl)-3-(pyridin-4-yl)-1H-pyrazol-5-amine (1.877 g, 6.79 mmol) in 30 mL of methanol was added 5 mL of trifluoroacetic acid and the resulting solution was stirred at room temperature under a nitrogen atmosphere for 108 hours. The mixture was partitioned between 200 mL of dichloromethane and 200 mL of saturated sodium bicarbonate solution. The organic phase was separated and the aqueous phase was extracted with an additional 100 mL of dichloromethane. The combined organic phases were dried over anhydrous magnesium sulfate, filtered, and the solvent was removed in vacuo. The resulting yellow/brown foam was purified by silica gel chromatography to provide 3.374g (84%) of (1S,4S)-5-{3-chloro-4-[3-(1H-indazol-4-yl)-2-pyridin-4-yl-pyrazolo[1,5-a]pyrimidin-7-yl]-phenyl}-2,5-diaza-bicyclo[2.2.1]heptane-2-carboxylic acid tert-butyl ester, as a yellow foam. MS: 619.3 [M+H].

### Example 97

### 7-[2-Chloro-4-((1S,4S)-2,5-diaza-bicyclo[2.2.1]hept-2-yl)-phenyl]-3-(1H-indazol-4-yl)-2-pyridin-4-yl-pyrazolo[1,5-a]pyrimidine bis-hydrochloride salt

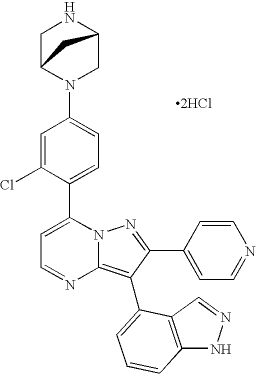

To a solution of (1S,4S)-5-{3-chloro-4-[3-(1H-indazol-4-yl)-2-pyridin-4-yl-pyrazolo[1,5-a]pyrimidin-7-yl]-phenyl}-2,5-diaza-bicyclo[2.2.1]heptane-2-carboxylic acid tert-butyl ester (3.370 g, 5.44 mmol) in 50 mL of methanol was added 25 mL of concentrated hydrochloric acid solution over 2-3 minutes. The resulting dark red solution was stirred at room temperature for 30 minutes. The solvents were removed in vacuo and the residue was digested with 50 mL of methanol. The resulting crystals were filtered and rinsed with fresh methanol. The damp product was quickly transferred to a desiccator and dried under high vacuum overnight to provide 2.638 g (82%) of 7-[2-chloro-4-((1S,4S)-2,5-diaza-bicyclo[2.2.1]hept-2-yl)-phenyl]-3-(1H-indazol-4-yl)-2-pyridin-4-yl-pyrazolo[1,5-a]pyrimidine bis-hydrochloride salt as yellow/orange crystals. MS: 280.6 [M+CH₃CN+2H].

### Example 98

### 7-[2-Chloro-4-((1S,4S)-5-methyl-2,5-diaza-bicyclo[2.2.1]hept-2-yl)-phenyl]-3-(1H-indazol-4-yl)-2-pyridin-4-yl-pyrazolo[1,5-a]pyrimidine

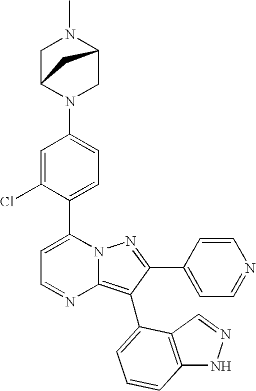

Step 1: 7-[2-Chloro-4-((1S,4S)-2,5-diaza-bicyclo[2.2.1]hept-2-yl)-phenyl]-3-(1H-indazol-4-yl)-2-pyridin-4-yl-pyrazolo[1,5-a]pyrimidine bis-hydrochloride salt (2.800 g, 4.73 mmol) was digested with 50 mL of half-saturated sodium bicarbonate solution taking care to avoid uncontrolled foaming. The resulting solid was filtered, washed with several portions of water and vacuum dried to give 2.287 g (93%) of 7-[2-chloro-4-((1S,4S)-2,5-diaza-bicyclo[2.2.1]hept-2-yl)-phenyl]-3-(1H-indazol-4-yl)-2-pyridin-4-yl-pyrazolo[1,5-a]pyrimidine free base as a yellow solid.

Step 2: To a solution of 7-[2-Chloro-4-((1S,4S)-2,5-diaza-bicyclo[2.2.1]hept-2-yl)-phenyl]-3-(1H-indazol-4-yl)-2-pyridin-4-yl-pyrazolo[1,5-a]pyrimidine (2.283 g, 4.40 mmol) in 20 mL of dimethylformamide was added 37% aqueous formaldehyde solution (1.10 mL, 14.77 mmol) followed by 3 drops of acetic acid and sodium borohydride (2.797 g, 13.15 mmol). The mixture was warmed slightly with a heat gun and diluted with an additional 20 mL of dimethylformamide to aid dissolution. After the removal of some insoluble material the mixture was stirred 2.5 hours at room temperature. It was then concentrated to dryness by rotary evaporation and the residue was digested with 20 mL of 7N methanolic ammonia solution. The resulting mixture was stirred 15 hours at room temperature. It was then filtered to remove small amounts of insoluble material and the filtrate was concentrated in vacuo. The residue was digested with 50 mL of dichloromethane and filtered. The solid was washed with several additional portions of dichloromethane. The combined filtrate and washings were concentrated in vacuo to give a yellow solid that was purified by silica gel chromatography to give 1.756 g of yellow powder. This material was partitioned between 150 mL of dichloromethane and 150 mL of half-saturated sodium bicarbonate solution. The organic phase was separated and the aqueous phase extracted with 50 mL of dichloromethane. The combined organic phases were dried over anhydrous magnesium sulfate, filtered, and concentrated in vacuo to give 1.746g of 7-[2-chloro-4-((1S,4S)-5-methyl-2,5-diaza-bicyclo[2.2.1]hept-2-yl)-phenyl]-3-(1H-indazol-4-yl)-2-pyridin-4-yl-pyrazolo[1,5-a]pyrimidine as a yellow powder. MS: 533.1 [M+H].

### Example 99

### tert-butyl(1S,4S)-5-{3,5-difluoro-4-[3-(1H-indazol-4-yl)-2-pyridin-4-ylpyrazolo[1,5-a]pyrimidin-7-yl]phenyl}-2,5-diazabicyclo[2.2.1]heptane-2-carboxylate

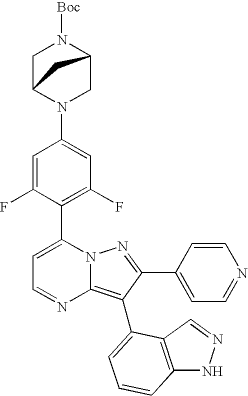

### Step 1: (1S,4S)-tert-Butyl 5-(4-acetyl-3,5-difluorophenyl)-2,5-diazabicyclo[2.2.1]heptane-2-carboxylate

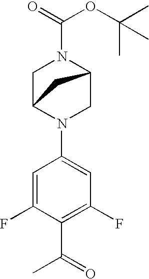

To a solution of 1-(2,4,6-trifluorophenyl)ethanone in 30 mL of hexamethylphosphoramide, (3.9 g, 22.5 mmol) (1S,4S)-tert-butyl 2,5-diazabicyclo[2.2.1]heptane-2-carboxylate (3.0 g, 15 mmol) and potassium carbonate (6.2 g, 45 mmol) were added. This solution was stirred at room temperature for 4 days. The mixture was diluted with 200 mL of diethyl ether and was washed with 200 mL water. The aqueous solution was extracted twice with diethyl ether twice. The combined organic layer was washed with water three times, then dried, and concentrated. The residue was purified by silica gel chromatography (isopropanol, hexanes) to give 3.3 g (62% yield) of (1S,4S)-tert-butyl 5-(4-acetyl-3,5-difluorophenyl)-2,5-diazabicyclo[2.2.1]heptane-2-carboxylate. MS: 353.1 [M+H].

### Step 2: (1S,4S)-tert-butyl 5-(4-((E)-3-(dimethylamino)acryloyl)-3,5-difluorophenyl)-2,5-diazabicyclo[2.2.1]heptane-2-carboxylate

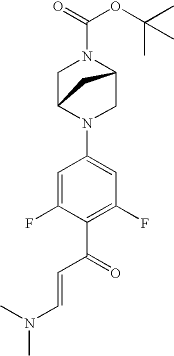

A mixture of (1S,4S)-tert-butyl 5-(4-acetyl-3,5-difluorophenyl)-2,5-diazabicyclo[2.2.1]heptane-2-carboxylate (3.3 g, 9.4 mmol) and 30 mL of 1,1-dimethoxy-N,N-dimethylmethanamine were refluxed for 35 hours. The reaction mixture was concentrated and the residue was purified by silica gel chromatography (isopropanol, dichloromethane) to give 3.8 (99%) of (1S,4S)-tert-butyl 5-(4-((E)-3-(dimethylamino)acryloyl)-3,5-difluorophenyl)-2,5-diazabicyclo[2.2.1]heptane-2-carboxylate. MS: 408.3 [M+H].

Step 3: tert-Butyl (1S,4S)-5-{3,5-difluoro-4-[3-(1H-indazol-4-yl)-2-pyridin-4-ylpyrazolo[1,5-a]pyrimidin-7-yl]phenyl}-2,5-diazabicyclo[2.2.1]heptane-2- carboxylate To a solution of 2,2,2-trifluoroacetic acid (0.54 mL) in 18 mL of methanol, (1S,4S)-tert-butyl 5-(4-((E)-3-(dimethylamino)acryloyl)-3,5-difluorophenyl)-2,5-diazabicyclo-[2.2.1]heptane-2-carboxylate (1.1 g, 2.6 mmol) and 4-(1H-indazol-4-yl)-3-(pyridin-4-yl)-1H-pyrazol-5-amine (0.72 g, 2.6 mmol) were added. This solution was stirred at room temperature for 3 days. The mixture was basified with methanolic ammonia, adsorbed onto silica gel, and purified by silica gel chromatography (isopropanol, dichloromethane) to give 1.4 g (87% yield) of tert-butyl (1S,4S)-5-{3,5-difluoro-4-[3-(1H-indazol-4-yl)-2-pyridin-4-ylpyrazolo[1,5-a]pyrimidin-7-yl]phenyl}-2,5-diazabicyclo[2.2.1]heptane-2-carboxylate. MS: 621.3 [M+H].

### Example 100

### 7-(4-((1S,4S)-2,5-diazabicyclo[2.2.1]heptan-2-yl)-2,6-difluorophenyl)-3-(1H-indazol-4-yl)-2-(pyridin-4-yl)pyrazolo[1,5-a]pyrimidine

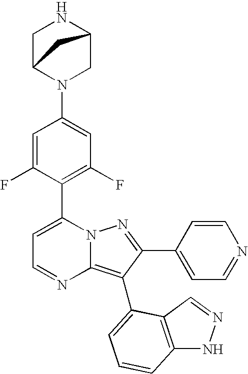

A solution of (1S,4S)-tert-butyl 5-(4-(3-(1H-indazol-4-yl)-2-(pyridin-4-yl)pyrazolo[1,5-a]pyrimidin-7-yl)-3,5-difluorophenyl)-2,5-diazabicyclo[2.2.1]heptane-2-carboxylate (1.4 g, 2.3 mmol) in 6N HCl/methanol (19 mL concentrated HCl and 19 mL methanol) was stirred for 1 hour. The mixture was concentrated, basified with methanolic ammonia, adsorbed onto silica gel, and purified with silica gel chromatography (ammonia, methanol, dichloromethane) to give 1.1 g (93% yield) of 7-(4-((1S,4S)-2,5-diazabicyclo[2.2.1]heptan-2-yl)-2,6-difluorophenyl)-3-(1H-4-yl)pyrazolo[1,5-a]pyrimidine. MS: 521.3 [M+H].

### Example 101

### 7-{2,6-difluoro-4-[(1S,4S)-5-methyl-2,5-diazabicyclo[2.2.1]hept-2-yl]phenyl}-3-(1H-indazol-4-yl )-2-pyridin-4-yl pyrazolo[1,5-a]pyrimidine

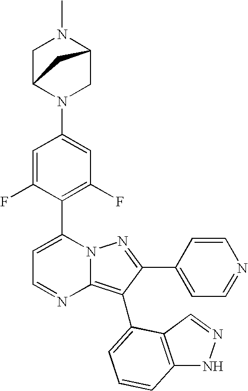

To a solution of 7-(4-((1S,4S)-2,5-diazabicyclo[2.2.1]heptan-2-yl)-2,6-difluorophenyl)-3-(1H-indazol-4-yl)-2-(pyridin-4-yl)pyrazolo[1,5-a]pyrimidine (1.1 g, 2.1 mmol) in 30 mL of DMF, formaldehyde (0.47 mL, 6.3 mmol) and sodium triacetoxyhydroborate (1.3 g, 6.3 mmol) were added. This solution was stirred at room temperature for 2 hours, then concentrated. The residue was stirred with 15 mL of 7 N methanolic ammonia overnight. The solution was concentrated and purified with silica gel chromatography (methanol, dichloromethane) to give 0.95 g (84% yield) of 7-(2,6-difluoro-4-((1S,4S)-5-methyl-2,5-diazabicyclo[2.2.1]heptan-2-yl)phenyl)-3-(1H-indazol-4-yl)-2-(pyridin-4-yl)pyrazolo[1,5-a]pyrimidine. MS: 535.4 [M+H].

### Example 102

### tert-Butyl(1S,4S)-5-{4-[3-(7-fluoro-1H-indazol-4-yl)-2-pyridin-4-ylpyrazolo[1,5-a]pyrimidin-7-yl]phenyl}-2,5-diazabicyclo[2.2.1]heptane-2-carboxylate

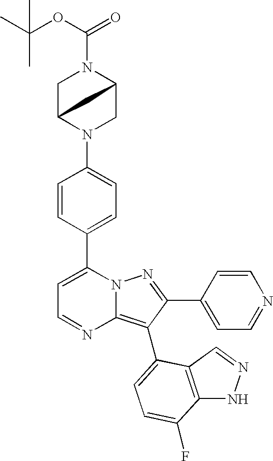

### Step 1: 6-Bromo-2,3-difluoro-benzaldehyde

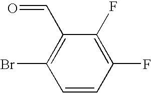

To a dry flask, under nitrogen, was added 9.24 mL of 2,2,6,6-tetramethylpiperidine (0.054 mol, 1.05 equiv) and 103.5 mL of tetrahydrofuran (THF), which was then cooled to −78° C. 21.7 mL of n-butyllithium (0.054 mol, 1.05 equiv) was added dropwise, followed by 5.86 mL of 1-bromo-3,4-difluorobenzene (0.052 mol, 1.0 equiv) in 6 mL of THF. The resulting mixture was stirred at −78° C. for 2 hours and 4.21 ml of N,N-dimethylformamide (DMF) (0.054 mol, 1.05 equiv) was added. The mixture was allowed to warm up to −20° C., then quenched with 44 ml of 1N HCl in a dropwise addition. This solution was then extracted with diethyl ether and the organic layer was washed with 1N HCl three times, followed by a brine wash. After drying the organic layer over magnesium sulfate (MgSO₄), it was concentrated in-vacuo to yield 11.12 g of 6-bromo-2,3-difluoro-benzaldehyde as a brown oil which was used without purification in the next reaction.

### Step 2: 4-Bromo-7-fluoro-1H-indazole

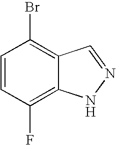

A 11.2 g portion of 6-bromo-2,3-difluoro-benzaldehyde was dissolved in 51 mL of dimethoxyethane. To this was added 51 mL of anhydrous hydrazine, followed by refluxing for 2.5 hours; reaction monitored by thin layer chromatography (TLC). Dimethoxyethane was evaporated off and the remaining residue cooled in an ice bath. Ice was added and the resulting white solid filtered off, and washed with cold water. The solid was then warmed in dichloromethane and filtered. The filtrate was evaporated to dryness and warmed up in dichloromethane and filtered again. In total, 6.19 g of 4-bromo-7-fluoro-1H-indazole was recovered as a white solid in 32% yield. MS: 215.0, 217.0 [M+H].

### Step 3: 7-Fluoro-1H-indazole-4-carbaldehyde

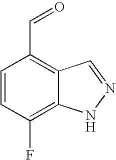

To 0.614 g of sodium hydride (0.015 mol, 1.1 equiv) in a dry flask, under nitrogen, was added 46 mL of THF. The mixture was cooled to 0° C. in an ice bath. To this mixture was added 3 g of 4-bromo-7-fluoro-1H-indazole (0.014 mol, 1.0 equiv), followed by stirring at 0° C. for 5 minutes. The resulting pink mixture was warmed to room temperature and stirred for another 15 minutes, after which the reaction turned brown. It was then cooled to −78° C. and 23 mL of 1.7M t-butyllithium in pentane was added slowly (0.039 mol, 2.8 equiv) while stirring at −78° C. for 10 minutes. To this mixture was added 2.16 mL of dimethylformamide in 7.5 mL of tetrahydrofuran dropwise. The mixture was stirred for 5 minutes at −78° C. then warmed to room temperature. After stirring for another 30 minutes, the product mixture was quenched with 2M hydrochloric acid (HCl) and extracted with ethyl acetate. The organic layer was dried over MgSO₄ and evaporated to dryness. A quantitative yield (2.51 g) of 7-fluoro-1H-indazole-4-carbaldehyde was obtained as a pink solid containing a small quantity of DMF. MS: 165.2 [M+H].

### Step 4: 1-Benzenesulfonyl-7-fluoro-1H-indazole-4-carbaldehyde

A 2.29 g portion of 7-fluoro-1H-indazole-4-carbaldehyde (0.014 mol, 1.0 equiv) was dissolved in 100 mL of THF. To this was added 0.614 g of NaH (0.015 mol, 1.1 equiv), followed by 20 minutes of stirring. To the reaction mixture, 3.6 mL of benzenesulfonylchloride (0.028 mol, 2.0 equiv) were added, and the reaction was stirred for another hour. The reaction was quenched with H₂O and extracted with dichloromethane. The organic layer was dried over MgSO₄ and evaporated to dryness to yield a wet orange solid, which was then washed with diethyl ether to produce 3.54 g of 1-benzenesulfonyl-7-fluoro-1H-indazole-4-carbaldehyde as a light orange solid in 83% yield. MS: 305.2 [M+H]. The procedures for the following 3 steps are based on examples disclosed in a copending application, U.S. Provisional Application No. 61/067,843.

### Step 5: 2-(1-benzenesulfonyl-7-fluoro-1H-indazol-4-yl)-1-pyridin-4-yl-ethanone (CAT1 788145) and 2-(7-fluoro-1H-indazol-4-yl)-1-pyridin-4-yl-ethanone

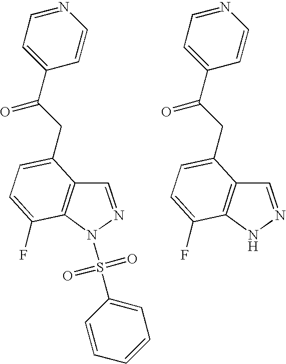

By the procedure of Example 44, step 6, 1-benzenesulfonyl-7-fluoro-1H-indazole-4-carbaldehyde was reacted with diphenyl(phenylamino)(pyridin-4-yl)methylphosphonate to provide a 4:1 mixture of 2-(1-benzenesulfonyl-7-fluoro-1H-indazol-4-yl)-1-pyridin-4-yl-ethanone and 2-(7-fluoro-1H-indazol-4-yl)-1-pyridin-4-yl-ethanone. This mixture was subsequently used without purification. Products: 2-(1-benzenesulfonyl-7-fluoro-1H-indazol-4-yl)-1-pyridin-4-yl-ethanone MS: 396.1 [M+H] and 2-(7-fluoro-1H-indazol-4-yl)-1-pyridin-4-yl-ethanone 256.3 [M+H].

### Step 6: 4-(7-Fluoro-1H-indazol-4-yl)-5-pyridin-4-yl-2H-pyrazol-3-ylamine

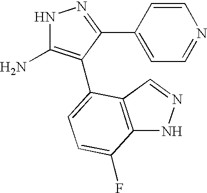

Following the procedure of Example 44, step 7, the mixture of 4:1 2-(1-benzenesulfonyl-7-fluoro-1H-indazol-4-yl)-1-pyridin-4-yl-ethanone and 2-(7-fluoro-1H-indazol-4-yl)-1-pyridin-4-yl-ethanone was converted to 4-(7-fluoro-1H-indazol-4-yl)-5-pyridin-4-yl-2H-pyrazol-3-ylamine. MS: 295.2 [M+H].

### Step 7: tert-butyl(1S,4S)-5-(4-acetylphenyl)-2,5-diazabicyclo[2.2.1]heptane-2-carboxylate

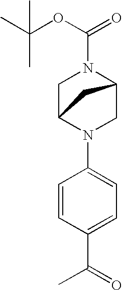

Following the procedure of Example 44, Step 1, (1S,4S)-tert-butyl 2,5-diazabicyclo[2.2.1]heptane-2-carboxylate was reacted with 1-(4-fluorophenyl)ethanone to provide tert-butyl(1S,4S)-5-(4-acetylphenyl)-2,5-diazabicyclo[2.2.1]heptane-2-carboxylate as an off-white solid. MS: 317.2 [M+H].

### Step 8: 5-[4-(3-Dimethylamino-acryloyl)-phenyl]-2,5-diaza-bicyclo[2.2.1]heptane-2-carboxylic acid tert-butyl ester

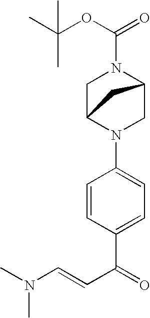

Following the procedure of Example 92, Step 2, tert-butyl(1S,4S)-5-(4-acetylphenyl)-2,5-diazabicyclo[2.2.1]heptane-2-carboxylate was reacted with tert-butoxybis(dimethylamino)methane to provide 5-[4-(3-dimethylamino-acryloyl)-phenyl]-2,5-diazabicyclo[2.2.1]heptane-2-carboxylic acid tert-butyl ester as a pale yellow solid. MS: 372.3 [M+H]. The procedure for the following step is based on an example disclosed in a copending application, U.S. Provisional Application No. 61/067,843.

### Step 9: tert-Butyl (1S,4S)-5-{4-[3-(7-fluoro-1H-indazol-4-yl)-2-pyridin-4-ylpyrazolo[1,5-a]pyrimidin-7-yl]phenyl}-2,5-diazabicyclo[2.2.1]heptane-2-carboxylate

Following the procedure of Example 98, Step 4, 4-(7-fluoro-1H-indazol-4-yl)-5-pyridin-4-yl-2H-pyrazol-3-ylamine (0.38 g, 1.3 mmol) and 5-[4-(3-dimethylamino-acryloyl)-phenyl]-2,5-diazabicyclo[2.2.1]heptane-2-carboxylic acid tert-butyl ester (0.53 g, 1.4 mmol) provided a mixture of 3.7:1 of the regioisomers 5-{4-[3-(7-fluoro-1H-indazol-4-yl)-2-pyridin-4-yl-pyrazolo[1,5-a]pyrimidin-7-yl]-phenyl}-2,5-diaza-bicyclo[2.2.1]-heptane-2-carboxylic acid tert-butyl ester and 5-{4-[3-(7-fluoro-1H-indazol-4-yl)-2-pyridin-4-yl-pyrazolo[1,5-a]pyrimidin-5-yl]-phenyl}-2,5-diazabicyclo[2.2.1]heptane-2-carboxylic acid tert-butyl ester. These isomers were separated by flash chromatography using a gradient of 1% to 3% MeOH/CH₂CI₂, providing tert-butyl(1S,4S)-5-{4-[3-(7-fluoro-1H-indazol-4-yl)-2-pyridin-4-ylpyrazolo[1,5-a]pyrimidin-7-yl]phenyl}-2,5-diazabicyclo[2.2.1]heptane-2-carboxylate as a yellow solid. MS: 603.3 [M+H].

### Example 103

### 7-[4-(2,5-Diaza-bicyclo[2.2.1]hept-2-yl)-phenyl]-3-(7-fluoro-1H-indazol-4-yl)-2-pyridin-4-yl-pyrazolo[1,5-a]pyrimidine

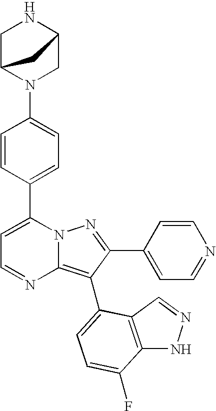

A portion of tert-butyl(1S,4S)-5-{4-[3-(7-fluoro-1H-indazol-4-yl)-2-pyridin-4-ylpyrazolo[1,5-a]pyrimidin-7-yl]phenyl}-2,5-diazabicyclo[2.2.1]heptane-2-carboxylate (0.32 g, 5.2 mmol) was dissolved in 10.8 mL of 4N HCl (concentrated HCl diluted with methanol) and followed by mass spectrometry until completion of the reaction. The mixture was concentrated in vacuo and purified by flash chromatography using 9:1:0.1 MeOH/CH₂Cl₂/ammonium hydroxide to provide 0.24 g of 7-[4-(2,5-diaza-bicyclo[2.2.1]hept-2-yl)-phenyl]-3-(7-fluoro-1H-indazol-4-yl)-2-pyridin-4-ylpyrazolo[1,5-a]pyrimidine as a yellow solid (92% yield). MS: 503.3 [M+H].

### Example 104

### 3-(7-fluoro-1H-indazol-4-yl)-7-{4-[(1S,4S)-5-methyl-2,5-diazabicyclo[2.2.1]hept-2-yl]phenyl}-2-pyridin-4-ylpyrazolo[1,5-a]pyrimidine hydrochloride

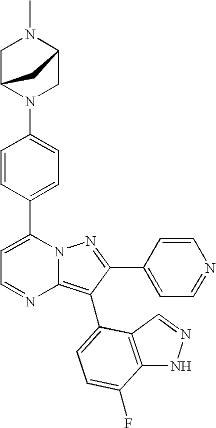

A 0.22 g portion of 7-[4-(2,5-diaza-bicyclo[2.2.1]hept-2-yl)-phenyl]-3-(7-fluoro-1H-indazol-4-yl)-2-pyridin-4-yl-pyrazolo[1,5-a]pyrimidine was dissolved in 7.8 mL of DMF, and 0.16 mL of 37% formaldehyde in H₂O solution was added (2.2 mmol, 5.0 equiv), followed by 0.05 mL of acetic acid (0.9 mmol, 2.0 equiv). This mixture was stirred for 15 minutes, then 0.46 g of sodium triacetoxyborohydride (Na(OAc)₃BH) (2.2 mmol, 5.0 equiv) was added and the reaction stirred for one hour. The product mixture was quenched with 5 mL of 7N ammonia in methanol and stirred for another 30 minutes. The reaction was then concentrated in vacuo and purified by flash chromatography using 9:1:0.1 MeOH/CH₂Cl₂/ammonium hydroxide. The yellow solid was then dissolved in dichloromethane and washed with saturated sodium bicarbonate, dried over MgSO₄ and evaporated to dryness to produce 0.22 g of a yellow solid. This solid was then dissolved in 5 mL of methanol and cooled in an ice bath. To this was added 0.34 mL of 3N methanolic HCl and the mixture was stirred at 0° C. for 15 minutes. The product mixture was concentrated in vacuo to yield 0.2 g of 3-(7-fluoro-1H-indazol-4-yl)-7-[4-(5-methyl-2,5-diaza-bicyclo[2.2.1]hept-2-yl)-phenyl]-2-pyridin-4-yl-pyrazolo[1,5-a]pyrimidine hydrochloric salt as an orange-red solid (83% yield). MS: 517.3 [M+H].

### Example 105

### tert-Butyl(1S,4S)-5-{3-fluoro-4-[3-(7-fluoro-1H-indazol-4-yl)-2-pyridin-4-ylpyrazolo[1,5-a]pyrimidin-7-yl]phenyl}-2,5-diazabicyclo[2.2.1]heptane-2-carboxylate

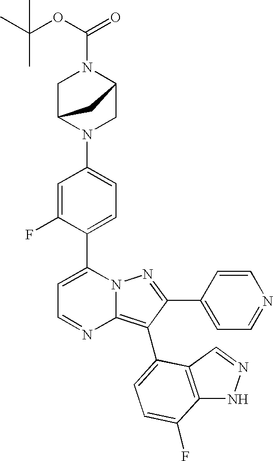

A 2.31 g portion of 5-[4-(3-dimethylamino-acryloyl)-3-fluoro-phenyl]-2,5-diazabicyclo[2.2.1]heptane-2-carboxylic acid tert-butyl ester (1.1 equiv, 5.9 mmol) was dissolved in 37 mL of methanol. To this was added 1.59 g of 4-(7-fluoro-1H-indazol-4-yl)-5-pyridin-4-yl-2H-pyrazol-3-ylamine (1.0 equiv, 5.4 mmol), followed by 1.1 mL of trifluoroacetic acid and the reaction was stirred at room temperature for 16 hours. The reaction was concentrated in vacuo, neutralized with saturated aqueous sodium bicarbonate (NaHCO₃) and filtered. The solid was washed with water and dried in vacuo. Purification was carried out by silica flash chromatography, eluting with 5%-10% methanol/ethyl acetate to yield 2.37 g of 5-{3-fluoro-4-[3-(7-fluoro-1H-indazol-4-yl)-2-pyridin-4-yl-pyrazolo[1,5-a]pyrimidin-7-yl]-phenyl}-2,5-diaza-bicyclo[2.2.1]heptane-2-carboxylic acid tert-butyl ester as a yellow solid (70.7% yield). MS: 621.3 [M+H].

### Example 106

### 7-{4-[(1S,4S)-2,5-diazabicyclo[2.2.1]hept-2-yl]-2-fluorophenyl}-3-(7-fluoro-1H-indazol-4-yl )-2-pyridin-4-yl pyrazolo[1 5-a]pyrimidine

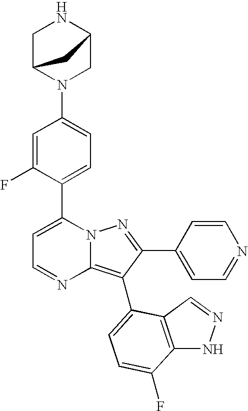

Following the procedure of Example 103, 5-{3-fluoro-4-[3-(7-fluoro-1H-indazol-4-yl)-2-pyridin-4-yl-pyrazolo[1,5-a]pyrimidin-7-yl]-phenyl}-2,5-diaza-bicyclo[2.2.1]heptane-2-carboxylic acid tert-butyl ester provided 7-[4-(2,5-diaza-bicyclo[2.2.1]hept-2-yl)-2-fluoro-phenyl]-3-(7-fluoro-1H-indazol-4-yl)-2-pyridin-4-yl-pyrazolo[1,5-a]pyrimidine. MS: 521.2 [M+H]; 261.2 [M+2H].

### Example 107

### 3-(7-fluoro-1H-indazol-4-yl)-7-{2-fluoro-4-[(1S,4S)-5-methyl-2,5-diazabicyclo[2.2.1]hept-2-yl]phenyl}-2-pyridin-4-ylpyrazolo[1,5-a]pyrimidine

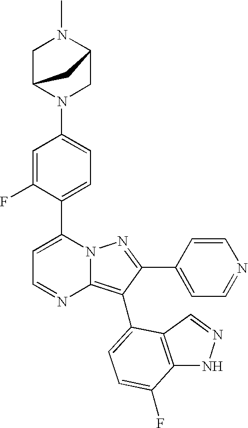

A 1.44 g portion of 7-[4-(2,5-diaza-bicyclo[2.2.1]hept-2-yl)-2-fluoro-phenyl]-3-(7-fluoro-1H-indazol-4-yl)-2-pyridin-4-yl-pyrazolo[1,5-a]pyrimidine (2.8 mmol, 1.0 equiv) was dissolved in 50 mL of DMF, and 1 mL of a 37% aqueous formaldehyde (13.8 mmol, 5.0 equiv) solution was added. To this mixture was added 0.32 mL of acetic acid (5.5 mmol, 2.0 equiv)) and the solution was stirred for 15 minutes. A 2.93 g portion of Na(OAc)₃BH (13.8 mmol, 5.0 equiv) was added, and the reaction stirred for one hour. The product mixture was quenched with 30 mL of 7N ammonia in MeOH and stirred for another 30 minutes. The reaction was then partitioned between water and ethyl acetate and the aqueous layer was extracted with ethyl acetate again. The organic layers were combined, dried over MgSO₄ and concentrated in vacuo to give a yellow oil. Upon the addition of water to this oil, a solid precipitated out. The resulting solid was filtered and dried to provide 1.3 g of 3-(7-fluoro-1H-indazol-4-yl )-7-[2-fluoro-4-(5-methyl-2,5-diaza-bicyclo[2.2.1]hept-2-yl)-phenyl]-2-pyridin-4-yl-pyrazolo[1,5-a]pyrimidine as a yellow solid (88% yield). MS: 535.2 [M+H].

### Example 108

### tert-Butyl(1S,4S)-5-{3,5-difluoro-4-[3-(7-fluoro-1H-indazol-4-yl)-2-pyridin-4-ylpyrazolo[1,5-a]pyrimidin-7-yl]phenyl}-2,5-diazabicyclo[2.2.1]heptane-2-carboxylate

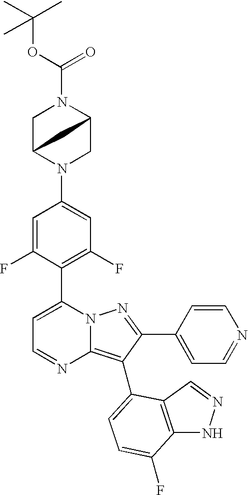

Following the procedure of Example 100, Step 3, (1S,4S)-tert-butyl 5-(3,5-difluoro-4-(3-(7-fluoro-1H-indazol-4-yl)-2-(pyridin-4-yl )pyrazolo[1,5-a]pyrimidin-7-yl)phenyl)-2,5-diazabicyclo[2.2.1]heptane-2-carboxylate was prepared from 4-(7-fluoro-1H-indazol-4-yl)-3-(pyridin-4-yl)-1H-pyrazol-5-amine and (1S,4S)-tert-butyl 5-(4-((E)-3-(dimethylamino)acryloyl)-3,5-difluorophenyl)-2,5-diazabicyclo[2.2.1]-heptane-2-carboxylate. MS: 639.2 [M+H].

### Example 109

### 7-{4-[(1S,4S)-2,5-Diazabicyclo[2.2.1]hept-2-yl]-2,6-difluorophenyl}-3-(7-fluoro-1H-indazol-4-yl )-2-pyridin-4-yl pyrazolo[1,5-a]pyrimidine

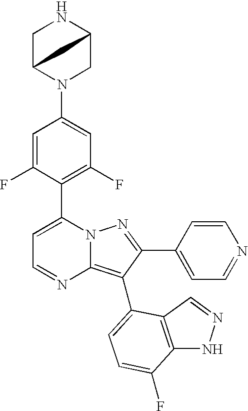

Following the procedure of Example 102, 7-(4-((1S,4S)-2,5-diazabicyclo[2.2.1]heptan-2-yl)-2,6-difluorophenyl)-3-(7-fluoro-1H-indazol-4-yl)-2-(pyridin-4-yl)pyrazolo[1,5-a]pyrimidine was prepared from (1S,4S)-tert-butyl 5-(3,5-difluoro-4-(3-(7-fluoro-1H-indazol-4-yl)-2-(pyridin-4-yl)pyrazolo[1,5-a]pyrimidin-7-yl)phenyl)-2,5-diazabicyclo[2.2.1]heptane-2-carboxylate. MS: 539.3 [M+H].

### Example 110

### 7-{2,6-Difluoro-4-[(1S,4S)-5-methyl-2,5-diazabicyclo[2.2.1]hept-2-yl]phenyl}-3-(7-fluoro-1H-indazol-4-yl)-2-pyridin-4-yl pyrazolo[1,5-a]pyrimidine

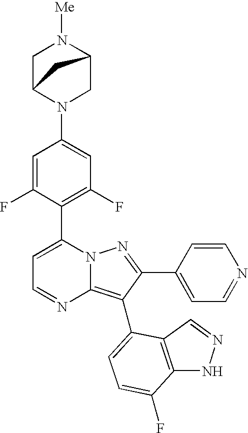

Following the procedure of Example 101, 7-(2,6-difluoro-4-((1S,4S)-5-methyl-2,5-diazabicyclo[2.2.1]heptan-2-yl)phenyl)-3-(7-fluoro-1H-indazol-4-yl)-2-(pyridin-4-yl)pyrazolo[1,5-a]pyrimidine was prepared from 7-(4-((1S,4S)-2,5-diazabicyclo[2.2.1]heptan-2-yl)-2,6-difluorophenyl)-3-(7-fluoro-1H-indazol-4-yl)-2-(pyridin-4-yl)pyrazolo[1,5-a]pyrimidine, MS: 553.2 [M+H].

Examples 111-210 are summarized in Table 1.

| | | MS, ESI |
|---|---|---|
| Example | Compound Name | (m/z) |
| 111 | ethyl 3-[3-(1H-indazol-4-yl)-2-pyridin-4-ylpyrazolo[1,5-a]pyrimidin-5- | 494 |
| | yl]-8-azabicyclo[3.2.1]octane-8-carboxylate | |
| 112 | 2-{3-[3-(1H-indazol-4-yl)-2-pyridin-4-ylpyrazolo[1,5-a]pyrimidin-7-yl]- | 466.3 |
| | 8-azabicyclo[3.2.1]oct-8-yl}ethanol | |
| 113 | 3-(1H-indazol-4-yl)-7-(8-isopropyl-8-azabicyclo[3.2.1]oct-3-yl)-2- | 464.3 |
| | pyridin-4-ylpyrazolo[1,5-a]pyrimidine | |
| 114 | 3-(1H-indazol-4-yl)-7-[8-(methylsulfonyl)-8-azabicyclo[3.2.1]oct-3-yl]- | 500.3 |
| | 2-pyridin-4-ylpyrazolo[1,5-a]pyrimidine | |
| 115 | 3-[3-(1H-indazol-4-yl)-2-pyridin-4-ylpyrazolo[1,5-a]pyrimidin-7-yl]-8- | 465.2 |
| | azabicyclo[3.2.1]octane-8-carboxamide | |
| 116 | 2-{3-[3-(1H-indazol-4-yl)-2-pyridin-4-ylpyrazolo[1,5-a]pyrimidin-7-yl]- | 507.3 |
| | 8-azabicyclo[3.2.1]oct-8-yl}-N,N-dimethyl-2-oxoethanamine | |
| 117 | {3-[3-(1H-indazol-4-yl)-2-pyridin-4-ylpyrazolo[1,5-a]pyrimidin-7-yl]-8- | 461.2 |
| | azabicyclo[3.2.1]oct-8-yl}acetonitrile | |
| 118 | N-ethyl-3-[3-(1H-indazol-4-yl)-2-pyridin-4-ylpyrazolo[1,5-a]pyrimidin- | 493.2 |
| | 7-yl]-8-azabicyclo[3.2.1]octane-8-carboxamide | |
| 119 | 7-(8-acetyl-8-azabicyclo[3.2.1]oct-3-yl)-3-(1H-indazol-4-yl)-2-pyridin- | 464.2 |
| | 4-ylpyrazolo[1,5-a]pyrimidine | |
| 120 | 3-[3-(1H-indazol-4-yl)-2-pyridin-4-ylpyrazolo[1,5-a]pyrimidin-7-yl]- | 493.2 |
| | N,N-dimethyl-8-azabicyclo[3.2.1]octane-8-carboxamide | |
| 121 | tert-butyl (1S,4S)-5-{[3-(4-chloro-3-methoxyphenyl)-2-pyridin-4- | 547.2; |
| | ylpyrazolo[1,5-a]pyrimidin-7-yl]methyl}-2,5- | |
| | diazabicyclo[2.2.1]heptane-2-carboxylate | |
| 122 | tert-butyl (1S,4S)-5-{4-[3-(7-fluoro-1H-indazol-4-yl)-2-pyridin-4- | 603.2 |
| | ylpyrazolo[1,5-a]pyrimidin-5-yl]phenyl}-2,5- | |
| | diazabicyclo[2.2.1]heptane-2-carboxylate | |
| 123 | tert-butyl (1S,4S)-5-{3-[3-(1H-indazol-4-yl)-2-pyridin-4- | 585.3 |
| | ylpyrazolo[1,5-a]pyrimidin-7-yl]phenyl}-2,5- | |
| | diazabicyclo[2.2.1]heptane-2-carboxylate | |
| 124 | 7-{3-[(1S,4S)-2,5-diazabicyclo[2.2.1]hept-2-yl]phenyl}-3-(1H-indazol- | 485.2 |
| | 4-yl)-2-pyridin-4-ylpyrazolo[1,5-a]pyrimidine | |
| 125 | tert-butyl (1S,4S)-5-{4-[3-(7-chloro-1H-indazol-4-yl)-2-pyridin-4- | 619.2 |
| | ylpyrazolo[1,5-a]pyrimidin-7-yl]phenyl}-2,5- | |
| | diazabicyclo[2.2.1]heptane-2-carboxylate | |
| 126 | 3-(1H-indazol-4-yl)-7-{3-[(1S,4S)-5-methyl-2,5- | 499.2 |
| | diazabicyclo[2.2.1]hept-2-yl]phenyl}-2-pyridin-4-ylpyrazolo[1,5- | |
| | a]pyrimidine | |
| 127 | 3-(7-chloro-1H-indazol-4-yl)-7-{4-[(1S,4S)-2,5- | 519.3 |
| | diazabicyclo[2.2.1]hept-2-yl]phenyl}-2-pyridin-4-ylpyrazolo[1,5- | |
| | a]pyrimidine | |
| 128 | tert-butyl (2S)-2-({3-[3-(1H-indazol-4-yl)-2-pyridin-4-ylpyrazolo[1,5- | 619.3; |
| | a]pyrimidin-7-yl]-8-azabicyclo[3.2.1]oct-8-yl}carbonyl)pyrrolidine-1- | |
| | carboxylate | |
| 129 | 3-(1H-indazol-4-yl)-7-(8-L-prolyl-8-azabicyclo[3.2.1]oct-3-yl)-2- | 519.3 |
| | pyridin-4-ylpyrazolo[1,5-a]pyrimidine | |
| 130 | 1-{3-[3-(1H-indazol-4-yl)-2-pyridin-4-ylpyrazolo[1,5-a]pyrimidin-7-yl]- | 478.2 |
| | 8-azabicyclo[3.2.1]oct-8-yl}propan-2-one | |
| 131 | ethyl 3-[3-(7-fluoro-1H-indazol-4-yl)-2-pyridin-4-ylpyrazolo[1,5- | 512.3 |
| | a]pyrimidin-5-yl]-8-azabicyclo[3.2.1]octane-8-carboxylate | |
| 132 | 7-{4-[(1S,4S)-2,5-diazabicyclo[2.2.1]hept-2-yl]-2-fluorophenyl}-3-(3- | 493.2 |
| | methoxyphenyl)-2-pyridin-4-ylpyrazolo[1,5-a]pyrimidine | |
| 133 | 3-(7-{4-[(1S,4S)-2,5-diazabicyclo[2.2.1]hept-2-yl]-2-fluorophenyl}-2- | 479.2 |
| | pyridin-4-ylpyrazolo[1,5-a]pyrimidin-3-yl)phenol | |
| 134 | 3-(7-{2-fluoro-4-[(1S,4S)-5-methyl-2,5-diazabicyclo[2.2.1]hept-2- | 493.2 |
| | yl]phenyl}-2-pyridin-4-ylpyrazolo[1,5-a]pyrimidin-3-yl)phenol | |
| 135 | 7-{4-[(1S,4S)-2,5-diazabicyclo[2.2.1]hept-2-yl]-2-methylphenyl}-3-(7- | 517.2 |
| | fluoro-1H-indazol-4-yl)-2-pyridin-4-ylpyrazolo[1,5-a]pyrimidine | |
| 136 | 3-(7-fluoro-1H-indazol-4-yl)-7-{2-methyl-4-[(1S,4S)-5-methyl-2,5- | 531.2 |
| | diazabicyclo[2.2.1]hept-2-yl]phenyl}-2-pyridin-4-ylpyrazolo[1,5- | |
| | a]pyrimidine | |
| 137 | 7-(8-azabicyclo[3.2.1]oct-3-yl)-3-(7-fluoro-1H-indazol-4-yl)-2-pyridin- | 440.2 |
| | 4-ylpyrazolo[1,5-a]pyrimidine | |
| 138 | {3-[3-(7-fluoro-1H-indazol-4-yl)-2-pyridin-4-ylpyrazolo[1,5- | 479.1 |
| | a]pyrimidin-7-yl]-8-azabicyclo[3.2.1]oct-8-yl}acetonitrile | |
| 139 | 3-(7-chloro-1H-indazol-4-yl)-5-{4-[(1S,4S)-2,5- | 519.0 |
| | diazabicyclo[2.2.1]hept-2-yl]phenyl}-2-pyridin-4-ylpyrazolo[1,5- | |
| | a]pyrimidine | |
| 140 | 3-(1H-indazol-4-yl)-7-[6-(8-methyl-3,8-diazabicyclo[3.2.1]oct-3- | 514.3 |
| | yl)pyridin-3-yl]-2-pyridin-4-ylpyrazolo[1,5-a]pyrimidine | |
| 141 | 3-(7-chloro-1H-indazol-4-yl)-7-{4-[(1S,4S)-5-methyl-2,5- | 533.3; |
| | diazabicyclo[2.2.1]hept-2-yl]phenyl}-2-pyridin-4-ylpyrazolo[1,5- | |
| | a]pyrimidine | |
| 142 | 7-(1,4-dioxaspiro[4.5]dec-8-yl)-3-(1H-indazol-4-yl)-2-pyridin-4- | 453.3; |
| | ylpyrazolo[1,5-a]pyrimidine | |
| 143 | 3-(1H-indazol-4-yl)-7-{4-[(1S,4S)-5-methyl-2,5- | 513.2 |
| | diazabicyclo[2.2.2]oct-2-yl]phenyl}-2-pyridin-4-ylpyrazolo[1,5- | |
| | a]pyrimidine | |
| 144 | 7-{4-[(1S,4S)-2,5-diazabicyclo[2.2.2]oct-2-yl]phenyl}-3-(1H-indazol- | 499.2 |
| | 4-yl)-2-pyridin-4-ylpyrazolo[1,5-a]pyrimidine | |
| 145 | 7-{2-[(1S,4S)-2,5-diazabicyclo[2.2.1]hept-2-yl]-4,6-difluorophenyl}-3- | 521.7 |
| | (1H-indazol-4-yl)-2-pyridin-4-ylpyrazolo[1,5-a]pyrimidine | |
| 146 | 5-[(1S,4S)-2,5-diazabicyclo[2.2.1]hept-2-yl]-2-[3-(1H-indazol-4-yl)-2- | 528.3 |
| | pyridin-4-ylpyrazolo[1,5-a]pyrimidin-7-yl]-N,N-dimethylaniline | |
| 147 | 7-{2,4-difluoro-6-[(1S,4S)-5-methyl-2,5-diazabicyclo[2.2.1]hept-2- | 535.2 |
| | yl]phenyl}-3-(1H-indazol-4-yl)-2-pyridin-4-ylpyrazolo[1,5-a]pyrimidine | |
| 148 | 2-[3-(1H-indazol-4-yl)-2-pyridin-4-ylpyrazolo[1,5-a]pyrimidin-7-yl]- | 542.3 |
| | N,N-dimethyl-5-[(1S,4S)-5-methyl-2,5-diazabicyclo[2.2.1]hept-2- | |
| | yl]aniline | |
| 149 | 7-{cis-4-[(1S,4S)-2,5-diazabicyclo[2.2.1]hept-2-yl]cyclohexyl}-3-(1H- | 491.5 |
| | indazol-4-yl)-2-pyridin-4-ylpyrazolo[1,5-a]pyrimidine | |
| 150 | 3-(1H-indazol-4-yl)-7-[cis-4-(3-oxa-8-azabicyclo[3.2.1]oct-8- | 506.5 |
| | yl)cyclohexyl]-2-pyridin-4-ylpyrazolo[1,5-a]pyrimidine | |
| 151 | 3-(1H-indazol-4-yl)-7-[trans-4-(3-oxa-8-azabicyclo[3.2.1]oct-8- | 506.5 |
| | yl)cyclohexyl]-2-pyridin-4-ylpyrazolo[1,5-a]pyrimidine | |
| 152 | 3-(1H-indazol-4-yl)-7-[cis-4-(8-oxa-3-azabicyclo[3.2.1]oct-3- | 506.2 |
| | yl)cyclohexyl]-2-pyridin-4-ylpyrazolo[1,5-a]pyrimidine | |
| 153 | 3-(1H-indazol-4-yl)-7-[trans-4-(8-oxa-3-azabicyclo[3.2.1]oct-3- | 506.2 |
| | yl)cyclohexyl]-2-pyridin-4-ylpyrazolo[1,5-a]pyrimidine | |
| 154 | 3-(1H-indazol-4-yl)-7-{cis-4-[(1S,4S)-2-oxa-5-azabicyclo[2.2.1]hept- | 492.5 |
| | 5-yl]cyclohexyl}-2-pyridin-4-ylpyrazolo[1,5-a]pyrimidine | |
| 155 | 3-(1H-indazol-4-yl)-7-{trans-4-[(1S,4S)-2-oxa-5- | 492.4 |
| | azabicyclo[2.2.1]hept-5-yl]cyclohexyl}-2-pyridin-4-ylpyrazolo[1,5- | |
| | a]pyrimidine | |
| 156 | 7-{trans-4-[(1S,4S)-2,5-diazabicyclo[2.2.1]hept-2-yl]cyclohexyl}-3- | 491.5 |
| | (1H-indazol-4-yl)-2-pyridin-4-ylpyrazolo[1,5-a]pyrimidine | |
| 157 | 7-{4-[(1S,4S)-2,5-diazabicyclo[2.2.1]hept-2-yl]-3- | 553.2 |
| | (trifluoromethyl)phenyl}-3-(1H-indazol-4-yl)-2-pyridin-4- | |
| | ylpyrazolo[1,5-a]pyrimidine | |
| 158 | 7-{4-[(1S,4S)-2,5-diazabicyclo[2.2.1]hept-2-yl]-1-naphthyl}-3-(1H- | 535.2 |
| | indazol-4-yl)-2-pyridin-4-ylpyrazolo[1,5-a]pyrimidine | |
| 159 | 3-(1H-indazol-4-yl)-7-{4-[(1S,4S)-5-methyl-2,5- | 567.2 |
| | diazabicyclo[2.2.1]hept-2-yl]-3-(trifluoromethyl)phenyl}-2-pyridin-4- | |
| | ylpyrazolo[1,5-a]pyrimidine | |
| 160 | 3-(1H-indazol-4-yl)-7-{4-[(1S,4S)-5-methyl-2,5- | 549.2 |
| | diazabicyclo[2.2.1]hept-2-yl]-1-naphthyl}-2-pyridin-4-ylpyrazolo[1,5- | |
| | a]pyrimidine | |
| 161 | 7-{4-[(1S,4S)-2,5-diazabicyclo[2.2.1]hept-2-yl]-3,5-difluorophenyl}-3- | 521.2 |
| | (1H-indazol-4-yl)-2-pyridin-4-ylpyrazolo[1,5-a]pyrimidine | |
| 162 | 7-{4-[(1S,4S)-2,5-diazabicyclo[2.2.1]hept-2-yl]-2,3-difluorophenyl}-3- | 521.2 |
| | (1H-indazol-4-yl)-2-pyridin-4-ylpyrazolo[1,5-a]pyrimidine | |
| 163 | 7-{4-[(1S,4S)-2,5-diazabicyclo[2.2.1]hept-2-yl]-2,5-difluorophenyl}-3- | 521.5 |
| | (1H-indazol-4-yl)-2-pyridin-4-ylpyrazolo[1,5-a]pyrimidine | |
| 164 | 7-{3,5-difluoro-4-[(1S,4S)-5-methyl-2,5-diazabicyclo[2.2.1]hept-2- | 535.2 |
| | yl]phenyl}-3-(1H-indazol-4-yl)-2-pyridin-4-ylpyrazolo[1,5-a]pyrimidine | |
| 165 | 7-{2,3-difluoro-4-[(1S,4S)-5-methyl-2,5-diazabicyclo[2.2.1]hept-2- | 535.2 |
| | yl]phenyl}-3-(1H-indazol-4-yl)-2-pyridin-4-ylpyrazolo[1,5-a]pyrimidine | |
| 166 | 7-{2,5-difluoro-4-[(1S,4S)-5-methyl-2,5-diazabicyclo[2.2.1]hept-2- | 535.2 |
| | yl]phenyl}-3-(1H-indazol-4-yl)-2-pyridin-4-ylpyrazolo[1,5-a]pyrimidine | |
| 167 | 7-{4-[(1S,4S)-5-ethyl-2,5-diazabicyclo[2.2.1]hept-2-yl]-2,6- | 549.3 |
| | difluorophenyl}-3-(1H-indazol-4-yl)-2-pyridin-4-ylpyrazolo[1,5- | |
| | a]pyrimidine | |
| 168 | 7-{2,6-difluoro-4-[(1S,4S)-5-isobutyl-2,5-diazabicyclo[2.2.1]hept-2- | 577.3 |
| | yl]phenyl}-3-(1H-indazol-4-yl)-2-pyridin-4-ylpyrazolo[1,5-a]pyrimidine | |
| 169 | 7-{2,6-difluoro-4-[(1S,4S)-5-isopropyl-2,5-diazabicyclo[2.2.1]hept-2- | 563.2 |
| | yl]phenyl}-3-(1H-indazol-4-yl)-2-pyridin-4-ylpyrazolo[1,5-a]pyrimidine | |
| 170 | 7-{4-[(1S,4S)-5-cyclobutyl-2,5-diazabicyclo[2.2.1]hept-2-yl]-2,6- | 575.2 |
| | difluorophenyl}-3-(1H-indazol-4-yl)-2-pyridin-4-ylpyrazolo[1,5- | |
| | a]pyrimidine | |
| 171 | 7-(1,4-dioxaspiro[4.5]dec-8-yl)-3-(7-fluoro-1H-indazol-4-yl)-2-pyridin- | 471.2 |
| | 4-ylpyrazolo[1,5-a]pyrimidine | |
| 172 | 7-{4-[(1S,4S)-2,5-diazabicyclo[2.2.2]oct-2-yl]-2-fluorophenyl}-3-(1H- | 517.2 |
| | indazol-4-yl)-2-pyridin-4-ylpyrazolo[1,5-a]pyrimidine | |
| 173 | 7-{2-fluoro-4-[(1S,4S)-5-methyl-2,5-diazabicyclo[2.2.2]oct-2- | 531.2 |
| | yl]phenyl}-3-(1H-indazol-4-yl)-2-pyridin-4-ylpyrazolo[1,5-a]pyrimidine | |
| 174 | 3-(1H-indazol-4-yl)-2-pyridin-4-yl-7-{2,3,5,6-tetrafluoro-4-[(1S,4S)-5- | 571.2 |
| | methyl-2,5-diazabicyclo[2.2.1]hept-2-yl]phenyl}pyrazolo[1,5- | |
| | a]pyrimidine | |
| 175 | tert-butyl (1S,4S)-5-{3-chloro-4-[3-(7-fluoro-1H-indazol-4-yl)-2- | 637.3 |
| | pyridin-4-ylpyrazolo[1,5-a]pyrimidin-7-yl]phenyl}-2,5- | |
| | diazabicyclo[2.2.1]heptane-2-carboxylate | |
| 176 | 7-{cis-4-[(1S,4S)-2,5-diazabicyclo[2.2.1]hept-2-yl]cyclohexyl}-3-(7- | 509.2 |
| | fluoro-1H-indazol-4-yl)-2-pyridin-4-ylpyrazolo[1,5-a]pyrimidine | |
| 177 | 3-(7-fluoro-1H-indazol-4-yl)-7-{cis-4-[(1S,4S)-2-oxa-5- | 510.3 |
| | azabicyclo[2.2.1]hept-5-yl]cyclohexyl}-2-pyridin-4-ylpyrazolo[1,5- | |
| | a]pyrimidine | |
| 178 | 3-(7-fluoro-1H-indazol-4-yl)-7-{trans-4-[(1S,4S)-2-oxa-5- | 510.3 |
| | azabicyclo[2.2.1]hept-5-yl]cyclohexyl}-2-pyridin-4-ylpyrazolo[1,5- | |
| | a]pyrimidine | |
| 179 | 3-(7-fluoro-1H-indazol-4-yl)-7-[cis-4-(8-oxa-3-azabicyclo[3.2.1]oct-3- | 524.3 |
| | yl)cyclohexyl]-2-pyridin-4-ylpyrazolo[1,5-a]pyrimidine | |
| 180 | 3-(7-fluoro-1H-indazol-4-yl)-7-[trans-4-(8-oxa-3-azabicyclo[3.2.1]oct- | 524.3 |
| | 3-yl)cyclohexyl]-2-pyridin-4-ylpyrazolo[1,5-a]pyrimidine | |
| 181 | 3-(7-fluoro-1H-indazol-4-yl)-7-[cis-4-(3-oxa-8-azabicyclo[3.2.1]oct-8- | 524.3 |
| | yl)cyclohexyl]-2-pyridin-4-ylpyrazolo[1,5-a]pyrimidine | |
| 182 | 3-(7-fluoro-1H-indazol-4-yl)-7-[trans-4-(3-oxa-8-azabicyclo[3.2.1]oct- | 524.3 |
| | 8-yl)cyclohexyl]-2-pyridin-4-ylpyrazolo[1,5-a]pyrimidine | |
| 183 | 7-{trans-4-[(1S,4S)-2,5-diazabicyclo[2.2.1]hept-2-yl]cyclohexyl}-3-(7- | 509.2 |
| | fluoro-1H-indazol-4-yl)-2-pyridin-4-ylpyrazolo[1,5-a]pyrimidine | |
| 184 | 7-{2-chloro-4-[(1S,4S)-2,5-diazabicyclo[2.2.1]hept-2-yl]phenyl}-3-(7- | 289.6 |
| | fluoro-1H-indazol-4-yl)-2-pyridin-4-ylpyrazolo[1,5-a]pyrimidine | |
| 185 | 7-{4-[(1S,4S)-2,5-diazabicyclo[2.2.1]hept-2-yl]-3-fluorophenyl}-3- | 503.2 |
| | (1H-indazol-4-yl)-2-pyridin-4-ylpyrazolo[1,5-a]pyrimidine | |
| 186 | 7-{4-[(1S,4S)-2,5-diazabicyclo[2.2.1]hept-2-yl]-2- | 553.2 |
| | (trifluoromethyl)phenyl}-3-(1H-indazol-4-yl)-2-pyridin-4- | |
| | ylpyrazolo[1,5-a]pyrimidine | |
| 187 | 7-{2-bromo-4-[(1S,4S)-2,5-diazabicyclo[2.2.1]hept-2-yl]phenyl}-3- | 563.1 |
| | (1H-indazol-4-yl)-2-pyridin-4-ylpyrazolo[1,5-a]pyrimidine | |
| 188 | 7-{3-fluoro-4-[(1S,4S)-5-methyl-2,5-diazabicyclo[2.2.1]hept-2- | 517.2 |
| | yl]phenyl}-3-(1H-indazol-4-yl)-2-pyridin-4-ylpyrazolo[1,5-a]pyrimidine | |
| 189 | 3-(1H-indazol-4-yl)-7-{4-[(1S,4S)-5-methyl-2,5- | 567.2 |
| | diazabicyclo[2.2.1]hept-2-yl]-2-(trifluoromethyl)phenyl}-2-pyridin-4- | |
| | ylpyrazolo[1,5-a]pyrimidine | |
| 190 | 7-{2-bromo-4-[(1S,4S)-5-methyl-2,5-diazabicyclo[2.2.1]hept-2- | 577.1 |
| | yl]phenyl}-3-(1H-indazol-4-yl)-2-pyridin-4-ylpyrazolo[1,5-a]pyrimidine | |
| 191 | 7-(1,4-dioxaspiro[4.5]dec-7-en-8-yl)-3-(1H-indazol-4-yl)-2-pyridin-4- | 451.2 |
| | ylpyrazolo[1,5-a]pyrimidine | |
| 192 | 7-{2,6-difluoro-4-[(1S,4S)-5-methyl-5-oxido-2,5- | 551.3 |
| | diazabicyclo[2.2.1]hept-2-yl]phenyl}-3-(1H-indazol-4-yl)-2-pyridin-4- | |
| | ylpyrazolo[1,5-a]pyrimidine | |
| 193 | 3-(1H-indazol-4-yl)-7-{5-[(1S,4S)-2-oxa-5-azabicyclo[2.2.1]hept-5- | 490.1 |
| | ylmethyl]furan-3-yl}-2-pyridin-4-ylpyrazolo[1,5-a]pyrimidine | |
| 194 | tert-butyl (1S,4S)-5-({4-[3-(1H-indazol-4-yl)-2-pyridin-4- | 589.1 |
| | ylpyrazolo[1,5-a]pyrimidin-7-yl]furan-2-yl}methyl)-2,5- | |
| | diazabicyclo[2.2.1]heptane-2-carboxylate | |
| 195 | 7-{5-[(1S,4S)-2,5-diazabicyclo[2.2.1]hept-2-ylmethyl]furan-3-yl}-3- | 489.2 |
| | (1H-indazol-4-yl)-2-pyridin-4-ylpyrazolo[1,5-a]pyrimidine | |
| 196 | tert-butyl (1S,4S)-5-({5-[3-(1H-indazol-4-yl)-2-pyridin-4- | 605.2 |
| | ylpyrazolo[1,5-a]pyrimidin-7-yl]thiophen-2-yl}methyl)-2,5- | |
| | diazabicyclo[2.2.1]heptane-2-carboxylate | |
| 197 | 3-(1H-indazol-4-yl)-7-{5-[(1S,4S)-2-oxa-5-azabicyclo[2.2.1]hept-5- | 506.1 |
| | ylmethyl]thiophen-2-yl}-2-pyridin-4-ylpyrazolo[1,5-a]pyrimidine | |
| 198 | 7-{5-[(1S,4S)-2,5-diazabicyclo[2.2.1]hept-2-ylmethyl]thiophen-2-yl}- | 505.1 |
| | 3-(1H-indazol-4-yl)-2-pyridin-4-ylpyrazolo[1,5-a]pyrimidine | |
| 199 | ethyl (3-endo)-3-[3-(1H-indazol-4-yl)-6-methyl-2-pyridin-4- | 508.2 |
| | ylpyrazolo[1,5-a]pyrimidin-7-yl]-8-azabicyclo[3.2.1]octane-8- | |
| | carboxylate | |
| 200 | 3-(1H-indazol-4-yl)-7-[6-(8-oxa-3-azabicyclo[3.2.1]oct-3-yl)pyridin-3- | 501.2 |
| | yl]-2-pyridin-4-ylpyrazolo[1,5-a]pyrimidine | |
| 201 | 3-(1H-indazol-4-yl)-7-{6-[(1S,4S)-2-oxa-5-azabicyclo[2.2.1]hept-5- | 487.2 |
| | yl]pyridin-3-yl}-2-pyridin-4-ylpyrazolo[1,5-a]pyrimidine | |
| 202 | tert-butyl (1S,4S)-5-{3-[3-(1H-indazol-4-yl)-2-pyridin-4- | 599.3 |
| | ylpyrazolo[1,5-a]pyrimidin-7-yl]benzyl}-2,5- | |
| | diazabicyclo[2.2.1]heptane-2-carboxylate | |
| 203 | tert-butyl (1S,4S)-5-{4-[3-(1H-indazol-4-yl)-2-pyridin-4- | 599.3 |
| | ylpyrazolo[1,5-a]pyrimidin-7-yl]benzyl}-2,5- | |
| | diazabicyclo[2.2.1]heptane-2-carboxylate | |
| 204 | 3-(1H-indazol-4-yl)-7-{4-[(1S,4S)-2-oxa-5-azabicyclo[2.2.1]hept-5- | 500.2 |
| | ylmethyl]phenyl}-2-pyridin-4-ylpyrazolo[1,5-a]pyrimidine | |
| 205 | 7-{4-[(1S,4S)-2,5-diazabicyclo[2.2.1]hept-2-ylmethyl]-2- | 517.2 |
| | fluorophenyl}-3-(1H-indazol-4-yl)-2-pyridin-4-ylpyrazolo[1,5- | |
| | a]pyrimidine | |
| 206 | 7-{2-fluoro-4-[(1S,4S)-2-oxa-5-azabicyclo[2.2.1]hept-5- | 518.2 |
| | ylmethyl]phenyl}-3-(1H-indazol-4-yl)-2-pyridin-4-ylpyrazolo[1,5- | |
| | a]pyrimidine | |
| 207 | 7-{2-fluoro-4-[(1R,4R)-2-oxa-5-azabicyclo[2.2.1]hept-5- | 518.2 |
| | ylmethyl]phenyl}-3-(1H-indazol-4-yl)-2-pyridin-4-ylpyrazolo[1,5- | |
| | a]pyrimidine | |
| 208 | 7-{3-[(1S,4S)-2,5-diazabicyclo[2.2.1]hept-2-ylmethyl]phenyl}-3-(1H- | 499.2 |
| | indazol-4-yl)-2-pyridin-4-ylpyrazolo[1,5-a]pyrimidine | |
| 209 | 7-{4-[(1S,4S)-2,5-diazabicyclo[2.2.1]hept-2-ylmethyl]phenyl}-3-(1H- | 499.3 |
| | indazol-4-yl)-2-pyridin-4-ylpyrazolo[1,5-a]pyrimidine | |
| 210 | 9-{3-fluoro-4-[3-(1H-indazol-4-yl)-2-pyridin-4-ylpyrazolo[1,5- | 548.3 |
| | a]pyrimidin-7-yl]benzyl}-3,7-dioxa-9-azabicyclo[3.3.1]nonane | |

### Standard Biological and Pharmacological Test Procedures

Evaluation of representative compounds of this invention in standard pharmacological test procedures indicated that the compounds of this invention possess significant anticancer activity and are inhibitors of Raf kinase. Based on the activity shown in the standard pharmacological test procedures, the compounds of this invention are therefore useful as antineoplastic agents. In particular, these compounds are useful in treating, inhibiting the growth of, or eradicating neoplasms such as those of the breast, kidney, bladder, thyroid, mouth, larynx, esophagus, stomach, colon, ovary, lung, pancreas, liver, prostate and skin. Compounds of the invention are useful as anti-inflammation agents and possess activity against inflammation associated with Raf kinases.

### Testing for Raf Kinase Inhibitors

Compounds of formula A were tested as Raf Kinase inhibitors for B-Raf kinase, mutant B-Raf kinase and C-Raf kinase, which are associated with inhibiting growth of tumor cells containing oncogenic forms of Receptor Tyrosine Kinases, K-Ras and Raf kinases.

### B-Raf Kinase

Reagents: Flag/GST-tagged recombinant human B-Raf produced in Sf9 insect cells, human non-active Mek-1-GST (recombinant protein produced in E. coli); and a phospho-MEK1 specific poly-clonal Ab from Cell Signaling Technology (Cat. #9121).

### Testing for B-Raf Kinase Inhibitors

B-Raf1 Kinase Assay Procedure: B-Raf-1 is used to phosphorylate GST-MEK1. MEK1 phosphorylation is measured by a phospho-specific antibody (from Cell Signaling Technology, Cat. #9121) that detects phosphorylation of two serine residues at positions 217 and 221 on MEK1.

The following Kinase Assay Protocol was employed in accordance with the invention:

### B-Raf Assay Stock Solutions

1. Assay Dilution Buffer (ADB): 20 mM MOPS, pH 7.2, 25 mM β-glycerol phosphate, 5 mM EGTA, 1 mM sodium orthovanadate, 1 mM dithiothreitol, 0.01% Triton X-100.
2. Magnesium/ATP Cocktail: ADB solution (minus Triton X-100) plus 200 μM cold ATP and 40 mM magnesium chloride.
4. Active Kinase: Active B-Raf: Used at 0.2 nM per assay point.
5. Non-active GST-MEK1: Used at 2.8 nM final concentration).
6. TBST—Tris (50 mM, pH 7.5), NaCl (150 mM), Tween-20 (0.05%)
7. Anti-GST Ab (GE)
8. Anti pMEK Ab (Upstate)
9. Anti-rabbit Ab/Europium conjugate (Wallac).

B-Raf Assay Procedure:
1. Added 25 μL of ADB containing B-Raf and Mek per assay (i.e. per well of a 96 well plate)
2. Added 25 μL of 0.2 mM ATP and 40 mM magnesium chloride in Magnesuium/ATP Cocktail.
3. Incubated for 45 minutes at RT with occasional shaking.
4. Transferred this mixture to an anti-GST Ab coated 96 well plate (Nunc Immunosorb plates coated o/n with a-GST. Plate freshly washed 3×with TBS-T before use.
5. Incubated o/n at 30° C. in cold room.
6. Washed 3× with TBST, ed Anti-Phospho MEK1 (1:1000, dilution depended upon lot)
7. Incubated for 60 minutes at RT in a shaking incubator
8. Washed 3× with TBST, add Anti-rabbit Ab/Europium conjugate (Wallac) (1:500, dilution depended upon lot)
9. Incubated for 60 minutes at RT on a platform shaker.
10. Washed plate 3× with TBS-T
11. Added 100 μL of Wallac Delfia Enhancement Solution and agitated for 10 minutes.
12. Read plates in Wallac Victor model Plate Reader.
13. Collected data and analyzed for single point and IC₅₀ determinations as described by Mallon R., et al. (2001) Anal. Biochem. 294:48.

### Testing for C-Raf Kinase Inhibitors

Assayed in a Raf-MEK-MAP kinase cascade assay as described previously (Mallon R, et al (2001) Anal. Biochem. 294:48.), except that C-Raf kinase was purchased from Upstate, Lake Placid, N.Y. and used at a concentration of 0.215 nM per assay point.

### Testing for Mutants of B-Raf Kinase Inhibitors

Assayed in a Raf-MEK-MAP kinase cascade assay as described previously (Mallon R, et al (2001) Anal. Biochem. 294:48.), except that B-Raf kinase mutants (V600 E) were used.

### Analysis of Results

B-Raf IC₅₀ determinations were performed on compounds of formula A from single point assays with >80 % inhibition. Single point assay: % inhibition at 10 mg/mL (% inhibition=1−sample treated with compound of Formula A/untreated control sample). The % inhibition was determined for each compound concentration. IC₅₀ determinations—Typically the B-Raf assay was run at compound concentrations from 1 μM to 3 nM or 0.1 μM to 300 pm in half log dilutions.

Selected compounds of formula A exhibited Raf kinase IC₅₀ values ranging from 1 μM to 0.1 nM, indicating that the compounds are effective inhibitors of Raf kinases, including B-Raf kinase, mutant B-Raf kinase and C-Raf kinase. The data is summarized in Table 2.

TABLE 2
B-Raf IC₅₀ Data for compounds of Formula A

| Example | Mean IC₅₀ (uM) |
|---|---|
| 1 | 0.002 |
| 2 | 0.002 |
| 3 | 0.001 |
| 4 | >1 |
| 5 | 0.006 |
| 6 | 0.057 |
| 7 | 0.087 |
| 8 | 0.001 |
| 9 | 0.004 |
| 10 | 0.054 |
| 11 | 0.002 |
| 12 | 0.0004 |
| 13 | 0.0003 |
| 14 | 0.002 |
| 15 | 0.01 |
| 16 | 0.009 |
| 17 | 0.044 |
| 18 | 0.0006 |
| 19 | 0.071 |
| 20 | 0.835 |
| 21 | >1 |
| 22 | >1 |
| 23 | >1 |
| 24 | NT |
| 25 | 0.001 |
| 26 | 0.01 |
| 27 | <0.0003 |
| 28 | 0.016 |
| 29 | 0.0005 |
| 30 | <0.003 |
| 31 | 0.0018 |
| 32 | 0.0087 |
| 33 | <0.0003 |
| 34 | 0.0023 |
| 35 | 0.0015 |
| 36 | 0.0082 |
| 37 | 0.021 |
| 38 | 0.0005 |
| 39 | 0.0005 |
| 40 | 0.0007 |
| 41 | <0.0003 |
| 42 | 0.0004 |
| 43 | 0.134 |
| 44 | 0.0006 |
| 45 | <0.0003 |
| 46 | 0.0032 |
| 47 | 0.0008 |
| 48 | >1.0 |
| 49 | 0.255 |
| 50 | >1.0 |
| 51 | 0.0008 |
| 52 | <0.0003 |
| 53 | <0.0003 |
| 54 | 0.15 |
| 55 | NT |
| 56 | <0.0003 |
| 57 | <0.0003 |
| 58 | <0.0003 |
| 59 | <0.0003 |
| 60 | <0.0003 |
| 61 | <0.0003 |
| 62 | <0.0003 |
| 63 | <0.0003 |
| 64 | 0.003 |
| 65 | 0.302 |
| 66 | 0.329 |
| 67 | >1.0 |
| 68 | 0.058 |
| 69 | NT |
| 70 | NT |
| 71 | NT |
| 72a | 0.566 |
| 72b | 0.417 |
| 73 | 0.002 |
| 74 | NT |
| 75 | 0.0003 |
| 76 | 0.004 |
| 77 | 0.017 |
| 78 | 0.0007 |
| 79 | 0.004 |
| 80 | 0.048 |
| 81 | 0.0004 |
| 82 | 0.002 |
| 83 | >1.0 |
| 84 | >1.0 |
| 85 | 0.036 |
| 86 | 0.27 |
| 87 | 0.032 |
| 88 | >1.0 |
| 89 | NT |
| 90 | 0.046 |
| 91 | 0.162 |
| 92 | NT |
| 93 | <0.0003 |
| 94 | 0.0001 |
| 95 | 0.00059 |
| 96 | NT |
| 97 | <0.0003 |
| 98 | <0.0003 |
| 99 | 0.0016 |
| 100 | <0.0002 |
| 101 | <0.0001 |
| 102 | 0.00204 |
| 103 | <0.0003 |
| 104 | 0.00024 |
| 105 | NT |
| 106 | <0.0003 |
| 107 | <0.0003 |
| 108 | NT |
| 109 | <0.0003 |
| 110 | <0.0001 |
| 111 | Not Tested |
| 112 | 0.0228 |
| 113 | 0.067 |
| 114 | 0.0049 |
| 115 | 0.0091 |
| 116 | 0.0618 |
| 117 | 0.0028 |
| 118 | 0.0067 |
| 119 | 0.0091 |
| 120 | 0.0064 |
| 121 | >0.100 |
| 122 | 0.0191 |
| 123 | Not Tested |
| 124 | 0.0376 |
| 125 | 0.0449 |
| 126 | 0.0612 |
| 127 | 0.0313 |
| 128 | 0.0394 |
| 129 | 0.0021 |
| 130 | 0.0759 |
| 131 | 0.001 |
| 132 | 0.0004 |
| 133 | <0.0003 |
| 134 | <0.0003 |
| 135 | <0.0003 |
| 136 | <0.0003 |
| 137 | 0.0038 |
| 138 | 0.0004 |
| 139 | 0.0012 |
| 140 | 0.0012 |
| 141 | 0.0023 |
| 142 | 0.0022 |
| 143 | 0.0007 |
| 144 | 0.0006 |
| 145 | 0.0744 |
| 146 | 0.0063 |
| 147 | >0.100 |
| 148 | 0.0073 |
| 149 | 0.0032 |
| 150 | 0.0399 |
| 151 | 0.0064 |
| 152 | 0.0064 |
| 153 | 0.0037 |
| 154 | 0.0114 |
| 155 | 0.0035 |
| 156 | 0.0006 |
| 157 | 0.0012 |
| 158 | 0.0209 |
| 159 | 0.002 |
| 160 | 0.0253 |
| 161 | 0.0027 |
| 162 | 0.0005 |
| 163 | <0.0003 |
| 164 | 0.0096 |
| 165 | 0.0006 |
| 166 | 0.0004 |
| 167 | <0.0003 |
| 168 | <0.0003 |
| 169 | <0.0003 |
| 170 | <0.0003 |
| 171 | Not Tested |
| 172 | 0.0003 |
| 173 | 0.0004 |
| 174 | 0.0004 |
| 175 | 0.001 |
| 176 | 0.001 |
| 177 | 0.004 |
| 178 | 0.0067 |
| 179 | 0.0086 |
| 180 | 0.0018 |
| 181 | 0.0021 |
| 182 | 0.0013 |
| 183 | <0.0003 |
| 184 | <0.0003 |
| 185 | 0.0017 |
| 186 | 0.0011 |
| 187 | <0.0003 |
| 188 | 0.0019 |
| 189 | 0.0014 |
| 190 | <0.0003 |
| 191 | 0.0561 |
| 192 | 0.0004 |
| 193 | 0.0188 |
| 194 | 0.0091 |
| 195 | 0.013 |
| 196 | 0.0115 |
| 197 | 0.0057 |
| 198 | 0.0036 |
| 199 | 0.0055 |
| 200 | 0.0058 |
| 201 | 0.0084 |
| 202 | >0.100 |
| 203 | 0.012 |
| 204 | 0.010 |
| 205 | 0.00035 |
| 206 | 0.0015 |
| 207 | 0.0005 |
| 208 | 0.042 |
| 209 | 0.0006 |
| 210 | 0.0007 |

NT = Not tested

What is claimed is:

1. A compound of formula A: 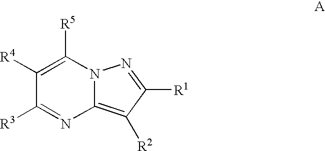
and pharmaceutically acceptable salts thereof;
wherein
R¹ is a 5-7 membered heterocyclic ring or heteroaryl ring, said ring comprising 1-3 heteroatoms selected from N, O or S, and said ring optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, —YR⁸R¹⁰, —YR⁸NR⁷R⁷ and —YR¹⁰;
R² is selected from an aryl ring, a 9-14 membered bicyclic aryl ring, a 5-7 membered heteroaryl ring and a 9-14 membered bicyclic heteroaryl ring, said heteroaryl ring comprising 1-3 heteroatoms selected from N, O and S, said ring optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, —OPO(OR⁷)₂, —YR⁸R¹⁰, —YR⁸NR⁷R⁷ and —YR¹⁰;
R³, R⁴ and R⁵ are each independently selected from carbon-linked R⁶, —X—W—R⁶, H, J, —C(O)OR⁷, —C(O)NR⁷R⁷, —NR⁷C(O)R⁷, —CN, alkyl of 1-6 carbon atoms, branched alkyl of 1-8 carbon atoms, cycloalkyl ring of 3-10 carbons, aryl ring, 5-7 membered heterocyclic ring, and 5-10 membered heteroaryl ring, said heterocyclic ring or heteroaryl ring comprising 1-3 heteroatoms selected from N, O and S, said alkyl of 1-6 carbon atoms, branched alkyl of 1-8 carbon atoms, aryl ring, 5-7 membered heterocyclic ring, and 5-10 membered heteroaryl ring is optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷R⁹OR⁷, —N(R⁷)R⁹NR⁷R⁷, —NR⁷C(O)R⁷, —C(O)R⁷, C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷—R⁸NR⁷C(O)NR⁷R⁷ and YR¹⁰, wherein at least one of R³, R⁴ and R⁵ comprises R⁶;
R⁶ is a 6-14 membered bridged, bicyclic heterocyclic ring or bicyclic spiro heterocyclic ring, said ring optionally substituted with one or more substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷or —R⁸NR⁷C(O)NR⁷R⁷and YR¹⁰;
R⁷ is H or is independently selected from alkyl of 1-6 carbon atoms, branched alkyl of 1-8 carbon atoms, alkenyl of 2-6 carbon atoms, alkynyl of 2-6 carbon atoms, aryl ring and a 5-10 membered heteroaryl ring, optionally substituted with one to four substituents selected from -J, —NO₂, —CN, —N₃, —CHO, —CF₃, —OCF₃, —R, —OR, —S(O)ₘR, —NRR, —NRS(O)ₘR, —OR⁹OR, —OR⁹NRR, —N(R)R⁹OR, —N(R)R⁹NRR, —NRC(O)R, —C(O)R, —C(O)OR, —C(O)NRR, —OC(O)R, —OC(O)OR, —OC(O)NRR, NRC(O)R, —NRC(O)OR, —NRC(O)NRR, —R⁸OR, —R⁸NRR, —R⁸S(O)ₘR, —R⁸C(O)R, —R⁸C(O)OR, —R⁸C(O)NRR, —R⁸OC(O)R, —R⁸OC(O)OR, —R⁸OC(O)NRR, —R⁸NRC(O)R, —R⁸NRC(O)OR, —R⁸NRC(O)NRR and ZR¹⁰, wherein R is selected from alkyl of 1-6 carbon atoms, branched alkyl of 1-8 carbon atoms, alkenyl of 2-6 carbon atoms, alkynyl of 2-6 carbon atoms, cycloalkyl of 3-10 carbon atoms, aryl of 6-10 carbon atoms and heteroaryl of 6-10 atoms, the heteroaryl comprising 1-3 heteroatoms selected from N, O and S;
R⁸ is a divalent group independently selected from alkyl of 1-6 carbon atoms, alkenyl of 2-6 carbon atoms, alkynyl of 2-6 carbon atoms, aryl, heteroaryl, cycloalkyl, and cycloheteroalkyl;
R⁹ is independently a divalent alkyl group of 2-6 carbon atoms;
R¹⁰ is independently selected from cycloalkyl ring of 3-10 carbons, bicycloalkyl ring of 3-10 carbons, aryl ring, heterocyclic ring, heteroaryl ring and a heteroaryl ring fused to one to three aryl or heteroaryl rings, each heterocyclic ring or heteroaryl ring comprising 1-3 heteroatoms selected from N, O and S, each optionally substituted with one to four substituents selected from —H, -aryl, —CH₂-aryl, —NH-aryl, —O-aryl, —S(O)ₘ-aryl, -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷—, —OC(O)OR⁷, —OC(O)NR⁷R⁷, —NR⁷C(O)R⁷, —NR⁷C(O)OR⁷, —NR⁷C(O)NR⁷R⁷, —R⁸OR⁷, R⁸NR⁷R⁷, —R⁸S(O)ₘR⁷, —R⁸C(O)R⁷, —R⁸C(O)OR⁷, —R⁸C(O)NR⁷R⁷, —RC(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, and —R⁸NR⁷C(O)NR⁷R⁷;
J is fluoro, chloro, bromo, or iodo;
m is an integer of 0-2;
Y is a divalent group independently selected from a bond, alkyl of 1-6 carbon atoms, alkenyl of 2-6 carbon atoms, alkynyl of 2-6 carbon atoms, O, and —NR⁷;
X is selected from a divalent alkyl group of 1-6 carbon atoms, alkenyl of 2-6 carbon atoms, alkynyl of 2-6 carbon atoms, cycloalkyl ring of 3-10 carbons, bicycloalkyl ring of 3-10 carbons, aryl ring, heterocyclic ring and a heteroaryl ring, each heterocyclic ring or heteroaryl ring comprising 1-3 heteroatoms selected from N, O or S; optionally substituted with one to four substituents selected from —H, -aryl, —CH₂-aryl, —NH-aryl, —O-aryl, —S(O)ₘ-aryl, -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —NR⁷C(O)R⁷, —C(O)R⁷, —C(O)OR⁷, —C(O)NR⁷R⁷, —OC(O)R⁷—, —OC(O)OR⁷, —OC(O)NR⁷R⁷, —NR⁷C(O)R⁷, —NR⁷C(O)OR⁷, —NR⁷C(O)NR⁷R⁷, —R⁸OR⁷, R⁸NR⁷R⁷, —R⁸S(O)ₘR⁷, —R⁸C(O)R⁷, —R⁸C(O)OR⁷, —R⁸C(O)NR⁷R⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, and —R⁸NR⁷C(O)NR⁷R⁷;
W is selected from a bond, —OZ—, —ZO—S(O)ₘZ—, —S(O)₂NR⁷Z—, —NR⁷S(O)ₘZ—, —NR⁷Z—, —ZNR⁷—, —C(O)Z—; —C(O)OZ—, —C(O)NR⁷Z—, —NR⁷C(O)Z—, —NR⁷C(O)NR⁷Z—, —OC(O)Z—, —NR⁷C(O)OZ—, and —OC(O) NR⁷Z—; and
Z is a bond or a divalent alkyl of 1-6 carbon atoms.

2. The compound of claim 1, wherein R¹ is 4-pyridinyl or 4-morpholinyl, optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, —OPO(OR⁷)₂, —YR⁸R¹⁰, —YR⁸NR⁷R⁷ and —YR¹⁰.

3. The compound of claim 2, wherein R² is an aryl ring or a bicyclic ring of formula 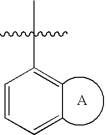
wherein 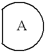
refers to a 5-7 membered heteroaryl ring comprising 1-3 heteroatoms selected from N, O and S, said ring optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, —YR⁸R¹⁰, —YR⁸NR⁷R⁷ and —YR¹⁰.

4. The compound of claim 2, wherein R²is a phenyl ring or an indazolyl ring, optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, —YR⁸R¹⁰, —YR⁸NR⁷R⁷ and —YR¹⁰.

5. The compound of claim 2, wherein R² is selected from halogen substituted phenyl, C₁-C₆ alkylsulfonamido substituted phenyl, carbamate substituted phenyl, C₁-C₆ alkoxy substituted phenylcarbamate, benzonitrile, hydroxyl substituted benzonitrile, C₁-C₆ alkoxy substituted benzonitrile, hydroxyphenyl, C₁-C₆ alkyl substituted hydroxyphenyl, halogen substituted hydroxyphenyl, C₁-C₆ alkoxyphenyl, halogen substituted C₁-C₆ alkoxyphenyl, hydroxypyridinyl, C₁-C₆ alkoxypyridinyl, amino phenyl, halogen substituted amino phenyl, hydroxyl substituted amino phenyl, formamide substituted phenyl, hydroxyl substituted phenylformamide, C₁-C₆ alkoxy substituted phenylformamide, C₁-C₆ alkoxy substituted amino phenyl, urea substituted phenyl, benzamido, C₁-C₆ alkyl substituted benzamido, halogen substituted benzamido, indazolyl, C₁-C₆ alkyl substituted indazolyl, halogen substituted indazolyl, halo C₁-C₆ alkyl substituted indazolyl, perfluoro C₁-C₆ alkyl substituted indazolyl, benzamidazolyl, halogen substituted benzamidazolyl, dihydro-pyrrolodinyl, substituted dihydro-pyrrolodinyl, dihydro-indolyl, substituted dihydro-indolyl and oxadiazolyl substituted phenyl.

6. The compound of claim 2, wherein R⁶ is a bridged, bicyclic heterocyclic ring selected from: 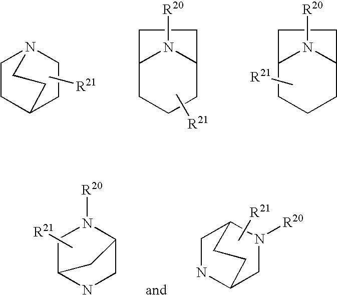
optionally substituted on nitrogen with R²⁰ and optionally substituted on one or more carbons of the bridged, bicyclic heterocyclic ring with R²¹, wherein
R²⁰ is selected from H, —C(O)OR⁷, —C(O)NR⁷R⁷, —C(O)R⁷, —S(O)ₘR⁷, alkyl of 1-6 carbon atoms, branched alkyl of 1-8 carbon atoms, cycloalkyl ring of 3-10 carbons, aryl ring, 5-7 membered heterocyclic ring and 5-10 membered heteroaryl ring, each heterocyclic ring or heteroaryl ring comprising 1-3 heteroatoms selected from N, O or S, each of the alkyl of 1-6 carbon atoms, branched alkyl of 1-8 carbon atoms, aryl ring, heterocyclic ring and heteroaryl ring optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)N R⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷ or —R⁸NR⁷C(O)NR⁷R⁷ and YR¹⁰; and
R²¹ is selected from H, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷ or —R⁸NR⁷C(O)NR⁷R⁷ and YR¹⁰.

7. The compound of claim 4, wherein R⁵ is R⁶.

8. The compound of claim 4, wherein R⁵ is X—W—R⁶, X is aryl or heteroaryl and W is a bond.

9. The compound of claim 4, wherein R⁵ is X—W—R⁶, X is aryl or heteroaryl and W is ZNR⁷ or NR⁷Z.

10. A compound of claim 4 wherein R⁵ is a bicyclic spiro heterocyclic ring comprising 1-3 heteroatoms selected from N, O and S, optionally substituted with one to four substituents selected from -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)N R⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, R⁸OC(O)NR⁷R⁷, —R⁸NR⁷C(O)R⁷, —R⁸NR⁷C(O)OR⁷, —R⁸NR⁷C(O)NR⁷R⁷ and YR¹⁰.

11. A compound selected from
Exemplary compounds of Formula A prepared by methods of the present invention include the following compounds: 3-(7-{6-[(1-azabicyclo[2.2.2]oct-4-ylmethyl)amino]pyridin-3-yl}-2-pyridin-4-ylpyrazolo[1,5-a]pyrimidin-3-yl)phenol, 3-(7-{6-[(3S)-1-azabicyclo[2.2.2]oct-3-ylamino]pyridin-3-yl}-2-pyridin-4-ylpyrazolo[1,5-a]pyrimidin-3-yl)phenol, 3-(7-{6-[(3R)-1-azabicyclo[2.2.2]oct-3-ylamino]pyridin-3-yl}-2-pyridin-4-ylpyrazolo[1,5-a]pyrimidin-3-yl)phenol, (3R)-N-{4-[3-(3-methoxyphenyl)-2-pyridin-4-ylpyrazolo[1,5-a]pyrimidin-7-yl]pyridin-2-yl}quinuclidin-3-amine, (3R)-N-{5-[3-(4-chloro-3-methoxyphenyl )-2-pyridin-4-ylpyrazolo[1,5-a]pyrimidin-7-yl]pyridin-2-yl}-quinuclidin-3-amine, 3-{7-[(1-azabicyclo[2.2.2]oct-4-ylmethyl)amino]-2-pyridin-4-ylpyrazolo[1,5-a]pyrimidin-3-yl}phenol, ethyl 3-[3-(3-methoxyphenyl)-2-pyridin-4-ylpyrazolo[1,5-a]pyrimidin-7-yl]-8-azabicyclo[3.2.1]octane-8-carboxylate, ethyl 3-[3-(3-hydroxyphenyl)-2-pyridin-4-ylpyrazolo[1,5-a]pyrimidin-7-yl]-8-azabicyclo[3.2.1]-octane-8-carboxylate, 3-[7-(8-azabicyclo[3.2.1]oct-3-yl)-2-pyridin-4-ylpyrazolo[1,5-a]pyrimidin-3-yl]phenol, ethyl 3-[3-(4-chloro-3-methoxyphenyl)-2-pyridin-4-ylpyrazolo[1,5-a]pyrimidin-7-yl]-8-azabicyclo[3.2.1]octane-8-carboxylate, ethyl 3-[3-(4-chloro-3-hydroxyphenyl)-2-pyridin-4-ylpyrazolo[1,5-a]pyrimidin-7-yl]-8-azabicyclo[3.2.1]octane-8-carboxylate, 5-[7-(8-azabicyclo[3.2.1]oct-3-yl)-2-pyridin-4-ylpyrazolo[1,5-a]pyrimidin-3-yl]-2-chlorophenol, 5-[7-(8-acetyl-8-azabicyclo[3.2.1]oct-3-yl)-2-pyridin-4-ylpyrazolo[1,5-a]pyrimidin-3-yl]-2-chlorophenol, 2-chloro-5-{7-[8-(methylsulfonyl)-8-azabicyclo[3.2.1]oct-3-yl]-2-pyridin-4-ylpyrazolo[1,5-a]pyrimidin-3-yl}phenol, 5-[7-(8-acetyl-8-azabicyclo[3.2.1]oct-3-yl )-2-pyridin-4-ylpyrazolo[1,5-a]pyrimidin-3-yl]-2-chlorophenyl acetate, 7-(8-azabicyclo[3.2.1]oct-3-yl)-3-(4-chloro-3-methoxyphenyl)-2-pyridin-4-ylpyrazolo[1,5-a]pyrimidine, 3-(4-chloro-3-methoxyphenyl)-7-(8-ethyl-8-azabicyclo[3.2.1]oct-3-yl)-2-pyridin-4-ylpyrazolo[1,5-a]pyrimidine, 2-chloro-5-[7-(8-ethyl-8-azabicyclo[3.2.1]oct-3-yl)-2-pyridin-4-ylpyrazolo[1,5-a]pyrimidin-3-yl]phenol, 7-(8-azabicyclo[3.2.1]oct-3-yl)-3-(3-methoxyphenyl)-2-pyridin-4-ylpyrazolo[1,5-a]pyrimidine, 7-(8-ethyl-8-azabicyclo[3.2.1]oct-3-yl)-3-(2-methoxypyridin-4-yl)-2-pyridin-4-ylpyrazolo[1,5-a]pyrimidine, 4-[7-(8-ethyl-8-azabicyclo[3.2.1]oct-3-yl)-2-pyridin-4-ylpyrazolo[1,5-a]pyrimidin-3-yl]pyridin-2-ol, 4-[7-(8-ethyl-8-azabicyclo[3.2.1]oct-3-yl)-2-pyridin-4-ylpyrazolo[1,5-a]pyrimidin-3-yl]aniline, 1-{4-[7-(8-ethyl-8-azabicyclo[3.2.1]oct-3-yl)-2-pyridin-4-ylpyrazolo[1,5-a]pyrimidin-3-yl]phenyl}urea, 3-(3-methoxyphenyl)-7-[4-(8-methyl-3,8-diazabicyclo[3.2.1]oct-3-yl)phenyl]-2-pyridin-4-ylpyrazolo[1,5-a]pyrimidine, 3-{7-[4-(8-methyl-3,8-diazabicyclo[3.2.1]oct-3-yl)phenyl]-2-pyridin-4-ylpyrazolo[1,5-a]pyrimidin-3-yl}phenol, 3-(4-chloro-3-methoxyphenyl)-7-[4-(8-methyl-3,8-diazabicyclo[3.2.1]oct-3-yl)phenyl]-2-pyridin-4-ylpyrazolo[1,5-a]pyrimidine, 2-chloro-5-{7-[4-(8-methyl-3,8-diazabicyclo[3.2.1]oct-3-yl)phenyl]-2-pyridin-4-ylpyrazolo[1,5-a]pyrimidin-3-yl}phenol, 7-{4-[(1S,4S)-2,5-diazabicyclo[2.2.1]hept-2-yl]phenyl}-3-(3-methoxyphenyl)-2-pyridin-4-ylpyrazolo[1,5-a]pyrimidine, 3-(7-{4-[(1S,4S)-2,5-diazabicyclo[2.2.1]hept-2-yl]phenyl}-2-pyridin-4-ylpyrazolo[1,5-a]pyrimidin-3-yl)phenol, 3-(7-{4-[(1S,4S)-5-methyl-2, 5-diazabicyclo[2.2.1]hept-2-yl]phenyl}-2-pyridin-4-ylpyrazolo[1,5-a]pyrimidin-3-yl)phenol, 3-(4-chloro-3-methoxyphenyl)-7-{4-[(1S,4S)-2,5-diazabicyclo[2.2.1]hept-2-yl]phenyl}-2-pyridin-4-ylpyrazolo[1,5-a]pyrimidine, 7-{4-[(1S,4S)-2, 5-diazabicyclo[2.2.1]hept-2-yl]phenyl}-3-(4-fluoro-3-methoxyphenyl)-2-pyridin-4-ylpyrazolo[1,5-a]pyrimidine, 5-(7-{4-[(1S,4S)-2,5-diazabicyclo[2.2.1]hept-2-yl]phenyl}-2-pyridin-4-ylpyrazolo[1,5-a]pyrimidin-3-yl)-2-fluorophenol, 3-(1H-indazol-4-yl)-7-{6-[(1S,4S)-5-methyl-2,5-diazabicyclo[2.2.1]hept-2-yl]pyridin-3-yl}-2-pyridin-4-ylpyrazolo[1,5-a]pyrimidine, 3-(1H-indazol-4-yl)-7-{4-[(1S,4S)-5-methyl-2,5-diazabicyclo[2.2.1]hept-2-yl]phenyl}-2-pyridin-4-ylpyrazolo[1,5-a]pyrimidine, 3-(3-methoxyphenyl)-7-{4-[(1S,4S)-5-methyl-2,5-diazabicyclo[2.2.1]-hept-2-yl]phenyl}-2-pyridin-4-ylpyrazolo[1,5-a]pyrimidine, 3-(4-fluoro-3-methoxyphenyl)-7-{4-[(1S,4S)-5-methyl-2,5-diazabicyclo[2.2.1]hept-2-yl]phenyl}-2-pyridin-4-ylpyrazolo[1,5-a]pyrimidine, 5-(7-{4-[(1S,4S)-5-ethyl-2, 5-diazabicyclo[2.2.1]-hept-2-yl]phenyl}-2-pyridin-4-ylpyrazolo[1,5-a]pyrimidin-3-yl)-2-fluorophenol, 5-(7-{4-[(1S,4S)-5-acetyl-2,5-diazabicyclo[2.2.1]hept-2-yl]phenyl}-2-pyridin-4-ylpyrazolo[1,5-a]pyrimidin-3-yl )-2-fluorophenol, 7-{6-[(1S,4S)-5-methyl-2,5-diazabicyclo[2.2.1]hept-2-yl]pyridin-3-yl}-3-(7-methyl-1H-indazol-4-yl)-2-pyridin-4-ylpyrazolo[1,5-a]pyrimidine, 3-(7-chloro-1H-indazol-4-yl )-7-{6-[(1S,4S)-5-methyl-2,5-diazabicyclo[2.2.1]hept-2-yl]pyridin-3-yl}-2-pyridin-4-ylpyrazolo[1,5-a]pyrimidine, 3-(7-fluoro-1H-indazol-4-yl)-7-{6-[(1S,4S)-5-methyl-2,5-diazabicyclo[2.2.1]hept-2-yl]pyridin-3-yl}-2-pyridin-4-ylpyrazolo[1,5-a]pyrimidine, 3-(7-{6-[(1S,4S)-5-methyl-2,5-diazabicyclo[2.2.1]hept-2-yl]pyridin-3-yl}-2-pyridin-4-ylpyrazolo[1,5-a]pyrimidin-3-yl)benzamide, 7-{4-[(1S,4S)-2,5-diazabicyclo[2.2.1]hept-2-yl]-2-methylphenyl}-3-(1H-indazol-4-yl)-2-pyridin-4-ylpyrazolo[1,5-a]pyrimidine, 3-(1H-indazol-4-yl)-7-{2-methyl-4-[(1S,4S)-5-methyl-2, 5-diazabicyclo[2.2.1]hept-2-yl]phenyl}-2-pyridin-4-ylpyrazolo[1,5-a]pyrimidine, 7-{2-[(1S,4S)-2,5-diazabicyclo[2.2.1]hept-2-yl]-4-fluorophenyl}-3-(1H-indazol-4-yl)-2-pyridin-4-ylpyrazolo[1,5-a]pyrimidine, 7-{4-fluoro-2-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, tert-butyl(1S,4S)-5-{2-[3-(4-chloro-3-methoxyphenyl)-2-pyridin-4-ylpyrazolo[1,5-a]pyrimidin-7-yl]-5-fluorophenyl}-2,5-diazabicyclo[2.2.1]heptane-2-carboxylate, 3-(4-chloro-3-methoxyphenyl)-7-{2-[(1S,4S)-2,5-diazabicyclo[2.2.1]hept-2-yl]-4-fluorophenyl}-2-pyridin-4-ylpyrazolo[1,5-a]pyrimidine, 3-(4-chloro-3-methoxyphenyl)-7-{4-fluoro-2-[(1S,4S)-5-methyl-2,5-diazabicyclo[2.2.1]hept-2-yl]phenyl}-2-pyridin-4-ylpyrazolo[1,5-a]pyrimidine, 7-{4-[(1S,4S)-2,5-diazabicyclo-[2.2.1]hept-2-yl]phenyl}-3-(1H-indazol-4-yl)-2-pyridin-4-ylpyrazolo[1,5-a]pyrimidine, 7-{4-[(1S,4S)-2, 5-diazabicyclo[2.2.1]hept-2-yl]-2-fluorophenyl}-3-(1H-indazol-4-yl)-2-pyridin-4-ylpyrazolo[1,5-a]pyrimidine, 7-{2-fluoro-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, 2-fluoro-5-(7-{4-[(1S,4S)-5-methyl-2,5-diazabicyclo[2.2.1]hept-2-yl]phenyl}-2-pyridin-4-ylpyrazolo[1,5-a]pyrimidin-3-yl)phenol, 3-(4-fluoro-3-methoxyphenyl)-7-{2-methyl-4-[(1S,4S)-5-methyl-2,5-diazabicyclo[2.2.1]hept-2-yl]phenyl}-2-pyridin-4-ylpyrazolo[1,5-a]pyrimidine, 2-fluoro-5-(7-{2-methyl-4-[(1S,4S)-5-methyl-2,5-diazabicyclo[2.2.1]hept-2-yl]phenyl}-2-pyridin-4-ylpyrazolo[1,5-a]pyrimidin-3-yl)phenol, 3-(7-{4-[(1S,4S)-2,5-diazabicyclo[2.2.1]hept-2-yl]-2-methylphenyl}-2-pyridin-4-ylpyrazolo[1,5-a]pyrimidin-3-yl)phenol, 3-(7-{2-methyl-4-[(1S,4S)-5-methyl-2,5-diazabicyclo[2.2.1]hept-2-yl]phenyl}-2-pyridin-4-ylpyrazolo[1,5-a]pyrimidin-3-yl)phenol, 3-(7-{2-chloro-4-[(1S,4S)-2,5-diazabicyclo[2.2.1]hept-2-yl]phenyl}-2-pyridin-4-ylpyrazolo[1,5-a]pyrimidin-3-yl)phenol, 3-(7-{2-chloro-4-[(1S,4S)-5-methyl-2,5-diazabicyclo[2.2.1]hept-2-yl]phenyl}-2-pyridin-4-ylpyrazolo[1,5-a]pyrimidin-3-yl)phenol, 7-{2-chloro-4-[(1S,4S)-2,5-diazabicyclo[2.2.1]hept-2-yl]phenyl}-3-(1H-indazol-4-yl)-2-pyridin-4-yl pyrazolo[1,5-a]pyrimidine, 7-{2-chloro-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, ethyl 3-[3-(3-hydroxy-4-methylphenyl)-2-pyridin-4-ylpyrazolo[1,5-a]pyrimidin-7-yl]-8-azabicyclo[3.2.1]octane-8-carboxylate, 5-[7-(8-ethyl-8-azabicyclo[3.2.1]oct-3-yl)-2-pyridin-4-ylpyrazolo[1,5-a]pyrimidin-3-yl]-2-fluorophenol, ethyl 3-[3-(2,3-difluorophenyl)-2-pyridin-4-ylpyrazolo[1,5-a]pyrimidin-7-yl]-8-azabicyclo[3.2.1]octane-8-carboxylate, ethyl 3-(3-{3-[(methylsulfonyl)amino]-phenyl}-2-pyridin-4-ylpyrazolo[1,5-a]pyrimidin-7-yl)-8-azabicyclo[3.2.1]octane-8-carboxylate, methyl {4-[7-(8-ethyl-8-azabicyclo[3.2.1]oct-3-yl)-2-pyridin-4-ylpyrazolo[1,5-a]pyrimidin-3-yl]phenyl}carbamate, 4-[7-(8-ethyl-8-azabicyclo[3.2.1]-oct-3-yl)-2-pyridin-4-ylpyrazolo[1,5-a]pyrimidin-3-yl]-2-hydroxybenzonitrile, tert-butyl {4-[7-(8-ethyl-8-azabicyclo[3.2.1]oct-3-yl)-2-pyridin-4-ylpyrazolo[1,5-a]pyrimidin-3-yl]-2-methoxyphenyl}-carbamate, 4-[7-(8-ethyl-8-azabicyclo[3.2.1]oct-3-yl)-2-pyridin-4-ylpyrazolo[1,5-a]pyrimidin-3-yl]-2-methoxyaniline, 2-amino-5-[7-(8-ethyl-8-azabicyclo[3.2.1]oct-3-yl)-2-pyridin-4-ylpyrazolo[1,5-a]pyrimidin-3-yl]phenol, N-{4-[7-(8-ethyl-8-azabicyclo[3.2.1]oct-3-yl)-2-pyridin-4-ylpyrazolo[1,5-a]pyrimidin-3-yl]-2-methoxyphenyl}formamide, ethyl 3-[3-(1H-indazol-1H-indazol-4-yl)-2-pyridin-4-ylpyrazolo[1,5-a]pyrimidin-7-yl]-8-azabicyclo[3.2.1]octane-8-carboxylate, 7-(8-azabicyclo[3.2.1]oct-3-yl)-3-(1H-indazol-4-yl)-2-pyridin-4-ylpyrazolo-[1,5-a]pyrimidine, ethyl 3-[3-(7-chloro-1H-indazol-4-yl)-2-pyridin-4-yl pyrazolo[1,5-a]pyrimidin-7-yl]-8-azabicyclo[3.2.1]octane-8-carboxylate, ethyl 3-{2-pyridin-4-yl-3-[7-(trifluoromethyl)-1H-indazol-4-yl]pyrazolo[1,5-a]pyrimidin-7-yl}-8-azabicyclo[3.2.1]-octane-8-carboxylate, 7-(8-ethyl-8-azabicyclo[3.2.1]oct-3-yl)-3-(7-methyl-1H-indazol-4-yl)-2-pyridin-4-ylpyrazolo[1,5-a]pyrimidine, ethyl 3-[3-(7-methyl-1H-indazol-4-yl)-2-pyridin-4-ylpyrazolo[1,5-a]pyrimidin-7-yl]-8-azabicyclo[3.2.1]octane-8-carboxylate, 3-(7-chloro-1H-indazol-4-yl)-7-(8-ethyl-8-azabicyclo[3.2.1]oct-3-yl)-2-pyridin-4-yl pyrazolo[1,5-a]pyrimidine, 7-(8-ethyl-8-azabicyclo[3.2.1]oct-3-yl)-2-pyridin-4-yl-3-[7-(trifluoromethyl)-1H-indazol-4-yl]pyrazolo[1,5-a]pyrimidine, ethyl 3-[3-(7-fluoro-1H-indazol-4-yl)-2-pyridin-4-ylpyrazolo[1,5-a]pyrimidin-7-yl]-8-azabicyclo[3.2.1]octane-8-carboxylate, 3-(7-chloro-6-fluoro-1H-indazol-4-yl)-7-(8-ethyl-8-azabicyclo[3.2.1]oct-3-yl)-2-pyridin-4-ylpyrazolo[1,5-a]pyrimidine, ethyl 3-[3-(2-oxo-2,3-dihydro-1H-benzimidazol-4-yl)-2-pyridin-4-ylpyrazolo[1,5-a]pyrimidin-7-yl]-8-azabicyclo[3.2.1]-octane-8-carboxylate, ethyl 3-[3-(1H-indol-4-yl)-2-pyridin-4-yl pyrazolo[1,5-a]pyrimidin-7-yl]-8-azabicyclo[3.2.1]octane-8-carboxylate, ethyl 3-[3-(1H-indol-6-yl)-2-pyridin-4-ylpyrazolo[1,5-a]pyrimidin-7-yl]-8-azabicyclo[3.2.1]octane-8-carboxylate, ethyl 3-[3-(2-oxo-2,3-dihydro-1H-pyrrolo[2,3-b]pyridin-4-yl)-2-pyridin-4-ylpyrazolo[1,5-a]pyrimidin-7-yl]-8-azabicyclo[3.2.1]octane-8-carboxylate, 7-(8-ethyl-8-azabicyclo-[3.2.1]oct-3-yl)-3-(1H-indol-6-yl)-2-pyridin-4-ylpyrazolo[1,5-a]pyrimidine, ethyl 3-[3-(2-oxo-2,3-dihydro-1H-indol-6-yl)-2-pyridin-4-yl pyrazolo[1,5-a]pyrimidin-7-yl]-8-azabicyclo[3.2.1]octane-8-carboxylate, 2-chloro-5-[7-(2,2-dimethyl-1,3-dioxolan-4-yl)-2-pyridin-4-ylpyrazolo[1,5-a]pyrimidin-3-yl]phenol, 7-(8-Ethyl-8-azabicyclo[3.2.1]-octan-3-yl)-3-(1H-indazol-4-yl)-2-(pyridin-4-yl)pyrazolo[1,5-a]pyrimidine, Ethyl 3-(3-(3-(1, 3,4-oxadiazol-2-yl)phenyl)-2-(pyridin-4-yl)pyrazolo[1,5-a]pyrimidin-7-yl)-8-azabicyclo[3.2.1]octane-8-carboxylate, tert-Butyl(1S,4S)-5-{3-fluoro-4-[3-(1H-indazol-4-yl)-2-pyridin-4-ylpyrazolo[1,5-a]pyrimidin-7-yl]phenyl}-2,5-diazabicyclo-[2.2.1]heptane-2-carboxylate, 7-{4-[(1S,4S)-2,5-diazabicyclo[2.2.1]hept-2-fluorophenyl}-3-(1H-indazol-4-yl)-2-pyridin-4-yl pyrazolo[1,5-a]pyrimidine, 7-{2-Fluoro-4-[(1S,4S)-5-methyl-2,5-diazabicyclo[2.2.1]hept-2-yl]phenyl}-3-(1H-indazol-4-yl)-2-pyridin-4-yl pyrazolo[1,5-a]pyrimidine, 7-{2-fluoro-4-[(1S,4S)-5-methyl-5-oxido-2,5-diazabicyclo[2.2.1]hept-2-yl]phenyl}-3-(1H-indazol-4-yl)-2-pyridin-4-ylpyrazolo-[1,5-a]pyrimidine, (1S,4S)-5-{3-Chloro-4-[3-(1H-indazol-4-yl)-2-pyridin-4-yl-pyrazolo[1,5-a]pyrimidin-7-yl]-phenyl}-2,5-diaza-bicyclo[2.2.1]heptane-2-carboxylic acid tert-butyl ester, 7-[2-Chloro-4-((1S,4S)-2,5-diaza-bicyclo[2.2.1]hept-2-yl)-phenyl]-3-(1H-indazol-4-yl)-2-pyridin-4-yl-pyrazolo[1,5-a]pyrimidine bis-hydrochloride salt, 7-[2-Chloro-4-((1S,4S)-5-methyl-2,5-diaza-bicyclo[2.2.1]hept-2-yl)-phenyl]-3-(1H-indazol-4-yl)-2-pyridin-4-yl-pyrazolo[1,5-a]pyrimidine, tert-butyl (1S,4S)-5-{3,5-difluoro-4-[3-(1H-indazol-4-yl)-2-pyridin-4-ylpyrazolo[1,5-a]pyrimidin-7-yl]phenyl}-2,5-diazabicyclo[2.2.1]heptane-2-carboxylate, 7-(4-((1S,4S)-2,5-diazabicyclo[2.2.1]-heptan-2-yl)-2,6-difluorophenyl)-3-(1H-indazol-4-yl)-2-(pyridin-4-yl)pyrazolo[1,5-a]pyrimidine, 7-{2,6-difluoro-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, tert-Butyl(1S,4S)-5-{4-[3-(7-fluoro-1H-indazol-4-yl)-2-pyridin-4-ylpyrazolo[1,5-a]pyrimidin-7-yl]phenyl}-2,5-diazabicyclo[2.2.1]heptane-2-carboxylate, 7-[4-(2,5-Diaza-bicyclo[2.2.1]hept-2-yl)-phenyl]-3-(7-fluoro-1H-indazol-4-yl)-2-pyridin-4-yl-pyrazolo[1,5-a]pyrimidine, 3-(7-fluoro-1H-indazol-4-yl)-7-{4-[(1S,4S)-5-methyl-2,5-diazabicyclo[2.2.1]hept-2-yl]phenyl}-2-pyridin-4-ylpyrazolo[1,5-a]pyrimidine hydrochloride, tert-Butyl(1S,4S)-5-{3-fluoro-4-[3-(7-fluoro-1H-indazol-4-yl)-2-pyridin-4-ylpyrazolo[1,5-a]pyrimidin-7-yl]phenyl}-2,5-diazabicyclo[2.2.1]heptane-2-carboxylate, 7-{4-[(1S,4S)-2,5-diazabicyclo[2.2.1]hept-2-yl]-2-fluorophenyl}-3-(7-fluoro-1H-indazol-4-yl)-2-pyridin-4-ylpyrazolo[1,5-a]pyrimidine, 3(7-fluoro-1H-indazol-4-yl)-7-{2-fluoro-4-[(1S,4S)-5-methyl-2,5-diazabicyclo[2.2.1]hept-2-yl]phenyl}-2-pyridin-4-ylpyrazolo[1,5-a]pyrimidine, tert-Butyl(1S,4S)-5-{3,5-difluoro-4-[3-(7-fluoro-1H-indazol-4-yl)-2-pyridin-4-ylpyrazolo[1,5-a]pyrimidin-7-yl]phenyl}-2,5-diazabicyclo[2.2.1]heptane-2-carboxylate, 7-{4-[(1S,4S)-2,5-Diazabicyclo[2.2.1]hept-2-yl]-2,6-difluorophenyl}-3-(7-fluoro-1H-indazol-4-yl)-2-pyridin-4-ylpyrazolo[1,5-a]pyrimidine, 7-{2,6-Difluoro-4-[(1S,4S)-5-methyl-2,5-diazabicyclo[2.2.1]hept-2-yl]phenyl}-3-(7-fluoro-1H-indazol-4-yl)-2-pyridin-4-ylpyrazolo[1,5-a]pyrimidine, ethyl 3-[3-(1H-indazol-4-yl)-2-pyridin-4-ylpyrazolo[1,5-a]pyrimidin-5-yl]-8-azabicyclo[3.2.1]-octane-8-carboxylate, 2-{3-[3-(1H-indazol-4-yl)-2-pyridin-4-ylpyrazolo[1,5-a]pyrimidin-7-yl]-8-azabicyclo[3.2.1]oct-8-yl}ethanol, 3-(1H-indazol-4-yl)-7-(8-isopropyl-8-azabicyclo[3.2.1]oct-3-yl)-2-pyridin-4-ylpyrazolo[1,5-a]pyrimidine, 3-(1H-indazol-4-yl)-7-[8-(methylsulfonyl)-8-azabicyclo[3.2.1]oct-3-yl]-2-pyridin-4-ylpyrazolo[1,5-a]pyrimidine, 3-[3-(1H-indazol-4-yl)-2-pyridin-4-ylpyrazolo[1,5-a]pyrimidin-7-yl]-8-azabicyclo[3.2.1]octane-8-carboxamide, 2-{3-[3-(1H-indazol-4-yl)-2-pyridin-4-ylpyrazolo[1,5-a]pyrimidin-7-yl]-8-azabicyclo[3.2.1]oct-8-yl}-N,N-dimethyl-2-oxoethanamine, {3-[3-(1H-indazol-4-yl)-2-pyridin-4-ylpyrazolo[1,5-a]pyrimidin-7-yl]-8-azabicyclo[3.2.1]oct-8-yl}acetonitrile, N-ethyl-3-[3-(1H-indazol-4-yl)-2-pyridin-4-ylpyrazolo[1,5-a]pyrimidin-7-yl]-8-azabicyclo[3.2.1]octane-8-carboxamide, 7-(8-acetyl-8-azabicyclo[3.2.1]oct-3-yl)-3-(1H-indazol-4-yl)-2-pyridin-4-yl pyrazolo[1,5-a]pyrimidine, 3-[3-(1H-indazol-4-yl)-2-pyridin-4-ylpyrazolo[1,5-a]pyrimidin-7-yl]-N,N-dimethyl-8-azabicyclo[3.2.1]octane-8-carboxamide, tert-butyl(1S,4S)-5-{[3-(4-chloro-3-methoxyphenyl)-2-pyridin-4-yl pyrazolo[1,5-a]pyrimidin-7-yl]methyl}-2,5-diazabicyclo[2.2.1]heptane-2-carboxylate, tert-butyl(1S,4S)-5-{4-[3-(7-fluoro-1H-indazol-4-yl)-2-pyridin-4-ylpyrazolo[1,5-a]pyrimidin-5-yl]phenyl}-2,5-diazabicyclo-[2.2.1]heptane-2-carboxylate, tert-butyl(1S,4S)-5-{3-[3-(1H-indazol-4-yl)-2-pyridin-4-ylpyrazolo[1,5-a]pyrimidin-7-yl]phenyl}-2,5-diazabicyclo[2.2.1]heptane-2-carboxylate, 7-{3-[(1S,4S)-2,5-diazabicyclo[2.2.1]hept-2-yl]phenyl}-3-(1H-indazol-4-yl)-2-pyridin-4-ylpyrazolo[1,5-a]pyrimidine, tert-butyl(1S,4S)-5-{4-[3-(7-chloro-1H-indazol-4-yl)-2-pyridin-4-ylpyrazolo[1,5-a]pyrimidin-7-yl]phenyl}-2,5-diazabicyclo[2.2.1]heptane-2-carboxylate, 3-(1H-indazol-4-yl)-7-{3-[(1S,4S)-5-methyl-2,5-diazabicyclo[2.2.1]hept-2-yl]phenyl}-2-pyridin-4-ylpyrazolo[1,5-a]pyrimidine, 3-(7-chloro-1H-indazol-4-yl)-7-{4-[(1S,4S)-2,5-diazabicyclo[2.2.1]hept-2-yl]phenyl}-2-pyridin-4-ylpyrazolo[1,5-a]pyrimidine, tert-butyl(2S)-2-({3-[3-(1H-indazol-4-yl)-2-pyridin-4-ylpyrazolo[1,5-a]pyrimidin-7-yl]-8-azabicyclo[3.2.1]oct-8-yl}carbonyl)pyrrolidine-1-carboxylate, 3-(1H-indazol-4-yl)-7-(8-L-prolyl-8-azabicyclo[3.2.1]oct-3-yl)-2-pyridin-4-ylpyrazolo[1,5-a]pyrimidine, 1-{3-[3-(1H-indazol-4-yl)-2-pyridin-4-ylpyrazolo[1,5-a]pyrimidin-7-yl]-8-azabicyclo[3.2.1]oct-8-yl}propan-2-one, ethyl 3-[3-(7-fluoro-1H-indazol-4-yl)-2-pyridin-4-ylpyrazolo[1,5-a]pyrimidin-5-yl]-8-azabicyclo[3.2.1]octane-8-carboxylate, 7-{4-[(1S,4S)-2,5-diazabicyclo[2.2.1]hept-2-yl]-2-fluorophenyl}-3-(3-methoxyphenyl)-2-pyridin-4-ylpyrazolo[1,5-a]pyrimidine, 3-(7-{4-[(1S,4S)-2,5-diazabicyclo[2.2.1]hept-2-yl]-2-fluorophenyl}-2-pyridin-4-ylpyrazolo[1,5-a]pyrimidin-3-yl)phenol, 3-(7-{2-fluoro-4-[(1S,4S)-5-methyl-2,5-diazabicyclo[2.2.1]hept-2-yl]phenyl}-2-pyridin-4-ylpyrazolo[1,5-a]pyrimidin-3-yl)phenol, 7-{4-[(1S,4S)-2,5-diazabicyclo[2.2.1]hept-2-yl]-2-methyl phenyl}-3-(7-fluoro-1H-indazol-4-yl)-2-pyridin-4-ylpyrazolo[1,5-a]pyrimidine, 3-(7-fluoro-1H-indazol-4-yl)-7-{2-methyl-4-[(1S,4S,)-5-methyl-2,5-diazabicyclo-[2.2.1]hept-2-yl]phenyl}-2-pyridin-4-ylpyrazolo[1,5-a]pyrimidine, 7-(8-azabicyclo[3.2.1]oct-3-yl)-3-(7-fluoro-1H-indazol-4-yl)-2-pyridin-4-yl pyrazolo[1,5-a]pyrimidine, {3-[3-(7-fluoro-1H-indazol-4-yl)-2-pyridin-4-yl pyrazolo[1,5-a]pyrimidin-7-yl]-8-azabicyclo[3.2.1]oct-8-yl}acetonitrile, 3-(7-chloro-1H-indazol-4-yl)-5-{4-[(1S,4S)-2,5-diazabicyclo[2.2.1]hept-2-yl]phenyl}-2-pyridin-4-ylpyrazolo[1,5-a]pyrimidine, 3-(1H-indazol-4-yl)-7-[6-(8-methyl-3,8-diazabicyclo[3.2.1]oct-3-yl)pyridin-3-yl]-2-pyridin-4-ylpyrazolo[1,5-a]pyrimidine, 3-(7-chloro-1H-indazol-4-yl)-7-{4-[(1S,4S)-5-methyl-2,5-diazabicyclo[2.2.1]hept-2-yl]phenyl}-2-pyridin-4-ylpyrazolo[1,5-a]pyrimidine, 7-(1,4-dioxaspiro[4.5]dec-8-yl)-3-(1H-indazol-4-yl)-2-pyridin-4-ylpyrazolo[1,5-a]pyrimidine, 3-(1H-indazol-4-yl)-7-{4-[(1S,4S)-5-methyl-2,5-diazabicyclo[2.2.2]oct-2-yl]phenyl}-2-pyridin-4-ylpyrazolo[1,5-a]pyrimidine, 7-{4-[(1S,4S)-2,5-diazabicyclo-[2.2.2]oct-2-yl]phenyl}-3-(1H-indazol-4-yl)-2-pyridin-4-ylpyrazolo[1,5-a]pyrimidine, 7-{2-[(1S,4S)-2,5-diazabicyclo[2.2.1]hept-2-yl]-4,6-difluorophenyl}-3-(1H-indazol-4-yl)-2-pyridin-4-ylpyrazolo[1,5-a]pyrimidine, 5-[(1S,4S)-2,5-diazabicyclo[2.2.1]hept-2-yl]-2-[3-(1H-indazol-4-yl)-2-pyridin-4-ylpyrazolo[1,5-a]pyrimidin-7-yl]-N,N-dimethylaniline, 7-{2,4-difluoro-6-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, 2-[3-(1H-indazol-4-yl)-2-pyridin-4-ylpyrazolo[1,5-a]pyrimidin-7-yl]-N,N-dimethyl-5-[(1S,4S)-5-methyl-2,5-diazabicyclo[2.2.1]hept-2-yl]aniline, 7-{cis-4-[1S,4S)-2,5-diazabicyclo[2.2.1]hept-2-yl]cyclohexyl}-3-(1H-indazol-4-yl)-2-pyridin-4-ylpyrazolo[1,5-a]pyrimidine, 3-(1H-indazol-4-yl)-7-[cis-4-(3-oxa-8-azabicyclo[3.2.1]oct-8-yl)cyclohexyl]-2-pyridin-4-ylpyrazolo[1,5-a]pyrimidine, 3-(1H-indazol-4-yl)-7-[trans-4-(3-oxa-8-azabicyclo[3.2.1]-oct-8-yl)cyclohexyl]-2-pyridin-4-ylpyrazolo[1,5-a]pyrimidine, 3-(1H-indazol-4-yl)-7-[cis-4-(8-oxa-3-azabicyclo[3.2.1]oct-3-yl)cyclohexyl]-2-pyridin-4-ylpyrazolo[1,5-a]pyrimidine, 3-(1H-indazol-4-yl)-7-[trans-4-(8-oxa-3-azabicyclo[3.2.1]oct-3-yl)cyclohexyl]-2-pyridin-4-ylpyrazolo[1,5-a]pyrimidine, 3-(1H-indazol-4-yl)-7-{cis-4-[(1S,4S)-2-oxa-5-azabicyclo[2.2.1]hept-5-yl]cyclohexyl}-2-pyridin-4-ylpyrazolo[1,5-a]pyrimidine, 3-(1H-indazol-4-yl)-7-{trans-4-[(1S,4S)-2-oxa-5-azabicyclo[2.2.1]hept-5-yl]cyclohexyl}-2-pyridin-4-ylpyrazolo[1,5-a]pyrimidine, 7-{trans-4-[(1S,4S)-2,5-diazabicyclo[2.2.1]hept-2-yl]cyclohexyl}-3-(1H-indazol-4-yl)-2-pyridin-4-yl pyrazolo[1,5-a]pyrimidine, 7-{4-[(1S,4S)-2,5-diazabicyclo[2.2.1]hept-2-yl]-3-(trifluoromethyl)phenyl}-3-(1H-indazol-4-yl)-2-pyridin-4-ylpyrazolo[1,5-a]pyrimidine, 7-{4-[(1S,4S)-2,5-diazabicyclo[2.2.1]hept-2-yl]-1-naphthyl}-3-(1H-indazol-4-yl)-2-pyridin-4-ylpyrazolo[1,5-a]pyrimidine, 3-(1H-indazol-4-yl)-7-{4-[(1S,4S)-5-methyl-2,5-diazabicyclo[2.2.1]hept-2-yl]-3-(trifluoromethyl)phenyl}-2-pyridin-4-ylpyrazolo[1,5-a]pyrimidine, 3-(1H-indazol-4-yl)-7-{4-[(1S,4S)-5-methyl-2,5-diazabicyclo[2.2.1]hept-2-yl]-1-naphthyl}-2-pyridin-4-yl pyrazolo[1,5-a]pyrimidine, 7-{4-[(1S,4S)-2,5-diazabicyclo[2.2.1]hept-2-yl]-3,5-difluorophenyl}-3-(1H-indazol-4-yl)-2-pyridin-4-ylpyrazolo[1,5-a]pyrimidine, 7-{4-[(1S,4S)-2,5-diazabicyclo[2.2.1]hept-2-yl]-2,3-difluorophenyl}-3-(1H-indazol-4-yl)-2-pyridin-4-ylpyrazolo[1,5-a]pyrimidine, 7-{4-[(1S,4S)-2,5-diazabicyclo[2.2.1]hept-2-yl]-2,5-difluorophenyl}-3-(1H-indazol-4-yl)-2-pyridin-4-ylpyrazolo[1,5-a]pyrimidine, 7-{3, 5-difluoro-4-[(1S,4S)-5-methyl-2,5-diazabicyclo[2.2.1]hept-2-yl]phenyl}-3-(1H-indazol-4-yl)-2-pyridin-4-yl pyrazolo[1,5-a]pyrimidine, 7-{2,3-difluoro-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, 7-{2,5-difluoro-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, 7-{4-[(1S,4S)-5-ethyl-2,5-diazabicyclo[2.2.1]-hept-2-yl]-2,6-difluorophenyl}-3-(1H-indazol-4-yl)-2-pyridin-4-ylpyrazolo[1,5-a]pyrimidine, 7-{2,6-difluoro-4-[(1S,4S)-5-isobutyl-2,5-diazabicyclo[2.2.1]hept-2-yl]phenyl}-3-(1H-indazol-4-yl)-2-pyridin-4-yl pyrazolo[1,5-a]pyrimidine, 7-{2,6-difluoro-4-[(1S,4S)-5-isopropyl-2,5-diazabicyclo[2.2.1]hept-2-yl]phenyl}-3-(1H-indazol-4-yl)-2-pyridin-4-ylpyrazolo[1,5-a]pyrimidine, 7-{4-[(1S,4S)-5-cyclobutyl-2,5-diazabicyclo[2.2.1]hept-2-yl]-2,6-difluorophenyl}-3-(1H-indazol-4-yl)-2-pyridin-4-ylpyrazolo[1,5-a]pyrimidine, 7-(1,4-dioxaspiro[4.5]dec-8-yl)-3-(7-fluoro-1H-indazol-4-yl)-2-pyridin-4-yl pyrazolo[1,5-a]pyrimidine, 7-{4-[(1S,4S)-2,5-diazabicyclo[2.2.2]oct-2-yl]-2-fluorophenyl}-3-(1H-indazol-4-ylpyrazolo[1,5-a]pyrimidine, 7-{2-fluoro-4-[(1S,4S)-5-methyl-2,5-diazabicyclo[2.2.2]oct-2-yl]phenyl}-3-(1H-indazol-4-yl)-2-pyridin-4-yl pyrazolo[1,5-a]pyrimidine, 3-(1H-indazol-4-yl)-2-pyridin-4-yl-7-{2,3,5,6-tetrafluoro-4-[(1S,4S)-5-methyl-2,5-diazabicyclo[2.2.1]hept-2-yl]phenyl}pyrazolo[1,5-a]pyrimidine, tert-butyl (1S,4S)-5-{3-chloro-4-[3-(7-fluoro-1H-indazol-4-yl)-2-pyridin-4-ylpyrazolo[1,5-a]pyrimidin-7-yl]phenyl}-2,5-diazabicyclo[2.2.1]heptane-2-carboxylate, 7-{cis-4-[(1S,4S)-2,5-diazabicyclo[2.2.1]hept-2-yl]cyclohexyl}-3-(7-fluoro-1H-indazol-4-yl)-2-pyridin-4-ylpyrazolo[1,5-a]pyrimidine, 3-(7-fluoro-1H-indazol-4-yl)-7-{cis-4-[(1S,4S)-2-oxa-5-azabicyclo[2.2.1]hept-5-yl]cyclohexyl}-2-pyridin-4-ylpyrazolo[1,5-a]pyrimidine, 3-(7-fluoro-1H-indazol-4-yl)-7-{trans-4-[(1S,4S)-2-oxa-5-azabicyclo[2.2.1]hept-5-yl]cyclohexyl}-2-pyridin-4-ylpyrazolo[1,5-a]pyrimidine, 3-(7-fluoro-1H-indazol-4-yl)-7-[cis-4-(8-oxa-3-azabicyclo[3.2.1]oct-3-yl)cyclohexyl]-2-pyridin-4-ylpyrazolo[1,5-a]pyrimidine, 3-(7-fluoro-1H-indazol-4-yl)-7-[trans-4-(8-oxa-3-azabicyclo[3.2.1]oct-3-yl)cyclohexyl]-2-pyridin-4-yl pyrazolo[1,5-a]pyrimidine, 3-(7-fluoro-1H-indazol-4-yl)-7-[cis-4-(3-oxa-8-azabicyclo[3.2.1]oct-8-yl)cyclohexyl]-2-pyridin-4-ylpyrazolo[1,5-a]pyrimidine, 3-(7-fluoro-1H-indazol-4-yl)-7-[trans-4-(3-oxa-8-azabicyclo[3.2.1]oct-8-yl)cyclohexyl]-2-pyridin-4-ylpyrazolo[1,5-a]pyrimidine, 7-{trans-4-[(1S,4S)-2,5-diazabicyclo[2.2.1]hept-2-yl]cyclohexyl}-3-(7-fluoro-1H-indazol-4-yl)-2-pyridin-4-ylpyrazolo[1,5-a]pyrimidine, 7-{2-chloro-4-[(1S,4S)-2,5-diazabicyclo[2.2.1]-hept-2-yl]phenyl}-3-(7-fluoro-1H-indazol-4-yl)-2-pyridin-4-ylpyrazolo[1,5-a]pyrimidine, 7-{4-[(1S,4S)-2,5-diazabicyclo[2.2.1]hept-2-yl]-3-fluorophenyl}-3-(1H-indazol-4-yl)-2-pyridin-4-ylpyrazolo[1,5-a]pyrimidine, 7-{4-[(1S,4S)-2,5-diazabicyclo[2.2.1]hept-2-yl]-2-(trifluoromethyl)phenyl}-3-(1H-indazol-4-yl)-2-pyridin-4-ylpyrazolo[1,5-a]pyrimidine, 7-{2-bromo-4-[(1S,4S)-2,5-diazabicyclo[2.2.1]hept-2-yl]phenyl}-3-(1H-indazol-4-yl)-2-pyridin-4-ylpyrazolo[1,5-a]pyrimidine, 7-{3-fluoro-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, 3-(1H-indazol-4-yl)-7-{4-[(1S,4S)-5-methyl-2,5-diazabicyclo[2.2.1]hept-2-yl]-2-(trifluoromethyl)phenyl}-2-pyridin-4-ylpyrazolo[1,5-a]pyrimidine, 7-{2-bromo-4-[(1S,4S)-5-methyl-2,5-diazabicyclo[2.2.1]hept-2-yl]phenyl}-3-(1H-indazol-4-yl)-2-pyridin-4-ylpyrazolo[1,5-a]pyrimidine, 7-(1,4-dioxaspiro[4.5]dec-7-en-8-yl)-3-(1H-indazol-4-yl)-2-pyridin-4-ylpyrazolo[1,5-a]pyrimidine, 7-{2,6-difluoro-4-[(1S,4S)-5-methyl-5-oxido-2,5-diazabicyclo[2.2.1]hept-2-yl]phenyl}-3-(1H-indazol-4-yl)-2-pyridin-4-ylpyrazolo[1,5-a]pyrimidine, 3-(1H-indazol-4-yl)-7-{5-[(1S,4S)-2-oxa-5-azabicyclo[2.2.1]hept-5-yl methyl]fu ran-3-yl}-2-pyridin-4-ylpyrazolo[1,5-a]pyrimidine, tert-butyl(1S,4S)-5-({4-[3-(1H-indazol-4-yl)-2-pyridin-4-ylpyrazolo[1,5-a]pyrimidin-7-yl]furan-2-yl}methyl)-2,5-diazabicyclo[2.2.1]heptane-2-carboxylate, 7-{5-[(1S,4S)-2,5-diazabicyclo[2.2.1]hept-2-ylmethyl]furan-3-yl}-3-(1H-indazol-4-yl)-2-pyridin-4-ylpyrazolo[1,5-a]pyrimidine, tert-butyl(1S,4S)-5-({5-[3-(1H-indazol-4-yl)-2-pyridin-4-ylpyrazolo[1,5-a]pyrimidin-7-yl]thiophen-2-yl}methyl)-2,5-diazabicyclo[2.2.1]heptane-2-carboxylate, 3-(1H-indazol-4-yl)-7-{5-[(1S,4S)-2-oxa-5-azabicyclo[2.2.1]hept-5-ylmethyl]thiophen-2-yl}-2-pyridin-4-ylpyrazolo[1,5-a]pyrimidine, 7-{5-[(1S,4S)-2,5-diazabicyclo[2.2.1]hept-2-ylmethyl]thiophen-2-yl}-3-(1H-indazol-4-yl)-2-pyridin-4-ylpyrazolo[1,5-a]pyrimidine, ethyl (3-endo)-3-[3-(1H-indazol-4-yl)-6-methyl-2-pyridin-4-ylpyrazolo[1,5-a]pyrimidin-7-yl]-8-azabicyclo[3.2.1]octane-8-carboxylate, 3-(1H-indazol-4-yl)-7-[6-(8-oxa-3-azabicyclo[3.2.1]oct-3-yl)pyridin-3-yl]-2-pyridin-4-yl pyrazolo[1,5-a]pyrimidine, 3-(1H-indazol-4-yl)-7-{6-[(1S,4S)-2-oxa-5-azabicyclo-[2.2.1]hept-5-yl]pyridin-4-ylpyrazolo[1,5-a]pyrimidine, tert-butyl(1S,4S)-5-{3-[3-(1H-indazol-4-yl)-2-pyridin-4-ylpyrazolo[1,5-a]pyrimidin-7-yl]benzyl}-2,5-diazabicyclo[2.2.1]heptane-2-carboxylate, tert-butyl(1S,4S)-5-{4-[3-(1H-indazol-4-yl)-2-pyridin-4-ylpyrazolo[1,5-a]pyrimidin-7-yl]benzyl}-2,5-diazabicyclo[2.2.1]-heptane-2-carboxylate, 3-(1H-indazol-4-yl)-7-{4-[(1S,4S)-2-oxa-5-azabicyclo[2.2.1]-hept-5-ylmethyl]phenyl}-2-pyridin-4-ylpyrazolo[1,5-a]pyrimidine, 7-{4-[(1S,4S)-2,5-diazabicyclo[2.2.1]hept-2-yl methyl]-2-fluorophenyl}-3-(1H-indazol-4-yl)-2-pyridin-4-ylpyrazolo[1,5-a]pyrimidine, 7-{2-fluoro-4-[(1S,4S)-2-oxa-5-azabicyclo[2.2.1]hept-5-ylmethyl]phenyl}-3-(1H-indazol-4-yl)-2-pyridin-4-ylpyrazolo[1,5-a]pyrimidine, 7-{2-fluoro-4-[(1 R,4R)-2-oxa-5-azabicyclo[2.2.1]hept-5-ylmethyl]phenyl}-3-(1H-indazol-4-yl)-2-pyridin-4-ylpyrazolo[1,5-a]pyrimidine, 7-{3-[(1S,4s)-2,5-diazabicyclo[2.2.1]hept-2-ylmethyl]phenyl}-3-(1H-indazol-4-yl)-2-pyridin-4-ylpyrazolo[1,5-a]pyrimidine, 7-{4-[(1S,4S)-2,5-diazabicyclo[2.2.1]hept-2-ylmethyl]phenyl}-3-(1H-indazol-4-yl)-2-pyridin-4-ylpyrazolo[1,5-a]pyrimidine, 9-{3-fluoro-4-[3-(1H-indazol-4-yl)-2-pyridin-4-ylpyrazolo[1,5-a]pyrimidin-7-yl]benzyl}-3,7-dioxa-9-azabicyclo[3.3.1]nonane and pharmaceutically acceptable salts thereof.

12. A method for making a compound of claim 1, comprising the steps of: (a) reacting a substituted ketone of formula 1 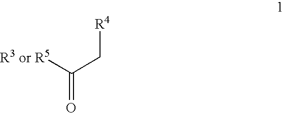
with an acetal of N,Ndialkylformamide or an acetal of N,N-dialkylacetamide, to provide an enaminone compound of formula 2 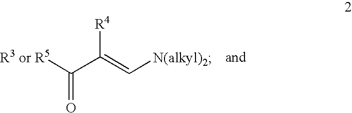
(b) reacting the enaminone compound of formula 2 with a substituted 3-aminopyrazole of formula 8

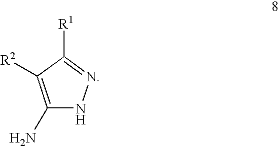

13. A method a method for making a compound of claim 1, comprising the steps of: (a) reacting an enaminone compound of formula 2 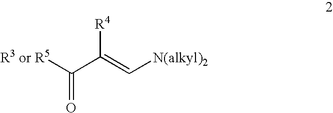
with an aminopyrazole of formula 8a 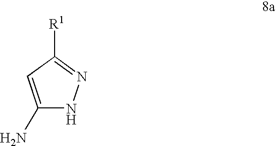
to provide compounds of formula 3c and 3d 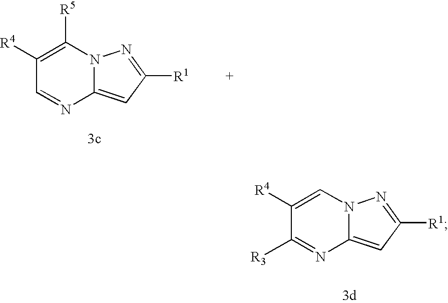
(b) halogenating one or both of the compounds of formula 3c and 3d to provide one or both of compounds of formula 3e and 3f 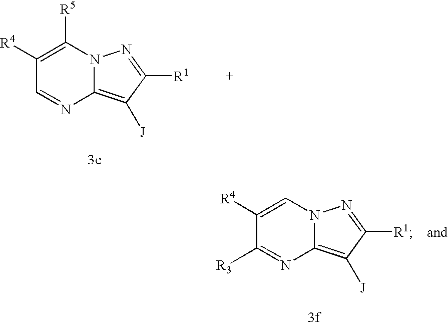
(c) subjecting one or both of the compounds of formula 3e and 3f to a palladium catalyzed, Suzuki coupling using aryl or heteroaryl boronic acids or corresponding boronate esters.

14. The method according to claim 13, comprising an additional step of separating compounds of formula 3c and 3d prior to step (b).

15. The method according to claim 13, comprising an additional step of separating compounds of formula 3e and 3f prior to step (c).

16. The method according to claim 13, comprising an additional step of separating compounds after step (c).

17. A pharmaceutical composition comprising a compound according to any of claims 1-10 and a pharmaceutically acceptable carrier.

18. A pharmaceutical composition comprising a compound according to any of claims 1-10 in combination with other kinase-inhibiting pharmaceutical compositions or chemotherapeutic agents, and a pharmaceutically acceptable carrier.

19. A pharmaceutical composition according to claims 16 or 17, that is capable of inhibiting Raf kinase activity.

20. A method of treating a disease associated with inhibiting Raf kinase activity in a mammal comprising administering to the mammal a pharmaceutically effective amount of a compound according to any one of claims 1-10.

21. The method of claim 20, wherein the disease is associated with a B-Raf kinase dependent condition, a mutant B-Raf kinase condition, or a C-Raf kinase dependent condition.

22. The method of claim 20, wherein the disease comprises inflammation or cancer.

23. The method of claim 22, wherein the cancer is selected from breast, kidney, bladder, thyroid, mouth, larynx, esophagus, stomach, colon, ovary, lung, pancreas, skin, liver, prostate and brain cancer.

24. A method of treating a B-Raf kinase-dependent cancer by administering to a patient a compound any one of claims 1-10.

25. The method of claim 24, wherein the cancer is selected from the group consisting of: breast, kidney, bladder, thyroid, mouth, larynx, esophagus, stomach, colon, ovary, lung, pancreas, skin, liver, prostate and brain cancer.
